(19) United States
(12) Patent Application Publication
(10) Pub. No.: US 2025/0018001 A1
(43) Pub. Date: Jan. 16, 2025

(54) GROWTH PROMOTING PEPTIDES, AND USES THEREOF

(71) Applicant: NURITAS LIMITED, South Dublin (IE)

(72) Inventors: Nora KHALDI, Dublin (IE); Cyril LOPEZ, Dublin (IE); Alessandro ADELFIO, Dublin (IE)

(73) Assignee: NURITAS LIMITED, South Dublin (IE)

(21) Appl. No.: 18/743,639

(22) Filed: Jun. 14, 2024

Related U.S. Application Data
(63) Continuation of application No. 18/330,109, filed on Jun. 6, 2023, now abandoned.
Application No. 18/330,109 is a division of application No. 17/153,587, filed on Jan. 20, 2021, now Pat. No. 11,707,500.
Application No. 17/153,587 is a continuation of application No. 16/504,872, filed on Jul. 8, 2019, now Pat. No. 10,925,922.
Application No. 16/504,872 is a continuation of application No. 15/744,390, filed on Jan. 12, 2018, now Pat. No. 10,905,734.

(30) Foreign Application Priority Data
Jul. 16, 2015 (EP) 15177017.9

(51) Int. Cl. A61K 38/01; A61F 13/00; A61K 8/64; A61K 9/00; A61K 38/00; A61P 17/02; A61P 35/00; A61Q 19/08; C07K 14/415; C12N 9/02

(52) U.S. Cl. CPC A61K 38/01; A61F 13/00063; A61K 8/64; A61K 9/0014; A61K 38/011; A61P 17/02; A61P 35/00; A61Q 19/08; C07K 14/415; C12N 9/0069; C12Y 113/11012; A61F 2013/00089; A61F 2013/00361; A61K 38/00

(57) ABSTRACT

A natural peptide comprising a cellular growth promoting fragment of a protein selected from SEQ ID NO's: 1 to 13, and a composition comprising a plurality of growth promoting peptides, is described. Also disclosed is the use of the peptides and compositions in prevention of ageing of human skin, treatment of diseases or conditions characterised by damaged epithelial cells or tissue such as colon cancer and peripheral inflammatory disorders, and wound treatment. Specific pea and rice protein derived peptides are described in SEQ ID NO's: 15 to 505, and 546 to 704.

### CROSS REFERENCE TO RELATED APPLICATION

This application is a continuation application under 35 U.S.C. § 120 of co-pending U.S. application Ser. No. 18/330,109, filed on Jun. 6, 2023, which is a divisional application of U.S. application Ser. No. 17/153,587, filed on Jan. 20, 2021, which issued as U.S. Pat. No. 11,707,500 on Jul. 25, 2023, which is a continuation application of U.S. application Ser. No. 16/504,872 filed on Jul. 8, 2019, which issued as U.S. Pat. No. 10,925,922 on Feb. 23, 2021, which is a continuation application of U.S. application Ser. No. 15/744,390 filed on Jan. 12, 2018, which issued as U.S. Pat. No. 10,905,734 on Feb. 2, 2021, which is a 35 U.S.C. § 371 National Phase Entry of the International Application No. PCT/EP2016/067097 filed on Jul. 18, 2016, which designates the U.S. and which claims benefit under 35 U.S.C. § 119 (b) of European Patent Application No. 15177017.9 filed Jul. 16, 2015, the contents of all of which are incorporated herein by reference in their entirety.

### SEQUENCE LISTING

The sequence listing of the present application has been submitted electronically via Patent Center as an XML formatted sequence listing with a file name “048262-091340USC3_SL3.xml”, creation date of Jul. 12, 2023 and a size of 977,105 bytes. The sequence listing submitted via Patent Center is part of the specification and is herein incorporated by reference in its entirety.

### BACKGROUND TO THE INVENTION

The growing will of maintaining a youthful appearance is leading to more and more research of new dermatological procedures for treatment of skin aging, especially when people keeps living longer and healthier. With age, structural and functional changes are be observed in human skin (Calleja-Agius J. et al. 2013) and many factors are responsible for them like environmental factors such as UV radiation from sunlight or internal factors such as changes in hormones due menopause (Affinito P. et al. 1999).

The alterations of connective tissue in the dermis and epidermis, especially the reduction of the extracellular matrix is highly responsible for skin wrinkling and sagging since they induce important changes in its mechanical properties. Furthermore, metabolism and synthesis of the extra-cellular matrix are affected by the ageing process through enzymatic activities.

There are mainly four solutions to fight against ageing process at the moment:
Diet and hormones management: fighting the ageing process by managing our diet by using supplements. This method is still controverted at the moment. Some studies suggest that antioxidant supplements like Vitamin C or lipoic acid for example could have anti-ageing properties.
Hormone treatment: a risky and controverted solution for anti-ageing purpose. One have to be really careful with anything related to hormones since it can have a broad spectrum of adverse effects. This solution isn't widely used and most of the studies are still on the animal stage.
Surgical anti-aging solutions: they are very effective on the short term but they are costly and can require long period of healing. Like in every surgical operation, there are risks but also side effects. For example, during surgical anti-aging solutions like eyelifts and facelifts, where an incision is made at the hairline, there are some risks of bruising, swelling, drooping eyelids, and secondary infections can occur.

Recently, there has been an increasing enthusiasm on minimally invasive treatments and techniques designed to deal with problems like wrinkles, volume loss and other skin damages. The most common topical anti-ageing solutions are creams and serums. Their active ingredients can be divided in several families:
Moisturiser ingredients will keep the skin hydrated. It's mostly big polar molecules that will make bonds with water like glycosaminoglycan (hyaluronic acid for instance).
Collagen and other extra-cellular matrix related ingredients will contribute to maintain a well organised skin structure by stimulating biosynthesis if they can trigger the right receptors.
Cell proliferation ingredients are important as well since they help to regenerate skin cells which will synthesise the component the skin needs like extra-cellular matrix and growth factors.

It is an object of the invention to provide an alternative minimally invasive treatment of skin ageing.

### STATEMENTS OF INVENTION

The pea genome codes for over 70,000 different proteins. The Applicant has identified six of these proteins, each of which contains one or more peptides capable of promoting cellular growth and/or proliferation (hereafter “growth promoting peptide” or “growth promoting fragment”). Likewise, out of the more than 60,000 proteins encoded by the rice genome, the Applicant has identified seven proteins, each of which contains one or more peptides that are bioactive, typically capable of promoting cellular growth and/or proliferation. Cellular growth promoting fragments of the fourteen identified proteins have been shown to have an effect on elastin and collagen production and cell proliferation (FIGS. 1 to 109). The specific plant proteins from which the natural peptides are derived are provided in SEQ ID NOs: 1-14 and 705 to 716. The specific pea proteins from which the peptides are derived include SEQ ID NOs: 1-2 and 7-10, and the specific rice proteins from which the peptides are derived include SEQ ID NOs: 3-6 and 11-13. Homologs of these proteins are described in SEQ ID NOs: 525 to 564. The specific peptides initially identified in the pea proteins are shown in SEQ ID NOs: 15-215 and 283-340. The specific peptides initially identified in the rice proteins are shown in SEQ ID NOs: 216-282 and 341-412. Additional peptides identified in the pea and rice proteins disclosed herein, and variants thereof, are provided in SEQ ID NOs: 418 to 515 and 546 to 704 and 717-775.

In a first aspect, the invention provides a peptide, typically 5 to 50 amino acids in length, and comprising (a) a fragment of a pea or rice protein, for example the pea and rice protein disclosed herein such as one selected from SEQ ID NOs: 1 to 14 and 705 to 716 and 717-732, or a homolog thereof, or (b) a variant of the fragment, or (c) a fragment of the peptide (hereafter “peptide of the invention”). In one embodiment the peptide is bioactive. In one embodiment, the peptide has cellular growth or proliferation promotion activity.

In one embodiment, the peptide of the invention comprises a sequence selected from SEQ ID NOs: 15-505 and 546-704 and 717-775.

In one embodiment, the peptide of the invention consists essentially of a sequence selected from SEQ ID NOs: 15-505 and 546-704 and 717-775.

In one embodiment, the peptide of the invention consists of about 3-50 amino acids. In one embodiment, the peptide of the invention consists of about 4-50 amino acids. In one embodiment, the peptide of the invention consists of about 5-50 amino acids. In one embodiment, the peptide of the invention consists of about 6-50 amino acids. In one embodiment, the peptide of the invention consists of about 7-50 amino acids.

In one embodiment, the fragment has 8 to 37 amino acids. In one embodiment, the fragment has a charge of between −10 and +4.

Preferably, the c-terminal amino acid is not cysteine (C) or methionine (M).

Preferably, the n-terminal amino acid is not cysteine (C), histidine (H), or proline (P).

Preferably, the c-terminal domain of the fragment does not contain cysteine (C).

Preferably, the n-terminal domain of the fragment does not contain cysteine (C).

Preferably, the fragment does not contain cysteine (C).

Preferably, the peptide does not contain cysteine (C).

In one embodiment of the invention, the peptide comprises a sequence selected from SEQ ID NOs: 15 to 505.

In one embodiment of the invention, the peptide consists essentially of a sequence selected from SEQ ID NOs: 15 to 505.

Preferably, the fragment is selected from SEQ ID NOs: 15-215 and 283-340, or a bioactive variant of the fragment.

Preferably, the fragment is selected from SEQ ID NOs: 216-282 and 341-412, or a bioactive variant of the fragment.

Preferably, the peptide consists of a fragment selected from SEQ ID NOs: 15 to 417, or a bioactive variant of the fragment.

Preferably, the peptide consists of a sequence selected from SEQ ID NOs: 15 to 417.

Preferably, the fragment is selected from SEQ ID NOs: 413-417, or a bioactive variant of the fragment.

In one embodiment, the peptide comprises or consists of a fragment selected from SEQ ID NOs: 246, 283, 284, 245 and 42, or a bioactive variant of the fragment.

In one embodiment, the peptide comprises a fragment selected from SEQ ID NOs: 247 to 268.

In one embodiment, the peptide of the invention is modified. In one embodiment the peptide is modified with a protecting group. In one embodiment, the peptide is modified to increase its lipophilicity. In none embodiment, the peptide is modified to increase its half-life. In one embodiment, an N or C-terminal amino acid of the peptide is modified. In one embodiment, the N or C-terminal amino acid of the peptide is modified with a protecting group.

### [SEQ ID NO: 1 (Pea Protein 1-P13918)]

Preferably, the peptide comprises a bioactive fragment of the protein of SEQ ID NO: 1 or a homolog thereof, or a bioactive variant of the fragment.

Preferably, the peptide comprises a bioactive fragment selected from SEQ ID NOs: 15 to 90, or a bioactive variant of the fragment.

The invention also provides a composition comprising at least 1, preferably at least 2, preferably at least 3, preferably at least 4, preferably at least 5, preferably at least 6, preferably at least 7, preferably at least 8, preferably at least 9, or preferably at least 10 bioactive peptides of the invention, each of which typically comprises a different a bioactive fragment of SEQ ID NO: 1 or a homolog thereof. Preferably, the composition comprises a first bioactive peptide comprising a first a bioactive fragment selected from SEQ ID NOs: 15 to 90 (or a bioactive variant of the fragment), and a second bioactive peptide comprising a second a bioactive fragment selected from SEQ ID NOs: 15-90 (or a bioactive variant of the fragment).

In one embodiment, the bioactive peptide, variant or fragment is a cellular growth or proliferation promoting peptide, variant or fragment, respectively.

Homologs of Pea Protein 1 (SEQ ID NO: 1) include Vicia fabia, Cicer arietinum and Lens culinaris homologs (SEQ ID NOs: 525 to 527).

### [SEQ ID NO: 2 (Pea Protein 2-Q9M3X6)]

Preferably, the peptide comprises a bioactive fragment of the protein of SEQ ID NO: 2, or a homolog thereof, or a bioactive variant of the fragment.

Preferably, the peptide comprises a bioactive fragment selected from SEQ ID NOs: 91 to 215, or a bioactive variant of the fragment.

The invention also provides a composition comprising at least 1, preferably at least 2, preferably at least 3, preferably at least 4, preferably at least 5, preferably at least 6, preferably at least 7, preferably at least 8, preferably at least 9, or preferably at least 10 bioactive peptides of the invention, each of which comprises a different a bioactive fragment of SEQ ID NO: 2 or a homolog thereof. Preferably, the composition comprises a first bioactive peptide comprising a bioactive fragment selected from SEQ ID NOs: 91-215, and a second bioactive peptide comprising a bioactive fragment selected from SEQ ID NOs: 91-215.

In one embodiment, the bioactive peptide, variant or fragment is a cellular growth or proliferation promoting peptide, variant or fragment, respectively.

Homologs of Pea Protein 2 (SEQ ID NO: 2) include Pisum abyssinicum, Lathyrus annuus, and Vicia villosa (SEQ ID NOs: 528 to 530).

### [SEQ ID NO: 3 (Rice Protein 1-Q0DEV5)]

Preferably, the peptide comprises a bioactive fragment of the protein of SEQ ID NO: 3, or a homolog thereof, or a bioactive variant of the fragment.

Preferably, the peptide comprises a bioactive fragment selected from SEQ ID NOs: 216-244, or a bioactive variant of the fragment.

The invention also provides a composition comprising at least 1, preferably at least 2, preferably at least 3, preferably at least 4, preferably at least 5, preferably at least 6, preferably at least 7, preferably at least 8, preferably at least 9, or preferably at least 10 bioactive peptides of the invention, each of which comprises a different bioactive fragment of SEQ ID NO: 3 or a homolog thereof. Preferably, the composition comprises a first bioactive peptide comprising a bioactive fragment selected from SEQ ID NOs: 216-244, and a second bioactive peptide comprising a bioactive fragment selected from SEQ ID NOs: 216-244.

In one embodiment, the bioactive peptide, variant or fragment is a cellular growth or proliferation promoting peptide, variant or fragment, respectively.

Homologs of Rice Protein 1 (SEQ ID NO: 15) include Oryza rufipogon, Oryza officinalis, Hordeum vulgare subsp. vulgare (SEQ ID NOs: 531 to 533).

### [SEQ ID NO: 4 (Rice Protein 2-P14323)]

Preferably, the peptide comprises a bioactive fragment of the protein of SEQ ID NO: 4, or a homolog thereof, or a bioactive variant of the fragment.

Preferably, the peptide comprises a bioactive fragment selected from SEQ ID NOs: 245-246, or a bioactive variant of the fragment.

The invention also provides a composition comprising at least one or more peptides of the invention that comprise different bioactive fragments of SEQ ID NO: 4 or a homolog thereof. Preferably, the composition comprises a first peptide comprising the bioactive fragment SEQ ID NO: 245 and a second peptide comprising the bioactive fragment SEQ ID NO: 246.

In one embodiment, the bioactive peptide, variant or fragment is a cellular growth or proliferation promoting peptide, variant or fragment, respectively.

Homologs of Rice Protein 2 (SEQ ID NO: 4) include Oryza brachyantha, and Zizania latifolia (SEQ ID NOs: 534 to 536).

### [SEQ ID NO: 5 (Rice Protein 3-P29835)]

Preferably, the peptide comprises a bioactive fragment of the protein of SEQ ID NO: 5, or a homolog thereof, or a bioactive variant of the fragment.

Preferably, the peptide comprises a bioactive fragment selected from SEQ ID NOs: 247-268, or a bioactive variant of the fragment.

The invention also provides a composition comprising at least 1, preferably at least 2, preferably at least 3, preferably at least 4, preferably at least 5, preferably at least 6, preferably at least 7, preferably at least 8, preferably at least 9, or preferably at least 10 bioactive peptides of the invention, each of which comprises a different bioactive fragment of SEQ ID NO: 5 or a homolog thereof. Preferably, the composition comprises a first bioactive peptide comprising a bioactive fragment selected from SEQ ID NOs: 247-268, and a second bioactive peptide comprising a bioactive fragment selected from SEQ ID NOs: 247-268.

In one embodiment, the bioactive peptide, variant or fragment is a cellular growth or proliferation promoting peptide, variant or fragment, respectively.

Homologs of Rice Protein 3 (SEQ ID NO: 5) include Zea Mays, Sorghum bicolor and Setaria italica (SEQ ID NOs: 537 to 539).

### [SEQ ID NO: 6 (Rice Protein 4-P14614)]

Preferably, the peptide comprises a bioactive fragment of the protein of SEQ ID NO: 6 or a homolog thereof, or a bioactive variant of the fragment.

Preferably, the peptide comprises a bioactive fragment selected from SEQ ID NOs: 269-282, or a bioactive variant of the fragment.

The invention also provides a composition comprising at least 1, preferably at least 2, preferably at least 3, preferably at least 4, preferably at least 5, preferably at least 6, preferably at least 7, preferably at least 8, preferably at least 9, or preferably at least 10 bioactive peptides of the invention, each of which comprises a different bioactive fragment of SEQ ID NO: 6 or a homolog thereof. Preferably, the composition comprises a first bioactive peptide comprising a bioactive fragment selected from SEQ ID NOs: 269-282, and a second bioactive peptide comprising a bioactive fragment selected from SEQ ID NOs: 269-282.

In one embodiment, the bioactive peptide, variant or fragment is a cellular growth or proliferation promoting peptide, variant or fragment, respectively.

Homologs of Rice Protein 4 (SEQ ID NO: 6) include Oryza sativa Japonica Group, Brachipodium distachyon (SEQ ID NOs: 540 to 542).

### [SEQ ID NO: 7 (Pea Protein 3-P09918)]

Preferably, the peptide comprises a bioactive fragment of the protein of SEQ ID NO: 7 or a homolog thereof, or a bioactive variant of the fragment.

Preferably, the peptide comprises a bioactive fragment of SEQ ID NO: 283, or a bioactive variant of the fragment.

The invention also provides a composition comprising at least 1, preferably at least 2, preferably at least 3, preferably at least 4, preferably at least 5, preferably at least 6, preferably at least 7, preferably at least 8, preferably at least 9, or preferably at least 10 bioactive peptides of the invention, each of which comprises a different bioactive fragment of SEQ ID NO: 7 or a homolog thereof. Preferably, the composition comprises a bioactive fragment SEQ ID NO: 283, or a bioactive variant of the fragment.

In one embodiment, the bioactive peptide, variant or fragment is a cellular growth or proliferation promoting peptide, variant or fragment, respectively.

Homologs of Pea Protein 3 (SEQ ID NO: 7) include Medicago truncatula, Glycine soja, and Phaseolus vulgaris (SEQ ID NOs: 543 to 545).

### [SEQ ID NO: 8 (Pea Protein 4-P02857)]

Preferably, the peptide comprises a bioactive fragment of the protein of SEQ ID NO: 8 or a homolog thereof, or a bioactive variant of the fragment.

Preferably, the peptide comprises a bioactive fragment selected from SEQ ID NOs: 284-307, or a bioactive variant of the fragment.

The invention also provides a composition comprising at least 1, preferably at least 2, preferably at least 3, preferably at least 4, preferably at least 5, preferably at least 6, preferably at least 7, preferably at least 8, preferably at least 9, or preferably at least 10 bioactive peptides of the invention, each of which comprises a different bioactive fragment of SEQ ID NO: 8 or a homolog thereof. Preferably, the composition comprises a first bioactive peptide comprising a bioactive fragment selected from SEQ ID NOs: 284-307, and a second bioactive peptide comprising a bioactive fragment selected from SEQ ID NOs: 284-307.

In one embodiment, the bioactive peptide, variant or fragment is a cellular growth or proliferation promoting peptide, variant or fragment, respectively.

Homologs of Pea Protein 4 (SEQ ID NO: 8) include Vicia sativa, Vicia narbonesis and Cicer arietinum (SEQ ID NOs: 546 to 548).

### [SEQ ID NO: 9 (Pea Protein 5-P02855)]

Preferably, the peptide comprises a bioactive fragment of the protein of SEQ ID NO: 9 or a homolog thereof, or a bioactive variant of the fragment.

Preferably, the peptide comprises a bioactive fragment selected from SEQ ID NOs: 308-339, or a bioactive variant of the fragment.

The invention also provides a composition comprising at least 1, preferably at least 2, preferably at least 3, preferably at least 4, preferably at least 5, preferably at least 6, preferably at least 7, preferably at least 8, preferably at least 9, or preferably at least 10 bioactive peptides of the invention, each of which comprises a different bioactive fragment of SEQ ID NO: 9 or a homolog thereof. Preferably, the composition comprises a first bioactive peptide comprising a bioactive fragment selected from SEQ ID NOs: 308-339, and a second bioactive peptide comprising a bioactive fragment selected from SEQ ID NOs: 308-339.

In one embodiment, the bioactive peptide, variant or fragment is a cellular growth or proliferation promoting peptide, variant or fragment, respectively.

Homologs of Pea Protein 5 (SEQ ID NO: 9) include Lathyrus hirsutus, Lathyrus cicero, Lathyrus sativus (SEQ ID NOs: 549 to 551).

### [SEQ ID NO: 10 (Pea Protein 6-D3VNE1)]

Preferably, the peptide comprises a bioactive fragment of the protein of SEQ ID NO: 10 or a homolog thereof, or a bioactive variant of the fragment.

Preferably, the peptide comprises the bioactive fragment SEQ ID NO: 340, or a bioactive variant of the fragment.

The invention also provides a composition comprising at least 1, preferably at least 2, preferably at least 3, preferably at least 4, preferably at least 5, preferably at least 6, preferably at least 7, preferably at least 8, preferably at least 9, or preferably at least 10 bioactive peptides of the invention, each of which comprises a different bioactive fragment of SEQ ID NO: 10 or a homolog thereof. Preferably, the composition comprises a first bioactive peptide comprising the bioactive fragment SEQ ID NO: 340.

In one embodiment, the bioactive peptide, variant or fragment is a cellular growth or proliferation promoting peptide, variant or fragment, respectively.

Homologs of Pea Protein 6 (SEQ ID NO: 10) include Medicago truncatula, Vicia peregrine, and Vicia lutea (SEQ ID NOs: 552 to 554).

### [SEQ ID NO: 11 (Rice Protein 5-P07728)]

Preferably, the peptide comprises a bioactive fragment of the protein of SEQ ID NO: 11 or a homolog thereof, or a bioactive variant of the fragment.

Preferably, the peptide comprises a bioactive fragment selected from SEQ ID NOs: 341-358, or a bioactive variant of the fragment.

The invention also provides a composition comprising at least 1, preferably at least 2, preferably at least 3, preferably at least 4, preferably at least 5, preferably at least 6, preferably at least 7, preferably at least 8, preferably at least 9, or preferably at least 10 bioactive peptides of the invention, each of which comprises a different bioactive fragment of SEQ ID NO: 11 or a homolog thereof. Preferably, the composition comprises a first bioactive peptide comprising a bioactive fragment selected from SEQ ID NOs: 341-358, and a second bioactive peptide comprising a bioactive fragment selected from SEQ ID NOs: 341-358.

In one embodiment, the bioactive peptide, variant or fragment is a cellular growth or proliferation promoting peptide, variant or fragment, respectively.

Homologs of Rice Protein 5 (SEQ ID NO: 11) include Oryza sativa Indica Group, Zizania latifolia, Avena sativa (SEQ ID NOs: 555 to 557).

### [SEQ ID NO: 12 (Rice Protein 6-P07730)]

Preferably, the peptide comprises a bioactive fragment of the protein of SEQ ID NO: 12 or a homolog thereof, or a bioactive variant of the fragment.

Preferably, the peptide comprises a bioactive fragment selected from SEQ ID NOs: 359-401, or a bioactive variant of the fragment.

The invention also provides a composition comprising at least 1, preferably at least 2, preferably at least 3, preferably at least 4, preferably at least 5, preferably at least 6, preferably at least 7, preferably at least 8, preferably at least 9, or preferably at least 10 bioactive peptides of the invention, each of which comprises a different bioactive fragment of SEQ ID NO: 12 or a homolog thereof. Preferably, the composition comprises a first bioactive peptide comprising a bioactive fragment selected from SEQ ID NOs: 359-401, and a second bioactive peptide comprising a bioactive fragment selected from SEQ ID NOs: 359-401.

In one embodiment, the bioactive peptide, variant or fragment is a cellular growth or proliferation promoting peptide, variant or fragment, respectively.

Homologs of Rice Protein 6 (SEQ ID NO: 12) include Oryza brachyantha, Brachipodium distachyon (SEQ ID NOs: 558 to 560).

### [SEQ ID NO: 13 (Rice Protein 7-Q0D7S0)]

Preferably, the peptide comprises a bioactive fragment of the protein of SEQ ID NO: 13 or a homolog thereof, or a bioactive variant of the fragment.

Preferably, the peptide comprises a bioactive fragment selected from SEQ ID NOs: 402-412, or a bioactive variant of the fragment.

The invention also provides a composition comprising at least 1, preferably at least 2, preferably at least 3, preferably at least 4, preferably at least 5, preferably at least 6, preferably at least 7, preferably at least 8, preferably at least 9, or preferably at least 10 bioactive peptides of the invention, each of which comprises a different bioactive fragment of SEQ ID NO: 13 or a homolog thereof. Preferably, the composition comprises a first bioactive peptide comprising a bioactive fragment selected from SEQ ID NOs: 402-412, and a second bioactive peptide comprising a bioactive fragment selected from SEQ ID NOs: 402-412.

In one embodiment, the bioactive peptide, variant or fragment is a cellular growth or proliferation promoting peptide, variant or fragment, respectively.

Homologs of Rice Protein 7 (SEQ ID NO: 13) include Oryza sativa Indica Group, Zizania latifolia, Avena sativa (SEQ ID NOs: 561 to 564).

[SEQ ID NO: 14 (Staphylococcus aureus Protein 1-P0C1U8)]

Preferably, the peptide comprises a fragment of the protein of SEQ ID NO: 14 or a homolog thereof.

Preferably, the peptide comprises a bioactive fragment selected from SEQ ID NOs: 413-417, or a bioactive variant of the fragment.

The invention also provides a composition comprising at least 1, preferably at least 2, preferably at least 3, preferably at least 4, preferably at least 5, preferably at least 6, preferably at least 7, preferably at least 8, preferably at least 9, or preferably at least 10 bioactive peptides of the invention, each of which comprises a different bioactive fragment of SEQ ID NO: 14 or a homolog thereof. Preferably, the composition comprises a first bioactive peptide comprising a bioactive fragment selected from SEQ ID NOs: 413-417, and a second bioactive peptide comprising a bioactive fragment selected from SEQ ID NOs: 413-417.

In one embodiment, the bioactive peptide, variant or fragment is a cellular growth or proliferation promoting peptide, variant or fragment, respectively.

Homologs of Staphylococcus Aureus Protein 1 (SEQ ID NO: 14) include:
>gi|580560623|gb|EVF84961.1| glutamyl endopeptidase [Staphylococcus aureus COAS6020]
>gi|580687002|gb|EVH10169.1| glutamyl endopeptidase [Staphylococcus aureus UCIM 6080]
>gi|751815683|gb|KIN24957.1| glutamyl endopeptidase [Staphylococcus aureus MRSA_CVM43477]
>gi|781884797|dbj|BAR08486.1| glutamyl endopeptidase precursor [Staphylococcus aureu
subsp. aureus]
>gi|781887762|dbj|BAR11210.1| glutamyl endopeptidase precursor [Staphylococcus aureus
subsp. aureus]

The invention also provides a composition comprising at least one and preferably a plurality of peptides of the invention, wherein each of the peptides of the invention comprises a bioactive fragment of a protein disclosed herein, for example selected from SEQ ID NO: 1 to 14 or a homolog thereof, or a bioactive variant of the fragment.

Typically, the or each peptide of the invention comprises, a bioactive fragment selected from, SEQ ID NOs: 15-505 and 546 to 704 and 717-775, or a bioactive variant of the fragment.

Typically, the or each peptide of the invention is selected from a bioactive fragment selected from, SEQ ID NOs: 15-505 and 546 to 704 and 717-775, or a bioactive variant of the fragment.

The invention also provides a composition comprising at least one and preferably a plurality of peptides of the invention, wherein the or each of the peptides of the invention comprise a bioactive fragment of a pea or rice protein disclosed herein, typically selected from SEQ ID NOs: 1-2 and 7-10. Typically, the or each peptide of the invention is selected from, or comprises a bioactive fragment selected from, SEQ ID NOs: 15-215 and 283 to 340, or a bioactive variant of the fragment.

The invention also provides a composition comprising at least one and preferably a plurality of peptides of the invention, wherein the or each of the peptides of the invention comprise a bioactive fragment of a protein disclosed herein typically selected from SEQ ID NOs: 3-6 and 11-13. Typically, the or each peptide of the invention is selected from, or comprises a bioactive fragment selected from, SEQ ID NOs: 216-282 and 341-412, or a bioactive variant of the fragment.

Preferably, the composition comprises at least two distinct growth promoting peptides of the invention.

Preferably, the composition comprises at least three distinct growth promoting peptides of the invention.

Preferably, the composition comprises at least four distinct growth promoting peptides of the invention.

Preferably, the composition comprises at least five distinct growth promoting peptides of the invention.

Preferably, the composition comprises at least six distinct growth promoting peptides of the invention.

Preferably, the composition comprises at least seven distinct growth promoting peptides of the invention.

Preferably, the composition comprises at least eight distinct growth promoting peptides of the invention.

Preferably, the composition comprises at least nine distinct growth promoting peptides of the invention.

Preferably, the composition comprises at least ten distinct growth promoting peptides of the invention.

In one embodiment, the composition comprises one or more of SEQ ID NOs: 247 to 268.

In one embodiment, the composition comprises one or more of SEQ ID NOs: 248, 249, 252, 253 and 257.

In one embodiment, the invention comprises a composition comprising substantially all of fragments SEQ ID NOs: 15-215 and 283-340, or growth promoting variants of the fragments, or a mixture of the growth promoting fragments and variants.

In one embodiment, the invention comprises a composition comprising substantially all of fragments SEQ ID NOs: 216-282 and 341-412, or growth promoting variants of the fragments, or a mixture of the growth promoting fragments and variants.

In one embodiment, the composition is enriched in peptides having a molecular weight of less than 10 KD.

In one embodiment, the composition is a powder.

The invention also relates to a plaster, bandage or dressing suitable for application to a wound and comprising a peptide or composition of the invention.

The invention also relates to a man-made cell culture media comprising a peptide of the invention. The invention also relates to a man-made cell culture media comprising a composition of the invention. In one embodiment, the cell culture media is formulated for culture of eukaryotic cells. In one embodiment, the cell culture media is formulated for culture of prokaryotic cells.

The invention also relates to a plaster, bandage or dressing suitable for application to a wound and comprising a peptide or composition of the invention.

The invention also relates to a comestible product comprising a growth promoting peptide of the invention. Preferably the comestible product is man-made.

The invention also relates to a comestible product comprising a composition of peptides of the invention. Preferably the comestible product is man-made.

Preferably, the comestible product is a food product for human or animal (mammalian) or cellular consumption.

In one embodiment the man-made comestible product is a beverage, In one embodiment the man-made comestible product is a bakery product. In one embodiment the man-made comestible product is a dairy product. In one embodiment the man-made comestible product is a snack product. In one embodiment the man-made comestible product is a baked extruded food product. In one embodiment the man-made comestible product is powdered milk. In one embodiment the man-made comestible product is an infant formula product. In one embodiment the man-made comestible product is a confectionary product. In one embodiment the man-made comestible product is a yoghurt. In one embodiment the man-made comestible product is a yoghurt drink. In one embodiment the man-made comestible product is an ice cream product. In one embodiment the man-made comestible product is a frozen food product. In one embodiment the man-made comestible product is a breakfast cereal. In one embodiment the man-made comestible product is a bread. In one embodiment the man-made comestible product is a flavoured milk drink. In one embodiment the man-made comestible product is a confectionary bar. In one embodiment the man-made comestible product is a tea or tea product. In one embodiment the man-made comestible product is a based extruded snack product. In one embodiment the man-made comestible product is a fried snack product. In one embodiment the man-made comestible product is a nutritional supplement. In one embodiment the man-made comestible product is a sports nutritional product. In one embodiment the man-made comestible product is a baby food product. In one embodiment the man-made comestible product is a speciality food product for immunocompromised individuals. In one embodiment the man-made comestible product is a food for geriatric patients.

The invention also relates to a peptide of the invention for use in promoting growth of a cell.

The invention also relates to a peptide of the invention for use in promoting growth of a cell culture.

The invention also relates to a peptide of the invention for use in promoting growth of a tissue.

The invention also relates to a peptide of the invention for use in promoting growth of dermal or epithelial tissue.

The invention also relates to a peptide of the invention for use in promoting growth of skin.

The invention also relates to a peptide of the invention for use in promoting growth of an organ.

The invention also relates to a peptide of the invention for use in promoting growth of an organism.

The invention also relates to a composition of the invention for use in promoting growth of a cell.

The invention also relates to a composition of the invention for use in promoting growth of a cell culture.

The invention also relates to a composition of the invention for use in promoting growth of a tissue.

The invention also relates to a composition of the invention for use in promoting growth of epithelial tissue.

The invention also relates to a composition of the invention for use in promoting growth of skin.

The invention also relates to a composition of the invention for use in promoting growth of an organ.

The invention also relates to a composition of the invention for use in promoting growth of an organism.

In one embodiment, the cell, tissue or organism has a normal pathology (for example ageing skin). In one embodiment of the invention, the cell, tissue or skin has abnormal pathology (for example tissue damaged due to trauma, drug use, or epithelial tissue in the GI tract damaged due to an inflammatory disorder).

The growth promoting uses may be in-vivo or in-vitro uses. The growth promoting uses may involve administration to mammal externally (i.e. to the skin) or internally (i.e. to the GI tract).

The invention also relates to a peptide of the invention for use in slowing or inhibiting ageing of human skin.

The invention also relates to a method of slowing or inhibiting ageing of human skin comprising a step of administering a peptide of the invention to the human skin. Typically, the peptide of the invention is administered topically to the skin. Administration may be by means of a plaster or patch or a formulation suitable for topical application.

The invention also relates to a composition of the invention for use in slowing or inhibiting ageing of human skin. The invention also relates to a peptide of the invention for use in preventing or slowing ageing of the human skin.

The invention also relates to a method of slowing or inhibiting ageing of human skin comprising a step of administering a composition of the invention to the human skin. Typically, the composition of the invention is administered topically to the skin.

The invention also relates to a peptide of the invention for use in treatment of a wound in a mammal.

The invention also relates to a composition of peptides of the invention for use in treatment of a wound in a mammal.

The invention also relates to a wound treatment composition or product of the invention for use in treatment of a wound in a mammal.

The invention also relates to a peptide of the invention for use in treatment or prevention of a disease or condition characterised by damaged epithelial cells or tissue.

The invention also relates to a composition of peptides of the invention for use in treatment or prevention of a disease or condition characterised by damaged dermal or epithelial cells or tissue.

In one embodiment, the disease or condition characterised by damaged dermal or epithelial cells or tissue is selected from cancer, trauma

The invention also relates to a peptide of the invention for use in maintaining or restoring gut health in a mammal.

The invention also relates to a composition of peptides of the invention for use in maintaining or restoring gut health in in a mammal.

The invention also relates to a peptide of the invention for use in maintaining or restoring muscle health (for example lean tissue mass) in a mammal.

The invention also relates to a composition of peptides of the invention for use in maintaining or restoring muscle health (for example lean tissue mass) in in a mammal.

The invention also relates to a pharmaceutical composition comprising a peptide of the invention in combination with a pharmaceutically acceptable carrier.

The invention also relates to a pharmaceutical composition comprising a composition of peptides of the invention in combination with a pharmaceutically acceptable carrier.

The invention also relates to a peptide of the invention for use in treatment or prevention of an inflammatory disorder in a mammal.

The invention also relates to a composition of the invention for use in treatment or prevention of an inflammatory disorder in a mammal.

The invention also relates to a comestible product, for example a food product comprising a peptide or composition of the invention, for example a dairy or non-dairy product, a solid food or a beverage, a food additive or supplement. The dairy product may be a milk, a cheese, or yoghurt. In one embodiment, the food product is a snack bar. The food product may comprise any amount of the composition of the invention, for example from 0.1% to 30% (w/w).

The peptides of the invention are used in the topical cosmetic or pharmaceutical composition of this invention at cosmetically or pharmaceutically effective concentrations to achieve the desired effect; in a preferred form with regards to the total weight of the composition, between 0.00000001% (in weight) and 20% (in weight); preferably between 0.000001% (in weight) and 15% (in weight), more preferably between 0.0001% (in weight) and 10% (in weight) and even more preferably between 0.0001% (in weight) and 5% (in weight). Ideally, the peptides of the present invention are preferably used from about 0.00001% w/w to about 0.5% w/w [0.1 to 5000 ppm], and more preferably from 0.00005 w/w to about 0.05 w/w [0.5 to 500 ppm], and most preferably from about 0.0001 w/w to about 0.01 w/w of the composition [1 to 100 ppm]. Ideally, the peptides of the present invention are preferably used from about 0.0001% w/w to about 0.004% w/w of the composition.

For compositions of peptides of the invention, a typical daily dosage may be 0.2 g to 100 g. However, when administered as a food for special medicinal purpose, or medical food, the daily dosage may be 50-500 g per day.

The dosage of compositions of the invention for use in food products and food supplements (i.e. comestible compositions) will be broadly in the 0.2-100 g/day range. In one embodiment, the daily dosage is 1-10 g/day, ideally about 3-8 g/day. In one embodiment, the daily dosage is 10-20 g/day. In one embodiment, the daily dosage is 20-30 g/day. In one embodiment, the daily dosage is 30-40 g/day. In one embodiment, the daily dosage is 10-100 g/day. In one embodiment, the daily dosage is about 5 g/day, ideally about 3-8 g/day. In one embodiment, the dosage is 2-1000 mg/day/kg body weight. In one embodiment, the dosage is 10-500 mg/day/kg body weight. In one embodiment, the dosage is 10-100 mg/day/kg body weight. In one embodiment, the dosage is 30-70 mg/day/kg body weight. The dosage of peptides of the invention for food supplements may be 0.00001 mg-0.01 mg per day or dose.

The food product may be a Food for Specific Medicinal Purposes (FSMP) which is defined as foods that are specifically formulated, processed and intended for the dietary management of diseases, disorders or medical conditions of individuals who are being treated under medical supervision. These foods are intended for the exclusive or partial feeding of people whose nutritional requirements cannot be met by normal foods.

The invention also relates to a man-made personal care composition comprising a peptide of the invention.

The invention also relates to a man-made personal care composition comprising a composition of peptides of the invention.

In one embodiment the personal care composition is a skincare product. In one embodiment the personal care composition is a product formulated for topical application to the skin of a human. In one embodiment the personal care composition is an anti-aging product. In one embodiment the personal care composition is a dentrifice product. In one embodiment the personal care composition is a perfumery product. In one embodiment the personal care composition is a deodorant product. In one embodiment the personal care composition is an anti-perspirant product. In one embodiment the personal care composition is a soap. In one embodiment the personal care composition is a liquid soap. In one embodiment the personal care composition is a cream. In one embodiment the personal care composition is a lotion. In one embodiment the personal care composition is a gel. In one embodiment the personal care composition is a powder.

The invention also relates to a man-made wound treatment composition comprising a peptide of the invention. The invention also relates to a man-made wound treatment composition comprising a composition of the invention. Typically, the wound treatment composition is formulated for topical application to a wound. In one embodiment, the composition comprises a cream, gel, lotion, powder.

The invention also provides topical composition comprising a peptide of the invention. It will be appreciated that the topical composition may comprise a plurality of peptides, fragments and/or variants. In one embodiment, the topical composition comprises substantially all the peptides. In one embodiment, the topical composition comprises substantially all the variants. The topical composition of the invention may be presented in a formulation selected from the group comprising creams, multiple emulsions, anhydrous compositions, aqueous dispersions, oils, milks, balsams, foams, lotions, gels, cream gels, hydro-alcoholic solutions, hydro-glycolic solutions, cosmetic, personal care product, hydrogels, liniments, sera, soaps, dusting powder, paste, semi solid formulations, liniments, serums, shampoo, conditioner, ointments, any rinse off formulation, talc, mousses, powders, sprays, aerosols, solutions, suspensions, emulsions, syrups, elixirs, polysaccharide films, patches, gel patches, bandages, an adhesive system, water-in-oil emulsions, oil-in-water emulsions, and silicone emulsions.

In an embodiment of the current invention, the emulsion contains a lipid or oil. The emulsion may be, but is not limited to, oil-in-water, water-in-oil, water-in-oil-in-water and oil-in-water-in-silcone emulsions. The emulsion may contain a humectant. The emulsion may contain an anti-foaming agent, such as silicone. The emulsion may have any suitable viscosity. Emulsions may further contain an emulsifier and/or an anti-foaming agent. Methods of preparing an emulsion are known to a person skilled in the art.

The topical composition of the invention may be incorporated into a medical device for administration. Such a device can include but is not limited to a fabric, patch, bandage, gauge, sock, tight, underwear, dressing, glove, mask, adhesive patches, non-adhesive patches, occlusive patches and microelectric patches or suitable adhesive system. In such an embodiment, the device is in direct contact with the keratinous layer such as the skin, thus releasing the peptides of the invention. It will be understood that the topical composition may be incorporated in any suitable form as detailed herein. For example, the topical composition or peptides of the invention can be incorporated into the device or be present on the surface of the device or can be in a cream, gel or wax formulation or any suitable formulation defined herein and incorporated into the device or on the surface of the device. The device may be adapted for adhesion or attachment to the skin.

In one embodiment the device is adapted to release a constant quantity of the composition or the peptides of the invention. It will be understood that the amount of the composition contained in the sustained release system will depend, for example, on where the composition is to be administered, the kinetics and duration of the release of the composition of the invention, as well as the nature of the condition, disorder and/or disease to be treated and/or cared for. The device may be such that the composition is released by biodegradation of the device, or by friction between the device and the body, due to bodily moisture, the skin's pH or body temperature

In an embodiment of the invention the topical composition may further comprise at least one cosmetically or pharmaceutically acceptable excipient. Excipient may be used interchangeably with functional ingredient or additive. It will be understood that although the topical compositions of the current invention can be administered alone, they will generally be administered in admixture with a cosmetic or pharmaceutical excipient. Cosmetically or pharmaceutically acceptable excipient are well known in the art and any known excipient, may be used provided that it is suitable for topical administration and is dermatologically acceptable without undue toxicity, incompatibility and/or allergic reaction.

Preferably any excipient included is present in trace amounts. The amount of excipient included will depend on numerous factors, including the type of excipient used, the nature of the excipient, the component(s) of the topical composition, the amount of active or peptide in the topical composition and/or the intended use of the topical composition. The nature and amount of any excipient should not unacceptably alter the benefits of the peptides of this invention.

In an embodiment of the invention the excipient may be a suitable diluent, carrier, binder, lubricant, suspending agent, coating agent, preservative, stabilisers, dyes, vehicle, solubilising agent, base, emollient, emulsifying agent, fragrance, humectant, and/or surfactants.

Examples of suitable diluents include, but are not limited to, any diluent disclosed in disclosed in US2014120131 or US2004132667. Examples include ethanol, glycerol and water.

Examples of suitable carriers include, but are not limited to, lactose, starch, glucose, methyl cellulose, magnesium stearate, mannitol, sorbitol and any suitable carrier disclosed in US2014120131 or US2004132667.

Examples of suitable binders include, but are not limited to, starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol and any suitable binder disclosed in US2014120131 or US2004132667.

Examples of suitable lubricants include, but are not limited to, sodium oleate, sodium stearate, magnesium stearate, sodium benzoate, sodium acetate, and sodium chloride and any suitable lubricant disclosed in US2014120131 or US2004132667.

The carrier may be any suitable carried known in the art or disclosed in US2014120131 or US2004132667. In some embodiments, the carrier may include, but is not limited to, a liquid, such as water, oils or surfactants, including those of petroleum, animal, plant or synthetic origin, polymer, oil, such as peanut oil, mineral oil, castor oil, soybean oil, alcohol, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, or digitonin. It will be understood that the carrier will be dermatologically acceptable. Preferred carriers contain an emulsion such as oil-in-water, water-in-oil, water-in-oil-in-water and oil-in-water-in-silicone emulsions. Emulsions may further contain an emulsifier and/or an anti-foaming agent.

In an embodiment of the invention, the topical composition may further comprise one or more additional ingredients. The topical composition of the invention may be administered consecutively, simultaneously or sequentially with the one or more other additional agents. Such additional ingredients may be those of benefit to include in a topical composition, or of benefit depending on the intended use of the topical composition. The additional ingredient may be active or functional or both.

Examples of such additional ingredients include, but are not limited to, one or more of cleaning agents, conditioning agents, sunscreen, pigment, moisturiser, thickening agents, gelling agents, essential oil, astringents, pigments, anti-caking agent, anti-foaming agent, binders, additives, buffers, chelating agents, external analgesics, film formers or materials, bulking agents, polymers, opacifying agents, pH adjusters, propellants, reducing agents, sequestrants, skin bleaching and lightening agents, skin conditioning agents, aloe vera, healing agents, soothing agents, smoothing agents, pantothenic acid, treating agents, thickeners, vitamins. colourants, pharmaceuticals, antiseptic agents, antifoaming agents, buffering agents, astringents, polymers, pH adjuster, deodorant or any other dermatologically acceptable carrier or surfactant.

It is to be understood that additional ingredients listed may provide more than one benefit. The classification given herein is for clarity and convenience only and not intended to limit the additional ingredient to that particular application or category listed.

Any additional ingredients should be suitable for application to the skin without undue toxicity, incompatibility and/or allergic reaction.

In some embodiments, the additional ingredient has glucose transport activity or aids glucose transport activity. In some embodiments, the additional ingredient has anti-inflammatory activity or aids anti-inflammatory activity. In some embodiments, the additional ingredient has anti-aging activity or aids anti-aging activity. In some embodiments, the additional ingredient is for keratinous layer health and/or development, skin health and/or development, and/or muscle health, recovery and/or development. The active agent may be a pharmacological enhancer. Such active agents are known and available on the market. In such cases, the topical composition of the invention may be administered consecutively, simultaneously or sequentially with the one or more other active agents.

In some embodiments, the additional ingredient may be farnesol ([2E, 6E], -3, 7, 11,-trimethyl-2, 6, 10, dodecatrien-1-ol), phytantriol (3, 7, 11, 15, tetramethylhexadecane-1, 2, 3, -triol), desquamation actives, enzymes, enzyme inhibitors, enzyme activators, botanical extracts and marine extracts, anti-acne actives, anti-wrinkle or anti atrophy actives, anti-oxidant/radical scavengers, chelators, flavonoids, anti-inflammatory agents, anti-cellulite agents, topical anaesthetics, tanning actives, skin lightening agents, skin healing agents, bisabolol, antimicrobial or antifungal active, sunscreen actives, particulate material, conditioning agents, structuring agents, thickening agent,

The desquamation active may be any suitable agent that enhances the skin appearance or texture of the skin and is as disclosed in US2014120131 or US2004132667.

Examples of anti-acne actives are as disclosed in US2014120131 or US2004132667 and include, resorcinol, salicylic acid, erythromycin, zine, sulfur, benzoyl peroxides.

Examples of thickening agents are as disclosed in US2014120131 or US2004132667 and include carboxylic acid polymers, crosslinked polyacrylate polymers, polyacrylamide polymers, polysaccharides.

Examples of conditioning agents are as disclosed in US2014120131 or US2004132667 and include humectants, moisturiser or skin conditioner.

Examples of structuring agents are as disclosed in US2014120131 or US2004132667 and include any agent that provide rheological characteristics to the composition and contributes to the stability of the composition.

Any suitable antimicrobial or antifungal active may be used and examples are as disclosed in US2014120131 or US2004132667. Such actives are capable of destroying microbes, preventing growth or action of microbes. Examples include but are not limited to β-lactam drugs, quinolone drugs, tetracycline, erythromycin, streptomycin sulfate, salicylic acid, benzoyl peroxide.

Examples of a particulate material include metallic oxide. Examples of anti-cellulite agents include xanthine agents. Examples of tanning actives includes 1, 3-dihydroxy-2-propanone and those disclosed in US2014120131 or US2004132667. Examples of topical anaesthetics include benzocaine, lidocaine and bupivacaine and those disclosed in US2014120131 or US2004132667.

Examples of skin lightening agents include any agent known in the art such as kojic acid, ascorbic acid and those disclosed in US2014120131 or US2004132667.

Examples of sunscreen actives include any suitable organic or inorganic sunscreen active. Examples include metallic oxides, 2-ethylhexyl-p-methoxycinnamate and those disclosed in US2014120131 or US2004132667.

Examples of skin healing agents includes panthenoic acid as disclosed in US2014120131 or US2004132667.

Examples of anti-inflammatory agents include any agent that enhances the skin appearance, tone or colour and include but are not limited to corticosteroids, hydrocortisone, non-steroidal agents such as ibuprofen and aspirin and those disclosed in US2014120131 or US2004132667.

Examples of flavonoids includes flavanones, methoxy flavonones, unsubstituted chalcone and mixtures thereof and those disclosed in US2014120131 or US2004132667.

Examples of enzymes include lipases, proteases, catalase, super oxide-dismutase, amylase, peroxidase, glucuronidase, ceramidases, hyaluronidases. Examples of enzyme inhibitors include trypsine inhibitors, Bowmann Birk inhibitors, chymotrypsin inhibitors, botanical extracts, flavonoids, quercetin chalcone and those disclosed in US2014120131 or US2004132667 and mixtures thereof. Examples of enzyme activators include coenzyme A, Q10 (ubiquinone), glycyrrhizin, berberine, chrysin and those disclosed in US2014120131 or US2004132667 and mixtures thereof

Examples of anti-wrinkle or anti atrophy actives include sulfur containing D and L amino acids, particular, N-acyl derivatives such as N-acetyl-L-cysteine, hydroxyl acids, phytic acid, lipoic acid, lysophosphatidic acid, skin peel agents, vitamin B3, retinoids and those disclosed in US2014120131 or US2004132667 and mixtures thereof.

The anti-oxidant/radical scavenger agent may be any agent that is useful for providing protection against UV radiation or other environmental agents which may cause skin damage such as those disclosed in US2014120131 or US2004132667. Examples of anti-oxidant/radical scavengers include ascorbic acid, its salts and derivatives (vitamin C), tocopherol its salts and derivatives (vitamin E), butylated hydroxyl benzoic acids and their salts, peroxides, gallic acids and alkyl esters, sorbic acid, lipoic acid, amines, lycine pidolate, arginine pilolate, nordihydroguaiaretic acid, bioflavonoids, curcumin, lysine, methionine, proline, superoxide dismutase, silymarin, tea extracts and mixtures thereof.

Examples of chelators include EDTA, NTA, hydoxamic acids, phytic acid, lactoferrin and those disclosed in US2014120131 or US2004132667 and mixtures thereof. A chelator means an agent capable of removing a metal ion by forming a complex so that the metal ion cannot participate in or catalyse chemical reactions. A chelator is useful for protection against UV radiation or other environmental agents that can cause skin damage.

It will be appreciated that a plurality of additional ingredients may be added. The amount of the additional ingredient may be from about 0.001% to about 50% weight of the composition, preferably, about 0.01% to about 20%, preferably about 0.1% to about 10%, about 0.5% to about 10%, about 1% to about 5%, preferably 2% weight of the composition. The amount of additional ingredient included will depend on numerous factors, including the type of additional ingredient used, the nature of the additional ingredient, the component(s) of the topical composition, the amount of active or peptide in the topical composition and/or the intended use of the topical composition. The nature and amount of any additional ingredient should not unacceptably alter the benefits of the peptides of this invention.

The topical composition may be alcohol free.

In some embodiments of the invention, the composition further comprises one or more additional active agents, in addition to the peptide of the invention (also known as the active of the composition). In addition, or alternatively, the composition may be administered with one or more other additional active agents. Typical said additional active agent is present in trace amounts only. In some embodiments, there may be no additional active agent present in the composition. The amount of additional active agent included will depend on numerous factors, including the type of additional active agent used, the nature of the additional active agent, the component(s) of the topical composition, the amount of active or peptide in the topical composition and/or the intended use of the topical composition. The nature and amount of any additional active agent should not unacceptably alter the benefits of the peptides of this invention.

It is to be understood that an ingredient that is considered to be an “active” ingredient in one product may be a “functional” or “excipient” ingredient in another and vice versa. It will also be appreciated that some ingredients play a dual role as both an active ingredient and as a functional or excipient ingredient.

Examples of the additional active agents include glucose transport promoting drugs, skin supplement, agent for treatment and/or care of the skin, anti-inflammatory agent, an anti-aging agent, a cellular growth promoting agent and pharmacological enhancers. Such agents are well known in the art and it will be appreciated that any suitable additional active agent may be used. Additional active agents for treatment and/or care of the skin may include collagen synthesis agents, retinoids, exfoliating agents, anti-cellulite agents, elastase inhibiting agents, melanin synthesis stimulating or inhibiting agents, self-tanning agents, antiaging agents, antimicrobial agents, antifungal agents, fungistatic agents, bactericidal agents, and healing agents. Active agents also include anti-inflammatory agents.

Any additional active agent should be suitable for application to the skin without undue toxicity, incompatibility and/or allergic reaction.

It will be understood that the classification given herein is for clarity and convenience only and not intended to limit the additional ingredient, excipient, or active to that particular application or category listed.

In a particularly preferred embodiment, the methods and uses of the invention involve administration of a peptide or composition of the invention in combination with one or more other active agents, for example, existing growth promoting drugs or pharmacological enhancers available on the market. In such cases, the compounds of the invention may be administered consecutively, simultaneously or sequentially with the one or more other active agents.

The effect of the current invention is accomplished by topical application or administration of the topical composition of the invention described herein to a person, animal or a patient in need of treatment or care. Topical delivery preferably means delivery to a keratinous layer such as the skin, hair and/or nails, but can also mean delivery to a body lumen lined with epithelial cells, for example the lungs or airways, the gastrointestinal tract, the buccal cavity. The effect may be confined to the surface of the skin or may be within the skin or a combination of both.

The topical composition of the invention is administered in a cosmetically or pharmaceutically effective amount. In other words, in an amount that is non-toxic but sufficient amount to provide the desired effect. It will be appreciated that a person skilled in the art would be capable of determining an appropriate dose of the topical compositions of the invention to administer without undue experimentation. Alternatively, a physician will determine the actual dose that is most suitable for a patient depending on the particular condition, disease or disorder to be treated or cared for and the age, body weight and/or health of the person. It will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can, of course, be individual instances where higher or lower dosage ranges are merited, and such are within the scope of this invention. For example, the composition may be administered at a dose of from 0.01 to 50 mg/kg body weight, such as from 0.1 to 30 mg/kg, more preferably from 0.1 to 20 mg/kg body weight, more preferably from 0.1 to 10 mg/kg body weight, preferably 0.1 to 5 mg/kg body weight. In an exemplary embodiment, one or more doses of 10 to 300 mg/day or more preferably, 10 to 150 mg/day, will be administered to the patient. The amount and the frequency is as best suited to the purpose. The frequency of application or administration can vary greatly, depending on the needs of each subject, with a recommendation of an application or administration range from once a month to ten times a day, preferably from once a week to four times a day, more preferably from three times a week to three times a day, even more preferably once or twice a day.

In preferred embodiments, repeated use of the topical composition is provided.

The topical composition may be applied by, but not limited to, rubbing, or massaging into the keratinous tissue, skin or area of the body to be treated or cared for. In some embodiments, the composition is left on or not removed from the area of the body. In other embodiments, the composition is removed after a period of time, such as, but not limited to, from about 2 minutes to 60 minutes, from about 5 minutes to about 30 minutes, preferably from about 10 minutes to about 20 minutes. The composition may be removed immediately after application. In some embodiments of the current invention, the composition of the invention may be applied to an area to be treated by means to achieve a greater penetration of the composition and/or peptide of the invention, such as, but not limited to, iontophoresis, sonophoresis, electroporation, microelectric patches, mechanical pressure, osmotic pressure gradient, occlusive cure, microinjections or needle-free injections by means of pressure, such as injections by oxygen pressure, or any combination thereof.

The peptides of the invention are used in the topical cosmetic or pharmaceutical composition of this invention at cosmetically or pharmaceutically effective concentrations to achieve the desired effect; in a preferred form with regards to the total weight of the composition, between 0.00000001% (in weight) and 20% (in weight); preferably between 0.000001% (in weight) and 15% (in weight), more preferably between 0.0001% (in weight) and 10% (in weight) and even more preferably between 0.0001% (in weight) and 5% (in weight).

In some embodiments of the current invention, the composition may be delivered via any one of liposomes, mixed liposomes, oleosomes, niosomes, ethosomes, millicapsules, capsules, macrocapsules, nanocapsules, nanostructured lipid carriers, sponges, cyclodextrins, vesicles, micelles, mixed micelles of surfactants, surfactant-phospholipid mixed micelles, millispheres, spheres, lipospheres, particles, nanospheres, nanoparticles, milliparticles, solid nanopartciles as well as microemulsions including water-in-oil microemulsions with an internal structure of reverse micelle and nanoemulsions microspheres, microparticles.

A variety of methods are available for preparing liposomes. See, e.g., Szoka et al., Ann. Rev. Biophys. Bioeng. 9:467 (1980), U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication No. WO 91/17424, Deamer & Bangham, Biochim. Biophys. Acta 443:629-634 (1976); Fraley, et al., PNAS 76:3348-3352 (1979); Hope et al., Biochim. Biophys. Acta 812:55-65 (1985); Mayer et al., Biochim. Biophys. Acta 858:161-168 (1986); Williams et al., PNAS 85:242-246 (1988); Liposomes (Ostro (ed.), 1983, Chapter 1); Hope et al., Chem. Phys. Lip. 40:89 (1986); Gregoriadis, Liposome Technology (1984) and Lasic, Liposomes: from Physics to Applications (1993)). Suitable methods include, for example, sonication, extrusion, high pressure/homogenization, microfluidization, detergent dialysis, calcium-induced fusion of small liposome vehicles and ether fusion methods, all of which are well known in the art.

These delivery systems may be adapted to achieve a greater penetration of the compound and/or peptides of the invention. This may improve pharmacokinetic and pharmacodynamics properties. The delivery system may be a sustained release system wherein the compound or peptide of the invention is gradually released during a period of time and preferably with a constant release rate over a period of time. The delivery systems are prepared by methods known in the art. The amount of peptide contained in the sustained release system will depend on where the composition is to be delivered and the duration of the release as well as the type of the condition, disease and/or disorder to be treated or cared for.

The topical composition of the invention may be for human or animal usage in human and veterinary medicine.

The topical composition of the invention may be used for pharmaceutical, personal care and/or cosmetic uses.

The composition can be used to treat or care for any disease, disorder or condition of the skin, including but not limited to, psoriasis, dermatitis, allergic dermatitis, eczema, spongiosis, edema, skin cancer, ulcers, acne, scars, cellulitis, elastosis, keratosis, rosacea, varicose veins, inflammatory disorders.

The topical composition may be used to for treating or caring for visible signs of aging including but not limited to wrinkles, stretch marks and dark circles, dryness, fine lines, age spots, red blotches, sagging skin, and conditions caused by sun exposure including sunburn, stress, pollution and/diet. The topical composition may also be used for delaying, slowing or inhibiting the skins or the onset of aging. The composition may be administered by a medical device, such as a plaster or a patch as described herein.

The topical composition may be used to treat or care for a wound in a mammal. In another embodiment, the topical composition is for use in the treatment or prevention of a disease or condition characterised by damaged epithelial cells or tissue, and/or damaged dermal or epithelial cells or tissue. The disease may be but is not limited to cancer and trauma.

The topical composition may be used to treat or care for any muscle condition, to improve, muscle status in a mammal, to promote recovery of muscle, typically following exercise, to maintain or restore muscle health (for example lean tissue mass) in a mammal, to enhance physical performance, in treatment or prevention of a disease or condition characterised by lethargy or low energy levels.

The topical composition may be used to promote growth of a tissue, promote growth of epithelial tissue, promote growth of skin, promote growth of an organ, promote growth of an organism. The skin can have a normal pathology and/or an abnormal pathology.

The topical composition may also be used to treat or care for any inflammatory disorder.

A further aspect of the invention relates to a pharmaceutical composition comprising a peptide of the invention or a composition of peptides of the invention, admixed with one or more pharmaceutically acceptable diluents, excipients or carriers. Even though the peptides and compositions of the present invention can be administered alone, they will generally be administered in admixture with a pharmaceutical carrier, excipient or diluent, particularly for human therapy. The pharmaceutical compositions may be for human or animal usage in human and veterinary medicine. Examples of such suitable excipients for the various different forms of pharmaceutical compositions described herein may be found in the “Handbook of Pharmaceutical Excipients, 2^{nd} Edition, (1994), Edited by A Wade and P J Weller. In particular, formulations for topical delivery are described in Topical drug delivery formulations edited by David Osborne and Antonio Aman, Taylor & Francis, the complete contents of which are incorporated herein by reference. Acceptable carriers or diluents for therapeutic use are well known in the pharmaceutical art, and are described, for example, in Remington's Pharmaceutical Sciences, Mack Publishing Co. (A. R. Gennaro edit. 1985). Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

The peptide or composition of the invention may be adapted for topical, oral, rectal, parenteral, intramuscular, intraperitoneal, intra-arterial, intrabronchial, subcutaneous, intradermal, intravenous, nasal, vaginal, buccal or sublingual routes of administration. For oral administration, particular use is made of compressed tablets, pills, tablets, gellules, drops, and capsules. Preferably, these compositions contain from 1 to 250 mg and more preferably from 10-100 mg, of active ingredient per dose. Other forms of administration comprise solutions or emulsions which may be injected intravenously, intra-arterial, subcutaneously, intradermally, intraperitoneally or intramuscularly, and which are prepared from sterile or sterilisable solutions. The pharmaceutical compositions of the present invention may also be in form of suppositories, vaginal rings, pessaries, suspensions, emulsions, lotions, ointments, creams, gels, sprays, solutions or dusting powders. The composition of the invention may be formulated for topical delivery. Topical delivery generally means delivery to the skin, but can also mean delivery to a body lumen lined with epithelial cells, for example the lungs or airways, the gastrointestinal tract, the buccal cavity. In particular, formulations for topical delivery are described in Topical drug delivery formulations edited by David Osborne and Antonio Aman, Taylor & Francis, the complete contents of which are incorporated herein by reference. Compositions or formulations for delivery to the airways are described in O′Riordan et al (Respir Care, 2002 November 47), EP2050437, WO2005023290, US2010098660, and US20070053845. Composition and formulations for delivering active agents to the iluem, especially the proximal iluem, include microparticles and microencapsulates where the active agent is encapsulated within a protecting matrix formed of polymer or dairy protein that is acid resistant but prone to dissolution in the more alkaline environment of the ileum. Examples of such delivery systems are described in EP1072600.2 and EP13171757.1. An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention. Depending upon the need, the agent may be administered at a dose of from 0.01 to 30 mg/kg body weight, such as from 0.1 to 10 mg/kg, more preferably from 0.1 to 1 mg/kg body weight. In an exemplary embodiment, one or more doses of 10 to 300 mg/day or more preferably, 10 to 150 mg/day, will be administered to the patient for the treatment of an inflammatory disorder.

In a particularly preferred embodiment, the methods and uses of the invention involve administration of a peptide or composition of the invention in combination with one or more other active agents, for example, existing anti-inflammatory drugs or pharmacological enhancers available on the market. In such cases, the compounds of the invention may be administered consecutively, simultaneously or sequentially with the one or more other active agents.

In one embodiment of the invention, the peptide of the invention may be administered in the form of a conjugate comprising the peptide, and may optionally include a linker, and a partner molecule, for example a protein such as an antibody molecule intended to increase the half-life of the conjugate in-vivo. In one embodiment, the peptide may be modified to substitute one or more amino acids with amino acids employed to attach partner molecules. For example, an amino acid may be substituted with a lysine residue for the purpose of conjugating a partner molecule such as a PEG molecule.

### Definitions

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

The term “human or animal” should be understood to means humans or mammalian or non-mammalian animals such as fish.

The term “composition” should be understood to mean a composition of matter made by the hand of man and not occurring in nature. Exemplary compositions include food compositions, beverage compositions, pharmaceutical compositions, nutritional supplement compositions, personal care compositions and healthcare compositions.

The term “peptide” used herein refers to a polymer composed of 5 to 50 amino acid monomers typically via peptide bond linkage. Peptides (including fragments and variants thereof) of and for use in the invention may be generated wholly or partly by chemical synthesis or by expression from nucleic acid. For example, the peptides of and for use in the present invention can be readily prepared according to well-established, standard liquid or, preferably, solid-phase peptide synthesis methods known in the art (see, for example, J. M. Stewart and J. D. Young, Solid Phase Peptide Synthesis, 2nd edition, Pierce Chemical Company, Rockford, Illinois (1984), in M. Bodanzsky and A. Bodanzsky, The Practice of Peptide Synthesis, Springer Verlag, New York (1984). When necessary, any of the peptides employed in the invention can be chemically modified to increase their stability. A chemically modified peptide or a peptide analog includes any functional chemical equivalent of the peptide characterized by its increased stability and/or efficacy in vivo or in vitro in respect of the practice of the invention. The term peptide analog also refers to any amino acid derivative of a peptide as described herein. A peptide analog can be produced by procedures that include, but are not limited to, modifications to side chains, incorporation of unnatural amino acids and/or their derivatives during peptide synthesis and the use of cross-linkers and other methods that impose conformational constraint on the peptides or their analogs. Examples of side chain modifications include modification of amino groups, such as by reductive alkylation by reaction with an aldehyde followed by reduction with NaBH₄; amidation with methylacetimidate; acetylation with acetic anhydride; carbamylation of amino groups with cyanate; trinitrobenzylation of amino groups with 2, 4, 6, trinitrobenzene sulfonic acid (TNBS); alkylation of amino groups with succinic anhydride and tetrahydrophthalic anhydride; and pyridoxylation of lysine with pyridoxa-5′-phosphate followed by reduction with NABH₄. The guanidino group of arginine residues may be modified by the formation of heterocyclic condensation products with reagents such as 2,3-butanedione, phenylglyoxal and glyoxal. The carboxyl group may be modified by carbodiimide activation via o-acylisourea formation followed by subsequent derivatization, for example, to a corresponding amide. Sulfhydryl groups may be modified by methods, such as carboxymethylation with iodoacetic acid or iodoacetamide; performic acid oxidation to cysteic acid; formation of mixed disulphides with other thiol compounds; reaction with maleimide; maleic anhydride or other substituted maleimide; formation of mercurial derivatives using 4-chloromercuribenzoate, 4-chloromercuriphenylsulfonic acid, phenylmercury chloride, 2-chloromercuric-4-nitrophenol and other mercurials; carbamylation with cyanate at alkaline pH. Tryptophan residues may be modified by, for example, oxidation with N-bromosuccinimide or alkylation of the indole ring with 2-hydroxy-5-nitrobenzyl bromide or sulphonyl halides. Tryosine residues may be altered by nitration with tetranitromethane to form a 3-nitrotyrosine derivative. Modification of the imidazole ring of a histidine residue may be accomplished by alkylation with iodoacetic acid derivatives or N-carbethoxylation with diethylpyrocarbonate. Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. Peptide structure modification includes the generation of retro-inverso peptides comprising the reversed sequence encoded by D-amino acids.

The term “modified peptide” is used interchangeably with the term derivative of the peptide. The modified peptide includes a peptide which has been substituted with one or more groups as defined herein. The modification may be any modified that provides the peptides and or the composition of the invention with an increased ability to penetrate a cell. The modification may be any modification that increases the half-life of the composition or peptides of the invention. In one embodiment, the group is a protecting group. The protecting group may be an N-terminal protecting group, a C-terminal protecting group or a side-chain protecting group. The peptide may have one or more of these protecting groups. The person skilled in the art is aware of suitable techniques to react amino acids with these protecting groups. These groups can be added by preparation methods known in the art, for example the methods as outlined in paragraphs to of US2014120141. The groups may remain on the peptide or may be removed. The protecting group may be added during synthesis. In an embodiment of the invention the peptides may be substituted with a group selected from one or more straight chain or branched chain, long or short chain, saturated, or unsaturated, substituted with a hydroxyl, amino, amino acyl, sulfate or sulphide group or unsubstituted having from 1 to 29 carbon atoms. N-acyl derivatives include acyl groups derived from acetic acid, capric acid, lauric acid, myristic acid, octanoic acid, palmitic acid, stearic acid, behenic acid, linoleic acid, linolenic acid, lipoic acid, oleic acid, isosteric acid, elaidoic acid, 2-ethylhexaneic acid, coconut oil fatty acid, tallow fatty acid, hardened tallow fatty acid, palm kernel fatty acid, lanolin fatty acid or similar acids. These may be substituted or unsubstituted. When substituted they are preferably substituted with hydroxyl, or sulphur containing groups such as but not limited to SO₃H, SH, or S—S. In an embodiment of the current invention, the peptide is R₁-X-R₂. R₁ and/or R₂ groups respectively bound to the amino-terminal (N-terminal) and carboxyl-terminal (C-terminal) of the peptide sequence. In one embodiment, the peptide is R₁-X. Alternatively, the peptide is X-R₂. Preferably, R₁ is H, C₁₋₄ alkyl, acetyl, benzoyl or trifluoroacetyl; X is the peptide of the invention; R₂ is OH or NH₂. In an embodiment, R₁ is selected from the group formed by H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, substituted or unsubstituted aralkyl, Tert-butyloxycarbonyl, 9-fluorenylmethyloxycarbonyl (Fmoc) and R₅—CO—, wherein R₅ is selected from the group formed by H, a non-cyclic substituted or unsubstituted aliphatic group, substituted or unsubstituted alicyclyl, substituted or unsubstituted aryl, substituted or unsubstituted aralkyl, substituted or unsubstituted heterocyclyl and substituted or unsubstituted heteroarylalkyl; R₂ is selected from the group formed by —NR₃R₄, —OR₃ and —SR₃, wherein R₃ and R₄ are independently selected from the group formed by H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, and substituted or unsubstituted aralkyl; and with the condition that R₁ and R₂ are not a-amino acids. In accordance with another preferred embodiment, R₂ is —NR₃R₊, —OR₃ or —SR₃ wherein R₃ and R₄ are independently selected from the group formed by H, substituted or unsubstituted C₁-C₂₄ alkyl, substituted or unsubstituted C₂-C₂₄ alkenyl, Tert-butyloxycarbonyl, 9-fluorenylmethyloxycarbonyl (Fmoc), substituted or unsubstituted C₂-C₂₄ alkynyl, substituted or unsubstituted C₃-C ₂₄ cycloalkyl, substituted or unsubstituted C₅-C₂₄ cycloalkenyl, substituted or unsubstituted C₈-C₂₄ cycloalkynyl, substituted or unsubstituted C₆-C₃₀ aryl, substituted or unsubstituted C₇-C₂₄ aralkyl, substituted or unsubstituted heterocyclyl ring of 3-10 members, and substituted or unsubstituted heteroarylalkyl of 2 to 24 carbon atoms and 1 to 3 atoms other than carbon wherein the alkyl chain is of 1 to 6 carbon atoms. Optionally, R₃ and R₄ can be bound by a saturated or unsaturated carbon-carbon bond, forming a cycle with the nitrogen atom. More preferably R₂ is —NR₃R₄ or —OR₃, wherein R₃ and R₄ are independently selected from the group formed by H, substituted or unsubstituted C₁-C₂₄ alkyl, substituted or unsubstituted C₂-C₂₄ alkenyl, substituted or unsubstituted C₂-C₂₄ alkynyl, substituted or unsubstituted C₃-C₁₀ cycloalkyl, substituted or unsubstituted C₆-C₁₅ aryl and substituted or unsubstituted heterocyclyl of 3-10 members, substituted or unsubstituted heteroarylalkyl with a ring of 3 to 10 members and an alkyl chain of 1 to 6 carbon atoms. More preferably R₃ and R₄ are selected from the group formed by H, methyl, ethyl, hexyl, dodecyl, or hexadecyl. Even more preferably R₃ is H and R₄ is selected from the group formed by H, methyl, ethyl, hexyl, dodecyl, or hexadecyl. In accordance with an even more preferred embodiment, R₂ is selected from —OH and —NH₂.

In accordance with another embodiment of this invention R₁ is selected from the group formed by H, acetyl, lauroyl, myristoyl or palmitoyl, and R₂ is —NR₃R₄ or —OR₃ wherein R₃ and R₄ are independently selected from H, methyl, ethyl, hexyl, dodecyl and hexadecyl, preferably R₂ is —OH or —NH₂. More preferably, R₁ is acetyl or palmitoyl and R₂ is —NH₂. In a preferred embodiment, the acyl group is bound to the N-terminal end of at least one amino acid of the peptide. In an embodiment of the invention, the peptide is modified to comprise a side chain protecting group. The side chain protecting group may be one or more of the group comprising benzyl or benzyl based groups, t-butyl-based groups, benzyloxy-carbonyl (Z) group, and allyloxycarbonyl (alloc) protecting group The side chain protecting group may be derived from an achiral amino acid such as achiral glycine. The use of an achiral amino acid helps to stabilise the resultant peptide and also facilitate the facile synthesis route of the present invention. Preferably, the peptide further comprises a modified C-terminus, preferably an amidated C-terminus. The achiral residue may be alpha-aminoisobutyric acid (methylalaine). It will be appreciated that the specific side chain protecting groups used will depend on the sequence of the peptide and the type of N-terminal protecting group used.

“Conjugate”: In one embodiment of the invention the peptide is conjugated, linked or fused to a binding partner, for example one or more polyethylene glycol polymers or other compounds, such as molecular weight increasing compounds or lipophilic groups. The molecular weight increasing compound is any compound that will increase the molecular weight, typically by 10% to 90%, or 20% to 50% of the resulting conjugate and may have a molecular weight of between 200 and 20,000, preferably between 500 and 10,000. The molecular weight increasing compound may be PEG, any water-soluble (amphiphilic or hydrophilic) polymer moiety, homo or co-polymers of PEG, a monomethyl-substituted polymer of PEG (mPEG) and polyoxyethylene glycerol (POG), polyamino acids such as poly-lysine, poly-glutamic acid, poly-aspartic acid, particular those of L conformation, pharmacologically inactive proteins such as albumin, gelatin, a fatty acid, polysaccharide, a lipid amino acid and dextran. The polymer moiety may be straight chained or branched and it may have a molecular weight of 500 to 40000Da, 5000 to 10000 Da, 10000 to 5000, Da. The compound (binding partner) may be any suitable cell penetrating compound, such as tat peptide, penetratin, pep-1. The compound (binding partner) may be an antibody molecule. The compound (binding partner) may be a lipophilic moiety or a polymeric moiety. The lipophilic substituent and polymeric substituents are known in the art. The lipophilic substituent includes an acyl group, a sulphonyl group, an N atom, an O atom or an S atom which forms part of the ester, sulphonyl ester, thioester, amide or sulphonamide. The lipophilic moiety may include a hydrocarbon chain having 4 to 30 C atoms, preferably between 8 and 12 C atoms. It may be linear or branched, saturated or unsaturated. The hydrocarbon chain may be further substituted. It may be cycloalkane or heterocycloalkane. The peptide may be modified at the N-terminal, C-terminal or both. The polymer or compound (binding partner) is preferably linked to an amino, carboxyl or thio group and may be linked by N-termini or C-termini of side chains of any amino acid residue. The polymer or compound (binding partner) may be conjugated to the side chain of any suitable residue. The polymer or compound (binding partner) may be conjugated via a spacer. The spacer may be a natural or unnatural amino acid, succinic acid, lysyl, glutamyl, asparagyl, glycyl, beta-alanyl, gamma-amino butanoyl. The polymer or compound (binding partner) may be conjugated via an ester, a sulphonyl ester, a thioester, an amide, a carbamate, a urea, a sulphonamide. A person skilled in the art is aware of suitable means to prepare the described conjugate.

“Fragment” means a segment of a protein selected from SEQ ID NOs: 1 to 14, the fragment typically being 7 to 37 contiguous amino acids in length, and generally having a charge of between −9 and +3; a c-terminal amino acid that typcially is not cysteine (C) or methionine (M); and an n-terminal amino acid that typcially is not cysteine (C), histidine (H), proline (P) or threonine (T). The charge of a peptide, fragment or region is determined using the method of Cameselle, J. C., Ribeiro, J. M., and Sillero, A. (1986). Derivation and use of a formula to calculate the net charge of acid-base compounds. Its application to amino acids, proteins and nucleotides. Biochem. Educ. 14, 131-136.

The term “natural” as applied to a peptide means a peptide that includes (a) a fragment of a plant protein, typically rice or pea protein, or variants of pea protein including lentil, sweet pea, or chick pea or variants of rice protein including oat, grass, corn, wild rice and bananas, or (b) a variant of the fragment of a plant protein, for example a fragment of a homolog of the plant protein. The peptides or fragments of the invention may be isolated from plant proteins or made synthetically using methods known to a person skilled in the art and described herein.

“C-terminal domain” as applied to a fragment means the first three amino acids at the c-terminus of the fragment.

“N-terminal domain” as applied to a fragment means the last three amino acids at the n-terminus of the fragment.

“Bioactive” as applied to a peptide or fragment means having a health promoting effect when administered a mammal, for example one or more of glucose transport promoting, anti-bacterial, anti-inflammatory, or cellular growth or proliferation promoting. In one embodiment, the term “bioactive” means cellular growth promoting.

“Growth promoting” or “growth promoting activity” as applied to a peptide or fragment means a peptide or fragment that is capable of increasing elastin production or cellular proliferation of human skin treated with a 20 μM solution of peptide or fragment as described in the assay below.

“Glucose transport promoting” or “glucose transport promoting activity” as applied to a peptide or variant or fragment means a peptide, variant or fragment that is capable of increasing GLUT4 translocation into skeletal muscle compared with an untreated control when employed at a concentration of 2 μM in the following in-vitro assay. L6-GLUT4myc cells were grown in 10% FBS and 2 μg/ml blasticidin. Cells were grown for 48-72 hours before being seeded in 24-well plates at 15,000 cells per well in 2% FBS and allowed to differentiate for 6 to 8 days prior to experimentation. L6-GLUT4myc cells were serum-starved for three hours prior to incubation with 100 nM of insulin for 30 mins, or 200, 20, 2.0 and 0.2 μM of SP, and 2, 1, 0.5 and 0.25 mg/ml of pepide/peptide composition for 3 hours respectively. A 3-hour incubation period was selected based on previous findings identifying that incubation with branch chain amino acid containing di-peptides for 3 hours increases glucose uptake in L6 myotubes 1. Treatments were staggered in order to determine GLUT4myc translocation at the same time point. The quantity of myc-tagged GLUT4 at the cell surface was measured by antibody-coupled colorimetric assay. Briefly, after incubation with either insulin for 30 mins or synthetic peptide or peptide composition for 3 hours respectively, L6-GLUT4myc cells were fixed via incubation with 3% paraformaldehyde (PFA). A 0.1 M glycine solution was then added to quench PFA and cells were blocked with 5% goat serum. The myotube monolayer was exposed to anti-myc antibody and then incubated with peroxidase conjugated donkey anti-mouse IgG. 1 mL of o-phenylenediamine dihydrochloride (OPD) reagent was added to each well and this reaction was stopped by adding 250 μl/well of 3 M HCL. To determine GLUT4 translocation to cell surface, a measured aliquot of each condition was determined spectrophotometrically on a plate reader using absorbance at 492 nm. Preferably the peptide or fragment is capable of increasing GLUT4 translocation compared with an untreated control by at least 50% (i.e a relative unit increase in GLUT4 translocation of 1% to 1.5%).

“Antibacterial” or “antibacterial activity” as applied to a peptide or fragment means a peptide or fragment that is capable of visibly inhibiting the growth of a bacteria in the following agar-plate based growth inhibition assay: Peptide stock=5 mg/mL dissolved in DMSO. Bacterial inoculums were adjusted to McFarland 0.5 standard and MHA plates swabbed. Blank disks were placed in the plates and 10 μL of each compound (at 64 μg/mL—maximum concentration tested) added. Plates were incubated at 37° C. for 16-18 hours. Appropriate controls (DMSO; Mueller-Hinton media alone; and two antibiotic discs—ciprofloxacin and tetracycline) were also performed.

“Anti-inflammatory” as applied to a peptide or fragment means a peptide or fragment that is capable of significantly reducing the secretion of TNFα by LPS-stimulated J774.2 macrophages (compared with untreated LPS-stimulated J774.2 macrophages) when the macrophages are treated with 100 μM of the peptide or fragment. J774.2 macrophages were treated with 100 μM of synthetic peptide for 24 hours and then stimulated with (A) LPS (10 ng/ml) for five hours or (B) LPS (10 ng/ml) for 5 hours followed by ATP (5 mM) for one hour. Supernatant was collected and levels of TNFα were determined by ELISA.

“Enriched in peptides having a molecular weight of less than 10 KD” as applied to a composition of the invention means that the dry weight % of peptides in the composition having a molecular weight of less than 10 KD is greater than the dry weight % of polypeptide/protein in the composition having a molecular weight of 10 KD or greater.

“Homolog” of a reference protein should be understood to mean a protein from a different species of plant having at least 60%, 70%, 80%, 90%, 91%, 92%, 93%, 94%, 95%, 96%, 97%, 98%, 99% sequence homology with the reference protein. Thus, for example, homologs of pea protein P13918 include:

>gi | 137584 | sp | P08438.1 | VCL_VICFA RecName: Full=Vicilin; Flags: Precursor [Vicia faba]
>gi | 22057 | emb | CAA68559.1 | vicilin [Vicia faba var. minor] >gi | 383931031 | gb | AFH56916.1 | vicilin
[Vicia faba]
>gi | 502105533 | ref | XP_004492829.1 | PREDICTED: vicilin-like isoform X1 [Cicer arietinum] ChickPea
>gi | 29539109 | emb | CAD87730.1 | allergen Len c 1.0101 [Lens culinaris] Lentil

A “variant” of a growth promoting fragment shall be taken to mean a fragment having an amino acid sequence that is substantially identical to the reference growth promoting fragment, and which has growth promoting activity as defined above. Thus, for example, the term should be taken to include fragments that are altered in respect of one or more amino acid residues. Preferably such alterations involve the insertion, addition, deletion and/or substitution of 5 or fewer amino acids, more preferably of 4 or fewer, even more preferably of 3 or fewer, most preferably of 1 or 2 amino acids only. Insertion, addition and substitution with natural and modified amino acids is envisaged. The variant may have conservative amino acid changes, wherein the amino acid being introduced is similar structurally, chemically, or functionally to that being substituted. Generally, the variant will have at least 70% amino acid sequence homology, preferably at least 80% sequence homology, more preferably at least 90% sequence homology, and ideally at least 95%, 96%, 97%, 98% or 99% sequence homology with the reference growth promoting fragment.

In this specification, the term “sequence identity” should be understand to comprise both sequence identity and similarity, i.e. a variant (or homolog) that shares 70% sequence identity with a reference sequence is one in which any 70% of aligned residues of the variant (or homolog) are identical to or conservative substitutions of the corresponding residues in the reference sequence across the entire length of the sequence. Sequence identity is the amount of characters which match exactly between two different sequences. Hereby, gaps are not counted and the measurement is relational to the shorter of the two sequences. In terms of “sequence homology”, the term should be understood to mean that a variant (or homolog) which shares a defined percent similarity or identity with a reference sequence when the percentage of aligned residues of the variant (or homolog) are either identical to, or conservative substitutions of, the corresponding residues in the reference sequence and where the variant (or homolog) shares the same function as the reference sequence. This alignment and the percent homology or sequence identity can be determined using software programs known in the art, for example, one alignment program is BLAST, using default parameters. Details of these programs can be found at the following Internet address: <www.ncbi.nlm.nih.gov/blast/Blast.cgi>

Variants of SEQ ID NO: 448 (QSFLLSGNQ)

Variants of SEQ ID NO: 448 (QSFLLSGNQ) including variants having 1 or 2 conservative amino acid substitutions, 1, 2 to 3 non-conservative amino acid substitutions, 1-2 amino acid additions, 1, 2 or 3 amino acid deletions, are provided below:

### One Conservative Amino Acid Substitution:

(SEQ ID NO: 418)
QSFILSGNE,
(SEQ ID NO: 419)
ESFLLSGNQ,
(SEQ ID NO: 420)
QSYLLSGNQ,
(SEQ ID NO: 421)
QSFLLSGDQ

### Two Conservative Amino Acid Substitutions:

(SEQ ID NO: 422)
QSYLLSGNE,
(SEQ ID NO: 423)
ESFLLSGNE,
(SEQ ID NO: 424)
ESYLLSGNQ,
(SEQ ID NO: 425)
QSFLLSGDE,
(SEQ ID NO: 426)
QSYLLSGDQ

### One Non-Conservative Amino Acid Substitution

(SEQ ID NO: 427)
QSFRLSGNQ,
(SEQ ID NO: 428)
QSFLLSYNQ,
(SEQ ID NO: 429)
QFFLLSGNQ,
(SEQ ID NO: 430)
QSFLLSGAQ,
(SEQ ID NO: 431)
QSFLLSGNP

### Two Non-Conservative Amino Acid Substitution

(SEQ ID NO: 432)
QSFRRSGNQ,
(SEQ ID NO: 433)
QSFLLSYIQ,
(SEQ ID NO: 434)
QFFLLSGNL,
(SEQ ID NO: 435)
QSFLLSGAQ,
(SEQ ID NO: 436)
QSFLLSGNP

### One or Two Amino Acid Additions

(SEQ ID NO: 437)
QSFLLSGNQQ,
(SEQ ID NO: 438)
QSFLLLSGNQ,
(SEQ ID NO: 439)
AQSFGLLSGNQ,
(SEQ ID NO: 440)
RQSFLLISGNQ,
(SEQ ID NO: 441)
QSFLLSGNQK

### One, Two or Three Amino Acid Deletions

(SEQ ID NO: 442)
QFLLSGNQ,
(SEQ ID NO: 443)
SFLLSGNQ,
(SEQ ID NO: 444)
QSFLLSGN,
(SEQ ID NO: 445)
QSFLLGNQ,
(SEQ ID NO: 446)
QSFLSGNQ,
(SEQ ID NO: 447)
QSLLSGNQ,
(SEQ ID NO: 448)
SFLLSGNQ,
(SEQ ID NO: 449)
QSFLLSGN,
(SEQ ID NO: 450)
SFLLSGN,
(SEQ ID NO: 451)
QSFSGNQ

The term “variant” also includes fragment of peptides of the invention. “Fragment of a peptide of the invention” or “peptide fragment” means a fragment of one of the peptides of the invention having at least 3, 4, 5, 6, 7, 8, 9, 10, 11, 12, 13, 14, 15, 16, 17, 18, 19, 20, 21 or 22 amino acids and that typically has a bioactivity, for example anti-inflammatory activity, anti-ageing activity, glucose transport promoting activity, or anti-bacterial activity. In one embodiment, the fragment consists of at least 30%, 40%, 50%, 60%, 70%, 80%, or 90% of the reference sequence. Thus, the invention also provides bioactive fragments of the peptides of the invention, and peptides comprising one or more of these fragments. In one embodiment, the fragments are bioactive. In one embodiment, the fragments are cellular growth or proliferation promoting fragments. Examples of fragments of peptides of the invention are provide in SEQ ID NOs: 494 to 524.

“Anti-ageing” means inhibiting or slowing the appearance of ageing of a human skin and/or reversing the appearance of ageing. “Slowing or inhibiting ageing of the skin” means slowing or inhibiting the ageing process in the skin, and/or reversing the appearance of ageing.

“Disease or condition characterised by damaged dermal or epithelial cells or tissue” means any condition or disease that results in damaged dermal or epithelial tissue or cells or organs. One example is trauma which often results in damaged skin. Another example is an inflammatory skin condition such as psoriasis or excezma which often results in damaged skin. Another example is an inflammatory disorder of the lower intestines which can result in damaged epithelial cells/tissue lining the lower intestines. Another example is damaged epithelial cells/tissue lining the lower intestines caused by ingestion of a toxic or damaging substance, for example toxic chemicals or drugs. Another example is cancer, for example bowel cancer, which can result in damaged epithelial tissue in the bowel. Another condition is a peripheral inflammatory disorder such as atopic dermatitis which can result in damage to the skin in humans.

“Inflammatory disorder” means an immune-mediated inflammatory condition that affects humans and is generally characterised by dysregulated expression of one or more cytokines. Examples of inflammatory disorders include skin inflammatory disorders, inflammatory disorders of the joints, inflammatory disorders of the cardiovascular system, certain autoimmune diseases, lung and airway inflammatory disorders, intestinal inflammatory disorders. Examples of skin inflammatory disorders include dermatitis, for example atopic dermatitis and contact dermatitis, acne vulgaris, and psoriasis. Examples of inflammatory disorders of the joints include rheumatoid arthritis. Examples of inflammatory disorders of the cardiovascular system are cardiovascular disease and atherosclerosis. Examples of autoimmune diseases include Type 1 diabetes, Graves disease, Guillain-Barre disease, Lupus, Psoriatic arthritis, and Ulcerative colitis. Examples of lung and airway inflammatory disorders include asthma, cystic fibrosis, COPD, emphysema, and acute respiratory distress syndrome. Examples of intestinal inflammatory disorders include colitis and inflammatory bowel disease. Other inflammatory disorders include cancer, hay fever, periodontitis, allergies, hypersensitivity, ischemia, depression, systemic diseases, post infection inflammation and bronchitis. The invention also relates to a peptide or composition of the invention for use in treating an inflammatory disorder in a mammal.

“Metabolic disorder” should be understood to include pre-diabetes, diabetes; Type-1 diabetes; Type-2 diabetes; metabolic syndrome; obesity; diabetic dyslipidemia; hyperlipidemia; hypertension; hypertriglyceridemia; hyperfattyacidemia; hypercholerterolemia; hyperinsulinemia, and MODY. The invention also relates to a peptide or composition of the invention for use in treating a metabolic disorder in a mammal.

“Disease or condition characterised by damaged dermal or epithelial cells or tissue” means any condition or disease that results in damaged dermal or epithelial tissue or cells or organs. One example is trauma which often results in damaged skin. Another example is an inflammatory skin condition such as psoriasis or excezma which often results in damaged skin. Another example is an inflammatory disorder of the lower intestines which can result in damaged epithelial cells/tissue lining the lower intestines. Another example is damaged epithelial cells/tissue lining the lower intestines caused by ingestion of a toxic or damaging substance, for example toxic chemicals or drugs. Another example is cancer, for example bowel cancer, which can result in damaged epithelial tissue in the bowel. Another condition is a peripheral inflammatory disorder such as atopic dermatitis which can result in damage to the skin in humans.

“Disease or condition characterised by bacterial infection” means any condition or disease characterised having a pathology caused by growth of bacteria or by bacterial infection, including for example MRSA, salmonella, listeria, bacterial pneumonia, Staphylococcal food poisoning, bacterial memingitis. Specific examples are provided in <en.wikipedia.org/wiki/List_of_infectious_diseases>

“Man-made” as applied to comestible products should be understood to mean made by a human being and not existing in nature.

“Maintaining or restoring gut health” means reducing and/or regulating the pro-inflammatory response in the gut and more specifically the epithelial cells. The healthy microbiome offers some protection against pathogenic viruses and bacteria, and their presence is needed to guide the development of our immune system. It has been shown that these bacteria can react to human signals of stress, sickness, or age which can be manifested by inflammation and as a consequence switch on their virulence genes and cause or contribute to disease. Having the ability to reduce and maintain at healthy levels the inflammatory response can help maintain the healthy bacteria. Digestive problems, which comprise the number one health problem in North America, appear to be occurring with more frequency in recent years. One way to maintain digestive health is to maintain proper inflammation and intestinal flora.

“Improving muscle status” means improving the muscle health, for example promoting skeletal muscle protein synthesis, skeletal glucose absorbtion, improving lean tissue mass in therapeutic or non-therapeutic context, promoting muscle recovery generally after activity exercise, or improving muscle performance. The methods or uses may be therapeutic or non-therapeutic. The term “improving lean tissue mass status” should be understood to mean increasing lean tissue mass, or inhibiting or preventing the rate of lean tissue mass degradation.

“Promoting muscle recovery” means causing an increase in absorbtion of glucose in skeletal muscle compared with untreated skeletal muscle.

“Disease or condition characterised by lethargy or low energy levels” means any condition or disease characterised by a feeling or tiredness or low energy. Examples include allergies, asthma, anaemia, cancer and its treatments, chronic pain, heart disease, infection, depression, eating disorders, grief, sleeping disorders, thyroid problems, medication side effects, alcohol use, or drug use.

“Maintaining or restoring muscle health” means helping retain or restore mammalian muscle health resulting from damage incurred during exercise. By promoting glucose transport in skeletal muscle the peptides promote recovery from exercise, and relieve muscle soreness/pain and injury connected with exercise. They can also be used to decrease and prevent muscle cramping, and to allow a faster recovery from muscle cramping. Cramping can result from physical stress, mental stress, and or Repetitive Strain Injury stress. By promoting glucose transport the peptides help reduce Myopathy of the muscle, and help prevent Sarcopenia in mammals, promote recovery from injuries during exercise, and relieve muscle soreness/pain and injury connected with exercise. The invention also relates to a peptide or composition of the invention for use in maintaining or restoring muscle health in a mammal.

In this specification, the term “substantially all” as applied to a list of peptides should be understood to mean at least 60%, 70%, 80%, 90% or 95% of the peptides.

“Man-made” as applied to comestible products should be understood to mean made by a human being and not existing in nature.

### BRIEF DESCRIPTION OF THE FIGURES

FIGS. 1 to 100: Effect of synthetic peptides of the invention on proliferation of Human Dermal Fibroblasts (HDF).

FIG. 101: Effect of synthetic peptide of the invention (SEQ ID NO: 42) on elastin synthesis of Human Dermal Fibroblasts (HDF).

FIG. 102: Effect of synthetic peptide of the invention (SEQ ID NO: 42) on collagen synthesis of Human Dermal Fibroblasts (HDF).

FIG. 103: Effect of synthetic peptide of the invention (SEQ ID NO: 701) on elastin synthesis of Human Dermal Fibroblasts (HDF).

FIG. 104: Effect of synthetic peptide of the invention (SEQ ID NO: 701) on collagen synthesis of Human Dermal Fibroblasts (HDF).

FIG. 105: Effect of synthetic peptide of the invention (SEQ ID NO: 246) on elastin synthesis of Human Dermal Fibroblasts (HDF).

FIG. 106: Effect of synthetic peptide of the invention (SEQ ID NO: 246) on collagen synthesis of Human Dermal Fibroblasts (HDF).

FIG. 107: Effect of synthetic peptide of the invention (SEQ ID NO: 284) on elastin synthesis of Human Dermal Fibroblasts (HDF).

FIG. 108: Effect of synthetic peptide of the invention (SEQ ID NO: 245) on elastin synthesis of Human Dermal Fibroblasts (HDF).

FIG. 109: Effect of synthetic peptide of the invention (SEQ ID NO: 245) on collagen synthesis of Human Dermal Fibroblasts (HDF).

FIG. 110. shows the integrity controls and viability controls for the assay system.

FIG. 111. % of elastin expression in superficial dermis compared to control (water or DMSO) for peptides P1, P2 and P3

* shows significant increases of elastin expression in superficial AND middle dermis.

FIG. 112. % of elastin expression in middle dermis compared to control (water or DMSO) for peptides P1, P2 and P3.

* shows significant increases of elastin expression in superficial AND middle dermis.

FIG. 113. % of cell proliferation in the basal layer of epidermis compared to control (water or DMSO) for peptides P6 and P8, and peptide compositions P9 and P10

* shows significant increases.

FIG. 114. Histological analysis of the elastic fibers (+catechin, ×200)

FIG. 115. Immunohistochemical evaluation of the mitotic index (Ki67, ×400)

### DETAILED DESCRIPTION OF THE INVENTION

### Example 1—Cell Proliferation Assay

BrDu is incorporated into newly synthesised DNA strands of actively proliferating cells. Following partial denaturation of double stranded DNA, Brdu is detected immunochemically allowing the assessment of the population of cells which are synthesizing DNA.

Human Dermal Fibroblasts (HDF—Sigma 10605a) were seeded in a 96 well plate at 10,000 cells per well in DMEM containing 10% fetal calf serum (FCS), 1% Pen/strep, 1% L-glutamine and allowed to adhere for 24 h.

Following the initial 24 h incubation the cells were incubated with 5 μg/ml, 0.5 μg/ml or 0.05 μg/ml synthetic peptide for 24 h respectively.

After 18 h incubation with synthetic peptides 20 μl BrDu reagent was added to each well.

At 24 h incubation the cell were fixed and the amount of 2-DG6P was measured using the BrdU Cell Proliferation Assay, all steps were carried out according to the manufacturer's instructions.

Results were calculated as a percentage of the untreated control. An increase in optical density reading indicates greatER incorporation of BrDu and increase cell proliferation.

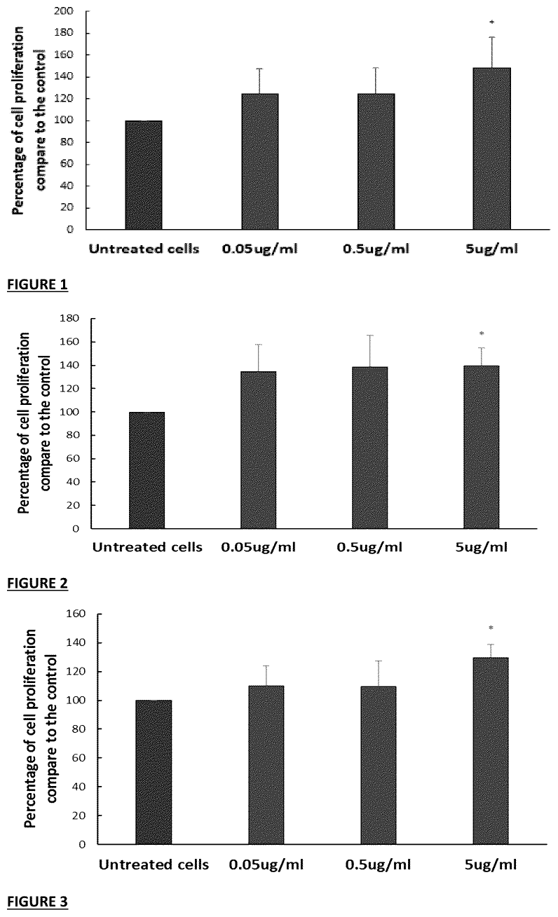

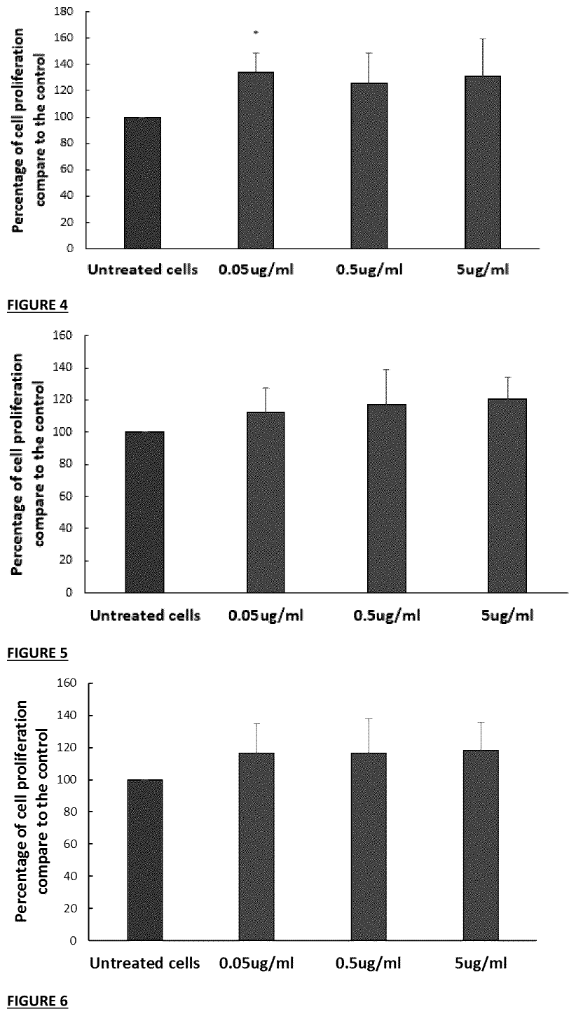

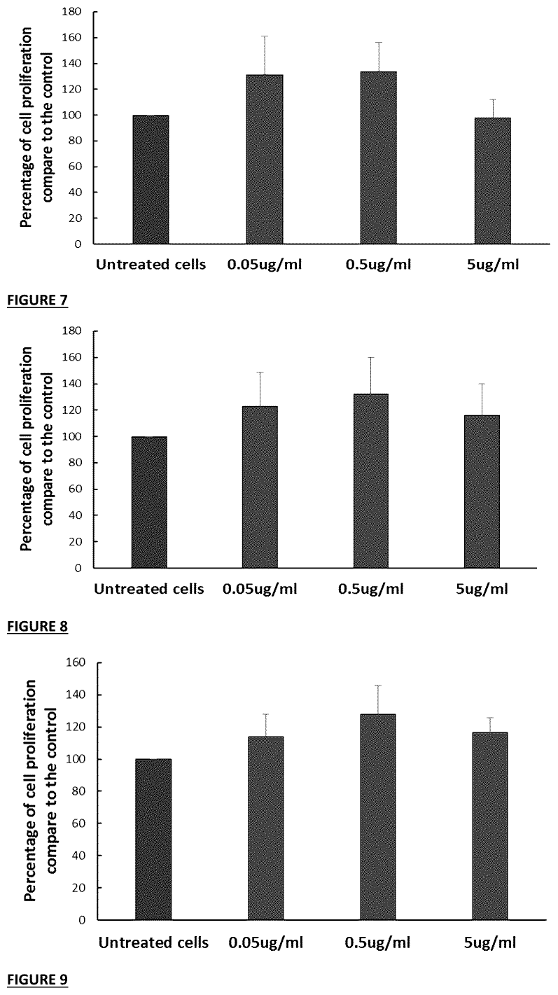

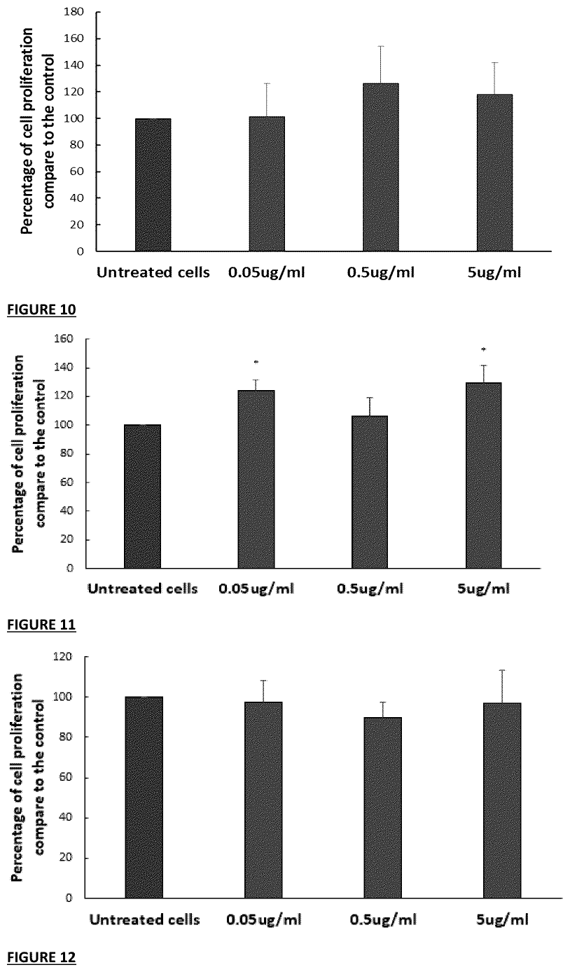

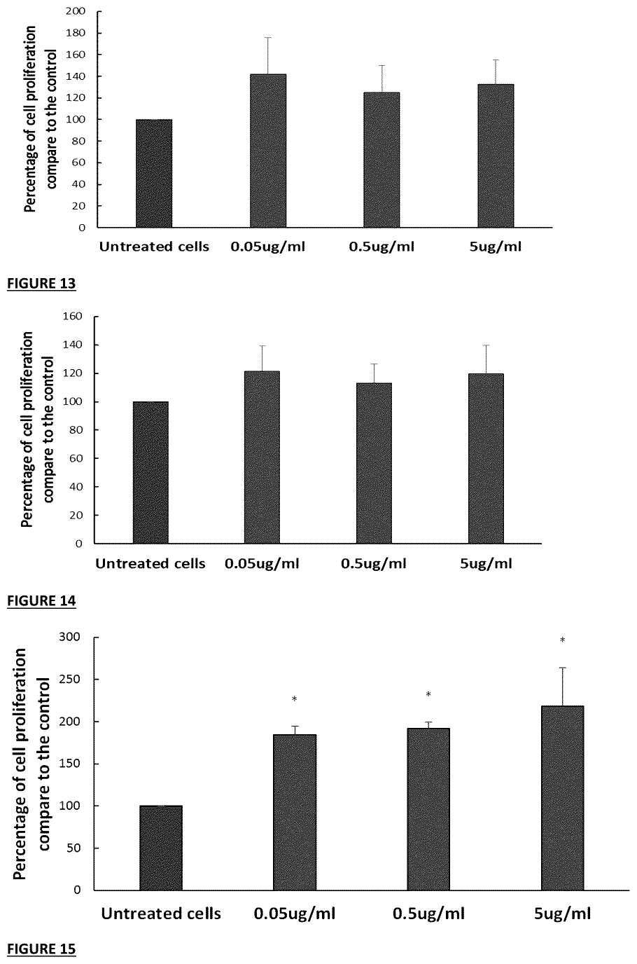

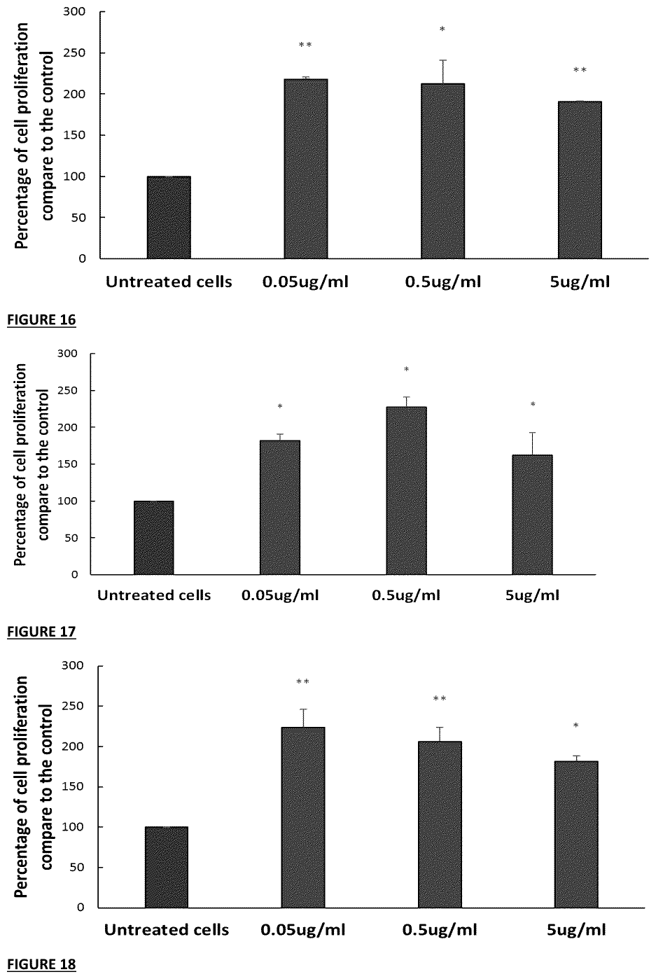

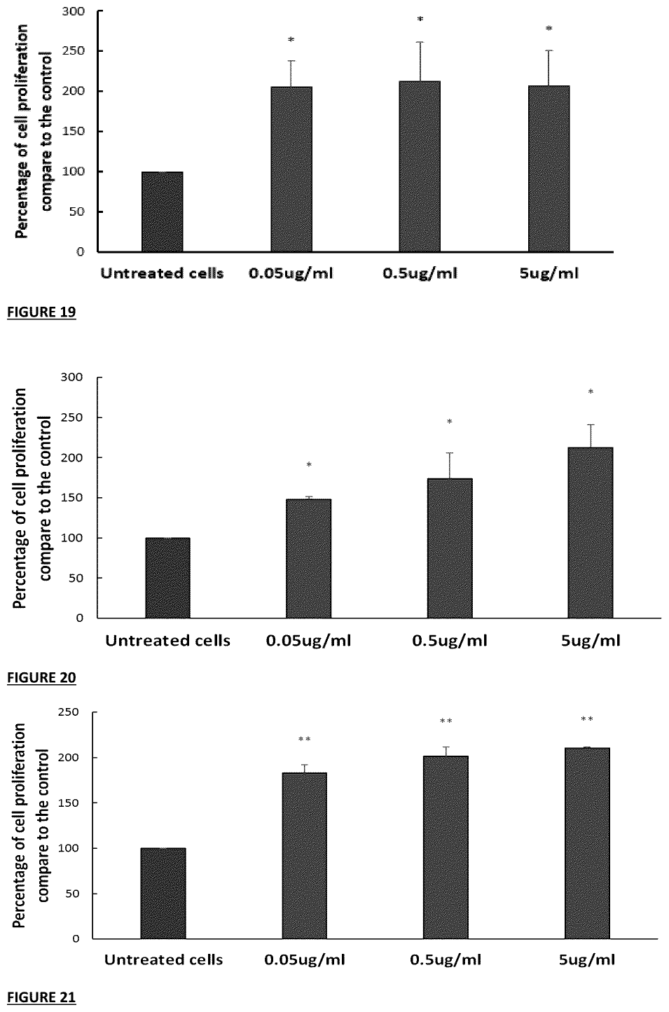

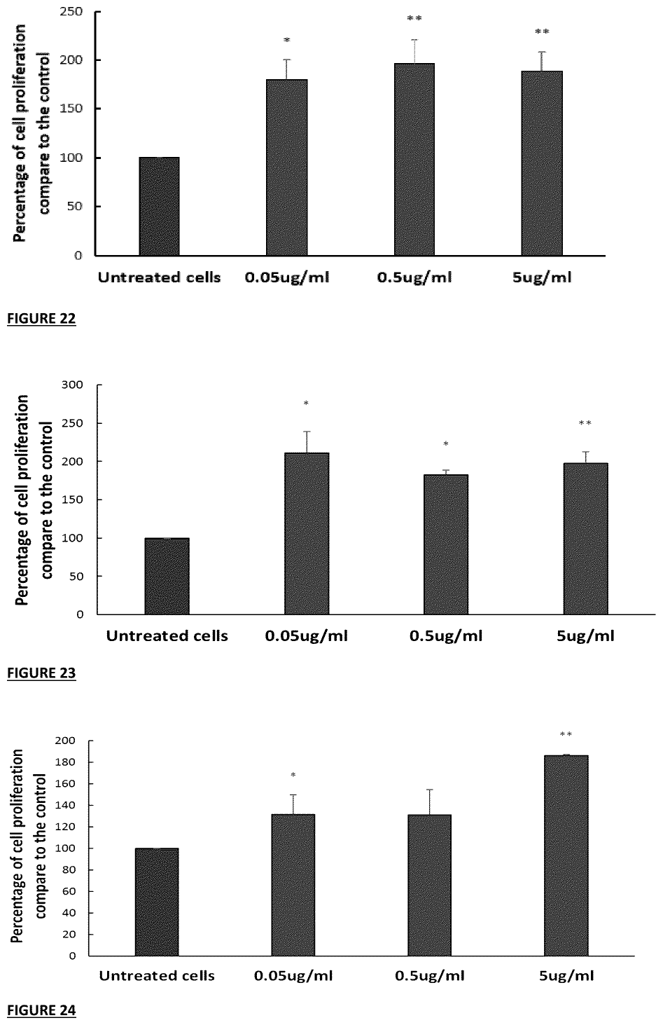

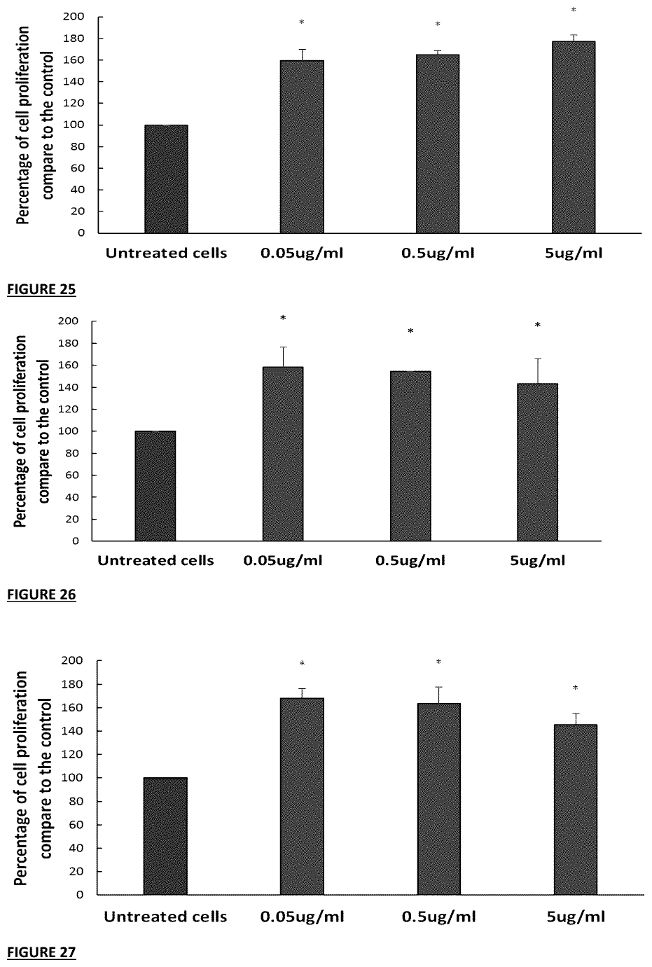

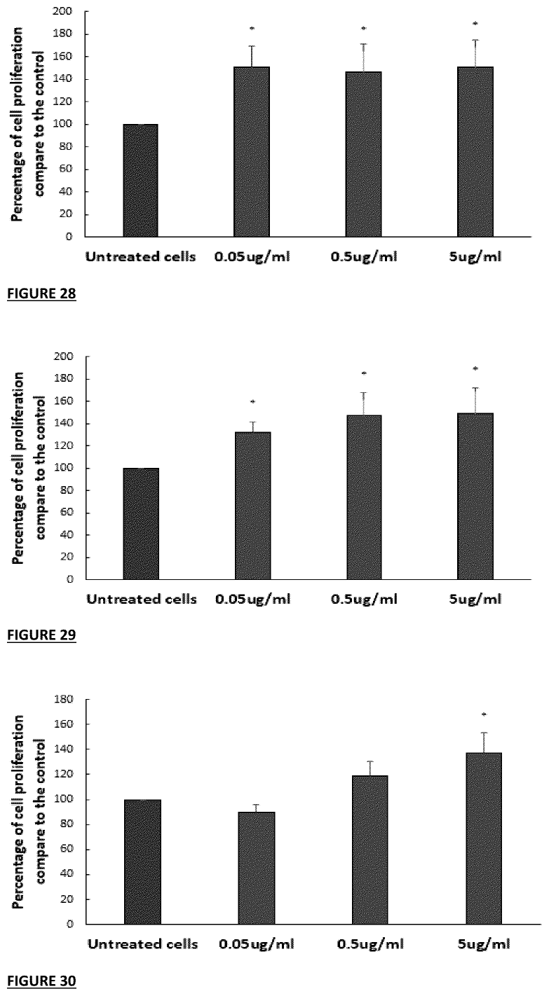

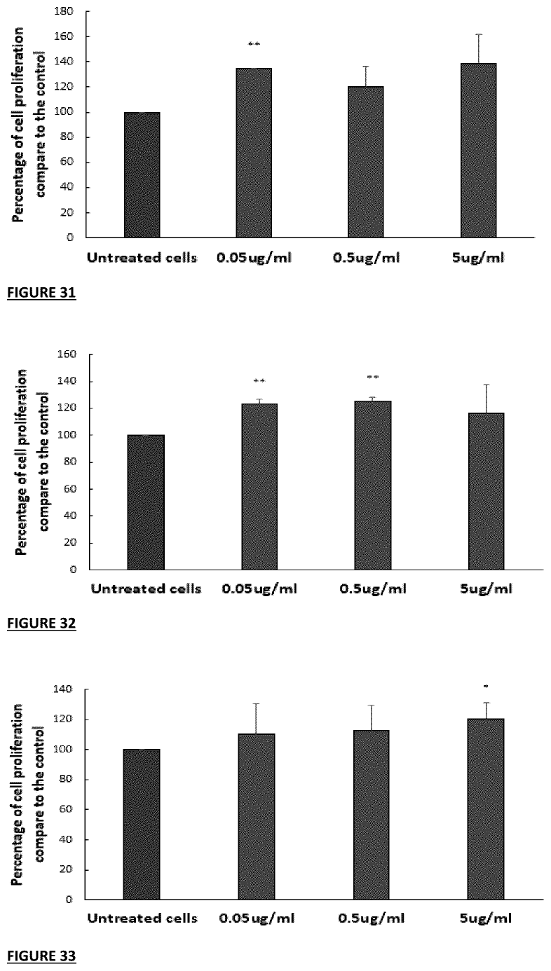

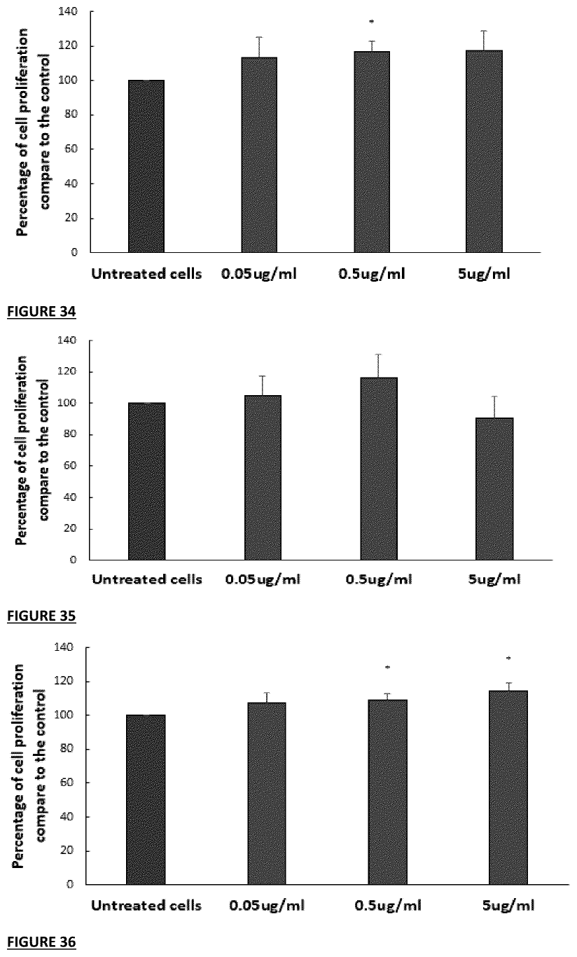

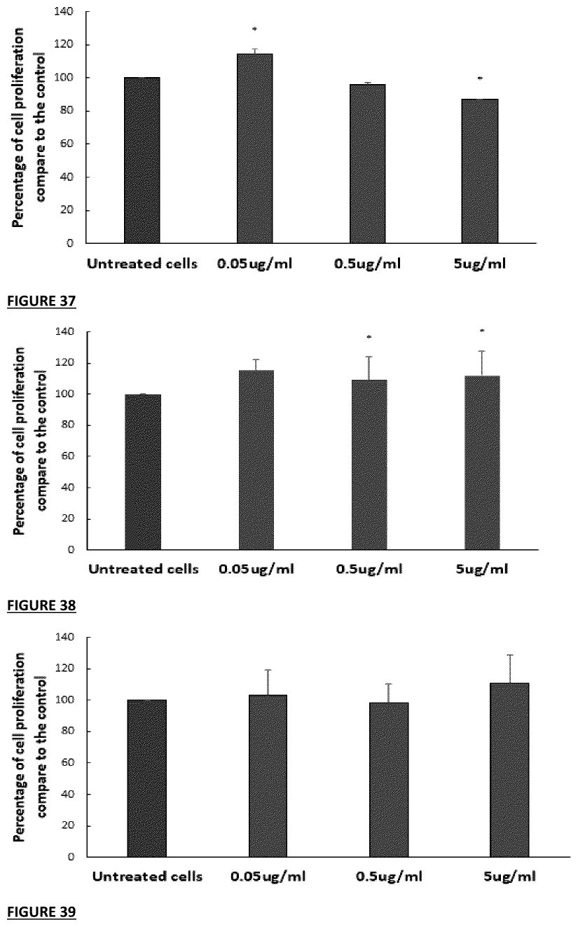

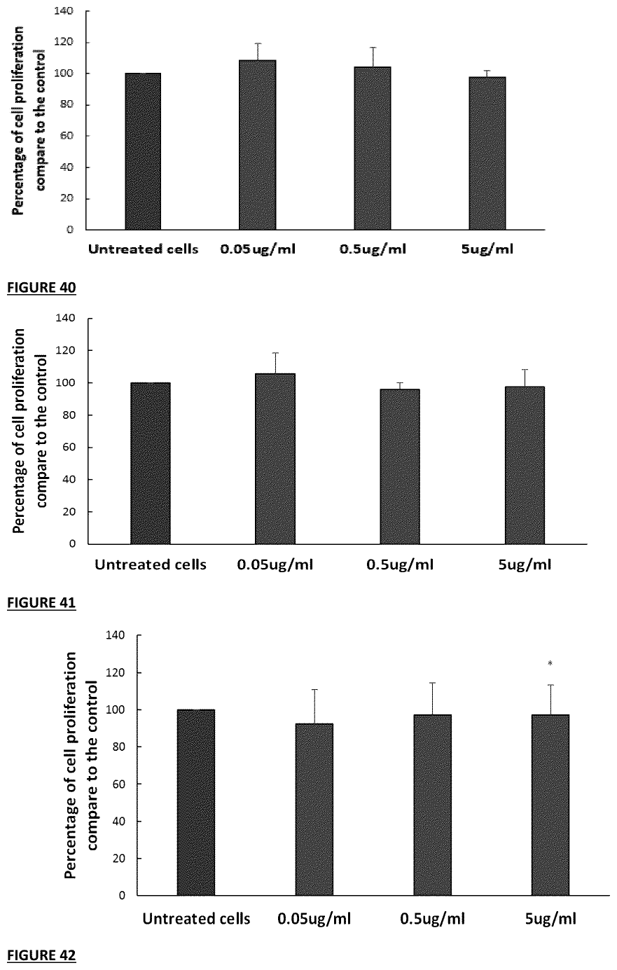

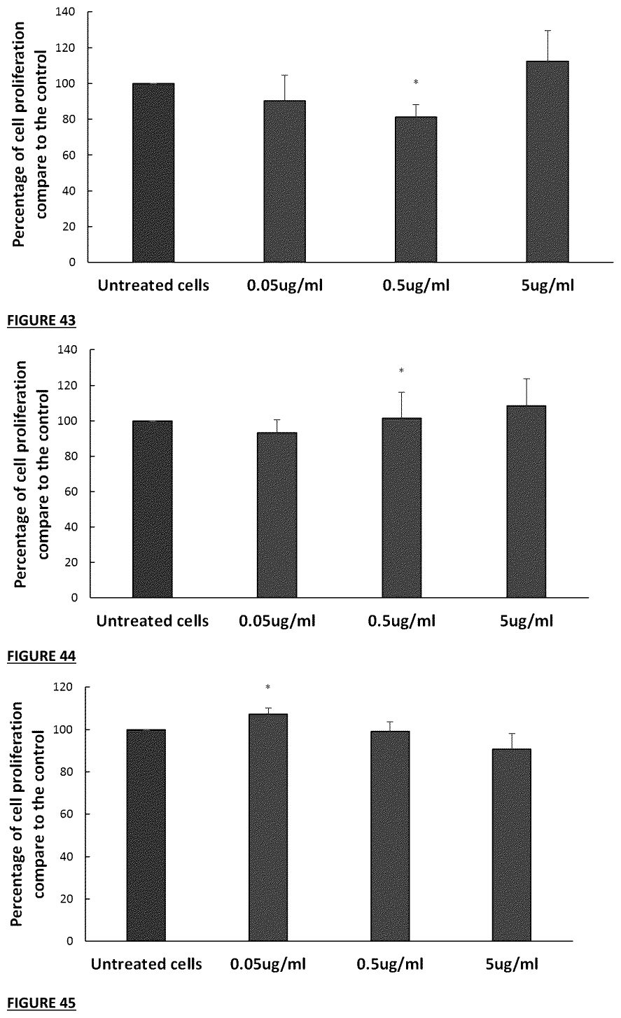

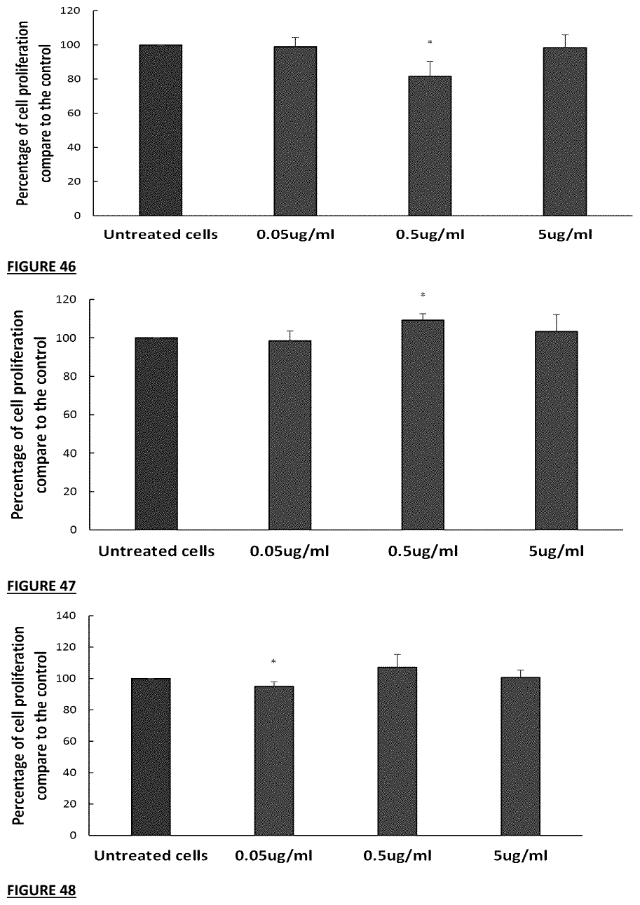

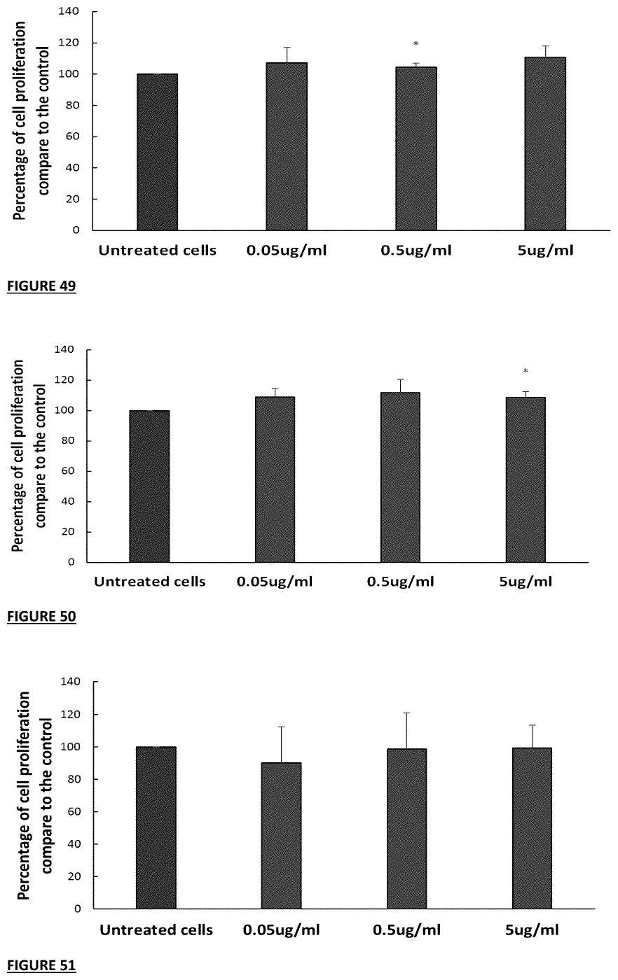

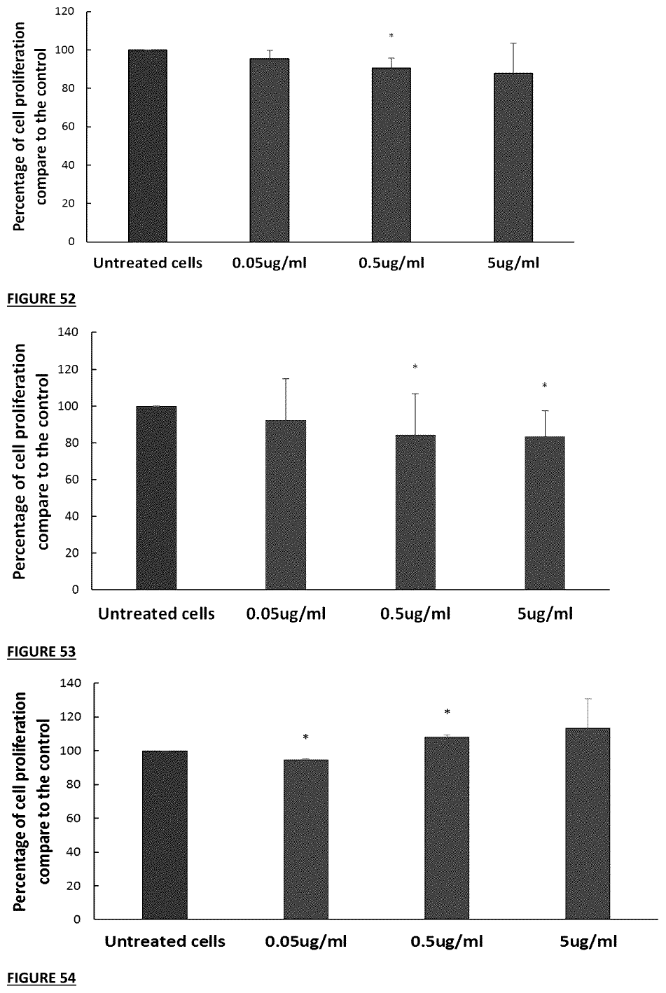

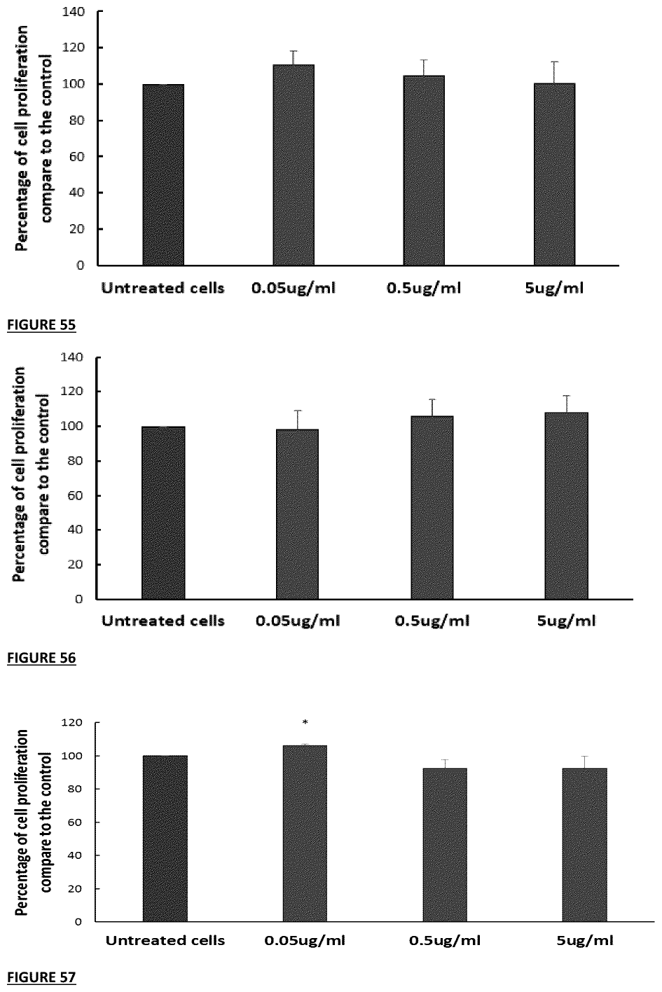

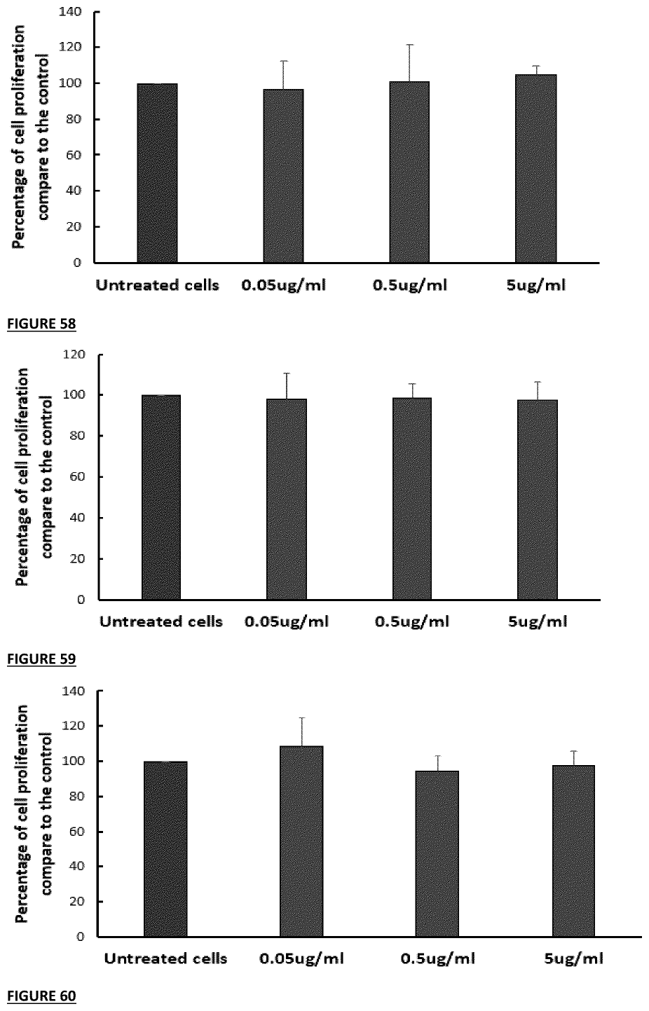

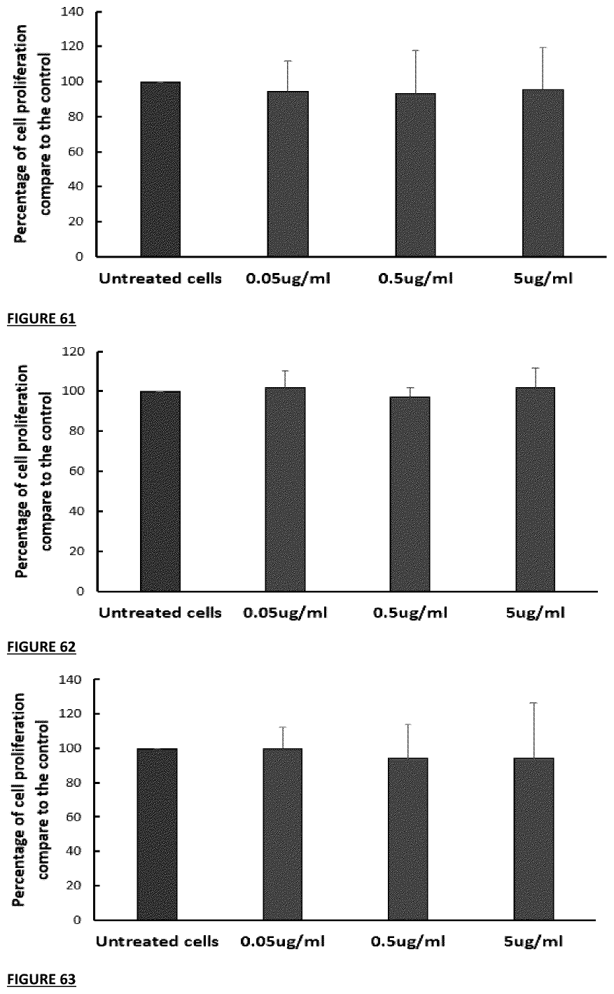

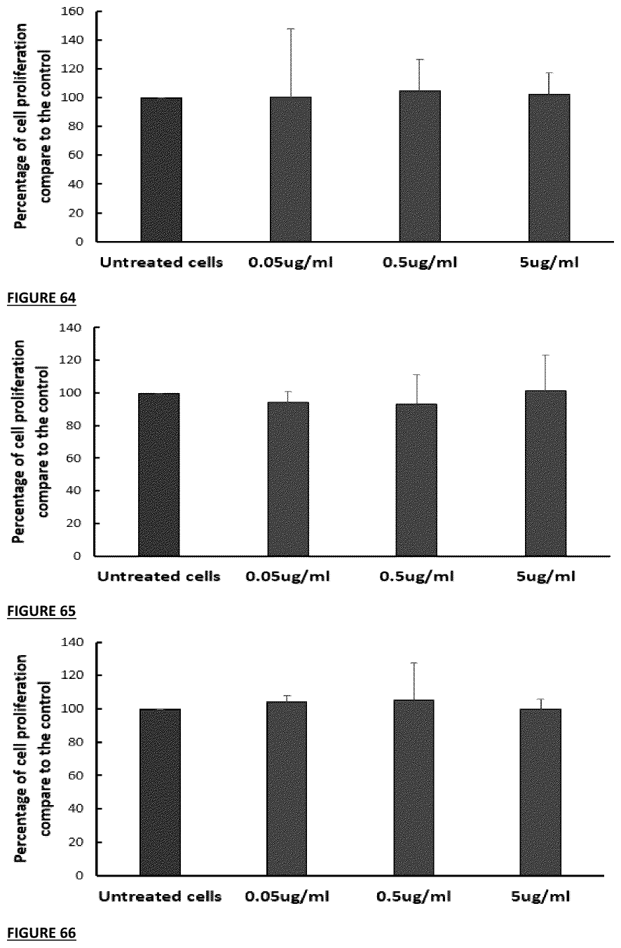

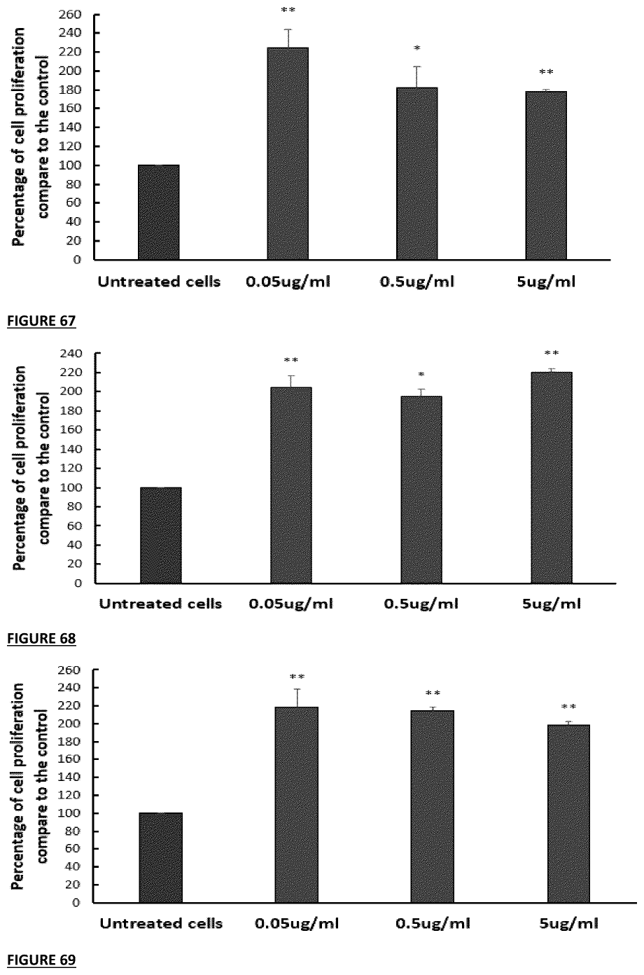

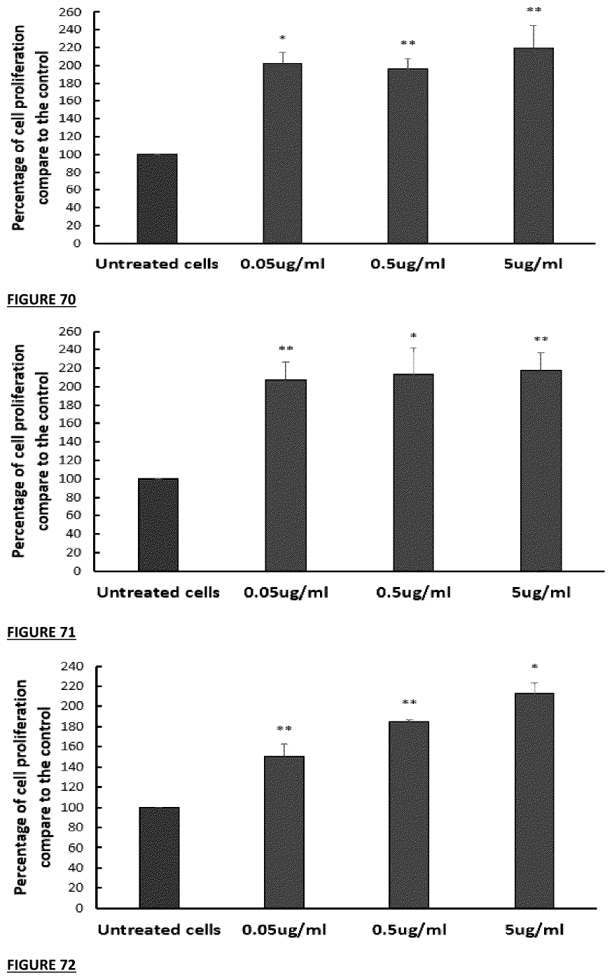

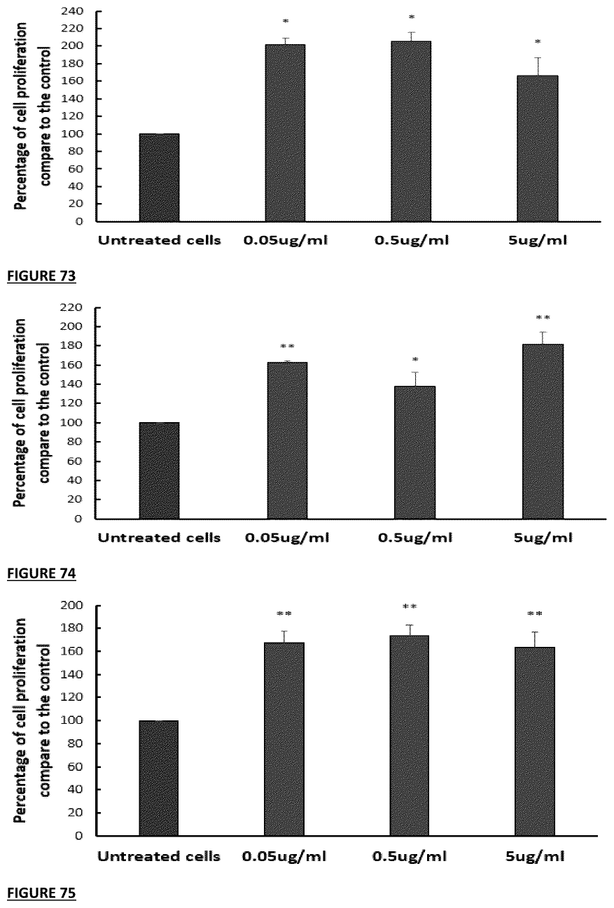

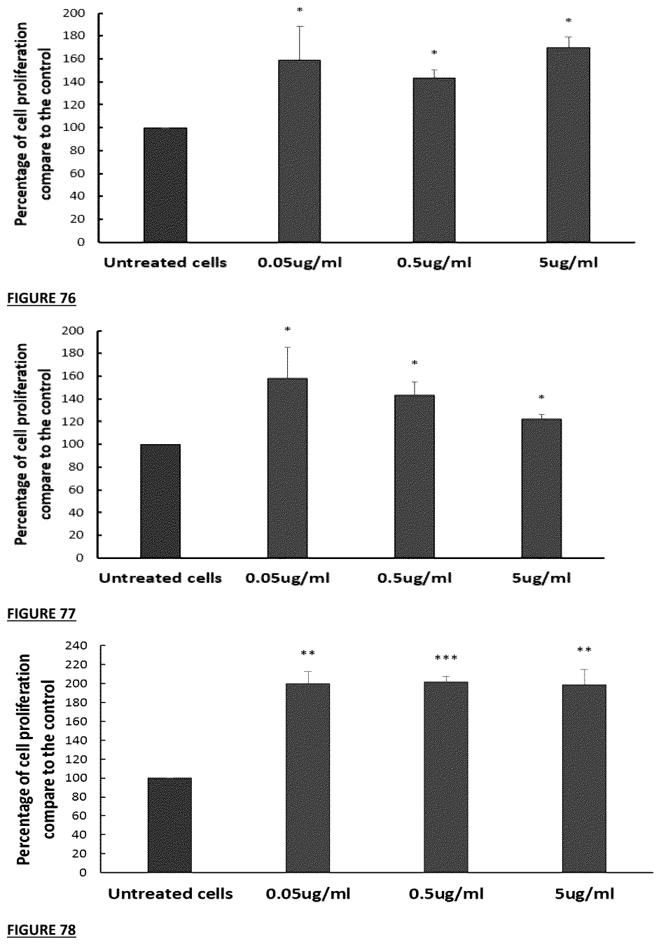

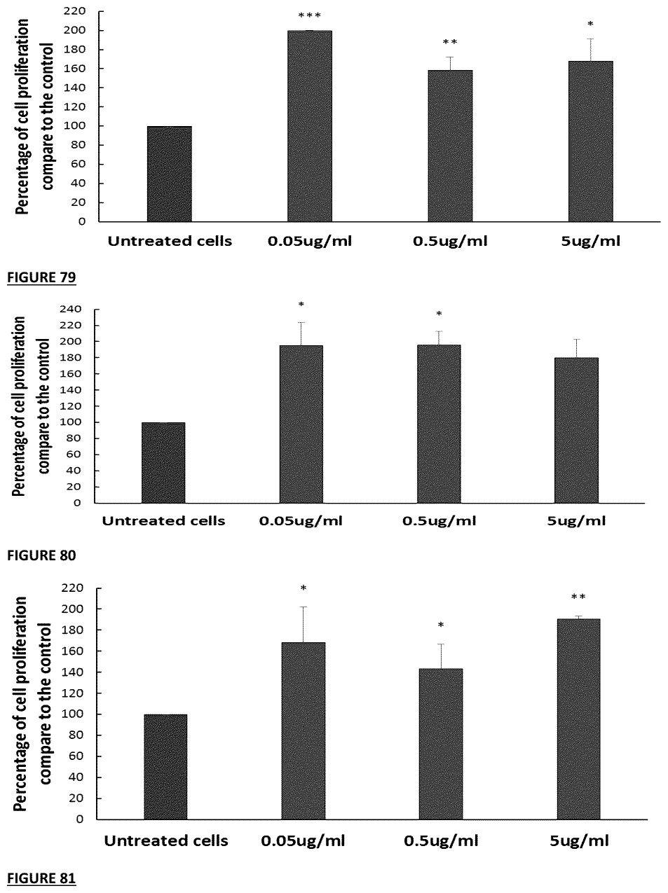

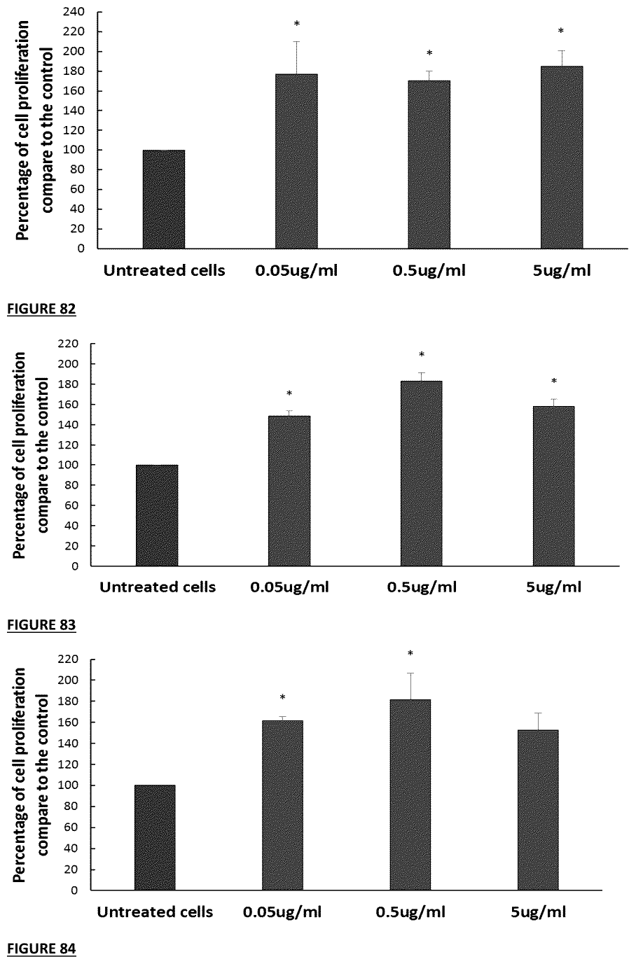

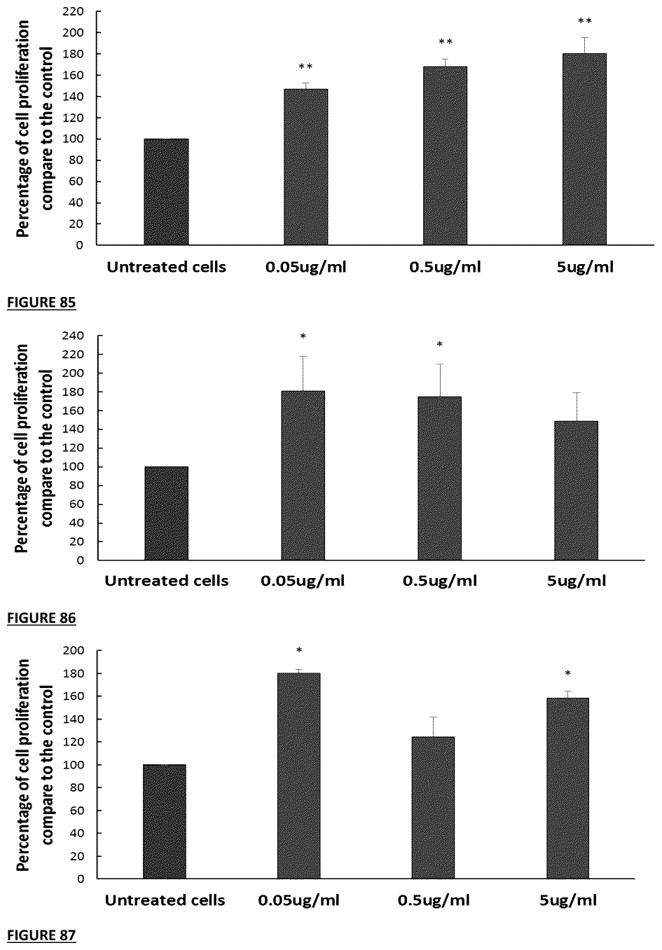

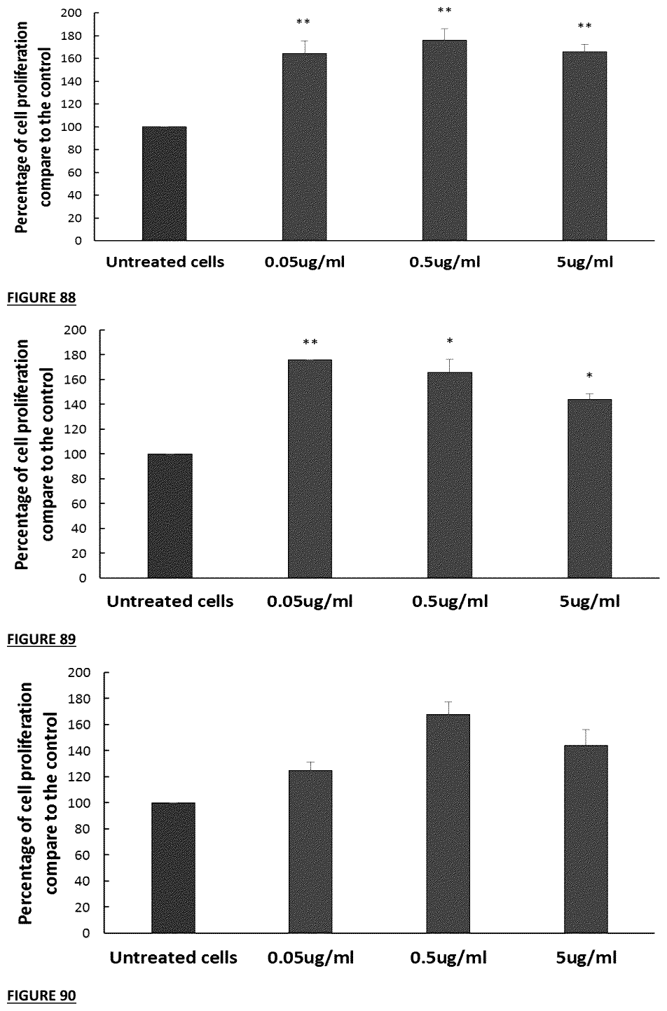

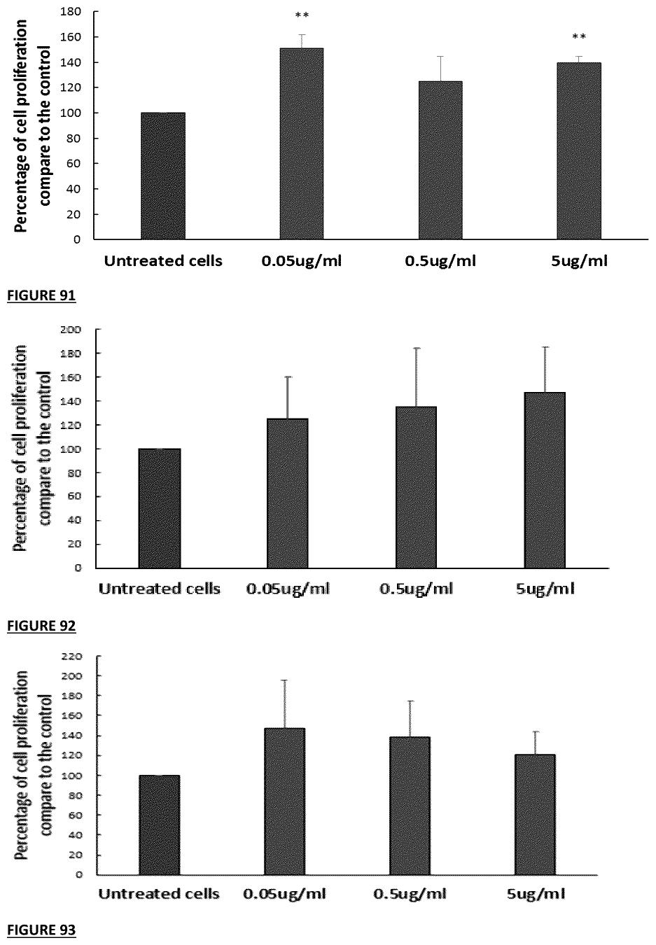

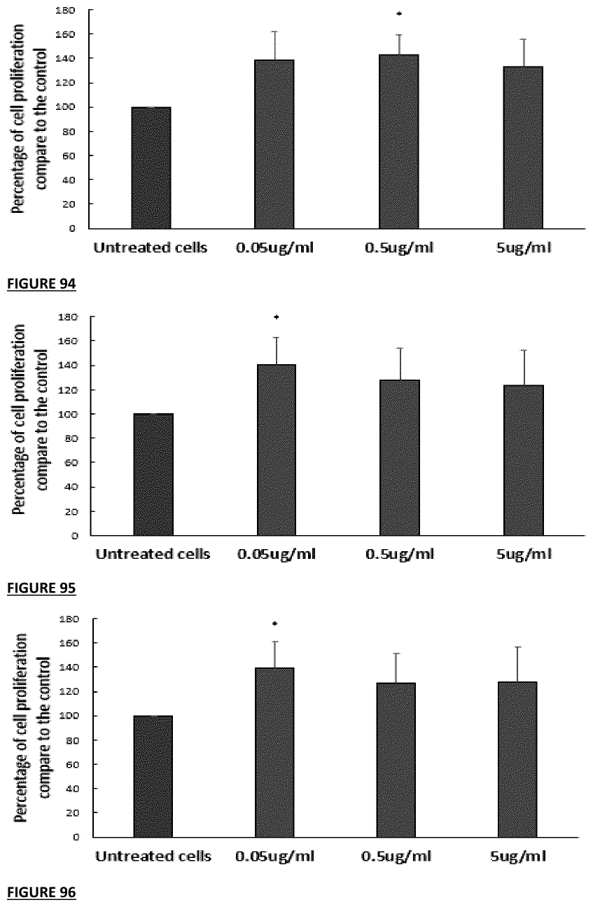

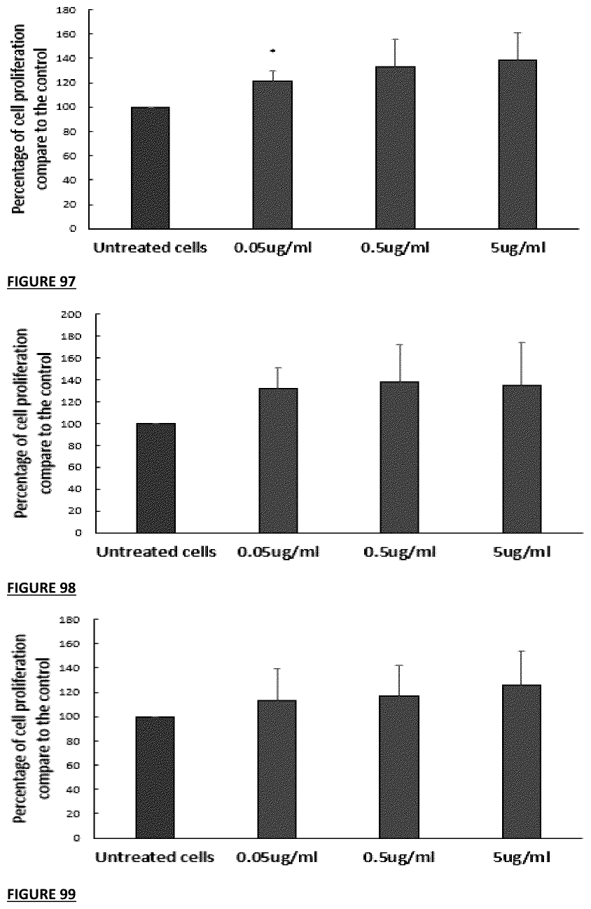

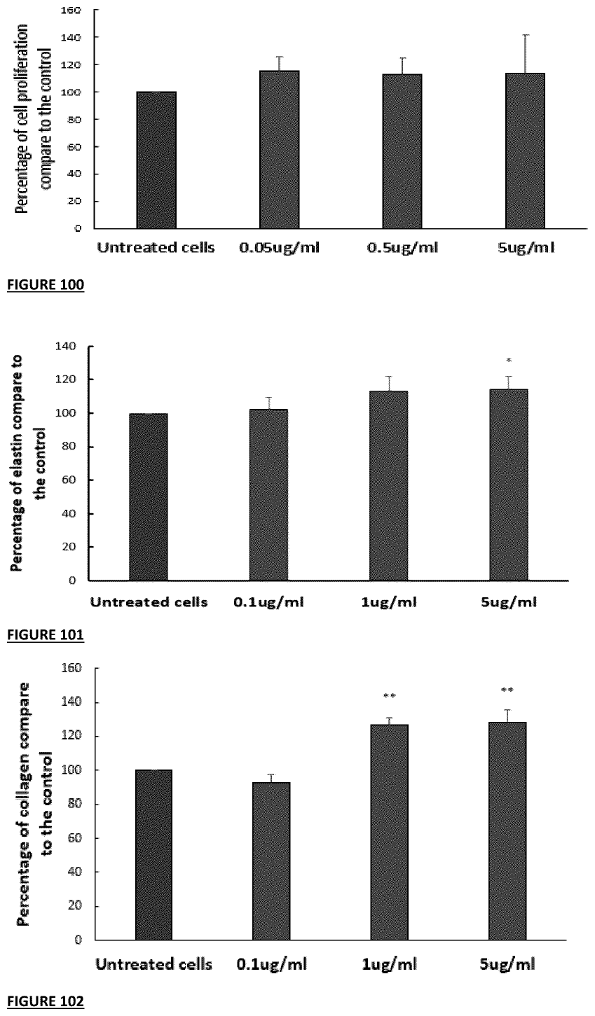

The results are shown in FIGS. 1-100 and Table 1 below.

TABLE 1

| FIG. | SEQ ID | INCREASE IN |
|---|---|---|
| NO | NO: | PROLIFERATION |
| 1 | 121 | 48% |
| 2 | 105 | 40% |
| 3 | 249 | 30% |
| 4 | 226 | 30% |
| 5 | 84 | 20% |
| 6 | 330 | 18% |
| 7 | 181 | 33% |
| 8 | 83 | 32% |
| 9 | 247 | 28% |
| 10 | 97 | 26% |
| 11 | 74 | 29% |
| 12 | | |
| 13 | 168 | |
| 14 | 151 | |
| 15 | 470 | 119% |
| 16 | 257 | 118% |
| 17 | 256 | 117% |
| 18 | 457 | 114% |
| 19 | 499 | 113% |
| 20 | 253 | 112% |
| 21 | 222 | 110% |
| 22 | 272 | 97% |
| 23 | 252 | 111% |
| 24 | 248 | 86% |
| 25 | 472 | 77% |
| 26 | 365 | 58% |
| 27 | 502 | 68% |
| 28 | 496 | 51% |
| 29 | 98 | 49% |
| 30 | 454 | 38% |
| 31 | 85 | 35% |
| 32 | 453 | 25% |
| 33 | 158 | 21% |
| 34 | 464 | 18% |
| 35 | 73 | 16% |
| 36 | 359 | 15% |
| 37 | 124 | 15% |
| 38 | 112 | 15% |
| 39 | 733 | |
| 40 | 728 | |
| 41 | 727 | |
| 42 | 730 | |
| 43 | 731 | |
| 44 | 148 | |
| 45 | 343 | |
| 46 | 345 | |
| 47 | 484 | |
| 48 | 729 | |
| 49 | 456 | |
| 50 | 494 | |
| 51 | 723 | |
| 52 | 722 | |
| 53 | 498 | |
| 54 | 475 | 13% |
| 55 | 718 | |
| 56 | 337 | 8% |
| 57 | 500 | 6% |
| 58 | 717 | |
| 59 | 297 | |
| 60 | 340 | |
| 61 | 719 | |
| 62 | 726 | |
| 63 | 725 | |
| 64 | 724 | |
| 65 | 720 | |
| 66 | 721 | |
| 67 | 503 | 125% |
| 68 | 474 | 121% |
| 69 | 504 | 119% |
| 70 | 114 | 119% |
| 71 | 505 | 118% |
| 72 | 482 | 113% |
| 73 | 479 | 106% |
| 74 | 477 | 81% |
| 75 | 410 | 73% |
| 76 | 475 | 69% |
| 77 | 497 | 58% |
| 78 | 480 | 102% |
| 79 | 463 | 100% |
| 80 | 465 | 96% |
| 81 | 467 | 90% |
| 82 | 461 | 85% |
| 83 | 341 | 83% |
| 84 | 468 | 82% |
| 85 | 285 | 81% |
| 86 | 496 | 81% |
| 87 | 146 | 80% |
| 88 | 478 | 76% |
| 89 | 452 | 76% |
| 90 | 495 | 68% |
| 91 | 403 | 51% |
| 92 | 455 | 47% |
| 93 | 270 | 47% |
| 94 | 501 | 43% |
| 95 | 473 | 41% |
| 96 | | 39% |
| 97 | 471 | 38% |
| 98 | 460 | 38% |
| 99 | 93 | 26% |
| 100 | 462 | 15% |

FIG. 1 shows an increase in HGF cell proliferation of 48% when incubated with SEQ ID NO: 121.

FIG. 2 shows an increase in HGF cell proliferation of 40% when incubated with SEQ ID NO: 105.

FIG. 3 shows an increase in HGF cell proliferation of 30% when incubated with SEQ ID NO: 249.

FIG. 4 shows an increase in HGF cell proliferation of 30% when incubated with SEQ ID NO: 226.

FIG. 5 shows an increase in HGF cell proliferation of 20% when incubated with SEQ ID NO: 84.

FIG. 6 shows an increase in HGF cell proliferation of 18% when incubated with SEQ ID NO: 330.

FIG. 7 shows an increase in HGF cell proliferation of 33% when incubated with SEQ ID NO: 181.

FIG. 8 shows an increase in HGF cell proliferation of 32% when incubated with SEQ ID NO: 83.

FIG. 9 shows an increase in HGF cell proliferation of 28% when incubated with SEQ ID NO: 247.

FIG. 10 shows an increase in HGF cell proliferation of 26% when incubated with SEQ ID NO: 97.

FIG. 11 shows an increase in HGF cell proliferation of 29% when incubated with SEQ ID NO: 74.

FIG. 15 shows an increase in HGF cell proliferation of 119% when incubated with SEQ ID NO: 470.

FIG. 16 shows an increase in HGF cell proliferation of 118% when incubated with SEQ ID NO: 257.

FIG. 17 shows an increase in HGF cell proliferation of 117% when incubated with SEQ ID NO: 256.

FIG. 18 shows an increase in HGF cell proliferation of 114% when incubated with SEQ ID NO: 457.

FIG. 19 shows an increase in HGF cell proliferation of 113% when incubated with SEQ ID NO: 499.

FIG. 20 shows an increase in HGF cell proliferation of 112% when incubated with SEQ ID NO: 253.

FIG. 21 shows an increase in HGF cell proliferation of 110% when incubated with SEQ ID NO: 222.

FIG. 22 shows an increase in HGF cell proliferation of 97% when incubated with SEQ ID NO: 272.

FIG. 23 shows an increase in HGF cell proliferation of 111% when incubated with SEQ ID NO: 252.

FIG. 24 shows an increase in HGF cell proliferation of 86% when incubated with SEQ ID NO: 248.

FIG. 25 shows an increase in HGF cell proliferation of 77% when incubated with SEQ ID NO: 472.

FIG. 26 shows an increase in HGF cell proliferation of 58% when incubated with SEQ ID NO: 365.

FIG. 27 shows an increase in HGF cell proliferation of 68% when incubated with SEQ ID NO: 502.

FIG. 28 shows an increase in HGF cell proliferation of 51% when incubated with SEQ ID NO: 496.

FIG. 29 shows an increase in HGF cell proliferation of 49% when incubated with SEQ ID NO: 98.

FIG. 30 shows an increase in HGF cell proliferation of 38% when incubated with SEQ ID NO: 454.

FIG. 31 shows an increase in HGF cell proliferation of 35% when incubated with SEQ ID NO: 85.

FIG. 32 shows an increase in HGF cell proliferation of 25% when incubated with SEQ ID NO: 453.

FIG. 33 shows an increase in HGF cell proliferation of 21% when incubated with SEQ ID NO: 158.

FIG. 34 shows an increase in HGF cell proliferation of 18% when incubated with SEQ ID NO: 464.

FIG. 35 shows an increase in HGF cell proliferation of 16% when incubated with SEQ ID NO: 73.

FIG. 36 shows an increase in HGF cell proliferation of 15% when incubated with SEQ ID NO: 359.

FIG. 37 shows an increase in HGF cell proliferation of 15% when incubated with SEQ ID NO: 124.

FIG. 38 shows an increase in HGF cell proliferation of 15% when incubated with SEQ ID NO: 112.

FIG. 54 shows an increase in HGF cell proliferation of 13% when incubated with SEQ ID NO: 475.

FIG. 56 shows an increase in HGF cell proliferation of 8% when incubated with SEQ ID NO: 337.

FIG. 57 shows an increase in HGF cell proliferation of 6% when incubated with SEQ ID NO: 500.

FIG. 67 shows an increase in HGF cell proliferation of 125% when incubated with SEQ ID NO: 503.

FIG. 68 shows an increase in HGF cell proliferation of 121% when incubated with SEQ ID NO: 474.

FIG. 69 shows an increase in HGF cell proliferation of 119% when incubated with SEQ ID NO: 504.

FIG. 70 shows an increase in HGF cell proliferation of 119% when incubated with SEQ ID NO: 114.

FIG. 71 shows an increase in HGF cell proliferation of 118% when incubated with SEQ ID NO: 505.

FIG. 72 shows an increase in HGF cell proliferation of 113% when incubated with SEQ ID NO: 482.

FIG. 73 shows an increase in HGF cell proliferation of 106% when incubated with SEQ ID NO: 479.

FIG. 74 shows an increase in HGF cell proliferation of 81% when incubated with SEQ ID NO: 477.

FIG. 75 shows an increase in HGF cell proliferation of 73% when incubated with SEQ ID NO: 410.

FIG. 76 shows an increase in HGF cell proliferation of 69% when incubated with SEQ ID NO: 475.

FIG. 77 shows an increase in HGF cell proliferation of 58% when incubated with SEQ ID NO: 497.

FIG. 78 shows an increase in HGF cell proliferation of 102% when incubated with SEQ ID NO: 480.

FIG. 79 shows an increase in HGF cell proliferation of 100% when incubated with SEQ ID NO: 463.

FIG. 80 shows an increase in HGF cell proliferation of 96% when incubated with SEQ ID NO: 465.

FIG. 81 shows an increase in HGF cell proliferation of 90% when incubated with SEQ ID NO: 467.

FIG. 82 shows an increase in HGF cell proliferation of 85% when incubated with SEQ ID NO: 461.

FIG. 83 shows an increase in HGF cell proliferation of 83% when incubated with SEQ ID NO: 341.

FIG. 84 shows an increase in HGF cell proliferation of 82% when incubated with SEQ ID NO: 468.

FIG. 85 shows an increase in HGF cell proliferation of 81% when incubated with SEQ ID NO: 485.

FIG. 86 shows an increase in HGF cell proliferation of 81% when incubated with SEQ ID NO: 496.

FIG. 87 shows an increase in HGF cell proliferation of 80% when incubated with SEQ ID NO: 146.

FIG. 88 shows an increase in HGF cell proliferation of 76% when incubated with SEQ ID NO: 478.

FIG. 89 shows an increase in HGF cell proliferation of 76% when incubated with SEQ ID NO: 452.

FIG. 90 shows an increase in HGF cell proliferation of 68% when incubated with SEQ ID NO: 495.

FIG. 91 shows an increase in HGF cell proliferation of 51% when incubated with SEQ ID NO: 403.

FIG. 92 shows an increase in HGF cell proliferation of 47% when incubated with SEQ ID NO: 455.

FIG. 93 shows an increase in HGF cell proliferation of 47% when incubated with SEQ ID NO: 270.

FIG. 94 shows an increase in HGF cell proliferation of 43% when incubated with SEQ ID NO: 501.

FIG. 95 shows an increase in HGF cell proliferation of 41% when incubated with SEQ ID NO: 473.

FIG. 97 shows an increase in HGF cell proliferation of 38% when incubated with SEQ ID NO: 471.

FIG. 98 shows an increase in HGF cell proliferation of 38% when incubated with SEQ ID NO: 460.

FIG. 99 shows an increase in HGF cell proliferation of 26% when incubated with SEQ ID NO: 93.

FIG. 100 shows an increase in HGF cell proliferation of 15% when incubated with SEQ ID NO: 462.

### Example 2—Collagen Production Assay

Hydroxyproline in tissue preparations is a direct measure of the amount of collagen present. FIRELISA Human Hydroxyproline ELISA kit assay is designed to measure hydroxyproline in tissue or peptide compositions.

Human Dermal Fibroblasts (HDF Sigma 10605a) were seeded in 24 well plates at 50,000 cells per well in DMEM containing 10% fetal calf serum (FCS), 1% Pen/strep, 1% L-glutamine and allowed to adhere for 24 h.

Following the initial 24 h incubation the cells were incubated with 5 μg/ml, 1 μg/ml or 0.1 μg/ml synthetic peptide for 96 h respectively.

After treatment the cells were lysed using 4 freeze thaw cycles in liquid nitrogen. The lysed cells were centrifuged and 50 μl/ml of each supernatant was assayed using the FIRELISA Human Hydroxyproline ELISA kit. All steps were carried out according to the manufacturer's instructions.

Results were calculated as a percentage of the untreated control. An increase in optical density reading indicates an increase collagen content.

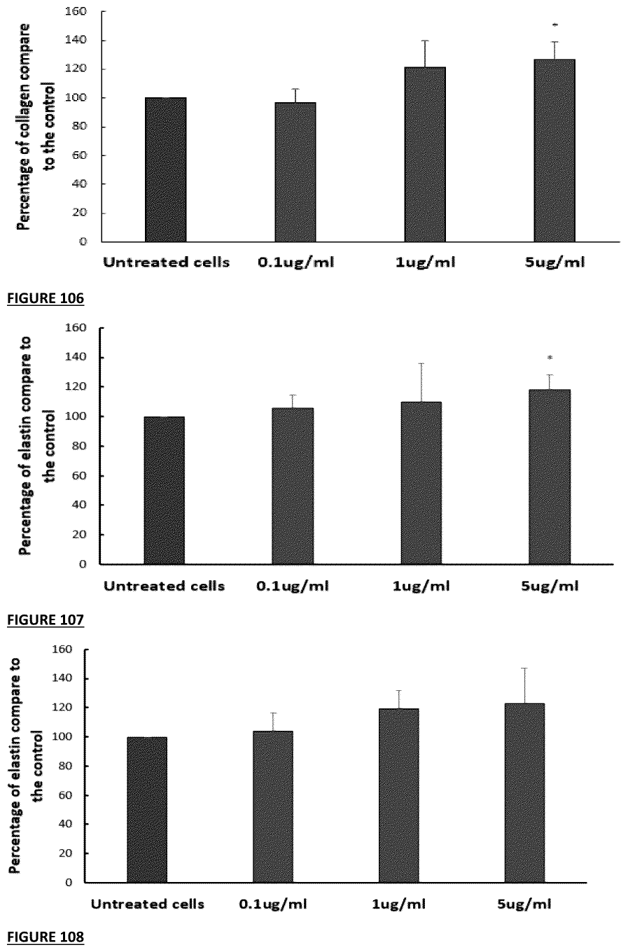

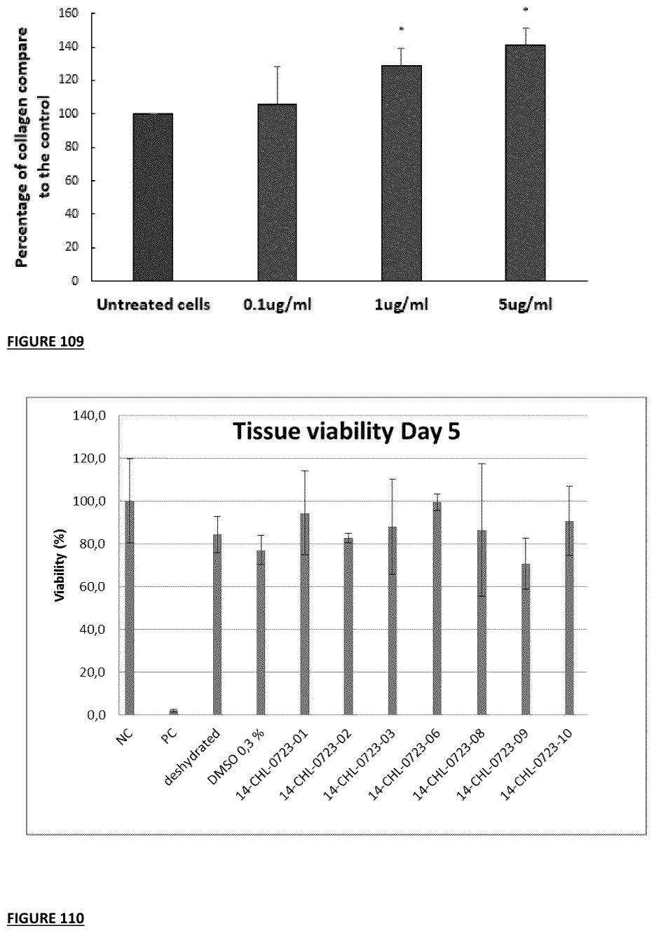

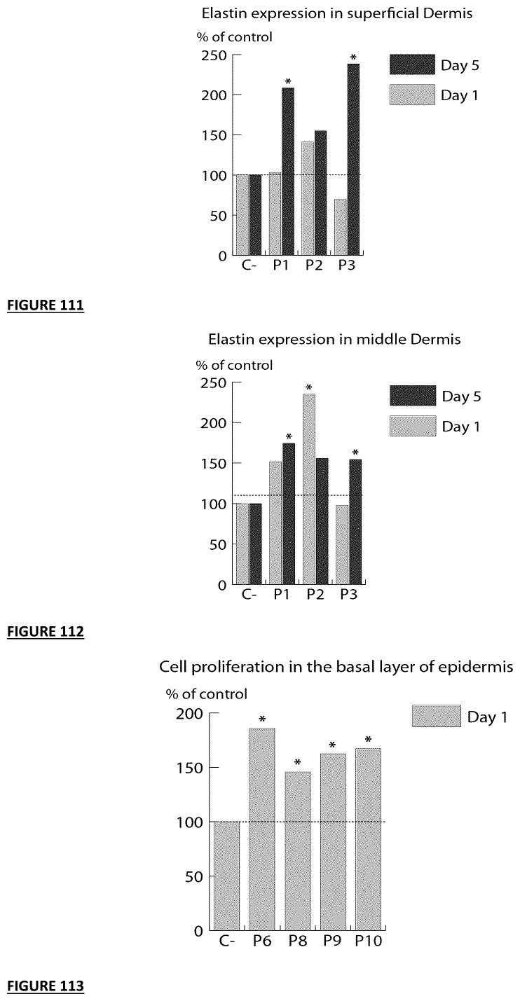

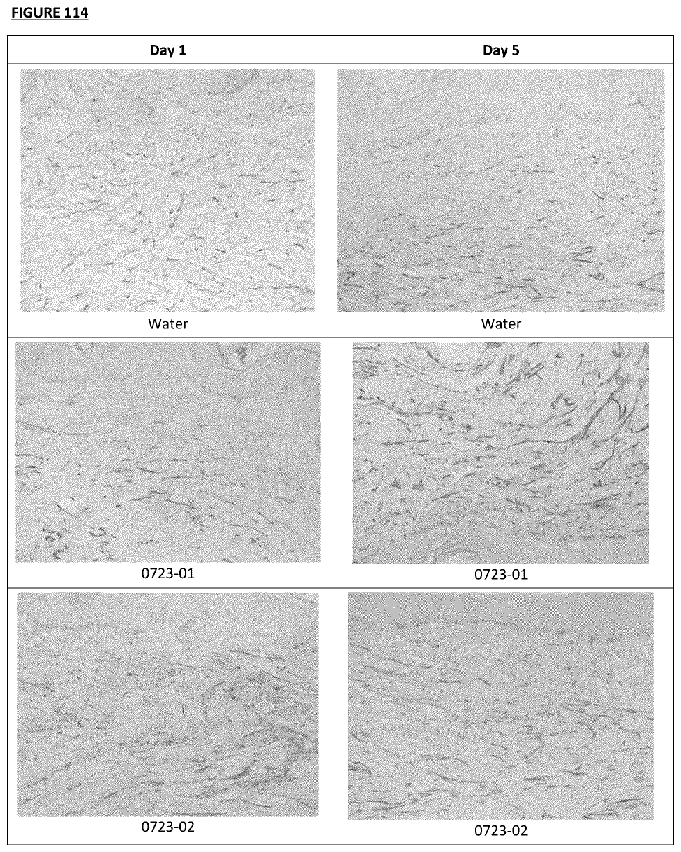

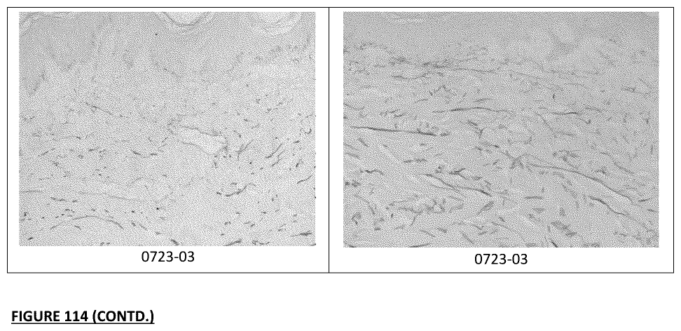

The results are shown in FIGS. 102, 104, 106 and 109

### Example 3—Elastin Production Assay

Elastin is a highly elastic protein in connective tissue and allows many tissues in the body to resume their shape after stretching or contracting. FIRELISA Human Elastin ELISA kit assay is designed to measure Elastin in tissue or protein/peptide compositions.

Human Dermal Fibroblasts (HDF) were seeded in 24 well plates at 50,000 cells per well in DMEM containing 10% fetal calf serum (FCS), 1% Pen/strep, 1% L-glutamine and allowed to adhere for 24 h.

Following the initial 24 h incubation the cells were incubated with 5 μg/ml, 1 μg/ml or 0.1 μg/ml synthetic peptide for 96 h respectively.

After treatment the cells were lysed using 4 freeze thaw cycles in liquid nitrogen. The lysed cells were centrifuged and 50 μl/ml of each supernatant was assayed using the FIRELISA Human Elastin ELISA kit. All steps were carried out according to the manufacturer's instructions.

Results were calculated as a percentage of the untreated control. An increase in optical density reading indicates an increase collagen content.

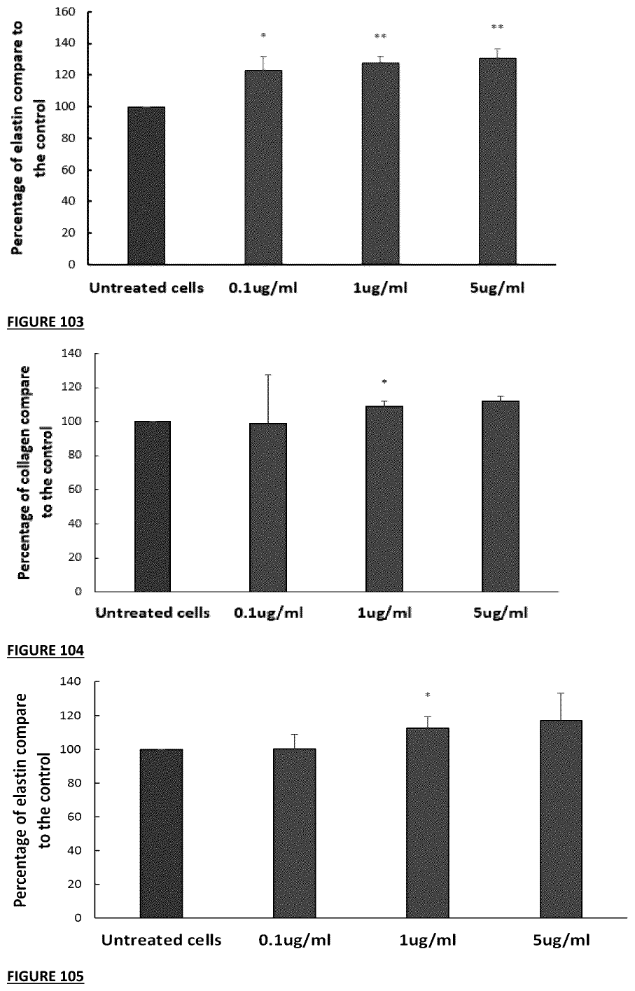

The results are shown in FIGS. 101, 103, 105, 107, 108 and 109.

### Example 4—Elastin and Cell Proliferation Assays

TABLE 2
Test items. Orange bands correspond to samples dissolved into DMSO 0.3%
instead of water.

| | | | | | | Intertek | | |
|---|---|---|---|---|---|---|---|---|
| Item | Denomination | Concentration | | Provider | Nature | reference | Solubility | Storage |
| Peptide 1 | E_280_PJ | 20 | μM | Nuritas | Peptide | 14-CHL- | Water | −80° C. |
| | | | | | 0723-01 | | | |
| Peptide 2 | I_222two_IN | 20 | μM | Nuritas | Peptide | 14-CHL- | Water | Ambient |
| | | | | | 0723-02 | | | |
| Peptide 3 | E_134_two_IN | 20 | μM | Nuritas | Peptide | 14-CHL- | Water | −80° C. |
| | | | | | 0723-03 | | | |
| Peptide 4 | E_30two_IN | 20 | μM | Nuritas | Peptide | 14-CHL- | Water | −80° C. |
| | | | | | 0723-04 | | | |
| Peptide 5 | E_121two_IN | 20 | μM | Nuritas | Peptide | 14-CHL- | Water | −80° C. |
| | | | | | 0723-05 | | | |
| Peptide 6 | I_10two_IN | 20 | μM | Nuritas | Peptide | 14-CHL- | DMSO | −80° C. |
| | | | | | 0723-06 | 0.3% | | |
| Peptide 7 | I_41two_IN | 20 | μM | Nuritas | Peptide | 14-CHL- | DMSO | −80° C. |
| | | | | | 0723-07 | 0.3% | | |
| Peptide 8 | E_41_PJ | 10 | μM* | Nuritas | Peptide | 14-CHL- | Water | −80° C. |
| | | | | | 0723-08 | | | |
| Composition | E_2_IN | 500 | μg/mL | Nuritas | Composition | 14-CHL- | Water | −80° C. |
| P9 | | | | of peptides | 0723-09 | | | |
| Composition | I_2_IN | 500 | μg/mL | Nuritas | Composition | 14-CHL- | Water | −80° C. |
| P10 | | | | of peptides | 0723-10 | | | |

### Equipment

Incubator, Flow Laminar Chamber, Sterile Polished Plastic Rod, Pipettor, Maintenance medium, Plate 6 well, Plate 24 well.

### Reagents

MTT, PBS, SDS, Formaldehyde, Xylene, Ethanol absolute, Dulbecco's phosphate-buffered saline (DPBS), Metal Enhanced DAB substrate kit, ABC peroxidase staining kit, Citric acid, Sodium hydroxide 2N, Hydrogen peroxide 30%, Anti-Filaggrin, Anti-rabbit IgG-Biotin, Tween 20.

### Test System

Nature: Human skin tissue 5 mm diameter

Batch number: EXP004050B009 and EXP004050B011

Provider: Laboratoire Biopredic International-8-18 rue Jean Pecker-35000 Rennes-France. Tel: +33 (0) 2.99.14.36.14-Fax: +33 (0) 2.99.54.44.72.

Certificates of analysis are present in Annex 1.

Two batches are used for the assay. Batch EXP004050B005 is used for experiment day 1, and Batch EXP004050B006 is used for experiment day 5.

### Maintenance Medium

Maintenance Medium: Batch no: MIL 218 C Provider: Laboratoire Biopredic International-8-18 rue Jean Pecker-35000 Rennes-France.

### Peptides Tested

P1:
SEQ ID NO: 283
P2:
SEQ ID NO: 246
P3:
SEQ ID NO: 284
P4:
RPYYSNAPQEIF
P5:
VLLEQQEQEPQH
P6:
SEQ ID NO: 245
P7:
QQYGIAASPFLQSAA
P8:
SEQ ID NO: 42

### Compositions Tested

P9 (14-CHL-0723-09) is the Pea composition (SEQ ID NOs: 50, 85, 74, 140, 82, 136, 189, 77, 169, 149, 171, 178, 143, 127, 190, 141, 147, 133, 186, 125, 122, 119, 87, 90, 86, 89, 138, 129, 123, 120, 117, 113, 110, 121, 105, 98, 55, 161, 19, 317, 135, 130, 146, 177, 160, 170, 188, 83, 78, 36, 96, 159, 26, 330, 168, 148, 184, 151, 151, 165, 114, 284)

P10 (14-CHL-0723-010) is the Rice composition (SEQ ID NOs: 245, 246, 263, 250, 257, 259, 276, 255, 251, 264, 256, 266, 274, 270, 269, 356, 245, 380, 262, 258, 356, 218, 252, 358, 271, 253, 344, 275, 272, 226, 224, 220, 248, 261, 265, 373, 375, 247, 249, 363, 273, 343, 273, 362)

### Application Method

Skin explants were prepared from abdominal plastic surgery. Some explants were delipidated with alcohol to obtain a dehydrated skin.

These explants were maintained in maintenance medium supplied by the provider Bioprédic International for 5 days. Test items are applied twice per day with 5 μL per explant.

At the end of the test, viabilities controls are realized with the MTT on two explants, the third explant is fixed in the formaldehyde 4% for histology and cell staining.

For each time of analysis (D1 and D5), histologies on delipidated explants, treated explants with test items, the DMSO 0.3% control and water control, are performed.

After receipt in the laboratory, each skin explant in the maintenance medium is delipidated with 5 μL alcohol during 3 hours.

After 3 hours, all skin explants are treated two per day with test items, and they are incubated at 37° C.+/−2° C., 5% CO2 for 1 day or 5 days.

Integrity of the system is realized at day 1 and day 5 with a viability control with MTT.

### Immunostaining

Histology is realized by the laboratory Gredeco and the immunostaining to elastin and Ki67 are realized by the same laboratory. Immunostaining to filaggrin is realized by the laboratory

### Intertek.

The detection of elastin (rabbit monoclonal antibody, clone P15502, LSBio) is performed using an immunoperoxidase technique two layers (ABC kit, Vector Laboratories) and revealed by AEC (3-amino-9-éthylcarbazole). The immunohistochemical staining intensity in the elastic fibers is evaluated using a semi-quantitative histological score.

Epithelial proliferation was analyzed by immunohistochemistry using anti-Ki67 antibody. Immunodetection was performed using an indirect immunoperoxidase technique three layers, amplified (DAKO kit) and revealed by AEC (3-Amino-9-ethylcarbazole). Counting the number of labeled cells (keratinocytes of the basal layer of the epidermis) is performed and provides the total number of basal cells to calculate the % of labeled cells.

The specific staining of filaggrin is performed with an immunoperoxidase staining (ABC kit, Fisher). The intensity of immunohistochemical marker in the epidermis is evaluated relative to the negative control of the solvent (Water or DMSO 0.3%).

### C: Results

### Viability Control

The integrity control and the viability control are present in FIG. 1. These controls do allow to validate the assay system. The viability is >50% for test items, and they do not show a cytotoxicity according to the test.

### Immunostaining

### Elastin Expression

The elastic fibers of the dermis were revealed by staining with the catechin and morphometrically quantified by analysis by computer-assisted image. The percentage area taken up by elastic fibers in the dermis was calculated in the dermis and the average superficial dermis. Results are presented in FIGS. 101, 105, 107, and 108.

TABLE 3
Morphometric quantification of elastic fibers in the superficial
dermis (%) and middle dermis. Orange bands correspond to
samples dissolved into DMSO 0.3% instead of water.

| | Morphometric | | Morphometric | |
|---|---|---|---|---|
| | quantification | | quantification | |
| | of elastic fibers | | of elastic fibers | |
| | in the superficial | | in the middle | |
| | dermis (%) | | dermis (%) | |
| Conditions | D1 | D5 | D1 | D5 |
| Dehydrated with alcohol | 4.31 | 4.9 | 5 | 5.83 |
| and hydrated with water | | | | |
| Dehydrated | 2.38 | 7.26 | 4.39 | 9.59 |
| EGF (Epidermal Growth | 3.64 | 5.61 | 5.68 | 6.61 |
| Factor) 10 ng/mL | | | | |
| Dehydrated with alcohol | 3.76 | 7.24 | 6 | 10.36 |
| and hydrated with | | | | |
| DMSO 0.3% | | | | |
| 0723.01 | 4.45 | 10.21 | 7.59 | 10.17 |
| 0723.02 | 6.09 | 7.59 | 11.75 | 9.08 |
| 0723.03 | 3 | 11.68 | 4.9 | 9 |
| 0723.04 | 3.28 | 8.94 | 5.22 | 9 |
| 0723.05 | 6.34 | 6.26 | 8.8 | 6.61 |
| 0723.06 | 3.8 | 4.03 | 4.54 | 8.67 |
| 0723.07 | 4 | 5.15 | 6.46 | 5.33 |
| 0723.08 | 2.7 | 5.32 | 3.52 | 7.27 |
| 0723.09 | 3.26 | 8.26 | 5.75 | 7.92 |
| 0723.10 | 4.1 | 8 | 5.73 | 8.34 |

Under the experimental conditions of the study, 0723-1 and 0723-3 samples show an increase by twice of elastic fibers in the superficial dermis compared to control water (Figure), and an increase in the middle dermis compared to the water control at D5.

The 0723-2 sample shows an increase doubled in the middle dermis at day 1 compared to control water and an increase at day 5.

### Ki67 Expression

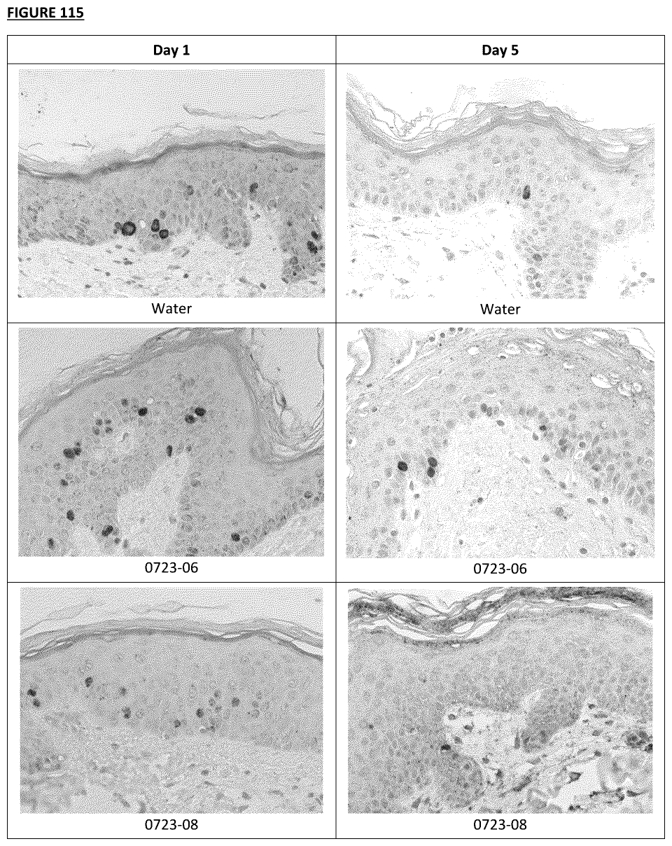

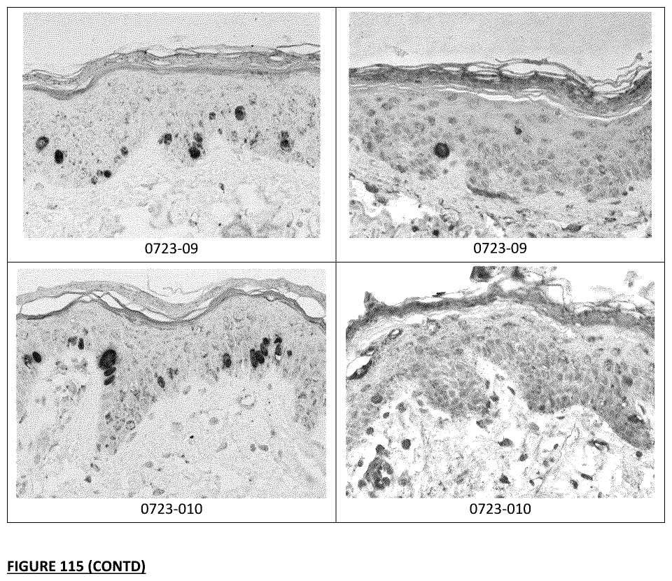

The results of the immunohistochemical analysis of Ki67 are reported in Table 4 and expressed as % of labelled at the basal layer of the epidermis. The FIG. 115 shows the percentage of Ki 67 cells compared to negative controls (water or DMSO). Immunohistochemical analysis of mitotic activity is shown in annex 4 with a reminder of the average for each analysed conditions.

TABLE 4
% of Ki67 positive cells in the basal layer of the epidermis. Orange bands
correspond to samples dissolved into DMSO 0.3% instead of water.

| Conditions | D1 | D5 |
|---|---|---|
| Dehydrated with alcohol and | 19.09 | 3.53 |
| hydrated with water | | |
| Dehydrated | 17.05 | 1.76 |
| EGF (Epidermal Growth Factor) 10 ng/mL | 25.11 | 4.2 |
| Dehydrated with alcohol and hydrated with | 17.2 | 2.61 |
| DMSO 0.3% | | |
| 0723.01 | 18.57 | 3.92 |
| 0723.02 | 19.61 | 6.73 |
| 0723.03 | 22.01 | 10.04 |
| 0723.04 | 14.97 | 11.36 |
| 0723.05 | 9.48 | 3.08 |
| 0723.06 | 31.97 | 5.04 |
| 0723.07 | 22.22 | 5.26 |
| 0723.08 | 27.83 | 5.72 |
| 0723.09 | 31.02 | 2.4 |
| 0723.10 | 31.94 | 3.57 |

Under the experimental conditions of the study, test item 0723-06, 0723-08, 0723-09 and 0723-010 show an increase in the number of mitotic cells compared to EGF at day 1. A decrease in the mitotic index was observed on day 5 compared to day 1 for all analysed conditions.

The decrease in this cell staining on day 5 is caused by the model. Indeed, after approximately 3 days cell turnover is exhausted on this model.

The invention is not limited to the embodiments hereinbefore described which may be varied in construction and detail without departing from the spirit of the invention.

SEQUENCES

PROTEIN: P13918-2-PEA
MAATTMKASFPLLMLMGISFLASVCVSSRSDPQNPFIFKSNKFQTLFENENGHIRLLQKFDQRSKIFENLQNYRLLEYKSKPHTIFLPQHTDADYI
LVVLSGKAILTVLKPDDRNSFNLERGDTIKLPAGTIAYLVNRDDNEELRVLDLAIPVNRPGQLQSFLLSGNQNQQNYLSGFSKNILEASFNTDYEE
IEKVLLEEHEKETQHRRSLKDKRQQSQEENVIVKLSRGQIEELSKNAKSTSKKSVSSESEPFNLRSRGPIYSNEFGKFFEITPEKNPQLQDLDIFVNS
VEIKEGSLLLPHYNSRAIVIVTVNEGKGDFELVGQRNENQQEQRKEDDEEEEQGEEEINKQVQNYKAKLSSGDVFVIPAGHPVAVKASSNLDLL
GFGINAENNQRNFLAGDEDNVISQIQRPVKELAFPGSAQEVDRILENQKQSHFADAQPQQRERGSRETRDRLSSV [SEQ ID 1]
PEPTIDE: QNYLSGFSKNILE [SEQ ID 15]
PEPTIDE: TIKLPAGTIAYLVNRDDNEE [SEQ ID 16]
PEPTIDE: LAIPVNRPGQLQSFL [SEQ ID 17]
PEPTIDE: AIPVNRPGQLQ [SEQ ID 18]
PEPTIDE: PAGHPVAVK [SEQ ID 19]
PEPTIDE: VQNYKAKLSSGDVFVIPAG [SEQ ID 20]
PEPTIDE: NNQRNFLAGDEDNVISQIQRPVKE [SEQ ID 21]
PEPTIDE: INKQVQNYKAKLSSGDVFVIPAG [SEQ ID 22]
PEPTIDE: LAIPVNRPGQ [SEQ ID 23]
PEPTIDE: NFLAGDEDNVISQIQRPVKE [SEQ ID 24]
PEPTIDE: DLAIPVNRPGQLQSF [SEQ ID 25]
PEPTIDE: VIPAGHPVAVK [SEQ ID 26]
PEPTIDE: DTIKLPAGTIAYLVNRDDNEE [SEQ ID 27]
PEPTIDE: LAIPVNRPGQLQSF [SEQ ID 28]
PEPTIDE: KQVQNYKAKLSSGDVFVIPAG [SEQ ID 29]
PEPTIDE: RGDTIKLPAGTIAYLVNRDDNEE [SEQ ID 30]
PEPTIDE: FLAGDEDNVISQIQRPVKE [SEQ ID 31]
PEPTIDE: LAIPVNRPGQLQS [SEQ ID 32]
PEPTIDE: VLDLAIPVNRPGQLQ [SEQ ID 33]
PEPTIDE: DLAIPVNRPGQLQ [SEQ ID 34]
PEPTIDE: VFVIPAGHPVAVK [SEQ ID 35]
PEPTIDE: TIFLPQHTDADYILVVLSGK [SEQ ID 36]
PEPTIDE: NQRNFLAGDEDNVISQIQRPVKE [SEQ ID 37]
PEPTIDE: LAIPVNRPGQLQ [SEQ ID 38]
PEPTIDE: HPVAVKASSNLDLLGFG [SEQ ID 39]
PEPTIDE: LAIPVNRPGQL [SEQ ID 40]
PEPTIDE: DLAIPVNRPGQL [SEQ ID 41]
PEPTIDE: SKPHTIFLPQHTDADYILVVLSGK [SEQ ID 42]
PEPTIDE: FVIPAGHPVAVK [SEQ ID 43]
PEPTIDE: DLAIPVNRPGQLQS [SEQ ID 44]
PEPTIDE: SGDVFVIPAGHPVAVKASSNLD [SEQ ID 45]
PEPTIDE: AIPVNRPGQLQSF [SEQ ID 46]
PEPTIDE: ELAFPGSAQEVDR [SEQ ID 47]
PEPTIDE: LAIPVNRPGQLQSFLLSG [SEQ ID 48]
PEPTIDE: VFVIPAGHPVAVKASSNLDLLGFG [SEQ ID 49]
PEPTIDE: AGHPVAVK [SEQ ID 50]
PEPTIDE: HPVAVKASSNLDLLGFGINAE [SEQ ID 51]
PEPTIDE: LAIPVNRPGQLQSFLLSGNQNQ [SEQ ID 52]
PEPTIDE: SGDVFVIPAG [SEQ ID 53]
PEPTIDE: GSLLLPHYNSRAIVIVTVNE [SEQ ID 54]
PEPTIDE: NFLAGDEDNVISQIQRPVK [SEQ ID 55]
PEPTIDE: SGDVFVIPAGHPVA [SEQ ID 56]
PEPTIDE: GSLLLPHYNSRAIVIV [SEQ ID 57]
PEPTIDE: RGDTIKLPAGTIAYLVNRDD [SEQ ID 58]
PEPTIDE: SGDVFVIPAGHPVAVK [SEQ ID 59]
PEPTIDE: LSSGDVFVIPAGHPVAVK [SEQ ID 60]
PEPTIDE: LDLAIPVNRPGQL [SEQ ID 61]
PEPTIDE: AIPVNRPGQL [SEQ ID 62]
PEPTIDE: LAIPVNRPGQLQSFLL [SEQ ID 63]
PEPTIDE: PHTIFLPQHTDADYILVVLSGK [SEQ ID 64]
PEPTIDE: VFVIPAGHPVAVKASSNLD [SEQ ID 65]
PEPTIDE: LAIPVNRPGQLQSFLLS [SEQ ID 66]
PEPTIDE: VLDLAIPVNRPGQLQSF [SEQ ID 67]
PEPTIDE: AIPVNRPGQLQS [SEQ ID 68]
PEPTIDE: DTIKLPAGTIAYLVNRDDNE [SEQ ID 69]
PEPTIDE: NYKAKLSSGDVFVIPAG [SEQ ID 70]
PEPTIDE: GKAILTVLKPDDRNSFNLE [SEQ ID 71]
PEPTIDE: YKSKPHTIFLPQHTDAD [SEQ ID 72]
PEPTIDE: ASSNLDLLGFG [SEQ ID 73]
PEPTIDE: DEEEEQGEEEINK [SEQ ID 74]
PEPTIDE: YKSKPHTIFLPQHTD [SEQ ID 75]
PEPTIDE: VLDLAIPVNR [SEQ ID 76]
PEPTIDE: FFEITPEKNPQLQDLDIFVNSVEIK [SEQ ID 77]
PEPTIDE: TIFLPQHTDADYIL [SEQ ID 78]
PEPTIDE: SFLLSGNQNQQNYLSG [SEQ ID 79]
PEPTIDE: SFLLSGNQNQQNYLSGFS [SEQ ID 80]
PEPTIDE: NQQEQRKEDDEEEEQGEEE [SEQ ID 81]
PEPTIDE: EEQGEEEINK [SEQ ID 82]
PEPTIDE: SRGPIYSNE [SEQ ID 83]
PEPTIDE: EDDEEEEQGEEEINK [SEQ ID 84]
PEPTIDE: DDEEEEQGEEEINK [SEQ ID 85]
PEPTIDE: KEDDEEEEQGEEEIN [SEQ ID 86]
PEPTIDE: KEDDEEEEQGEE [SEQ ID 87]
PEPTIDE: QRKEDDEEEEQGEEE [SEQ ID 88]
PEPTIDE: KEDDEEEEQGEEEINK [SEQ ID 89]
PEPTIDE: KEDDEEEEQGEEE [SEQ ID 90]
PROTEIN: Q9M3X6-3-PEA
MATTIKSRFPLLLLLGIIFLASVVCVTYANYDEGSEPRVPAQRERGRQEGEKEEKRHGEWRPSYEKEEDEEEGQRERGRQEGEKEEKRHGEWR
PSYEKQEDEEEKQKYRYQREKEDEEEKQKYQYQREKKEQKEVQPGRERWEREEDEEQVDEEWRGSQRREDPEERARLRHREERTKRDRRHQ
REGEEEERSSESQERRNPFLFKSNKFLTLFENENGHIRLLQRFDKRSDLFENLQNYRLVEYRAKPHTIFLPQHIDADLILVVLSGKAILTVLSPNDR
NSYNLERGDTIKLPAGTTSYLVNQDDEEDLRLVDLVIPVNGPGKFEAFDLAKNKNQYLRGFSKNILEASYNTRYETIEKVLLEEQEKDRKRRQQG
EETDAIVKVSREQIEELKKLAKSSSKKSLPSEFEPINLRSHKPEYSNKFGKLFEITPEKKYPQLQDLDLFVSCVEINEGALMLPHYNSRAIVVLLVNEG
KGNLELLGLKNEQQEREDRKERNNEVQRYEARLSPGDVVIIPAGHPVAITASSNLNLLGFGINAENNERNFLSGSDDNVISQIENPVKELTFPGS
VQEINRLIKNQKQSHFANAEPEQKEQGSQGKRSPLSSILGTFY [SEQ ID 2]
PEPTIDE: HPVAITASSNLNLLG [SEQ ID 91]
PEPTIDE: ASSNLNLLGFG [SEQ ID 92]
PEPTIDE: ITASSNLNLLGFG [SEQ ID 93]
PEPTIDE: ITASSNLNLLGFGINAE [SEQ ID 94]
PEPTIDE: SSNLNLLGFG [SEQ ID 95]
PEPTIDE: VDLVIPVNGPGKF [SEQ ID 96]
PEPTIDE: LVIPVNGPGKFE [SEQ ID 97]
PEPTIDE: LVIPVNGPGKFEA [SEQ ID 98]
PEPTIDE: LRLVDLVIPVNGPGKFE [SEQ ID 99]
PEPTIDE: YRAKPHTIFLPQHIDAD [SEQ ID 100]
PEPTIDE: HPVAITASSNLNLLGFGINAE [SEQ ID 101]
PEPTIDE: SNLNLLGFG [SEQ ID 102]
PEPTIDE: HPVAITASSNLNLLGFGINAENNE [SEQ ID 103]
PEPTIDE: LVDLVIPVNGPGKFE [SEQ ID 104]
PEPTIDE: LVIPVNGPGKF [SEQ ID 105]
PEPTIDE: TIKLPAGTTSYLVNQDDE [SEQ ID 106]
PEPTIDE: DLRLVDLVIPVNGPGKFE [SEQ ID 107]
PEPTIDE: EDLRLVDLVIPVNGPGKFE [SEQ ID 108]
PEPTIDE: HPVAITASSNLNLLGFG [SEQ ID 109]
PEPTIDE: LVDLVIPVNGPGKFEAFDLAK [SEQ ID 110]
PEPTIDE: DNVISQIENPVKE [SEQ ID 111]
PEPTIDE: VVIIPAGHPVAITASSNLNLLGFG [SEQ ID 112]
PEPTIDE: LVDLVIPVNGPGKFEAF [SEQ ID 113]
PEPTIDE: YPQLQDLDL [SEQ ID 114]]
PEPTIDE: VIPVNGPGKF [SEQ ID 115
PEPTIDE: SKKSLPSE [SEQ ID 116]
PEPTIDE: LPQHIDADLILVVLSGK [SEQ ID 117]
PEPTIDE: RGDTIKLPAGTTSYLVNQD [SEQ ID 118]
PEPTIDE: IPVNGPGKF [SEQ ID 119]
PEPTIDE: LPQHIDADL [SEQ ID 120]
PEPTIDE: LVIPVNGPGK [SEQ ID 121]
PEPTIDE: IFLPQHIDAD [SEQ ID 122]
PEPTIDE: LPQHIDAD [SEQ ID 123]
PEPTIDE: VIPVNGPGK [SEQ ID 124]
PEPTIDE: IFLPQHIDA [SEQ ID 125]
PEPTIDE: TIKLPAGTTSYLVNQDDEE [SEQ ID 126]
PEPTIDE: HGEWRPSYEKEEDEEEGQRER [SEQ ID 127]
PEPTIDE: EKRHGEWRPSYEKEEDEEEGQRE [SEQ ID 128]
PEPTIDE: LPAGTTSYLVNQDDEEDLR [SEQ ID 129]
PEPTIDE: PSYEKEEDEEEGQRER [SEQ ID 130]
PEPTIDE: EKRHGEWRPSYE [SEQ ID 131]
PEPTIDE: TIKLPAGTTSYLVNQDDEED [SEQ ID 132]
PEPTIDE: HGEWRPSYEKQEDEEEK [SEQ ID 133]
PEPTIDE: EWRPSYEKEEDEEE [SEQ ID 134]
PEPTIDE: PSYEKEEDEEEGQR [SEQ ID 135]
PEPTIDE: EKEEDEEEGQR [SEQ ID 136]
PEPTIDE: EWRPSYEKEEDEEEGQRE [SEQ ID 137]
PEPTIDE: KEEDEEEGQR [SEQ ID 138]
PEPTIDE: VQPGRERWEREEDEEQVDE [SEQ ID 139]
PEPTIDE: DVVIIPAGHPVA [SEQ ID 140]
PEPTIDE: HGEWRPSYEKQEDE [SEQ ID 141]
PEPTIDE: EEDEEEGQR [SEQ ID 142]
PEPTIDE: HGEWRPSYEKEEDEEEGQR [SEQ ID 143]
PEPTIDE: EEWRGSQRREDPEE [SEQ ID 144]
PEPTIDE: REEDEEQVDEEWRGSQRREDPEE [SEQ ID 145]
PEPTIDE: RHGEWRPSY [SEQ ID 146]
PEPTIDE: HGEWRPSYEKQEDEE [SEQ ID 147]
PEPTIDE: VVIIPAGHPVA [SEQ ID 148]
PEPTIDE: HGEWRPSYE [SEQ ID 149]
PEPTIDE: KEEDEEEGQRER [SEQ ID 150]
PEPTIDE: VVIIPAGHPVAIT [SEQ ID 151]
PEPTIDE: EKRHGEWRPSYEKEEDE [SEQ ID 152]
PEPTIDE: QVDEEWRGSQRREDPEE [SEQ ID 153]
PEPTIDE: GDTIKLPAGTTSYLVNQDDEEDLR [SEQ ID 154]
PEPTIDE: GSEPRVPAQRE [SEQ ID 155]
PEPTIDE: EEKRHGEWRPSYEKE [SEQ ID 156]
PEPTIDE: EWRPSYEKEEDEE [SEQ ID 157]
PEPTIDE: NYDEGSEPRVPAQRE [SEQ ID 158]
PEPTIDE: VIIPAGHPVAIT [SEQ ID 159]
PEPTIDE: RHGEWRPSYEK [SEQ ID 160]
PEPTIDE: NYDEGSEPR [SEQ ID 161]
PEPTIDE: WRPSYEKEEDEE [SEQ ID 162]
PEPTIDE: WRPSYEKQEDEEE [SEQ ID 163]
PEPTIDE: EKRHGEWRPSYEKQEDEEE [SEQ ID 164]
PEPTIDE: VVIIPAGHPVAITA [SEQ ID 165]
PEPTIDE: KRHGEWRPSYE [SEQ ID 166]
PEPTIDE: GSDDNVISQIENPVKE [SEQ ID 167]
PEPTIDE: VVIIPAGHPV [SEQ ID 168]
PEPTIDE: HGEWRPSY [SEQ ID 169]
PEPTIDE: RPSYEKEEDEEEGQR [SEQ ID 170]
PEPTIDE: HGEWRPSYEK [SEQ ID 171]
PEPTIDE: KRHGEWRPSYEKEE [SEQ ID 172]
PEPTIDE: VVIIPAGHPVAITAS [SEQ ID 173]
PEPTIDE: RGDTIKLPAGTTSYLVNQDDEED [SEQ ID 174]
PEPTIDE: KRHGEWRPSYEKQEDEEE [SEQ ID 175]
PEPTIDE: DEEQVDEEWRGSQRREDPEE [SEQ ID 176]
PEPTIDE: RHGEWRPSYE [SEQ ID 177]
PEPTIDE: HGEWRPSYEKE [SEQ ID 178]
PEPTIDE: KRHGEWRPSYEKEEDEEE [SEQ ID 179]
PEPTIDE: EKRHGEWRPSYEKEEDEEE [SEQ ID 180]
PEPTIDE: TIKLPAGTTSYLVNQDDEEDLRLVD [SEQ ID 181]
PEPTIDE: WRPSYEKEEDEEEGQRE [SEQ ID 182]
PEPTIDE: KRHGEWRPSYEKEEDEE [SEQ ID 183]
PEPTIDE: VVIIPAGHPVAI [SEQ ID 184]
PEPTIDE: EWRGSQRREDPEE [SEQ ID 185]
PEPTIDE: HGEWRPSYEKQEDEEEKQK [SEQ ID 186]
PEPTIDE: SGSDDNVISQIENPVKE [SEQ ID 187]
PEPTIDE: RPSYEKEEDEEEGQRER [SEQ ID 188]
PEPTIDE: EKEEDEEEGQRER [SEQ ID 189]
PEPTIDE: HGEWRPSYEKQ [SEQ ID 190]
PEPTIDE: WRPSYEKEEDEEE [SEQ ID 191]
PEPTIDE: LAKNKNQYLRGFS [SEQ ID 192]
PEPTIDE: NKNQYLRGFS [SEQ ID 193]
PEPTIDE: LRGFSKNILE [SEQ ID 194]
PEPTIDE: LAKNKNQYLRGFSKN [SEQ ID 195]
PEPTIDE: TVLSPNDRNSY [SEQ ID 196]
PEPTIDE: QYLRGFSKNILE [SEQ ID 197]
PEPTIDE: GKAILTVLSPNDRNSYNLE [SEQ ID 198]
PEPTIDE: RGFSKNILE [SEQ ID 199]
PEPTIDE: NKNQYLRGFSKNILE [SEQ ID 200]
PEPTIDE: ASSNLNLLGFGINAE [SEQ ID 201]
PEPTIDE: ASSNLNLLGF [SEQ ID 202]
PEPTIDE: LAKNKNQYLRGFSK [SEQ ID 203]
PEPTIDE: RGDTIKLPAGTTSYLVNQDDEE [SEQ ID 204]
PEPTIDE: ARLSPGDVVIIPAGHPVAITASSN [SEQ ID 205]
PEPTIDE: VQRYEARLSPGD [SEQ ID 206]
PEPTIDE: ARLSPGDVVIIPAGHPVAIT [SEQ ID 207]
PEPTIDE: RGDTIKLPAGTTSYLVNQDDE [SEQ ID 208]
PEPTIDE: ARLSPGDVVIIPAGHPVA [SEQ ID 209]
PEPTIDE: GALMLPHYNSRAIVVLLVNE [SEQ ID 210]
PEPTIDE: ARLSPGDVVIIPAGHPVAITASS [SEQ ID 211]
PEPTIDE: LSPGDVVIIPAGHPVAITASSNLNLLGFGINAENNER [SEQ ID 212]
PEPTIDE: ARLSPGDVVIIPAGHPVAITAS [SEQ ID 213]
PEPTIDE: LSPGDVVIIPAGHPVAITASSNL [SEQ ID 214]
PEPTIDE: ARLSPGDVVIIPAGHPVAITA [SEQ ID 215]
PROTEIN: Q0DEV5-5-RICE
MSALTTSQLATSATGFGIADRSAPSSLLRHGFQGLKPRSPAGGDATSLSVTTSARATPKQQRSVQRGSRRFPSVVVYATGAGMNVVFVGAEM
APWSKTGGLGDVLGGLPPAMAANGHRVMVISPRYDQYKDAWDTSVVAEIKVADRYERVRFFHCYKRGVDRVFIDHPSFLEKVWGKTGEKIY
GPDTGVDYKDNQMRFSLLCQAALEAPRILNLNNNPYFKGTYGEDVVFVCNDWHTGPLASYLKNNYQPNGIYRNAKVAFCIHNISYQGRFAFE
DYPELNLSERFRSSFDFIDGYDTPVEGRKINWMKAGILEADRVLTVSPYYAEELISGIARGCELDNIMRLTGITGIVNGMDVSEWDPSKDKYITA
KYDATTAIEAKALNKEALQAEAGLPVDRKIPLIAFIGRLEEQKGPDVMAAAIPELMQEDVQIVLLGTGKKKFEKLLKSMEEKYPGKVRAVVKFNA
PLAHLIMAGADVLAVPSRFEPCGLIQLQGMRYGTPCACASTGGLVDTVIEGKTGFHMGRLSVDCKVVEPSDVKKVAATLKRAIKVVGTPAYEE
MVRNCMNQDLSWKGPAKNWENVLLGLGVAGSAPGIEGDEIAPLAKENVAAP [SEQ ID 3]
PEPTIDE: NWENVLLGLGVAGSAPGIEGDEIAPLAK [SEQ ID 216]
PEPTIDE: YDQYKDAWDTSVVAEIK [SEQ ID 217]
PEPTIDE: SSFDFIDGYDTPVEGR [SEQ ID 218]
PEPTIDE: GPDTGVDYKDNQM [SEQ ID 219]
PEPTIDE: ILNLNNNPYFK [SEQ ID 220]
PEPTIDE: VVGTPAYEE [SEQ ID 221]
PEPTIDE: IDGYDTPVEGR [SEQ ID 222]
PEPTIDE: VVGTPAYE [SEQ ID 223]
PEPTIDE: IYGPDTGVDYK [SEQ ID 224]
PEPTIDE: VAGSAPGIEGDE [SEQ ID 225]
PEPTIDE: IYGPDTGVDYKDNQMR [SEQ ID 226]
PEPTIDE: VVGTPAYEEMVR [SEQ ID 227]
PEPTIDE: DFIDGYDTPVEGR [SEQ ID 228]
PEPTIDE: LGLGVAGSAPGIEGDEIAPLAK [SEQ ID 229]
PEPTIDE: FNAPLAHLIMAGADVLAVPSR [SEQ ID 230]
PEPTIDE: LGLGVAGSAPGIEGDE [SEQ ID 231]
PEPTIDE: LGLGVAGSAPGIEGDEIAPL [SEQ ID 232]
PEPTIDE: VLTVSPYYAEELISGIAR [SEQ ID 233]
PEPTIDE: EALQAEAGLPVDR [SEQ ID 234]
PEPTIDE: LGLGVAGSAPGIEGD [SEQ ID 235]
PEPTIDE: IMAGADVLAVPSR [SEQ ID 236]
PEPTIDE: GLGVAGSAPGIEGDE [SEQ ID 237]
PEPTIDE: EALQAEAGLPVDRK [SEQ ID 238]
PEPTIDE: TGGLGDVLGGLPPAMAANGHR [SEQ ID 239]
PEPTIDE: LEEQKGPDVMA [SEQ ID 240]
PEPTIDE: LGVAGSAPGIEGDEIAPLAK [SEQ ID 241]
PEPTIDE: GLGVAGSAPGIEGDEIAPLAK [SEQ ID 242]
PEPTIDE: TGGLGDVLGGLPPAM [SEQ ID 243]
PEPTIDE: NVLLGLGVAGSAPGIEGDE [SEQ ID 244]
PROTEIN: P14323-6-RICE
MASSVFSRFSIYFCVLLLCHGSMAQLFNPSTNPWHSPRQGSFRECRFDRLQAFEPLRKVRSEAGVTEYFDEKNELFQCTGTFVIRRVIQPQGLL
VPRYTNIPGVVYIIQGRGSMGLTFPGCPATYQQQFQQFSSQGQSQSQKFRDEHQKIHQFRQGDIVALPAGVAHWFYNDGDAPIVAVYVYDV
NNNANQLEPRQKEFLLAGNNNRAQQQQVYGSSIEQHSGQNIFSGFGVEMLSEALGINAVAAKRLQSQNDQRGEIIHVKNGLQLLKPTLTQQ
QEQAQAQDQYQQVQYSERQQTSSRWNGLEENFCTIKVRVNIENPSRADSYNPRAGRITSVNSQKFPILNLIQMSATRVNLYQNAILSPFWNV
NAHSLVYMIQGRSRVQVVSNFGKTVFDGVLRPGQLLIIPQHYAVLKKAEREGCQYIAIKTNANAFVSHLAGKNSVFRALPVDVVANAYRISRE
QARSLKNNRGEEHGAFTPRFQQQYYPGLSNESESETSE [SEQ ID 4]
PEPTIDE: TVFDGVLRPGQL [SEQ ID 245]
PEPTIDE: RLQSQNDQRGEIIHVK [SEQ ID 246]
PROTEIN: P29835-5-RICE
MASKVVFFAAALMAAMVAISGAQLSESEMRFRDRQCQREVQDSPLDACRQVLDRQLTGRERFQPMFRRPGALGLRMQCCQQLQDVSRE
CRCAAIRRMVRSYEESMPMPLEQGWSSSSSEYYGGEGSSSEQGYYGEGSSEEGYYGEQQQQPGMTRVRLTRARQYAAQLPSMCRVEPQQC
SIFAAGQY [SEQ ID 5]
PEPTIDE: EGYYGEQQQQPGMTR [SEQ ID 247]
PEPTIDE: GYYGEQQQQPGMTR [SEQ ID 248]
PEPTIDE: EEGYYGEQQQQPGMTR [SEQ ID 249]
PEPTIDE: YYGGEGSSSEQGYYGEGSSE [SEQ ID 250]
PEPTIDE: YGGEGSSSEQGYYGEGSSE [SEQ ID 251]
PEPTIDE: SSEEGYYGEQQQQPGMTR [SEQ ID 252]
PEPTIDE: SEEGYYGEQQQQPGMTR [SEQ ID 253]
PEPTIDE: QGYYGEGSSEE [SEQ ID 254]
PEPTIDE: YGGEGSSSEQGYYGEGSSEEGY [SEQ ID 255]
PEPTIDE: YGEQQQQPGMTR [SEQ ID 256]
PEPTIDE: YYGEQQQQPGMTR [SEQ ID 257]
PEPTIDE: SYEESMPMPLEQGWSSSSSE [SEQ ID 258]
PEPTIDE: YYGEGSSEEGYYGEQQQQPGMTR [SEQ ID 259]
PEPTIDE: QQQQPGMTRV [SEQ ID 260]
PEPTIDE: GEQQQQPGMTR [SEQ ID 261]
PEPTIDE: SYEESMPMPLEQGWSSSSSEY [SEQ ID 262]
PEPTIDE: YYGGEGSSSEQGYYGEGSSEEGY [SEQ ID 263]
PEPTIDE: YGEQQQQPGMTRVR [SEQ ID 264]
PEPTIDE: GEGSSEEGYYGEQQQQPGMTR [SEQ ID 265]
PEPTIDE: YGEGSSEEGYYGEQQQQPGMTR [SEQ ID 266]
PEPTIDE: QQQQPGMTRVR [SEQ ID 267]
PEPTIDE: QYAAQLPSMCRVEPQQCSIFAAGQY [SEQ ID 268]
PROTEIN: P0C1U8-5-Staphylococcus aureus
MKGKFLKVSSLFVATLTTATLVSSPAANALSSKAMDNHPQQTQSSKQQTPKIQKGGNLKPLEQREHANVILPNNDRHQITDTTNGHYAPVTYI
QVEAPTGTFIASGVVVGKDTLLTNKHVVDATHGDPHALKAFPSAINQDNYPNGGFTAEQITKYSGEGDLAIVKFSPNEQNKHIGEVVKPATMS
NNAETQVNQNITVTGYPGDKPVATMWESKGKITYLKGEAMQYDLSTTGGNSGSPVFNEKNEVIGIHWGGVPNEFNGAVFINENVRNFLKQ
NIEDIHFANDDQPNNPDNPDNPNNPDNPNNPDEPNNPDNPNNPDNPDNGDNNNSDNPDAA [SEQ ID 14]
PEPTIDE: APTGTFIASGVVVGKD [SEQ ID 413]
PEPTIDE: LAIVKFSPNEQNKHIGE [SEQ ID 414]
PEPTIDE: RHQITDTTNGHYAPVTYIQVE [SEQ ID 415]
PEPTIDE: GDLAIVKFSPNEQNKHIGE [SEQ ID 416]
PEPTIDE: NPDNPDNPNNPDNPNNPD [SEQ ID 417]
PROTEIN: P14614-4-RICE
MATIAFSRLSIYFCVLLLCHGSMAQLFGPNVNPWHNPRQGGFRECRFDRLQAFEPLRRVRSEAGVTEYFDEKNEQFQCTGTFVIRRVIEPQGL
LVPRYSNTPGMVYIIQGRGSMGLTFPGCPATYQQQFQQFLPEGQSQSQKFRDEHQKIHQFRQGDIVALPAGVAHWFYNEGDAPVVALYVF
DLNNNANQLEPRQKEFLLAGNNNREQQMYGRSIEQHSGQNIFSGFNNELLSEALGVNALVAKRLQGQNDQRGEIIRVKNGLKLLRPAFAQQ
QEQAQQQEQAQAQYQVQYSEEQQPSTRCNGLDENFCTIKARLNIENPSHADTYNPRAGRITRLNSQKFPILNLVQLSATRVNLYQNAILSPF
WNVNAHSLVYIVQGHARVQVVSNLGKTVFNGVLRPGQLLIIPQHYVVLKKAEHEGCQYISFKTNANSMVSHLAGKNSIFRAMPVDVIANAYR
ISREQARSLKNNRGEELGAFTPRYQQQTYPGFSNESENEALE [SEQ ID 6]
PEPTIDE: TVFNGVLRPGQL [SEQ ID 269]
PEPTIDE: TVFNGVLRPGQLL [SEQ ID 270]
PEPTIDE: SGFNNELLSEALGVNALVAK [SEQ ID 271]
PEPTIDE: NGVLRPGQL [SEQ ID 272]
PEPTIDE: ALVAKRLQGQN DQRG El [SEQ ID 273]
PEPTIDE: VPRYSNTPGM [SEQ ID 274]
PEPTIDE: PRYSNTPGMV [SEQ ID 275]
PEPTIDE: YSNTPGMVY [SEQ ID 276]
PEPTIDE: LVPRYSNTPGM [SEQ ID 277]
PEPTIDE: FYNEGDAPVV [SEQ ID 278]
PEPTIDE: FYNEGDAPVVAL [SEQ ID 279]
PEPTIDE: FEPLRRVRSEAGVTE [SEQ ID 280]
PEPTIDE: FYNEGDAPVVALY [SEQ ID 281]
PEPTIDE: FYNEGDAPVVA [SEQ ID 282]
PROTEIN: P09918-14-Pisum sativum
MFSGVTGILNRGHKIKGTVVLMRKNVLDINSLTTVGGVIGQGFDILGSTVDNLTAFLGRSVSLQLISATKPDATGKGKLGKATFLEGIISSLPTLG
AGQSAFKIHFEWDDDMGIPGAFYIKNFMQTEFFLVSLTLDDIPNHGSIYFVCNSWIYNAKHHKIDRIFFANQTYLPSETPAPLVHYREEELNNLR
GDGTGERKEWERIYDYDVYNDLGNPDSGENHARPVLGGSETYPYPRRGRTGRKPTRKDPNSESRSDYVYLPRDEAFGHLKSSDFLTYGLKAVS
QNVVPALESVFFDLNFTPNEFDSFDEVHGLYEGGIKLPTNILSQISPLPVLKEIFRTDGENTLKYPPPKVIQVSRSGWMTDEEFAREMLAGVNPN
VICCLQEFPPRSKLDSQIYGDHTSKISKEHLEPNLEGLTVEEAIQNKKLFLLDHHDSIMPYLRRINSTSTKAYATRTILFLNNNQNLKPLAIELSLPHP
QGDEHGAVSYVYQPALEGVESSIWLLAKAYVIVNDSCYHQLVSHWLNTHAVVEPFVIATNRHLSCLHPIYKLLYPHYRDTMNINSLARLSLVND
GGIIEKTFLWGRYSMEMSSKVYKNWVFTEQALPADLIKRGMAIEDPSSPCGVKLVVEDYPYAVDGLEIWAIIKTWVQDYVSLYYTSDEKLRQD
SELQAWWKELVEVGHGDKKNEPWWPKMQTREDLIEVCSIVIWTASALHAAVNFGQYSYGGLILNRPTLSRRFMPEKGSAEFEELVKSPQKA
YLKTITPKFQTLIDLSVIEILSRHASDELYLGERDNPNWTSDKRALEAFKKFGNKLAEIEKKLTQRNNDEKLRNRHGPVEMPYTLLYPSSKEGLTFR
GIPNSISI [SEQ ID 7]
PEPTIDE: HGPVEMPYTLLYPSSK [SEQ ID 283]
PROTEIN: P02857-1-PEA
MAKLLALSLSFCFLLLGGCFALREQPQQNECQLERLDALEPDNRIESEGGLIETWNPNNKQFRCAGVALSRATLQRNALRRPYYSNAPQEIFIQ
QGNGYFGMVFPGCPETFEEPQESEQGEGRRYRDRHQKVNRFREGDIIAVPTGIVFWMYNDQDTPVIAVSLTDIRSSNNQLDQMPRRFYLAG
NHEQEFLQYQHQQGGKQEQENEGNNIFSGFKRDYLEDAFNVNRHIVDRLQGRNEDEEKGAIVKVKGGLSIISPPEKQARHQRGSRQEEDED
EEKQPRHQRGSRQEEEEDEDEERQPRHQRRRGEEEEEDKKERGGSQKGKSRRQGDNGLEETVCTAKLRLNIGPSSSPDIYNPEAGRIKTVTSL
DLPVLRWLKLSAEHGSLHKNAMFVPHYNLNANSIIYALKGRARLQVVNCNGNTVFDGELEAGRALTVPQNYAVAAKSLSDRFSYVAFKTNDR
AGIARLAGTSSVINNLPLDVVAATFNLQRNEARQLKSNNPFKFLVPARESENRASA [SEQ ID 8]
PEPTIDE: LDALEPDNR [SEQ ID 284]
PEPTIDE: DALEPDNR [SEQ ID 285]
PEPTIDE: HGSLHKNAMFVPHYNLNANSIIYA [SEQ ID 286]
PEPTIDE: LAGTSSVINNLPLDVVAATF [SEQ ID 287]
PEPTIDE: FREGDIIAVPTGIVFW [SEQ ID 288]
PEPTIDE: GTSSVINNLPLDVVAATFNLQRNE [SEQ ID 289]
PEPTIDE: KGAIVKVKGGLSIISPPE [SEQ ID 290]
PEPTIDE: RLAGTSSVINNLPLD [SEQ ID 291]
PEPTIDE: AGTSSVINNLPLDVVAATFNLQRNE [SEQ ID 292]
PEPTIDE: AGTSSVINNLPL [SEQ ID 293]
PEPTIDE: LAGTSSVINNLPLDVVA [SEQ ID 294]
PEPTIDE: AGTSSVINNLPLDV [SEQ ID 295]
PEPTIDE: AGRIKTVTSLDLPVLRW [SEQ ID 296]
PEPTIDE: AGRIKTVTSLDLPVLR [SEQ ID 297]
PEPTIDE: FREGDIIAVPTGIVF [SEQ ID 298]
PEPTIDE: AGTSSVINNLPLD [SEQ ID 299]
PEPTIDE: LAGTSSVINNLPL [SEQ ID 300]
PEPTIDE: LAGTSSVINNLPLDVV [SEQ ID 301]
PEPTIDE: EGDIIAVPTGIVF [SEQ ID 302]
PEPTIDE: LAGTSSVINNLPLDV [SEQ ID 303]
PEPTIDE: AGRALTVPQNYAVAAKSLSD [SEQ ID 304]
PEPTIDE: AGRALTVPQNYA [SEQ ID 305]
PEPTIDE: LAGTSSVINNLPLD [SEQ ID 306]
PEPTIDE: RAGIARLAGTSSVINNLPLDVVA [SEQ ID 307]
PROTEIN: P02855-4-PEA
DNAEIEKILLEEHEKETHHRRGLRDKRQQSQEKNVIVKVSKKQIEELSKNAKSSSKKSVSSRSEPFNLKSSDPIYSNQYGKFFEITPKKNPQLQDLD
IFVNYVEIKEGSLWLPHYNSRAIVIVTVNEGKGDFELVGQRNENQQGLREEDDEEEEQREEETKNQVQSYKAKLTPGDVFVIPAGHPVAVRAS
SNLNLLGFGINAENNQRNFLAGEEDNVISQIQKQVKDLTFPGSAQEVDRLLENQKQSYFANAQPQQRETRSQEIKEHLYSILGAF [SEQ ID 9]
PEPTIDE: RASSNLNLLGFGINAE [SEQ ID 308]
PEPTIDE: VTVNEGKGDFEL [SEQ ID 309]
PEPTIDE: VRASSNLNLLGFGINAE [SEQ ID 310]
PEPTIDE: VRASSNLNLLGFG [SEQ ID 311]
PEPTIDE: HPVAVRASSNLNLLGFG [SEQ ID 312]
PEPTIDE: TKNQVQSYKAKLTPGD [SEQ ID 313]
PEPTIDE: HPVAVRASSNLNLLG [SEQ ID 314]
PEPTIDE: KAKLTPGDVFVIPAG [SEQ ID 315]
PEPTIDE: DLTFPGSAQEVDRLLENQK [SEQ ID 316]
PEPTIDE: PAGHPVAVR [SEQ ID 317]
PEPTIDE: AKLTPGDVFVIPAGHPVA [SEQ ID 318]
PEPTIDE: SYKAKLTPGDVFVIPAGHPVA [SEQ ID 319]
PEPTIDE: LTPGDVFVIPAGHPVAVR [SEQ ID 320]
PEPTIDE: VQSYKAKLTPGDVFVIPAG [SEQ ID 321]
PEPTIDE: YKAKLTPGDVFVIPAGHPVA [SEQ ID 322]
PEPTIDE: FVIPAGHPVAVR [SEQ ID 323]
PEPTIDE: YKAKLTPGDVFVIPAG [SEQ ID 324]
PEPTIDE: DLTFPGSAQEVDR [SEQ ID 325]
PEPTIDE: AKLTPGDVFVIPAGHPVAVR [SEQ ID 326]
PEPTIDE: LTPGDVFVIPAG [SEQ ID 327]
PEPTIDE: SYKAKLTPGDVFVIPAG [SEQ ID 328]
PEPTIDE: SYKAKLTPGDVFVIPAGHPVAVR [SEQ ID 329]
PEPTIDE: VIPAGHPVAVR [SEQ ID 330]
PEPTIDE: QVQSYKAKLTPGDVFVIPAG [SEQ ID 331]
PEPTIDE: AKLTPGDVFVIPAG [SEQ ID 332]
PEPTIDE: HPVAVRASSNLNLLGFGINAE [SEQ ID 333]
PEPTIDE: YKAKLTPGDVFVIPAGHPVAVR [SEQ ID 334]
PEPTIDE: TKNQVQSYKAKLTPGDVFVIPAG [SEQ ID 335]
PEPTIDE: PFNLKSSDPIYS [SEQ ID 336]
PEPTIDE: IEKILLEE [SEQ ID 337]
PEPTIDE: SRSEPFNLKSSDPIYS [SEQ ID 338]
PEPTIDE: HPVAVRASSNLNL [SEQ ID 339]
PROTEIN: D3VNE1-5-PEA
MAATPIKPLMLLAIAFLASVCVSSRSDQENPFIFKSNRFQTLYENENGHIRLLQKFDKRSKIFENLQNYRLLEYKSKPRTLFLPQYTDADFILVVLSG
KATLTVLKSNDRNSFNLERGDTIKLPAGTIAYLANRDDNEDLRVLDLTIPVNKPGQLQSFLLSGTQNQPSLLSGFSKNILEAAFNTNYEEIEKVLLE
QQEQEPQHRRSLKDRRQEINEENVIVKVSREQIEELSKNAKSSSKKSVSSESGPFNLRSRNPIYSNKFGKFFEITPEKNQQLQDLDIFVNSVDIKE
GSLLLPNYNSRAIVIVTVTEGKGDFELVGQRNENQGKENDKEEEQEEETSKQVQLYRAKLSPGDVFVIPAGHPVAINASSDLNLIGFGINAENN
ERNFLAGEEDNVISQVERPVKELAFPGSSHEVDRLLKNQKQSYFANAQPLQRE [SEQ ID 10]
PEPTIDE: TLFLPQYTDADFILVVLSGK [SEQ ID 340]
PROTEIN: P07728-1-RICE
MASINRPIVFFTVCLFLLCNGSLAQQLLGQSTSQWQSSRRGSPRECRFDRLQAFEPIRSVRSQAGTTEFFDVSNEQFQCTGVSVVRRVIEPRGL
LLPHYTNGASLVYIIQGRGITGPTFPGCPESYQQQFQQSGQAQLTESQSQSQKFKDEHQKIHRFRQGDVIALPAGVAHWCYNDGEVPVVAIY
VTDLNNGANQLDPRQRDFLLAGNKRNPQAYRREVEERSQNIFSGFSTELLSEALGVSSQVARQLQCQNDQRGEIVRVEHGLSLLQPYASLQE
QEQGQVQSRERYQEGQYQQSQYGSGCSNGLDETFCTLRVRQNIDNPNRADTYNPRAGRVTNLNTQNFPILSLVQMSAVKVNLYQNALLSPF
WNINAHSVVYITQGRARVQVVNNNGKTVFNGELRRGQLLIIPQHYAVVKKAQREGCAYIAFKTNPNSMVSHIAGKSSIFRALPNDVLANAYRI
SREEAQRLKHNRGDEFGAFTPIQYKSYQDVYNAAESS [SEQ ID 11]
PEPTIDE: EVEERSQNIF [SEQ ID 341]
PEPTIDE: VEERSQNIFSGF [SEQ ID 342]
PEPTIDE: ASLQEQEQGQVQ [SEQ ID 343]
PEPTIDE: QEQEQGQVQSR [SEQ ID 344]
PEPTIDE: ASLQEQEQGQVQSR [SEQ ID 345]
PEPTIDE: EVEERSQNIFSGF [SEQ ID 346]
PEPTIDE: VTDLNNGANQLDPRQRD [SEQ ID 347]
PEPTIDE: VEHGLSLLQPYASL [SEQ ID 348]
PEPTIDE: IYVTDLNNGANQLDPRQRDFL [SEQ ID 349]
PEPTIDE: VTDLNNGANQLDPRQRDFL [SEQ ID 350]
PEPTIDE: VEHGLSLLQPYASLQEQEQGQVQSR [SEQ ID 351]
PEPTIDE: VTDLNNGANQLDPR [SEQ ID 352]
PEPTIDE: IYVTDLNNGANQLDPRQRD [SEQ ID 353]
PEPTIDE: YVTDLNNGANQLDPRQRDFL [SEQ ID 354]
PEPTIDE: YVTDLNNGANQLDPR [SEQ ID 355]
PEPTIDE: STELLSEALGVSSQVAR [SEQ ID 356]
PEPTIDE: HGLSLLQPYASLQEQE [SEQ ID 357]
PEPTIDE: SGFSTELLSEALGVSSQVAR [SEQ ID 358]
PROTEIN: P07730-2-RICE
MASINRPIVFFTVCLFLLCDGSLAQQLLGQSTSQWQSSRRGSPRGCRFDRLQAFEPIRSVRSQAGTTEFFDVSNELFQCTGVSVVRRVIEPRGLL
LPHYTNGASLVYIIQGRGITGPTFPGCPETYQQQFQQSGQAQLTESQSQSHKFKDEHQKIHRFRQGDVIALPAGVAHWCYNDGEVPVVAIYV
TDINNGANQLDPRQRDFLLAGNKRNPQAYRREVEEWSQNIFSGFSTELLSEAFGISNQVARQLQCQNDQRGEIVRVERGLSLLQPYASLQEQ
EQGQMQSREHYQEGGYQQSQYGSGCPNGLDETFCTMRVRQNIDNPNRADTYNPRAGRVTNLNSQNFPILNLVQMSAVKVNLYQNALLSP
FWNINAHSIVYITQGRAQVQVVNNNGKTVFNGELRRGQLLIVPQHYVVVKKAQREGCAYIAFKTNPNSMVSHIAGKSSIFRALPTDVLANAYR
ISREEAQRLKHNRGDEFGAFTPLQYKSYQDVYNVAESS [SEQ ID 12]
PEPTIDE: GAFTPLQYKSYQD [SEQ ID 359]
PEPTIDE: GLLLPHYTNGASLVY [SEQ ID 360]
PEPTIDE: FLLAGNKRNPQAYRRE [SEQ ID 361]
PEPTIDE: ALPTDVLANAYR [SEQ ID 362]
PEPTIDE: DFLLAGNK [SEQ ID 363]
PEPTIDE: DVLANAYR [SEQ ID 364]
PEPTIDE: GAFTPLQYK [SEQ ID 365]
PEPTIDE: QGDVIALPAGVAHW [SEQ ID 366]
PEPTIDE: FGAFTPLQYKSY [SEQ ID 367]
PEPTIDE: FLLAGNKRNPQAYR [SEQ ID 368]
PEPTIDE: FGAFTPLQYKSYQ [SEQ ID 369]
PEPTIDE: GLSLLQPYASLQEQE [SEQ ID 370]
PEPTIDE: AFTPLQYK [SEQ ID 371]
PEPTIDE: FGAFTPLQYKSYQD [SEQ ID 372]
PEPTIDE: GDEFGAFTPLQYK [SEQ ID 373]
PEPTIDE: FGAFTPLQYKSYQDV [SEQ ID 374]
PEPTIDE: FGAFTPLQYK [SEQ ID 375]
PEPTIDE: FGAFTPLQYKS [SEQ ID 376]
PEPTIDE: VYIIQGRGITGPTF [SEQ ID 377]
PEPTIDE: YIIQGRGITGPTF [SEQ ID 378]
PEPTIDE: KTNPNSMVSHIAGK [SEQ ID 379]
PEPTIDE: TNPNSMVSHIAGK [SEQ ID 380]
PEPTIDE: PNSMVSHIAGKS [SEQ ID 381]
PEPTIDE: NIDNPNRADTYNPRAGRVTN [SEQ ID 382]
PEPTIDE: QRDFLLAGNKR [SEQ ID 383]
PEPTIDE: LLQPYASLQEQE [SEQ ID 384]
PEPTIDE: QRDFLLAGNK [SEQ ID 385]
PEPTIDE: QEQEQGQMQSR [SEQ ID 386]
PEPTIDE: SLLQPYASLQEQE [SEQ ID 387]
PEPTIDE: ASLQEQEQGQM [SEQ ID 388]
PEPTIDE: ASLQEQEQGQMQSR [SEQ ID 389]
PEPTIDE: DFLLAGNKR [SEQ ID 390]
PEPTIDE: QAFEPIRSVRSQAGTTEF [SEQ ID 391]
PEPTIDE: KTNPNSMVSHIAGKSSIF [SEQ ID 392]
PEPTIDE: VRRVIEPRGLLLPHYTNGASL [SEQ ID 393]
PEPTIDE: FGAFTPLQYKSYQDVYN [SEQ ID 394]
PEPTIDE: IALPAGVAHW [SEQ ID 395]
PEPTIDE: RVRQNIDNPNRADTYNPRAGRVTNL [SEQ ID 396]
PEPTIDE: NIDNPNRADTYNPRAGRVTNL [SEQ ID 397]
PEPTIDE: GAFTPLQYKSYQDVYN [SEQ ID 398]
PEPTIDE: PNSMVSHIAGKSSIFR [SEQ ID 399]
PEPTIDE: RLQAFEPIRSVRSQAGTTE [SEQ ID 400]
PEPTIDE: TNPNSMVSHIAGKSSIFR [SEQ ID 401]
PROTEIN: Q0D7S0-3-RICE
MASNKVVFSVLLLAVVSVLAATATMAEYHHQDQVVYTPGPLCQPGMGYPMYPLPRCRALVKRQCVGRGTAAAAEQVRRDCCRQLAAVDD
SWCRCEAISHMLGGIYRELGAPDVGHPMSEVFRGCRRGDLERAAASLPAFCNVDIPNGGGGVCYWLARSGY [SEQ ID 13]
PEPTIDE: ELGAPDVGHPM [SEQ ID 402]
PEPTIDE: LGAPDVGHPM [SEQ ID 403]
PEPTIDE: ELGAPDVGHPMSEVF [SEQ ID 404]
PEPTIDE: ELGAPDVGHPMS [SEQ ID 405]
PEPTIDE: ELGAPDVGHPMSEVFR [SEQ ID 406]
PEPTIDE: ELGAPDVGHPMSEV [SEQ ID 407]
PEPTIDE: ELGAPDVGHPMSE [SEQ ID 408]
PEPTIDE: LGAPDVGHPMSE [SEQ ID 409]
PEPTIDE: YRELGAPDVGHPMSE [SEQ ID 410]
PEPTIDE: LGAPDVGHPMSEV [SEQ ID 411]
PEPTIDE: RELGAPDVGHPMSE [SEQ ID 412]
ADDITIONAL PEPTIDES (AND SOURCE PROTEIN)
HPRPPKPDAPR [SEQ ID 452]-Q0D9DO
LQQAPPPPQR [SEQ ID 453]-Q6AVS5
VGWGEQPWSPY [SEQ ID 454]-Q8H920
FHMPP [SEQ ID 455]-O49927
FRRP [SEQ ID 456]-P29835
FWM [SEQ ID 457]-P15838
HMPPS [SEQ ID 458]-049927
PVEMPTLLYPS [SEQ ID 459
HMPSS [SEQ ID 460]-O49927
HRFR [SEQ ID 461]-Q712V4
HRRS [SEQ ID 462]-P13918
HSPR [SEQ ID 463]-P14323
HWF [SEQ ID 464-P14323
MFRR [SEQ ID 465]-Q41000
MFRRP [SEQ ID 466-P29835
MPPS [SEQ ID 467]-049927
MPRR [SEQ ID 468]-P15838
NMPS [SEQ ID 469]-P20698
PHMP [SEQ ID 470]-O49927
PHMPS [SEQ ID 471-O49927
PRRF [SEQ ID 472]-P15838
WMK [SEQ ID 473]-O04434
DSINALEPDHR [SEQ ID 474]-P05692
ELTFPGSVQ [SEQ ID 475]-Q9M3X6
ELTFPGSVQE [SEQ ID 476]-Q9M3X6
IFEDAITIPGR [SEQ ID 477]-P09886
LDALEPDNRIESE [SEQ ID 478]-P02857
KTLDYWPSLR [SEQ ID 479]-P08688
RHGEWGPSY [SEQ ID 480
ILVDGSHDIER [SEQ ID 481]-Q5N725
LVSHPIAAHEGR [SEQ ID 482]-P14614
NLAQAPAQALL [SEQ ID 483]-Q0DJ38
FLPQHTD [SEQ ID 484]-P13918
LEPDNR [SEQ ID 485]-P15838
LQSQND [SEQ ID 486]-P14323
LQSQNDQRGEI [SEQ ID 487]-P14323
QSQNDQRGEIIHVK [SEQ ID 488]-P14323
RGEIIHVK [SEQ ID 489]-P14323
RLQSQNDQ [SEQ ID 490]-P14323
RLQSQNDQRG [SEQ ID 491]-P14323
RLQSQNDQRGEIIH [SEQ ID 492]-P14323
VFDGVLRPG [SEQ ID 493]-P14323
HNPR [SEQ ID 494]-P14614
HPMS [SEQ ID 495]-Q0D7S0
HPSF [SEQ ID 496]-Q0DEV5
MPMP [SEQ ID 497]-P29835
PMPL [SEQ ID 498]-P29835
PNSM [SEQ ID 499]-P07728
WDP [SEQ ID 500]-B5A8N6
LRGFSK [SEQ ID 501]-Q9M3X6
RSQNIF [SEQ ID 502]-P07728
YLRGFS [SEQ ID 503]-Q9M3X6
GALMLPHYN [SEQ ID 504]-Q9M3X6
GALMLPHYNSR [SEQ ID 505]-Q9M3X6
KNPQLQDLDIFVNYVEIK [SEQ ID 700]P02855
PGQLQSFLLSGNQNQQNYLSGFSK [SEQ ID 701]P13918
RGPQQYAEWQINEK [SEQ ID 702]Q6K7K6
VLDLAIPVNRPGQL [SEQ ID 703]Q6K508
GYVGLTFPGCPATHQQQFQLFEQR [SEQ ID 704]P09918
LDVTPLSLGL [SEQ ID 717]
EEGIQLVAEAIR [SEQ ID 718]
YSLKPLVPR [SEQ ID 719]
WHT [SEQ ID 720]
WHN [SEQ ID 721]
NNPF [SEQ ID 722]
MRFR [SEQ ID 723]
MPPSS [SEQ ID 724]
HMPS [SEQ ID 725]
HMPPS [SEQ ID 726]
GHPM [SEQ ID 727]
FWN [SEQ ID 728]
FHMP [SEQ ID 729]
WTIVQGLPIDE [SEQ ID 730]
GYPMYPLPR [SEQ ID 731]
HGGEGGRPY [SEQ ID 732]
LRGFSK [SEQ ID 733]
GALMLPHYN [SEQ ID 734]
GALMLPHYNSR [SEQ ID 735]
VFDGVLRPG [SEQ ID 736]
LQSQND [SEQ ID 737]
LQSQNDQRGEI [SEQ ID 738]
QSQNDQRGEIIHVK [SEQ ID 739]
RGEIIHVK [SEQ ID 740]
RLQSQNDQ [SEQ ID 741]
RLQSQNDQRG [SEQ ID 742]
RLQSQNDQRGEIIH [SEQ ID 743]
MPMP [SEQ ID 744]
PMPL [SEQ ID 745]
LEPDNR [SEQ ID 746]
GIARLAGTSSVIN [SEQ ID 747]
RSQNIF [SEQ ID 748]
PNSM [SEQ ID 749]
GHPM [SEQ ID 750]
HPMS [SEQ ID 751]
FLPQHTD [SEQ ID 752]
EWQINEK [SEQ ID 753]
GPQQYAEWQINEK [SEQ ID 754]
PQQYAEWQ [SEQ ID 755]
RGPQQYA [SEQ ID 756]
HNPR [SEQ ID 757]
WHN [SEQ ID 758]
WDP [SEQ ID 759]
HPSF [SEQ ID 760]
PGQLQSFLLSGNQNQQNYLSGF [SEQ ID 761]
QLQSFLLSGNQNQQNYLSGFSK [SEQ ID 762]
QSFLLSGNQNQQ [SEQ ID 763]
PGQLQSFLLSGN [SEQ ID 764]
QSFLLSGNQ [SEQ ID 765]
QNQQNYLSGFSK [SEQ ID 766]
YLRGFS [SEQ ID 767]
PVEMPTLLYPS [SEQ ID 768]
RGPQQYAEWQINE [SEQ ID 769]
GYVGLTFPGCPATHQQQFQLFEQR [SEQ ID 770]
KNPQLQDLDIFVNYVEIK [SEQ ID 771]
PGQLQSFLLSGNQNQQNYLSGFSK [SEQ ID 772]
RGPQQYAEWQINEK [SEQ ID 773]
VLDLAIPVNRPGQL [SEQ ID 774]
RGPQQYAEWQINEK [SEQ ID 775]
PROTEIN P02855 [SEQ ID 546-552]
PEPTIDE: SRAIVIVTVNE
PEPTIDE: AKLTPGDV
PEPTIDE: IVIVTVNEGK
PEPTIDE: LDALEPDNRIESEGGL (also in P02857)
PEPTIDE: RPYYSNAPQE (also in P02857)
PEPTIDE: LDALEPDNRIESEGGLIETWNPNNK (also in P02857)
PEPTIDE: AIVIVTVNEGK (also in P13918)
PROTEIN P02857 [SEQ ID 553-565
PEPTIDE: LQVVNCNGNTVFDGEL
PEPTIDE: QVVNCNGNTVFDGEL
PEPTIDE: IIAVPTGIVF
PEPTIDE: GRRYRDRHQKVNRFRE
PEPTIDE: RPYYSNAPQEI
PEPTIDE: RLDALEPDNRIE
PEPTIDE: RLDALEPDNRIESE
PEPTIDE: LDALEPDNRIESEGGLIETW
PEPTIDE: LDALEPDNRIE
PEPTIDE: LDALEPDNRIESEGGLIE
PEPTIDE: LDALEPDNRIESEGGL (also in P02855)
PEPTIDE: RPYYSNAPQE (also in P02857)
PEPTIDE: LDALEPDNRIESEGGLIETWNPNNK (also in P02855)
PROTEIN P07728 [SEQ ID 566-609]
PEPTIDE: VEHGLSLLQPYASLQEQEQGQVQSRER
PEPTIDE: RSQNIFSGF
PEPTIDE: GITGPTFPGCPESY
PEPTIDE: CNGS
PEPTIDE: SPREC
PEPTIDE: PREC
PEPTIDE: PRECR
PEPTIDE: CPES
PEPTIDE: SGCS
PEPTIDE: CSNG
PEPTIDE: RSQNIFSGFSTE
PEPTIDE: VEEWSQNIFSGFST
PEPTIDE: WSQNIFSGFSTEL
PEPTIDE: WSQNIFSGFSTE
PEPTIDE: STSQWQSSRR
PEPTIDE: NRPI
PEPTIDE: CDGS
PEPTIDE: PRGC
PEPTIDE: PRGCR
PEPTIDE: RGCR
PEPTIDE: GCRF
PEPTIDE: PTFP
PEPTIDE: PGCPE
PEPTIDE: GCPE
PEPTIDE: CPET
PEPTIDE: AHWC
PEPTIDE: HWCY
PEPTIDE: SGCP
PEPTIDE: SGCPN
PEPTIDE: GCPN
PEPTIDE: CPNG
PEPTIDE: TFCTM
PEPTIDE: FCTM
PEPTIDE: FCTMR
PEPTIDE: CTMR
PEPTIDE: EGCA
PEPTIDE: SQNIFSGFSTELL
PEPTIDE: SQNIFSGFSTE
PEPTIDE: QNDQRGEIVR
PEPTIDE: SQNIFSGFSTEL (also in P07730)
PEPTIDE: QLQCQNDQRGEI (also in P07730)
PEPTIDE: LGQSTSQWQSSR (also in P07730)
PEPTIDE: QQLLGQSTSQWQSSR (also in P07730)
PEPTIDE: LLGQSTSQWQSSR (also in P07730)
PROTEIN P07730 [SEQ ID 610-619]
PEPTIDE: NDQRGEIVR
PEPTIDE: GQSTSQWQSSR
PEPTIDE: STSQWQSSR
PEPTIDE: GITGPTFPGCPET
PEPTIDE: GITGPTFPGCPETY
PEPTIDE: SQNIFSGFSTEL (also in P07728)
PEPTIDE: QLQCQNDQRGEI (also in P07728)
PEPTIDE: LGQSTSQWQSSR (also in P07728)
PEPTIDE: QQLLGQSTSQWQSSR (also in P07728)
PEPTIDE: LLGQSTSQWQSSR (also in P07728)
PROTEIN P09918 [620-630]
PEPTIDE: IFFANQTYL
PEPTIDE: EHLEPNLEGLTVEE
PEPTIDE: IFFANQTYLPSETPAPLVHYREEELNNLRGDGTGER
PEPTIDE: IHFEWDDDMGIPGAFYIK
PEPTIDE: IFFANQTYLPSETPAPLVHYREEELNNLR
PEPTIDE: TEQALPADLIK
PEPTIDE: EHLEPNLEGLTVEEAIQNKK
PEPTIDE: ISKEHLEPNLEGLTVEEAIQNKK
PEPTIDE: LSLPHPQGDEHGAVSY
PEPTIDE: ISKEHLEPNLEGLTVEEAIQNK
PEPTIDE: EHLEPNLEGLTVEEAIQNK
PROTEIN P0C1U8 [SEQ ID 631]
PEPTIDE: LSTTGGNSGSPVFNEKNE
PROTEIN P13918 [SEQ ID 632-639]
PEPTIDE: QSFLLSGNQNQQNYLSG
PEPTIDE: VLDLAIPVNRPGQLQS
PEPTIDE: VLDLAIPVNRPGQLQSFL
PEPTIDE: FLLSGNQNQQNYLSG
PEPTIDE: FLLSGNQNQQNYLSGFSK
PEPTIDE: DPQNPFIFKSNKFQTLFE
PEPTIDE: ELAFPGSAQEVDRILENQK
PEPTIDE: AIVIVTVNEGK (also in P02855)
PROTEIN P14323 [SEQ ID 640-667]
PEPTIDE: INAVAAKRLQSQNDQRGE
PEPTIDE: NRAQQQQVYGSSIE
PEPTIDE: PSTNPWHSPR
PEPTIDE: CHGS
PEPTIDE: CHGSM
PEPTIDE: PWHS
PEPTIDE: FREC
PEPTIDE: RECR
PEPTIDE: ECRF
PEPTIDE: CRFD
PEPTIDE: CRFDR
PEPTIDE: CTGT
PEPTIDE: FPGC
PEPTIDE: FPGCP
PEPTIDE: PGCP
PEPTIDE: PGCPA
PEPTIDE: PGCPAT
PEPTIDE: GCPA
PEPTIDE: CPAT
PEPTIDE: ENFC
PEPTIDE: NFCT
PEPTIDE: NFCTI
PEPTIDE: FCTI
PEPTIDE: AQQQQVYGSSIEQH
PEPTIDE: AQQQQVYGSSIEQHSGQNIFSGF
PEPTIDE: AAKRLQSQNDQRGE
PEPTIDE: QARSLKNNRGEE (also in P14614)
PEPTIDE: FNPSTNPWHSPRQGS (also in Q0DEV5)
PROTEIN P14614 [SEQ ID 668]
PEPTIDE: QARSLKNNRGEE (also in P14323)
PROTEIN Q0D7S0 [SEQ ID 669]
PEPTIDE: AAASLPAFCNVDIPNGGGGVCYWLAR
PROTEIN Q0DEV5 [SEQ ID 670-680]
PEPTIDE: VAGSAPGIEGDEIAPLAK
PEPTIDE: LGVAGSAPGIEGDEIAPLAKEN
PEPTIDE: GSAPGIEGDEIAPLAKE
PEPTIDE: VAGSAPGIEGDEIAP
PEPTIDE: GVAGSAPGIEGDEIAPLAK
PEPTIDE: GVAGSAPGIEGDEIAPLAKEN
PEPTIDE: VAGSAPGIEGDEIAPLAKEN
PEPTIDE: SAPGIEGDEIAPLAK
PEPTIDE: GSAPGIEGDEIAPLAK
PEPTIDE: SAPGIEGDEIAPLAKEN
PEPTIDE: FNPSTNPWHSPRQGS (also in P14323)
PROTEIN Q9M3X6 [SEQ ID 681-699]
PEPTIDE: VDLVIPVNGPGK
PEPTIDE: LVDLVIPVNGPGK
PEPTIDE: IKLPAGTTSY
PEPTIDE: IKLPAGTTSYL
PEPTIDE: RRNPFLFKSNKF
PEPTIDE: IENPVKELTFPGSVQEINR
PEPTIDE: RRNPFLFKSNKFLT
PEPTIDE: AKPHTIFLPQH IDA
PEPTIDE: AKPHTIFLPQHIDAD
PEPTIDE: KQKYRYQRE
PEPTIDE: KQKYQYQRE
PEPTIDE: MLPH
PEPTIDE: RRNPFLFKSNKFLTLFENE
PEPTIDE: PFLFKSNKFLTLFE
PEPTIDE: SQERRNPFLFKSNKFLTLFE
PEPTIDE: RRNPFLFKSNKFLTLFE
PEPTIDE: SQERRNPFLFKSNKFLTLFENE
PEPTIDE: LTFPGSVQE
PEPTIDE: ELTFPGSVQEINR
Rice Protein: Q0D9D0
MLCLTSSSSSAPAPLLPSLADRPSPGIAGGGGNVRLSVVSSPRRSWPGKVKTNFSVPATA
RKNKTMVTVVEEVDHLPIYDLDPKLEEFKDHFNYRIKRYLDQKCLIEKHEGGLEEFSKGY
LKFGINTVDGATIYREWAPAAQEAQLIGEFNNWNGAKHKMEKDKFGIWSIKISHVNGKPA
IPHNSKVKFRFRHGGGAWVDRIPAWIRYATFDASKFGAPYDGVHWDPPACERYVFKHPRP
PKPDAPRIYEAHVGMSGEEPEVSTYREFADNVLPRIRANNYNTVQLMAIMEHSYYASFGY
HVTNFFAVSSRSGTPEDLKYLVDKAHSLGLRVLMDVVHSHASNNVTDGLNGYDVGQNTHE
SYFHTGDRGYHKLWDSRLFNYANWEVLRFLLSNLRYWMDEFMFDGFRFDGVTSMLYHHHG
INKGFTGNYKEYFSLDTDVDAIVYMMLANHLMHKLLPEATIVAEDVSGMPVLCRPVDEGG
VGFDFRLAMAIPDRWIDYLKNKEDRKWSMSEIVQTLTNRRYTEKCIAYAESHDQSIVGDK
TIAFLLMDKEMYTGMSDLQPASPTINRGIALQKMIHFITMALGGDGYLNFMGNEFGHPEW
IDFPREGNNWSYDKCRRQWSLVDTDHLRYKYMNAFDQAMNALEEEFSFLSSSKQIVSDMN
EKDKVIVFERGDLVFVFNFHPNKTYKGYKVGCDLPGKYRVALDSDALVFGGHGRVGHDVD
HFTSPEGMPGVPETNFNNRPNSFKVLSPPRTCVAYYRVDEDREELRRGGAVASGKIVTEY
IDVEATSGETISGGWKGSEKDDCGKKGMKFVFRSSDEDCK [SEQ ID 705]
Rice protein: Q6AVS5
MDRYQRVERPRPESAIEENEIRITAQGLIRNYVSYATSLLQDRRIKEIVLKAMGQAISKS
VAVAEIIKKRVPGLYQDTNISSVSITDVWEPIEEGLVPLEMTRHVSMISITLSPRDLDKN
SPGYQTPVYVEQPRQQPRLQQAPPPPQRQVRQPPPDYEDSYVRGRGRGRGRGRGRGWGRG
GYGGYGGYGNNQGGYNQGGGYYDNQGGYGGYDNQGGYGGYDNQGGYGGGGYGYNQGRYGN
YQENGGYNRGRGGMRGRGNWNYRGGYERGRGGGFPGGRGYGGRGRGRMGGRGGRGN
[SEQ ID 706]
Rice Protein: Q8H920
MALEHLGRRNVAGSLLLLNLLMYVFLLGFAGWALNSSIKNAGADVGVGWGEQPWSPYYRQ
SAWFASRFHLATFAALAGALGVAAKASAAYHGGRSGASWRPQGLAAAASLGTAAWAATAL
AFGVACREIHDAAAAGPAGAARGWRMRALEGLTVTLAFTQLLYVLLLHAAVAGERCGLAC
AADA [SEQ ID 707]
Pea protein: O49927
MAIKTKLSLTIFLFFLLALLCSNLAVGRKEKDPELTTCKDQCDMQRQYDEEDKRICMERC
DDYIKKKQERQKHKEHEEEEEQEQEEDENPYVFEDNDFETKIDTKDGRVLILNKFNEKSK
LLKNIENYGLAVLEIKANAFLSPHHYDSEAILFNIKGRGIIGLVAEDRTERFNLEEGDIM
RVPAGTPMYLVNRDENEKLYIAAFHMPPSSGSAPVNLEPFFESAGRKPESVLNTFSSKVL
QAALKSSKGELETVLDEQKKGRIFKIEKEDVRGLAPKKSLWPFGGPFKSPFNIFSNNPAF
SNKFGSLFEVGPSQEKSGLEGLNLMLTLANITKGSMSTIHYNTNANKIALVIDGEGELEM
ACPHMPSSSSNSRQKKSSISYHNINAKLRPGVMFVVPAGHPFVNIASKKKNLIVVCFEVN
AQRNKKLALAGKKNIVSALDKAAKEVAFDIAAEKVDEVFERKEEFFFPYDNEERKEEHGR
AVV [SEQ ID 708]
Pea protein: Q41000
MFRRATSTFLSRASATRRFSTDVATPATNSSFVEAWRKVSPNIDPPKTPLEFLKTRPPVP
STIPTKLTVNFVLPYSSQLAAKEVDSVIIPATTGEMGVLPGHVATIAELKPGVLTVQEGT
DTTKYFVSSGFRFIHANSVADIIAVEAVPVNQLDRDLVQKGLQEFTQKLNSATTDLEKRE
AQIGIDVDSALNSALTG [SEQ ID 709]
Rice Protein: P20698
MKIIFFFALLAIAACSASAQFDAVTQVYRQYQLQPHLMLQQQMLSPCGEFVRQQCSTVAT
PFFQSPVFQLRNCQVMQQQCCQQLRMIAQQSHCQAISSVQAIVQQLRLQQFASVYFDQSQ
AQAQAMLALNMPSICGIYPSYNTAPCSIPTVGGIWY [SEQ ID 710]
Pea Protein: O04434
MQDSSTMKFSPLDLMTAIIKGKFNPSNDSSQAPASIIFENREFVMILTTSIAVLIGCVVV
LIWRRSNSNKSKQIEVPKLVIKKLPELDVDDGKKKVTVFFGTQTGTAEGFAKAIAEEAKA
RYEKAKFRVVDMDDYAADDDEYLEKLKRETMALFFLATYGDGEPTDNAARFYKWFTEEYE
GEEDSFKNLSYGVFGLGNRQYEHFNKVAKVVDDKLLEQGGKRLVPVGLGDDDQCIEDDFT
AWKEELWPALDQLLRDEDDTPVATPYTAAVSEYRVVIHDPLDATVDEKKRHNVNGHAVVD
AQHPVRANVAVRRELHTPASDRSCTHLEFDISGTGVVYETGDHVGVYCENLSDTVEEAER
ILGLSPDTYLSIHTDDEEGKPLGGSSLPPPFPPCTLRTALTKYADLLSSPKKSALVALAA
HASDPSEADRLRHLASPAGKDEYAEWVISSQRSLLEVMAEFSSAKPPIGVFFAAVAPRLQ
PRYYSISSSPRMAPSRIHVTCALVHDKMPTGRIHKGVCSTWMKNSVPLEKNQDCSWAPIF
VRQSNFRLPADNKVPVIMIGPGTGLAPFRGFLQERLALKEDGAELGPSVLFFGCRNRQVD
YIYEDELNHFVNGGALSELIVAFSRDGPTKEYVQHKMMEKASDIWNMISQGAYVYVCGDA
KGMARDVHRTLHTILQEQGSLDSSKTESMVKNLQMTGRYLRDVW [SEQ ID 711]
Rice Protein: Q5N725
MSAYCGKYKDELIKNAAYIGTPGKGILAADESTGTIGKRFASINVENVEENRRSLRELLF
TTPGALQHLSGVILFEETLYQKTKDGKPFVDVLKEGGVLPGIKVDKGTVEVAGTNKETTT
QGHDDLGKRCAKYYEAGARFAKWRAVLKIGPNEPSQLSIDLNAQGLARYAIICQENGLVP
IVEPEILVDGSHDIERCAYVTEKVLAACYKALNEHHVLLEGSLLKPNMVTPGSESKKVSP
QLIAEYTVRALQRTVPAAVPAIVFLSGGQSEEEATVNLNAMNKLSTKKPWALSFSFGRAL
QQSTLKAWGGKTENVVKAQKAFITRCKANSEATLGTYQGDAVLGEGASESLHVKDYKY
[SEQ ID 712]
Pea Protein: B5A8N6
MASLQTQMISFYAIFLSILLTTILFFKVNSTETTSFLITKFSPDQQNLIFQGDGYTTKEK
LTLTKAVKNTVGRALYSSPIHIWDRETGNVANFVTSFTFVINAPNSYNVADGFTFFIAPV
DTKPQTGGGYLGVFNSAEYDKTTQTVAVEFDTFYNAAWDPSNRDRHIGIDVNSIKSVNTK
SWKLQNGEEANVVIAFNAATNVLTVSLTYPNSLEEENVTSYTLSDVVSLKDVVPEWVRIG
FSATTGAEYAAHEVLSWSFHSELSGTSSSKQAADA [SEQ ID 713]
Rice Protein: Q6K508
MATTTSLLSSCLCALLLAPLFSQGVDAWESRQGASRQCRFDRLQAFEPLRKVRSEAGDTE
YFDERNEQFRCAGVFVIRRVIEPQGLVVPRYSNTPALAYIIQGKGYVGLTFPGCPATHQQ
QFQLFEQRQSDQAHKFRDEHQKIHEFRQGDVVALPASVAHWFYNGGDTPAVVVYVYDIKS
FANQLEPRQKEFLLAGNNQRGQQIFEHSIFQHSGQNIFSGFNTEVLSEALGINTEASKRL
QSQNDQRGDIIRVKHGLQLLKPTLTQRQEEHRQYQQVQYREGQYNGLDENFCTIKARVNI
ENPSRADYYNPRAGRITLLNNQKFPILNLIGMGAARVNLYQNALLSPFWNINAHSVVYII
QGSVRVQVANNQGRSVFNGVLHQGQLLIIPQNHAVIKKAEHNGCQYVAIKTISDPTVSWV
AGKNSILRALPVDVIANAYRISRDEARRLKNNRADEIGPFTPRFPQKSQRGYQFLTEGLS
LIGM [SEQ ID 714]
PEA PROTEIN: Q712V4
QKAPRKQLATKAARKSAPATGGVKKPHRFRPGTVALREIRKYQKSTELLIRKLPFQRLVR
EIAQDFKTDLRFQSSAVSALQEAAEAYLVGLFEDTNLCAIHAKRVTIMPKDIQLARRIRG
ERA [SEQ ID 715]
Pea Protein: P09886
MAQSVSLSTIASPILSQKPGSSVKSTPPCMASFPLRRQLPRLGLRNVRAQAGGDGDNKDN
SVEVHRVNKDDQGTAVERKPRRSSIDISPFGLLDPWSPMRSMRQMLDTMDRIFEDAITIP
GRNIGGGEIRVPWEIKDEEHEIRMRFDMPGVSKEDVKVSVEDDVLVIKSDHREENGGEDC
WSRKSYSCYDTRLKLPDNCEKEKVKAELKDGVLYITIPKTKIERTVIDVQ1Q [SEQ ID 716]
Examples of homologs for each protein
P13918 (Pea)
>gi|137584|sp|P08438.1| VCL_VICFA RecNa me: Full = Vicilin; Flags: Precursor [Vicia faba]
>gi|22057|emb|CAA68559.1| vicilin [Vicia faba var. minor]
>gi|383931031|gb|AFH56916.1| vicilin [Vicia faba]
MAATTLKDSFPLLTLLGIAFLASVCLSSRSDQDNPFVFESNRFQTLFENENGHIRLLQKFDQHSKLLENLQNYRLLEYKSKPHTIFLPQQTDADFIL
VVLSGKAILTVLLPNDRNSFSLERGDTIKLPAGTIGYLVNRDDEEDLRVLDLVIPVNRPGEPQSFLLSGNQNQPSILSGFSKNILEASFNTDYKEIEK
VLLEEHGKEKYHRRGLKDRRQRGQEENVIVKISRKQIEELNKNAKSSSKKSTSSESEPFNLRSREPIYSNKFGKFFEITPKRNPQLQDLNIFVNYVEI
NEGSLLLPHYNSRAIVIVTVNEGKGDFELVGQRNENQQGLREEYDEEKEQGEEEIRKQVQNYKAKLSPGDVLVIPAGYPVAIKASSNLNLVGFGI
NAENNQRYFLAGEEDNVISQIHKPVKELAFPGSAQEVDTLLENQKQSHFANAQPRERERGSQEIKDHLYSILGSF [SEQ ID 506]
>gi|502105533|ref|XP_004492829.1| PREDICTED: vicilin-like isoform X1 [Cicer arietinum] ChickPea
MAIKARFPLLVLLGIVFLASVCAKSDKENPFFFKSNNCQTLFENENGHVRLLQRFDKRSQLFENLQNYRLMEYNSKPHTLFLPQHNDADFILVVL
RGRAILTVLNPNDRNTFKLERGDTIKLPAGTIAYLANRDDNEDLRVLDLAIPVNRPGQFQSFSLSGNENQQSYFQGFSKKILEASFNSDYEEIERV
LLEEQEQKPEQRRGHKGRQQSQETDVIVKISREQIEELSKNAKSNCKKSVSSESEPFNLRSRSPIYSNRFGNFFEITPEKNPQLKDLDIFVNSVEIK
EGSLLLPHFNSRATVILVVNEGKGEVELVGLRNENEQENKKEDEEEEEDRNVQVQRFQSKLSSGDVVVIPASHPFSINASSDLFLLGFGINAQN
NQRNFLAGEEDNVISQIQRPVKEVAFPGSAEEVDRLLKNQRQSHFANAQPQQKRKGSQRIRSPF [SEQ ID 507]
>gi|29539109|emb|CAD87730.1| allergen Len c 1.0101 [Lens culinaris] Lentil
SRSDQENPFIFKSNRFQTIYENENGH1RLLQRFDKRSKIFENLQNYRLLEYKSKPHTIFLPQFTDADFILVVLSGKAILTVLNSNDRNSFNLERGDTI
KLPAGTIAYLANRDDNEDLRVLDLAIPVNRPGQLQSFLLSGTQNQPSFLSGFSKNILEAAFNTEYEElEKVLLEEQEQKSQHRRSLRDKRQEITNE
DVIVKVSREQIEELSKNAKSSSKKSVSSESEPFNLRSRNPIYSNKFGKFFEITPEKNPQLQDLDIFVNSVEIKEGSLLLPNYNSRAIVIVTVNEGKGDF
ELVGQRNENQQEQREENDEEEGQEEETTKQVQRYRARLSPGDVLVIPAGHPVAINASSDLNLIGFG1NAKNNQRNFLAGEEDNVISQIQRPV
KELAFPGSSREVDRLLTNQKQSHFANAQPLQIE [SEQ ID 508]
Q9M3X6 (Pea)
>gi|164512526|emb|CAP06312.1| cvc [Pisum abyssinicum]
MATTVESRFPLLLFPGIIFLASVCVTYANYDEGSETRVPGQRERGRQEGEKEEKRHGEWRPSYEKEEDEEEKQKYRYQREKEDEEEKQKYRYQR
EKKEEKEVQPGRERWEREEDEEQVDEEWRGSQRRQDPEERARLRHREERTKRDRRHKREGEEEERSSESQEQRNPFLFKSNKFLTLFENENG
HIRRLQRFDKRSDLFENLQNYRLVEYRAKPHTIFLPQH1DADLILVVLNGKAILTVLSPNDRNSYNLERGDTIKIPAGTTSYLVNQDDEEDLRVVD
FVIPVNRPGKFEAFGLSENKNQYLRGFSKNILEASLNTKYETIEKVLLEEQEKKPQQLRDRKRRQQGGERDAIIKVSREQIEELRKLAKSSSKKSLPS
EFEPFNLRSHKPEYSNKFGKLFEITPEKKYPQLQDLDILVSCVEINKGALMLPHYNSRAIVVLLVNEGKGNLELLGLKNEQQEREDRKERNNEVQ
RYEARLSPGDVVIIPAGHPVAISASSNLNLLGFGTNAENNQRNFLSGSDDN [SEQ ID 509]
>gi|164512538|emb|CAP06318.1| cvc [Lathyrus annuus]
MATTIKSRFPLLLLLGIIFLASVCVTWANYDEGSEPRVPGQRERGRQEGEKEEKRHGEWRPSYEEEYDEGLEPKVPGKRERGRQEGEKEEKRHE
EWRPSYEKEEDEEEKQKYNYQREKKEHKEVQPGRERWERKQDEKQVEEDEEPGEEQWRGSKRHEDPEERARLRHREEKTKSYVEDNEETSS
KEGRNPFLFKSNKFLTLFENENGHIRRLQRFDERSDIFENLQNYRLVEYRAKPHTMFLPQHIDADLILVVLNGKAILTVLSPNDRNSYNLERGDTV
KLPAGTTSYLVNQDDEEDLRVVDLAIPVNRPGKFEAFGLSANKNQYLRGFSKNILEASLNTKYETIEKVLLEERRDQKGRQQGQETNAIVKVSRE
QIEELRKLAKSSSKKSLLSESEPLNLRSQNPKYSNKFGKFFEITPQKKYPQLQDLDVSISCVEINKGALLLPHYNSRSIGILLVNEGKGNLELVGFKNE
QQRQRENEETNKKLQRYEARLSSGDVVVIPEGHPVAISASSNLNLLGFGINAANNQRNFLTGSDDN [SEQ ID 510]
>gi11645125581embICAP06328.11 cvc [Vicia villosa]
MA-MKSRFPVLLLLGIIFLTSVCVTYANYDEGREPSVPGQRERGRQEGEKEEKRHGEWRPSEEDEEEKYKYEEGRVPGQRERGRQEGEKEEKR
HGKWRPSEEEDEEEKYRYEEGSEPRGPGQRETGRQEGEKEKQRPEREPSYEKEEDEEEKQKYQYHREKKEQREVRPGRERFERHEDEEQWRG
IQRHEDPEERARERYRAEIAKRQVEEEREERDIPHEREQRNPFLFKSNKFQTLFQNENGYIRRLQRFDKRSDLFENLQNYRLVEYRAKPHTIFLPQ
HIDADLIIVVLSGRAILTVLSPDDRNSYNLERGDTIKLPAGTTSYLVNQDDEEDLRVVDLAIPVNRPGKVESFLLSGNKNQYLRGFSKNILEASFNT
NYETIERVLLEEQDKESQQSIGQKRRSQRQETNALVKVSREQLEDLKRLAKSSSQEGLSSQFEPINLRSQNPKYSNKFGKVFEITPEKKYPQLQDL
DLFVSSVDIKEGALMLPHYNSRAIVVLLVNEGRGNLELVGLKNEQQEQREKEDEQQERNNQVQRYEARLSPGDVVIIPAGHPVAVRASSDLNL
LAFGINAENNQRNFLAGSDDN [SEQ ID 511]
P09918 (Pea)
>gi|357454557|ref|XP_003597559.1| Seed lipoxygenase-3 [Medicago truncatula]
>gi|355486607|gb|AES67810.1| seed linoleate 95-lipoxygenase [Medicago truncatula]
MFSGVTGILNRGHKIKGTVVLMRKNVLDINSLTSVGGVIGQGFDILGSTLDNLTAFLGRSVSLQLISATKPDANGKGKLGKATFLEGIITSLPTLG
AGQSAFKIHFEWDDDMGIPGAFYIKNFMQTEFFLVSLTLEDIPNHGSIYFVCNSWIYNAKHHKLDRIFFANKAYLPSETPAPLVHYREEELNNLR
GDGTGERKEWERIYDYDVYNDLGNPEKGDNHARPVLGGSDTYPYPRRGRTGRKPNPKDPKSESRSDFVYLPRDEAFGHLKSSDFLTYGLKAVS
QNVVPALESVIFDLNFTPNEFDSFDEVHGLYEGGIKLPTDVLSKISPLPVLKEIFRTDGEQFLKYPPPKVLQVSRSAWMTDEEFAREMLAGVNPN
VICCLQEFPPRSKLDSQVYGDHTSKITKEHLEPNLEGLTVEEAIQNKKLFLLDHHDSIMPYLRRINSTPTKAYATRTILFLSSDKTLKPLAIELSLPHP
DGDEHGAVSHVYQPALEGVESTIWLLAKAYVVVNDSCYHQLVSHWLNTHAVVEPFVIATNRHLSYLHPIYKLLYPHYRDTMNINSLARQSLVN
DGGIIEKTFLWGRYSMEMSSKVYKNWTLPGQALPADLIKRGMAIEEPSSPCGVKLVVEDYPYAHDGLEIWAAIKTWVQDYVSLYYTTDDILRQ
DSELQAWWKELVEVGHGDKKNEPWWPKMQAREELVEVCTTVIWIASALHAAVNFGQYSYGGLILNRPTLSRRFMPEKGSAEYNELVKSPQ
KAYLKTITPKFQTLIDLSVIEILSRHASDEVYLGERDNPNWTSDTRALEAFKKFGNKLAEIEKNLAQRNNDEKLRHRLGPVQMPYTLLHPSSEEGL
TFRGI PNSISI [SEQ ID 512]
>gi|734403888|gb|KHN32710.1| Seed linoleate 95-lipoxygenase-3 [Glycine soja]
MLGGLLHRGHKIKGTVVLMRKNVLDVNSVTSVGGIIGQGLDLVGSTLDTLTAFLGRSVSLQLISATKADANGKGKLGKATFLEGIITSLPTLGAG
QSAFKINFEWDDGSGIPGAFYIKNFMQTEFFLVSLTLEDIPNHGSIHFVCNSWIYNAKLFKSDRIFFANQTYLPSETPAPLVKYREEELHNLRGDG
TGERKEWERIYDYDVYNDLGDPDKGENHARPVLGGNDTFPYPRRGRTGRKPTRKDPNSESRSNDVYLPRDEAFGHLKSSDFLTYGLKSVSQN
VLPLLQSAFDLNFTPREFDSFDEVHGLYSGGIKLPTDIISKISPLPVLKEIFRTDGEQALKFPPPKVIQVSKSAWMTDEEFAREMLAGVNPNLIRCL
KDFPPRSKLDSQVYGDHTSQITKEHLEPNLEGLTVDEAIQNKRLFLLDHHDPIMPYLRRINATSTKAYATRTILFLKNDGTLRPLAIELSLPHPQGD
QSGAFSQVFLPADEGVESSIWLLAKAYVVVNDSCYHQLVSHWLNTHAVVEPFIIATNRHLSVVHPIYKLLHPHYRDTMNINGLARLSLVNDGG
VIEQTFLWGRYSVEMSAVVYKDWVFTDQALPADLIKRGMAIEDPSCPHGIRLVIEDYPYAVDGLEIWDAIKTWVHEYVFLYYKSDDTLREDPEL
QACWKELVEVGHGDKKNEPWWPKMQTREELVESCAIIIWTASALHAAVNFGQYPYGGLILNRPTLSRRFMPEKGSAEYEELRKNPQKAYLKT
ITPKFQTLIDLSVIEILSRHASDEVYLGERDNPNWTSDTRALEAFKRFGNKLAQIENKLSERNNDEKLRNRCGPVQMPYTLLLPSSKEGLTFRGIP
NSISI [SEQ ID 513]
>gi|593700103|ref|XP_007150490.1| hypothetical protein PHAVU_005G 157000g [Phaseolus vulgaris]
>gi|561023754|gb|ESW22484.1| hypothetical protein PHAVU_005G157000g [Phaseolus vulgaris]
MFSGVSGLINRGHKLKGTVVLMRKNVLDVNSVTSVGGIVGQGLDILGSTVDNLTAFLGRSVSLQLISATKPDANGKGKLGKATFLEGIITSLPTL
GAGQSAFKIHFEWDDEMGIPGAFYIKNFMQTEFYLVSLTLEDIPNHGSLHFLCNSWIYNAKHFKNDRIFFVNQIYLPSETPAPLVKYREEELVN
MRGDGTGERKEWDRIYDYDVYNDLGDPDKGENNARPILGGSDTLPYPRRGRTGRRPTRKDPKSESRSSDIYLPRDEAFGHLKSSDFLTYGLKS
VSQNFLPALQSAFDLNFTPNEFDSFEEVHGLYSGGIKLPTDVLSKISPLPVLKEIFRTDGEQTLKFPPPKVVQDSKSAWMTDEEFAREMICGVNP
NLIRLLQDFPPQSKLDSQVYGDHTSQITKENLEPNLEGLTVDEAIQSKRLFLLDHHDSIMPYLRRINATSSKAYATRTILFLKKDRTLKPLAIELSLP
HPGGDKSGVVSQVFLPADEGVESSVWLLAKSYVIVNDSSYHQLVSHWLNTHAVVEPFVIATNRHLSVVHPIYKLLHPHYRDTMNINALARGD
LVNHGGIIEKTFVWGRYSMEMSAVIYKDWVFTDQALPADLIKRGIATEDPECPHGLRLFIEDYPYAVDGLEIWDAIKTWVHEYVFLYYKSDDTL
KEDPELQAWWKELVEVGHGDKKNEPWWPKMQTREELVEACSIVIWTASALHAAVNFGQYPYGGLILNRPTLSRRFMPEEGSAEYEELKKSP
QKALLKTITPKFQTLVDLSVIEILSRHASDEVYLGERDNPNWTSDTRALEAFKRFGKKLSEIEKKLSQRNNDEKLRNRYGPVMMPYTLLFPSSDE
GLTFRGIPNSISI [SEQ ID 514]
P02857 (Pea)
>gi|483449|emb|CAA83677.1| legumin A [Vicia sativa]
MAKLLALSLSFCFLLFSSCFALREQSQQNECQLERINALEPDNRIESEGGLIETWNPNNRQFRCARVALSRATLQRNALRRPYYSNAPQEIYIQQ
GNGYFGMVFPGCPETHEEPQQSEQGEGRRYRDSHQKVNRFREGDIIAVPTGIAFWMYNDQDTPVIAISLTDTGSSNNQLDQMPRRFYLAG
NQEQEFLRYQHQQGGKQEQDNDGNNIFSGFKRDFLEDAFNVNRHIVDRLQGRNEDEEKGAIVKVKGGLSIIAPPERQARHERGSRQEEDED
EKEERQPSHHKSRRDEDEDDKEKRHSQKGQSRRQGDNGLEETVCTAKLRANIGSSPSPDIYNPQAGRIKTVTSLDLPVLRWLKLSAEHGSLHK
NAMFVPHYNLNANSVIYALKGRARLQVVNCNGNTVFDGELEAGRALTVPQNYAVAAKSLSERFTYVAFKTDDRASIARLAGTSSVIDDLPLDV
VAATFNMQRNEARQLKSNNPFKFLVPPRQSEMRASA [SEQ ID 515]
>gi|600108|emb|CAA86824.1| legumin A precursor [Vicia narbonensis]
MAKLLALSLSLCFLLFSNSFALREQSQQNECQLERLDALEPDNRIESEGGLIETWNPNNRQFRCAGVALSRVTLQRNALRRPYYSNAPQEIYIQQ
GNGYFGVVFPGCPETFEEPQESEQRERRRYRDSHQKVNRFREGDIIAVPTGNVLWMYNDQDTPVIAISLTDTGSSNNQLDQIPRRFYLAGNQ
EQEFLRYQREQGGKQEQENDGNNIFSGFKRDFLEDALNVNRHIVDRLQGRNEDEEKGAIVKVKGGLSIITPPERQRGSRQEEDEDEKEERQPS
RRRDESQKGESRRHGDNGLEETVCTAKLRVNIGSSPSPDIYNPQAGRINTVTSLDLPVLRWLKLSAEHGSLRKNALIVPHYNRNANSVIYALKG
RARLQVVNCNGNTVFDGELEAGRALTVPQNYAVAAKSLSERFTYVAFKTNDRDGIARLAGTSSVINDLPLDVVAATFNLQRNEARQLKSNNPF
KLLVPPRESEKRASA [SEQ ID 516]
>gi|502110016|ref|XP_004493779.1| PREDICTED: legumin-like [Cicer arietinum]
MAKLLALSLSFCFLLFGSCFALRDQPEQNECQLEHLNALEPDNRIKSEGGLIETWNPSNKQFRCAGVALSRATLQPNSLRRPFYTNAPQEIFIQQ
GNGYFGMVFPGCVETFEEPRESEQGEGSKFRDSHQKVNRFREGDIIAVPTGVVFWMFNDQDTPVIAVSLIDTSSFQNQLDQMPRRFYLAGN
HEXXXXXXXXQQEGSEEEENEGGNIFSGFKRDFLEDALNVNRRIVNKLQGRNEDEEKGAIVKVKGGLSIITPPEKEPRQKRGSRQEEDEDEDEK
RQPHRHSRQDEDEDEKRQPRRHSRGGSKSQRDNGFEETICTARLHQNIGSSSSPDIYNPQAGRIKTVTSFDLPALRFLKLSAEFGSLHKNAMFV
PHYNLNANSILYALKGRARLQIVNCKGNSVFDGELEAGRALIVPQNFAIAAKSLSDRFSYVAFKTNDRAAIGRLLGASSLINGMPEEVVAAAFN
MERNEARQLKFNSPFSFLVPPRSDSDNKAAA [SEQ ID 517]
P02855 (Pea)
>gi|164512536|emb|CAP06317.1| cvc [Lathyrus hirsutus]
MAIIIKSRFPLLLLLGIIFLASVCATWANYDEGSEPRVPGQRERGRQEGEKAEKSHEKWRPSYEEEYDEGSEPRVPGKRERGRQEGEKEEKRHGE
WRPSHEEEYDEGSEPRVPTHGERGRQEGEKEEKRHEEWRPSYEKEEDEEEKEKYKYQREKKEQKEVQPGREKWERKQDEKHVEEDEDQEEE
QWRGSKRREDPEERARLRYREERTKSNVEEETEERRNPFLFKSNKFLTLFENENGHIRRLQRFDERSDIFENLQNYRLVEYKAKPHTMFLPQHID
ADLIIVVLNGKAILTVLSPNDRNSYNLERGDTIKLPAGTTSYLVNQDDEEDLRVVDLAIPVNRPGKFEAFGLSANKNQYLRGFSKNILEAFLNTKY
ETIEKVLLEEQERRDRKGRQQGQETNAIVKVSREQIEELRKLAKSSSKKSLLSESEPINLRSQNPKYSNKFGKLFEITPEKKYPQLQDLDVSISCVEI
NEGAPLLPHYNSRAIVLLLVNEGKGNLELVGFKNEQQRQRENEERNKKVQRYEARLSPGDVVVIPAGHPVAISASLNLNLVGFGVNAENNQR
NFLTGSDDN [SEQ ID 518
>gi|164512542|emb|CAP06320.1| cvc [Lathyrus cicera]
MATIIKSRFPLLLLLGIIFLASVCVTLANYDEGSEPRVPAQRERGRQEGEKEEKRHGEWRPSHEKEYDEGSEPRVPGRRERGRQEGEKEEKRHGE
WRPSYEKEYDEGSEPRVPGRRERGRQEGEKEEKRHGEWRPSYEKEYDEEEKQKYQYEREKEEQKEVQPGRERWERKEDEEKEEDQWRGSQ
RHEDPEERARLRYRKERTKKYVEEDTEETSSESQGRRNPFLFKSNKFLTLFENENGYIRRLQRFDERSDIFENLQNYRLVEYRAKPHTIFLPQHIDA
DLILVILNGKAILTVLSPNDRNSYNLERGDTIKLPAGTTSYLVNEDDEEDLRVVDLVIPVNRPGKFEAFDLNQYLGGFSKSVLEASLNTKYETIEKVL
LEEQQKQGQETNAIVKVSREQIEELRKLAKSSSKKSLLSELEPVNLRSHSPKYSNKFGKFFEITPEKKYPQLQDLDVSISCVEINEGALLLPHYNSRA
IVVVLVNEGKGNLELLGVQNEDEQQERKERNKEVQRYEARLSPGDVVIIPSGHPVAVSASSNLNLLGFGINAENNQRNFLSGSDDN [SEQ ID 519]
>gi|164512544|emb|CAP06321.1| convicilin [Lathyrus sativus]
MATIIKSRFPLLLLLGIIFLASVCVTYANYDEGSEPRVPAQRERGRQEGEKEEKRHGEWRPSSEKEYDEGSEPRVPGRRERGRQEGEKEEKRHGE
WRPSYEKEYDEEEKQKYQYEREKKEQKEVEPGRERWERKEDEEKEEDQWRGSQRHEDPEERARLRYRKERTKKYVEEDTEETSSESQGRRNP
FLFKSNKFLTLFENENGYIRRLQRFDERSDLFENLQNYRLVEYRAKPHTIFLPQHIDADLILVILNGKAILTVLSPNDRNSYNLERGDTIKLPAGTTS
YLVNEDDEEDLRVVDLVIPVNRPGKFEAFDLNQYLGGFSKSVLKASLNTKYETIEKVLLEEQQKQGQETNAIVKVSREQIEELRKLAKSSSKKSLLS
ELEPVNLRSHSPKYSNKFGKFFEITPEKKYPQLQDLDVSISCVEINEGALLLPHYNSRAIVVLLVNEGKGNLELLGVQDEDEQQERKKRNKEVQRY
EARLSPSDVVIIPAGHPVAVSASSNLNLLGFGINAENNERNFLSGSDDN [SEQ ID 520]
D3VNE1 (Pea)
>gi|357507721|ref|XP_003624149.1| Provicilin [Medicago truncatula]
>gi|87162569|gb|ABD28364.1| Cupin, RmlC-type
[Medicago truncatula] >gi|355499164|gb|AES80367.1| vicilin 47 kDa protein [Medicago truncatula]
MAIKAPFQLLMLLGIFFLASVCVSSRDDRHDQENPFFFNANHFQTLFENENGHIRLLQRFDKRSKIFENLQNYRLLEYHSKPHTLFLPQHNDAD
FlLAVLSGKAILTVLNPDNRNSFNLERGDTIKLPAGSIAYLANRDDNEDLRVLDLAIPVNRPGKFQSFSLSGSQNQQSFFSGFSKNILEAAFNANY
EEIERVLIEEHEQEPQHRRGLRKDRRQQSQDSNVIVKVSREQIEELSRHAKSSSRRSGSSESAPFNLRSREPIYSNEFGNFFEITPEKNPQLKDLDIL
VNYAEIREGSLLLPHFNSRATVIVVVDEGKGEFELVGQRNENQQEQREEDEQQEEERSQQVQRYRARLSPGDVYVIPAGHPTVVSASSDLSLL
GFGINAENNERNFLAGEEDNVISQIERPVKEVAFPGSAQDVESLLKNQRQSYFANAQPQQREREEGRSQRQRELISSILGVF [SEQ ID 521]
>gi|164512560|emb|CAP06329.1| convicilin [Vicia peregrina]
MATTFKSRFSLLLLLGIIFLAFVCVTCANYDEGSEPRVPGQRERGRQEGEKEEQSRERHPQREPSREKEEDEEEKQKYDEGTEPRVPGQRERGR
QEGEKEEQRRERHPGQREPSQEEDEEREESDRRQEGSSKSEEQRNPFLFKSNKFLTLFQNGNGHIRLLQRFDKRSDLFENLQNYRLLEYRAKPH
TIFLPQHIDADLILVVLSGRAILTVLSPDDRNSYNLERGDTIKLPAGTTSYPLNQDDEEDLRVVDLAISVNRPGKVESFNLSGNKNQYLRGFSENIL
EASFNTKYETIEKVLLEEQDKESQQPRGQRLQRQETNALVKVSREQVEELKRLARTSSKKGVSSEFEPFNLRSHGPKYSNKFGKFFEITPEKKYPQ
LQDLDISVSSVEINEGALFLPHYNSRAIVVVLVDEGKGNLELVGFKNEQQEQREKEDEQEERNKQVQRYEAKLSPGDVVIIPAGHPVAVSASSN
LNLLGFGINAENNQRNFLTGSDDN [SEQ ID 522]
>gi|164512562|emb|CAP06330.1| convicilin [Vicia lutea]
MATTIKLRFPLLLLLGVILLASVCVTCANYDEGSEPRVPGRPEGEKEEKHRGKLRPSYEKEEDEGEKQRYHYEKKEQKEAQPRREKKEQKEEEKQ
VEEESRESQRYEDPGERARERYRAEIIKRQVEKEREERDRRHQREGEEEEGSSKSRNPFLFKSNNFLTLFENENGHIRLLQRFDKRSDLFENLQNY
RLVEYRAKPHTIFLPQHIDADLILVVLSGKAILTVLSPNNRNSYNLKRGDTIKLPAGTTSYLLNSDDEEDLRMVDLAISVNRPGKVESFNLSGNKN
QYLRGFSKNILEASFNTKYETIEKVLLEEQDKESQQSIGQKRISQRQETNALVKVSREQIEEPKRLARSSSRKGVSSEFEPINLRSQRPKYSNKFGKF
YEISPEKKYPQLQDLDVSVSSVEINEGALLLPHYNSRAIVTVLVNEGKGNLELIGFQNEQQGQREKEDEQQHERNKQVQRYDARLSSGDVVIIP
AGHPVAVSASSNLDLLGFGINAENSQRNFLTGSDDN [SEQ ID 523]
P07728 (Rice)
>gi|531874314|gb|AGT59174.1| glutelin, partial [Oryza sativa Indica Group]
CRFDRLQAFEPIRSVRSQAGTTEFFDVSNEQFQCTGVSAVRRVIEPRGLLLPHYTNGASLVYIIQGRGITGPTFPGCPESYQQQFQQSGQAQLT
ESQSQSHKFKDEHQKIHRFRQGDVIALPAGVAHWCYNDGEVPVVAIYVTDLNNGANQLDPRQRDFLLAGNKRNPQAYRREVEERSQNIFSG
FSTELLSEALGVSSQVARQLQCQNDQRGEIVRVEHGLSLLQPYASLQEQEQGQVQSRERYQEGQYQQSQYGSGCSNGLDETFCTMKVRQNI
DNPNRADTYNPRAGRVTNLNTQNFPILNLVQMSAVKVNLYQNALLSPFWNINAHSVVYITQGRARVQVVNNNGKTVFNGELRRGQLLIIPQ
HYAVVKKAQREGCAYIAFKTNPNSMVSHIAGKSSIFRALPNDVLANAYRISREEAQRLKHNRGDEFGAFTPIQYKSYQDVYNAAESS [SEQ ID
524]
>gi|109894635|gb|ABG47337.1| glutelin precursor [Zizania latifolia]
MNMATINGPTIFFTVCLFLLCHGSLAQLLGQSTSQWQSSHRGSSRQCRFDRLQAFEPVRSVRSQAGTTEFFDASNELFQCAGVSIVRRIIEPRG
LLLPQYTNGATIMYIIQGRGITGQTFPGCPESYQQQFQQSMQAQLTGSQSQSQKFKDEHQKINRFRQGDVIALPAGVAHWCYNDGEVPVVA
IYVIDINNAANQLDPRQRDFLLAGNMRSPQAYRREVENQSQNIFSGFSAELLSEALGISTGVARQLQCQNDQRGEIVRVEHGLSLLQPYASLQE
QEQKQEQPRERYQVTQHQQSQYGGGCSNGLDETFCAMRIWQNIDNPNLADTYNPRAGRVTNLNSQKFPILNLIQMSAVKVNLYQNALLSP
FWNINSHSVVYVTQGCARVQVVNNNGKTVFNGELRRGQLLIIPQHYVVVKKAQREGCAYIAFKTNPNSMVSHIVGKSSIFRALPTDVLANAY
RISREDAQRLKHNRGDELGAFTPLQYKSYQDVSSVAASS [SEQ ID 525]
>gi|472867|emb|CAA52764.1| 11S globulin [Avena sativa]
MATTSFPSMLFYFCIFLLFHGSMAQLFGQSSTPWQSSRQGGLRGCRFDRLQAFEPLRQVRSQAGITEYFDEQNEQFRCTGVSVIRRVIEPQGL
VLPQYHNAPALVYILQGRGFTGLTFPGCPATFQQQFQPFDQSQFAQGQRQSQTIKDEHQRVQRFKQGDVVALPAGIVHWCYNDGDAPIVA
IYVFDVNNNANQLEPRQKEFLLAGNNKREQQSGNNIFSGLSVQLLSEALGISQQAAQRIQSQNDQRGEIIRVSQGLQFLKPIVSQQVPGEQQV
YQPIQTQEGQATQYQVGQSTQYQVGKSTPYQGGQSSQYQAGQSWDQSFNGLEENFCSLEARKNIENPQHADTYNPRAGRITRLNSKNFPIL
NIVQMSATRVNLYQNAILSPFWNINAHSVIYMIQGHARVQVVNNNGQTVFNDILRRGQLLIVPQHFVVLKKAEREGCQYISFKTNPNSMVSH
IAGKSSILRALPIDVLANAYRISRQEARNLKNNRGEEFGAFTPKLTQKGFQSYQDIEEGSSSPVRASE [SEQ ID 526]
P14614 (Rice)
>gi|115445309|ref|NP_001046434.1| Os02g0248800 [Oryza sativa Japonica Group]
>gi|37993738|gb|AAR06952.1|
glutelin type-B [Oryza sativa Japonica Group] >gi|47497729|dbj|BAD19794.1|
glutelin type-B [Oryza sativa Japonica
Group] >gi|113535965|dbj|BAF08348.1| Os02g0248800 [Oryza sativa Japonica Group]
>gi|215768942|dbj|BAH01171.1|
unnamed protein product [Oryza sativa Japonica Group] >gi|284431772|gb|ADB84627.1|
glutelin [Oryza sativa Japonica Group]
MTISVFSRFSIYFCVLLLCNGSMAQLFDPATNQWQTHRQGSFRECRFERLQAFEPLQNVRSEAGVTEYFDETNELFQCTGTFVIRRVIQPQGLL
IPRYANTPGMVYIIQGRGSMGLTFPGCPATYQQQSQQFLFQGESQSQKFIDEHQKIHQFRQGDIVVLPTGVAHWFYNDGDTPVVALYVYDI
NNSANQLEPRHREFLLAGKNNRVQQVYGRSIQQHSGQNIFNGFSVEPLSEALN1NTVTTKRLQSQNDQRGEIIHVKNGLQLLKPTLTQRQEQE
QAQYQEVQYSEKPQTSSRWNGLEENLCTIKTRLNIENPSRADSYDPRAGRITSLDSQKFPILNIIQMSATRVNLYQNAILTPFWNVNAHSLMYV
IRGRARVQVVSNFGKTVFDGVLRPEQLLIIPQNYVVLKKAQHEGCQYIAINTNANAFVSHLAGVDSVFHALPVDVIANAYCISREEARRLKNNR
GDEYGPFPPRLQQQIYPEFSNESKGETSE[SEQ ID 527
>gi|428674402|gb|AFZ41188.1| glutelin, partial [Oryza sativa Japonica Group]
LLCHGSMAQIFSLGINPWQNPRQGGSRECRFDRLQAFEPLRKVRHEAGVTEYFDEKNEQFQCTGTLVIRRIIEPQGLLLPRYSNTPGLVYIIQGT
GVLGLTFPGCPATYQKQFRHFGLEGGSQRQGKKLRDENQKIHQFRQGDVVALPSGIPHWFYNEGDTPVVALFVFDVNNNANQLEPRQKEFL
LAGNNIEQQVSNPSINKHSGQNIFNGFNTKLLSEALGVNIEVTRRLQSQNDRRGDIIRVKNGLRLIKPTITQQQEQTQDQYQQIQYHREQRSTS
KYNGLDENFCAIRARLNIENPNHADTYNPRAGRITNLNSQKFSILNLVQMSATRVNLYQNAILSPFWNINAHSLVYTIQGRARVQVVSNHGKA
VFNGVLRPGQLLIIPQNYVVMKKAELEGFQFIAFKTNPNAMVNHIAGKNSVLRAMPVDVIANAYRISRQEARSLKNNRGEEIGAFTPRYQQQ
KIHQEYSNPNESETQ [SEQ ID 528]
>gi|226510|prf| 11515394A seed storage globulin
MATTRFPSLLFYSCIFLLCNGSMAQLFGQSFTPWQSSRQGGLRGCRFDRLQAFEPLRQVRSQAGITEYFDEQNEQFRCAGVSVIRRVIEPQGLL
LPQYHNAPGLVYILQGRGFTGLTFPGCPATFQQQFQPFDQARFAQGQSKSQNLKDEHQRVHHIKQGDVVALPAGIVHWCYNDGDAPIVAV
YVFDVNNNANQLEPRQKEFLLAGNNKREQQFGQNIFSGFSVQLLSEALGISQQAAQKIQSQNDQRGEIIRVSQGLQFLKPFVSQQGPVEHQA
YQPIQSQQEQSTQYQVGQSPQYQEGQSTQYQSGQSWDQSFNGLEENFCSLEARQNIENPKRADTYNPRAGRITHLNSKNFPTLNLVQMSA
TRVNLYQNAILSPYWNINAHSVMHMIQGRARVQVVNNHGQTVFNDILRRGQLLIIPQHYVVLKKAEREGCQYISFKTTPNSMVSYIAGKTSIL
RALPVDVLANAYRISRQESQNLKNNRGEEFGAFTPKFAQTGSQSYQDEGESSSTEKASE [SEQ ID 529]
P07730 (Rice)
>gi|225959|prf| 11404367A glutelin
MASTNRPIVFFTVCLFLLCDGSLAQQLLGQSTSQWQSSRRGSPRGCRFDRLQAFEPIRSVRSQAGTTEFFDVSNELFQCTGVSVVRRVIEPRGL
LLPHYTNGASLVYIIQGRGITGPTFPGCPETYQQQFQQSGQAGLTESQSQSHKFKDEHQKIHRFRQGDVIALPAGVAHWCYNDCEVPVVAIYV
TDINNGANQLDPRQRDFLLAGNKRNPQAYRREVEEWSQNIFSGFSTELLSEAFGISNQVARQLQCQNDQKGEIVRVERGLSLLQPYASLQEQ
EQGQMQSREHYQEGGYQQSQYGSGCPNGLDETFCVNKVRQNIDNPNRADTYNPRAGRVTNLSQNFPILNLVQMSAVKVNLYQNTDTWIS
MGQEENALLSPFWNINAHSIVYITQGRAQVQVLRRGQLLIVPQHYVVVKKAQREGCAYIAFKTNPNSMVSHIAGKSSIFRALPTDVLANAYRIS
REEAQRLKHNRGDEFGAFTPLQYKSYQDVYNVAESS [SEQ ID 530]
>gi|573943558|ref|XP_006654150.1| PREDICTED: glutelin type-A 3-like [Oryza brachyantha]
MKSSIVFSTICLVLLCHGSLAQLLSQSTSQWQSSRRGSPRQCRFDQLQAFEPIRTVRSQAGVTEFYDVSNELFQCTGVSVVRRVIEPRGLLLPHY
SNGATLVYIIQGRGITGPTFPGCPETYQQQFQQSGEAQPFEGQSHKFRDEHQKIHRFRQGDVVALPAGVAHWCYNDGEVPIVAIYVTDIYNS
ANQLDPRHRDFFLAGNNKVAQQLYRSEARENSKNIFGGFSVELLSEALGISRGVARQLQCQNDQRGEIVRVEHGLALLQPYASVQEQQQEQV
QSRDYEQTQYQQKQPQGSCSNGLDETFCTMRLRQNIDNPNLADTYNPKAGRITYLNGQKFPILNLVQMSAVKVNLYQNAVLSPFWNINAHS
VVYITQGRARVQVVNNNGKTVFDGELRQGQLLIIPQHHVVLKKAQREGCSYIALKTNPNSIVSHIAGKNSIFRALPGDVVTNAYRISREEAKRIK
HNRGDESGVFAPSHAYRSYQDMSVAA [SEQ ID 531]
>gi|721641733|ref|XP_010231907.1| PREDICTED: 12S seed storage globulin 1-like
[Brachypodium distachyon]
MAHTSFSSFLSYFCLFLLFHGSMAQVLGQVSTWQSSRQGGSRDCSFDRLQAIEPVTQVRSQAGLTEYFDEQNEQFRCAGVFVIRRVIEPRGLL
LPRYHNTPGLVYILQGNGFVGLTFPGCPETFREQFQQFRQTQSTLGQSQCQSQKLGDVHQRVHQFTQGDVVALPTGVAHWIYNGGDAPVV
IVYVFDVNNNANQLEPRQKEFLLGGNYNGVLQYGQNIFSGFNAQLLSQAFGINEQTSQRIQNQNDGRGDIIRVDNGLQFLKPVVTQQQPEQ
PFMPIQHQTGQSSRNGLEENFCSLEPRQNIEDPNRADTYNPRAGSITRLNGQNFPILNLVQMSATRVNLQKNAILSPFWNINAHSVVYVIQG
HALVQVVNNQGHNVFNGLLHRGQLLIIPQNYVVLKKAESEGYQYIAFKTNANSMVSHIAGKNSILRALPVDVIANAYRISRQEAQNLKNNRGE
ETGVLTPNFSQSTCQSYQTEDVQSLRPMSHWSE [SEQ ID 532]
>gi|169244463|gb|ACA50505.1| seed allergenic protein RAG2 [Oryza sativa Japonica Group]
MASNKVVFSALLLIIVSVLAATATMADHHKDQVVYSLGERCQPGMGYPMYSLPRCRAVVKRQCVGHGAPGGAVIDEQLRQDCCRQLAAVID
DSWCRCSALNHMVGGIYRELGATDVGHPMAXVFPGCRRGIDLERAAASLPAFCNVIDIPNGTGGVCYWLGYPRTPRTGH [SEQ ID 533]
>gi|5777592|emb|CAA44001.1| low molecular weight globulin [Oryza sativa]
MASNKVVFSALLLIIVSVLRRDGTMADHHKDQVVYSLGERCQPGMGYPMYSLPRCRAVVKRQCVGHGAPGAVDEQLRQDCCRQLAAVDD
SWCRCSALNHMVGGIYRELGATDVGHPMAEVFPGCRRGDLERAAASLPAFCNVDIPNGTGGVCYWLGYPRTPRTGH [SEQ ID 534]
>gi|115471175|ref|NP_001059186.1| Os07g0214600 [Oryza sativa Japonica Group]
>gi|23616954|dbj|BAC20657.1|
allergen RA16 [Oryza sativa Japonica Group] >gi|113610722|dbj|BAF21100.1|
Os07g0214600 [Oryza sativa Japonica
Group] >gi|125557687|gb|EAZ03223.1| hypothetical protein Osl_25372 [Oryza sativa Indica Group]
MASNKVVISALLVVVVSVLAATTTMADHHQEQVVYTPGQLCQPGIGYPTYPLPRCRAFVKRQCVAPGTVDEQVRRGCCRQLAAIDSSWCRC
DALNHMLRI1YRESGAADAGHPMAEVFRGCRRGDIERAAASLPAFCNVDIPNGVGGVCYWLPGTGY [SEQ ID 535]
Q0DEV5 (Rice)
>gi|83375868|gb|ABC17777.1| waxy [Oryza rufipogon]
MSALTTSQLATSATGFGIADRSAPSSLLRHGFQGLKPRSPAGGDATSLSVTTSARATPKQQRSVQRGSRRFPSVVVYATGAGMNVVFVGAEM
APWSKTGGLGDVLGGLPPAMAANGHRVMVISPRYDQYKDAWDTSVVAEIKVADRYERVRFFHCYKRGVDRVFVDHPSFLEKVWGKTGEKI
YGPDTGVDYKDNQMRFSLLCQAPRILNLNNNPYFKGTYGEDVVFVCNDWHTGPLASYLKNNYQPNGIYRNAKVAFCIHNISYQGRFAFEDYP
ELNLSERFRSSFDFIDGYDTPVEGRKINWMKAGILEADRVLTVSPYYAEELISGIARGCELDNIMRLTGITGIVNGMDVSEWDPSKDKYITAKYD
ATTAIEAKALNKEALQAEAGLPVDRKIPLIAFIGRLEEQKGPDVMAAAIPELMQEDVQIVLLGTGKKKFEKLLKSMEEKYPGKVRAVVKFNAPLA
HLIMAGADVLAVPSRFEPCGLIQLQGMRYGTPCACASTGGLVDTVIEGKTGFHMGRLSVDCKVVEPSDVKKVAATLKRAIKVVGTPAYEEMV
RNCMNQDLSWKGPAKNWENVLLGLGVAGSAPGIEGDEIAPLAKENVAAP [SEQ ID 536]
>gi|297614332|gb|AD148504.1| glycogen synthetase [Oryza officinalis]
MSALTTSQLATSATGFGIADRSAPSSLLRHGFQGLKPRSPAGGDASSLSVTTSARATPKQQRSVQRGSRRFPSVVVYATGAGMNVVFVGAEM
APWSKTGGLGDVLGGLPPAMAANGHRVMVISPRHDQYKDAWDTSVVAEIKVADRYERVRFFHCYKRGVDRVFIDHPSFLEKVWGKTGEKI
YGPDTGVDYKDNQMRFSLLCQAALEAPRILNLNNNPYFKGTYGEDVVFVCNDWHTGPLPSYLKNNYQPNGIYRNAKVAFCIHNISYQGRFAF
EDYPELNLSERFRSSFDFIDGYDTPVEGRKINWMKAGILESDRVLTVSPYYAEELISGIARGCELDNIMRLTGITGIVNGMDVSEWDPSKDKYIA
AKYDATTAIEAKALNKEALQAEAGLPVDRKIPLIAFIGRLEEQKGPDVMAAAIPELMQENVQIVLLGTGKKKFEKLLKSMEEKYPGKVRAVVKF
NAPLAHLIMAGADVLAVPSRFEPCGLIQLQGMRYGTPCACASTGGLVDTVIEGKTGFHMGRLSVDCKVVEPSDVQKVATTLKRAIKIVGTPAY
NEMVRNCMNQDLSWKGPAKNWENVLLGLGVAGSAPGVEGEEIAPLAKENVAAP [SEQ ID 537]
>gi|389620054|gb|AFK93486.1| granule-bound starch synthase [Hordeum vulgare subsp. vulgare]
MAALATSQLATSGTVLGVTDRFRRPGFQGLRPRNPADAALGMRTIGASAAPKQSRKAHRGSRRCLSVVVRATGSGMNLVFVGAEMAPWS
KTGGLGDVLGGLPPAMAANGHRVMVVSPRYDQYKDAWDTSVISEIKVADEYERVRFFHCYKRGVDRVFIDHPWFLEKVRGKTKEKIYGPDA
GTDYEDNQQRFSLLCQAALEAPRILNLNNNPYFSGPYGEDVVFVCNDWHTGLLACYLKSNYQSNGIYRTAKVAFCIHNISYQGRFSFDDFAQL
NLPDRFKSSFDFIDGYDKPVEGRKINWMKAGILQADKVLTVSPYYAEELISDEARGCELDNIMRLTGITGIVNGMDVSEWDPTKDKFLAVNYDI
TTALEAKALNKEALQAEVGLPVDRKVPLVAFIGRLEEQKGPDVMIAAIPEILKEEDVQIILLGTGKKKFEKLLKSMEEKFPGKVRAVVRFNAPLAH
QMMAGADLLAVTSRFEPCGLIQLQGMRYGTPCVCASTGGLVDTIVEGKTGFHMGRLSVDCNVVEPADVKKVATTLKRAVKVVGTPAYQEM
VKNCMIQDLSWKGPAKNWEDVLLELGVEGSEPGIVGEEIAPLAMENVAAP [SEQ ID 538]
P14323 (Rice)
>gi|573918992|ref|XP_006647120.1| PREDICTED: glutelin type-B 2-like [Oryza brachyantha]
MATTVFSRFSTYFCVLLLCHGSMAQLFNPSTNPWHNPRQGSSRECRFDRLQPFEPLRKVRSEAGVTEYFDEKNELFQCTGTFVIRRVIQPQGLL
VPRYTNAPGLVYIIQGRGSIGLTFPGCPATYQQQFQQFLPQEQSQSQKFRDEHQKIHQFRQGDIVALPAGVAHWFYNDGDAPVVAVYVYDV
KNSANQLEPRQREFLLGGNNMRAQQVYGSSAEQHSRQNIFSGFGVEILSEALGISTVTTKRLQSQNDQRGEIIHVKNGLQFLKPTLTQQQEQA
QAQYQEVQYSEQQQTSSRWNGLDENFCTIKARMNIENTSRADTYNPRAGRTTSLNSQKFPILNLVQMSATRVNLYQNAILSTFWNVNAHSL
VYTIQGRARVQVVSNFGKTVFDGELRPGQLLIIPQHYVVLKKAQREGFRYIAIKTNANAFVSQLVGKNSVFRSLPVDVIANVYRISREQARSLKN
NRGEEHGAFAPRSQQQSYPGFSNQSESETSE [SEQ ID 539]
>gi|573919041|ref|XP_006647142.1| PREDICTED: glutelin type-B 4-like [Oryza brachyantha]
MATTTFSRFSIYFCVLLLCHGSMAQLFSPTLNPWHSSRRGGSRDCRFDRLQAFEPLRRVRSEAGVTEYFDERNEQFQCTGTFVIRRVIEPQGLL
VPRYTNTPGVVYIMQGRGSMGLTFPGCPATYQQQFQQFLPEGQSQSQKFRDEHQKIHQFRQGDIVALPAGVAHWFYNEGDTPVVALYVFD
INNSANQLEPRQKDFLLAGNNNREQQVYGRSIEKHSGQN1FSGFNHELLSEALGISTLAAKRLQGQNDHRGEIIRVRNGLQLLKPTFTQQQEQ
AQSQYQVQYSEKQQESTRCNGLDENFCTINARLNIENPSRADTYNPRAGRITHLNNQKFPILNLVQMSATRVNLYQNAILSPYWNVNAHSLV
YMVQGHARVQVVSNLGKTVFNSVLRPGQLLIIPQHYVVLKKAEREGCQYIAFKTNANSIVSQLAGKNSILRAMPVDVVANAYRISREQARDLK
NNRGEELGAFTPKFEQQSYPGLSNESESEASE [SEQ ID 540]
>gi|109894635|gb|ABG47337.1| glutelin precursor [Zizania latifolia]
MNMATINGPTIFFTVCLFLLCHGSLAQLLGQSTSQWQSSHRGSSRQCRFDRLQAFEPVRSVRSQAGTTEFFDASNELFQCAGVSIVRRIIEPRG
LLLPQYTNGATIMYIIQGRGITGQTFPGCPESYQQQFQQSMQAQLTGSQSQSQKFKDEHQKINRFRQGDVIALPAGVAHWCYNDGEVPVVA
IYVIDINNAANQLDPRQRDFLLAGNMRSPQAYRREVENQSQNIFSGFSAELLSEALGISTGVARQLQCQNDQRGEIVRVEHGLSLLQPYASLQE
QEQKQEQPRERYQVTQHQQSQYGGGCSNGLDETFCAMRIWQNIDNPNLADTYNPRAGRVTNLNSQKFPILNLIQMSAVKVNLYQNALLSP
FWNINSHSVVYVTQGCARVQVVNNNGKTVFNGELRRGQLLIIPQHYVVVKKAQREGCAYIAFKTNPNSMVSHIVGKSSIFRALPTDVLANAY
RISREDAQRLKHNRGDELGAFTPLQYKSYQDVSSVAASS [SEQ ID 541]
P29835 (Rice)
>gi|226508602|ref|NP_001152635.1| globulin precursor [Zea mays] >gi|195658363|gb|ACG48649.1|
globulin precursor [Zea mays]
MAKIAAAAAAAAALCFAALVAVAVCQGEVERQRLRDLQCWQEVQESPLDACRQVLDRQLTGGGVGGPFRWGTGLRMRCCQQLQDVSRE
CRCAAIRSMVRGYEEAMPPLEKGWWPWGRQQQPPPQGGGGGQGGYYYPCSRAGEGYQTQMYPPCRPGTTGPRIGRVRLTKAREYAAGL
PMMCRLSEPQECSIFSGGDQY [SEQ ID 542]
>gi|242090997|ref|XP_002441331.1| hypothetical protein SORBIDRAFT_09g024570 [Sorghum bicolor]
>gi|241946616|gb|EES19761.1| hypothetical protein SORBIDRAFT_09g024570 [Sorghum bicolor]
MAKIAAVAATAALCLAALVAVAVGQGVVERQRLKDLQCWQEVQENPLGACRQVLDRQLTGGMRYGIGPFRWGTGLRMRCCQQLQDVSR
ECRCAAIRSMVRGYEETMPPLEKGWWGQQPQPGYDYPCSQAGEGYGYGESGQQQMYPPCRPGTGQKIARVKLTKARQYAAGMPMMCR
LSEPQECSVFSGGDQYY [SEQ ID 543]
>gi|514748428|ref|XP_004961615.1| PREDICTED: 19 kDa globulin-like [Setaria italica]
MAKFVVAAATAALCLAALVAMAAGQSGFERQRLRDLRCQREVEENPLWACRQVLDRQLTGGMRYGVGPFRWGTGLRMRCCQQLQDVS
RECRCSAVRRMVRGYEEAMPPLEEGPYGYGGEQGEGYYGGGEGGEGYLPFPPRRIGRVRLTKARQYAAGLPMMCRLEPQECSVFSGDQYK
[SEQ ID 544]
P0C1U8 (Bacterial) GLUC (Staphylococcus aureus)
>gi|446599182|ref|WP_000676528.1| glutamyl endopeptidase [Staphylococcus aureus]
>gi|253729369|gb|EES98098.1|
trypsin [Staphylococcus aureus subsp. aureus TCH130] >gi|341844549|gb|EGS85761.1|
glutamyl endopeptidase
[Staphylococcus aureus subsp. aureus 21259] >gi|537390486|gb|AGU61109.1|
Glutamyl endopeptidase precursor
[Staphylococcus aureus subsp. aureus CN1] >gi|564714561|gb|ETD14665.1|
glutamyl endopeptidase [Staphylococcus
aureus subsp. aureus KPL1845] >gi|577466329|gb|EUG79766.1|
glutamyl endopeptidase [Staphylococcus aureus M0139]
>gi|580560623|gb|EVF84961.1| glutamyl endopeptidase [Staphylococcus aureus CoA56020]
>gi|580687002|gb|EVH10169.1| glutamyl endopeptidase [Staphylococcus aureus UCIM6080]
>gi|751815683|gb|KIN24957.1| glutamyl endopeptidase [Staphylococcus aureus MRSA_CVM43477]
>gi|781884797|dbj|BAR08486.1| glutamyl endopeptidase precursor [Staphylococcus aureus subsp. aureus]
>gi|781887762|dbj|BAR11210.1| glutamyl endopeptidase precursor [Staphylococcus aureus subsp. aureus]
MKGKFLKVSSLFVATLTTATLVSSPAANALSSKAMDNHPQQSQSSKQQTPKIQKGGNLKPLEQREHANVILPNNDRHQITDTTNGHYAPVTYi
QVEAPTGTFIASGVVVGKDTLLTNKHVVDATHGDPHALKAFPSAINQDNYPNGGFTAEQITKYSGEGDLAIVKFSPNEQNKHIGEVVKPATMS
NNAETQVNQNITVTGYPGDKPVATMWESKGKITYLKGEAMQYDLSTTGGNSGSPVFNEKNEVIGIHWGGVPNEFNGAVFINENVRNFLKQ
NIEDIHFANDDQPNNPDNPDNPNNPDNPNNPDEPNNPDNPNNPDNPDNGDNNNSDNPDAA [SEQ ID 545]

What is claimed is:

1.-19. (canceled)

20. A peptide comprising SEQ ID NO: 222, or a substantially similar growth promoting sequence having 1 to 3 amino acid alterations compared SEQ ID NO: 222, in which the or each amino acid alteration is independently selected from insertion, addition, deletion and substitution of an amino acid, wherein the peptide is up to 50 amino acids in length.

21. The peptide of claim 20, wherein the peptide is growth promoting.

22. The peptide of claim 20, wherein the peptide consists of SEQ ID NO: 222.

23. The peptide of claim 20, wherein the peptide is a modified peptide.

24. The peptide of claim 20, wherein the peptide is chemically modified by modifications to side chains, incorporation of a protecting group, incorporation of unnatural amino acids and/or their derivatives during peptide synthesis, and imposing conformational constraint on the peptide.

25. A conjugate comprising a peptide of claim 20, conjugated, linked or fused to a binding partner, in which the binding partner is optionally selected from a polyethylene glycol (PEG) polymer, a molecular weight increasing compound, a lipophilic group, or an antibody molecule.

26. A composition comprising the peptide of claim 20.

27. The composition of claim 26, wherein the composition in the form of a powder.

28. The composition of claim 26, wherein the composition is formulated for topical application to the skin of a human.

29. The composition of claim 26, wherein the composition is in the form of a serum, gel, cream, lotion, ointment, cream, emulsion, anhydrous composition, aqueous dispersion, oil, milk, balsam, foam, hydro-alcoholic solution, hydro-glycolic solution, cosmetic, personal care product, hydrogel, liniment, sera, soap, dusting powder, paster, semi solid formulation, shampoo, conditioner, any rinse off formulation, talc, mousse, power, spray, aerosol, solution, suspension, syrup, elixir, polysaccharide film, patches, gel patches, bandages, an adhesive system, water in oil emulsion, oil in water emulsion and silicone emulsion.

30. The composition of claim 26, wherein the composition further comprises a cosmetically or pharmaceutically acceptable excipient, optionally selected from the group consisting of an emollient, diluent, carrier, binder, lubricant, suspending agent, coating agent, preservative, stabiliser, dye, vehicle, solubilising agent, base, emulsifying agent, fragrance, humectant, and surfactant.

31. The composition of claim 26, wherein the composition is a personal care composition, a pharmaceutical composition, a cosmetic composition, a food or beverage or a nutritional supplement.

32. A method of slowing ageing of human skin, or to treat visible signs of ageing, wherein the method comprises topically administering to the skin of a human the peptide of claim 20.

33. A method for treating a wound in a mammal, wherein the method comprises topically administering to the wound the peptide of claim 20.

34. A wound dressing comprising the peptide of claim 20, wherein the dressing is optionally selected from the group consisting of a bandage or a plaster.


SEQUENCE LISTING (778 sequences)

SEQ ID NO: 1 - protein, 459 aa, Pisum sativum
MAATTMKASFPLLMLMGISFLASVCVSSRSDPQNPFIFKSNKFQTLFENENGHIRLLQKFDQRSKIFENLQNYRLLEYKSKPHTIFLPQHTDADYILVVLSGKAILTVLKPDDRNSFNLERGDTIKLPAGTIAYLVNRDDNEELRVLDLAIPVNRPGQLQSFLLSGNQNQQNYLSGFSKNILEASFNTDYEEIEKVLLEEHEKETQHRRSLKDKRQQSQEENVIVKLSRGQIEELSKNAKSTSKKSVSSESEPFNLRSRGPIYSNEFGKFFEITPEKNPQLQDLDIFVNSVEIKEGSLLLPHYNSRAIVIVTVNEGKGDFELVGQRNENQQEQRKEDDEEEEQGEEEINKQVQNYKAKLSSGDVFVIPAGHPVAVKASSNLDLLGFGINAENNQRNFLAGDEDNVISQIQRPVKELAFPGSAQEVDRILENQKQSHFADAQPQQRERGSRETRDRLSSV

SEQ ID NO: 2 - protein, 613 aa, Pisum sativum
MATTIKSRFPLLLLLGIIFLASVVCVTYANYDEGSEPRVPAQRERGRQEGEKEEKRHGEWRPSYEKEEDEEEGQRERGRQEGEKEEKRHGEWRPSYEKQEDEEEKQKYRYQREKEDEEEKQKYQYQREKKEQKEVQPGRERWEREEDEEQVDEEWRGSQRREDPEERARLRHREERTKRDRRHQREGEEEERSSESQERRNPFLFKSNKFLTLFENENGHIRLLQRFDKRSDLFENLQNYRLVEYRAKPHTIFLPQHIDADLILVVLSGKAILTVLSPNDRNSYNLERGDTIKLPAGTTSYLVNQDDEEDLRLVDLVIPVNGPGKFEAFDLAKNKNQYLRGFSKNILEASYNTRYETIEKVLLEEQEKDRKRRQQGEETDAIVKVSREQIEELKKLAKSSSKKSLPSEFEPINLRSHKPEYSNKFGKLFEITPEKKYPQLQDLDLFVSCVEINEGALMLPHYNSRAIVVLLVNEGKGNLELLGLKNEQQEREDRKERNNEVQRYEARLSPGDVVIIPAGHPVAITASSNLNLLGFGINAENNERNFLSGSDDNVISQIENPVKELTFPGSVQEINRLIKNQKQSHFANAEPEQKEQGSQGKRSPLSSILGTFY

SEQ ID NO: 3 - protein, 609 aa, Oryza sativa
MSALTTSQLATSATGFGIADRSAPSSLLRHGFQGLKPRSPAGGDATSLSVTTSARATPKQQRSVQRGSRRFPSVVVYATGAGMNVVFVGAEMAPWSKTGGLGDVLGGLPPAMAANGHRVMVISPRYDQYKDAWDTSVVAEIKVADRYERVRFFHCYKRGVDRVFIDHPSFLEKVWGKTGEKIYGPDTGVDYKDNQMRFSLLCQAALEAPRILNLNNNPYFKGTYGEDVVFVCNDWHTGPLASYLKNNYQPNGIYRNAKVAFCIHNISYQGRFAFEDYPELNLSERFRSSFDFIDGYDTPVEGRKINWMKAGILEADRVLTVSPYYAEELISGIARGCELDNIMRLTGITGIVNGMDVSEWDPSKDKYITAKYDATTAIEAKALNKEALQAEAGLPVDRKIPLIAFIGRLEEQKGPDVMAAAIPELMQEDVQIVLLGTGKKKFEKLLKSMEEKYPGKVRAVVKFNAPLAHLIMAGADVLAVPSRFEPCGLIQLQGMRYGTPCACASTGGLVDTVIEGKTGFHMGRLSVDCKVVEPSDVKKVAATLKRAIKVVGTPAYEEMVRNCMNQDLSWKGPAKNWENVLLGLGVAGSAPGIEGDEIAPLAKENVAAP

SEQ ID NO: 4 - protein, 499 aa, Oryza sativa
MASSVFSRFSIYFCVLLLCHGSMAQLFNPSTNPWHSPRQGSFRECRFDRLQAFEPLRKVRSEAGVTEYFDEKNELFQCTGTFVIRRVIQPQGLLVPRYTNIPGVVYIIQGRGSMGLTFPGCPATYQQQFQQFSSQGQSQSQKFRDEHQKIHQFRQGDIVALPAGVAHWFYNDGDAPIVAVYVYDVNNNANQLEPRQKEFLLAGNNNRAQQQQVYGSSIEQHSGQNIFSGFGVEMLSEALGINAVAAKRLQSQNDQRGEIIHVKNGLQLLKPTLTQQQEQAQAQDQYQQVQYSERQQTSSRWNGLEENFCTIKVRVNIENPSRADSYNPRAGRITSVNSQKFPILNLIQMSATRVNLYQNAILSPFWNVNAHSLVYMIQGRSRVQVVSNFGKTVFDGVLRPGQLLIIPQHYAVLKKAEREGCQYIAIKTNANAFVSHLAGKNSVFRALPVDVVANAYRISREQARSLKNNRGEEHGAFTPRFQQQYYPGLSNESESETSE

SEQ ID NO: 5 - protein, 186 aa, Oryza sativa
MASKVVFFAAALMAAMVAISGAQLSESEMRFRDRQCQREVQDSPLDACRQVLDRQLTGRERFQPMFRRPGALGLRMQCCQQLQDVSRECRCAAIRRMVRSYEESMPMPLEQGWSSSSSEYYGGEGSSSEQGYYGEGSSEEGYYGEQQQQPGMTRVRLTRARQYAAQLPSMCRVEPQQCSIFAAGQY

SEQ ID NO: 6 - protein, 500 aa, Oryza sativa
MATIAFSRLSIYFCVLLLCHGSMAQLFGPNVNPWHNPRQGGFRECRFDRLQAFEPLRRVRSEAGVTEYFDEKNEQFQCTGTFVIRRVIEPQGLLVPRYSNTPGMVYIIQGRGSMGLTFPGCPATYQQQFQQFLPEGQSQSQKFRDEHQKIHQFRQGDIVALPAGVAHWFYNEGDAPVVALYVFDLNNNANQLEPRQKEFLLAGNNNREQQMYGRSIEQHSGQNIFSGFNNELLSEALGVNALVAKRLQGQNDQRGEIIRVKNGLKLLRPAFAQQQEQAQQQEQAQAQYQVQYSEEQQPSTRCNGLDENFCTIKARLNIENPSHADTYNPRAGRITRLNSQKFPILNLVQLSATRVNLYQNAILSPFWNVNAHSLVYIVQGHARVQVVSNLGKTVFNGVLRPGQLLIIPQHYVVLKKAEHEGCQYISFKTNANSMVSHLAGKNSIFRAMPVDVIANAYRISREQARSLKNNRGEELGAFTPRYQQQTYPGFSNESENEALE

SEQ ID NO: 7 - protein, 861 aa, Pisum sativum
MFSGVTGILNRGHKIKGTVVLMRKNVLDINSLTTVGGVIGQGFDILGSTVDNLTAFLGRSVSLQLISATKPDATGKGKLGKATFLEGIISSLPTLGAGQSAFKIHFEWDDDMGIPGAFYIKNFMQTEFFLVSLTLDDIPNHGSIYFVCNSWIYNAKHHKIDRIFFANQTYLPSETPAPLVHYREEELNNLRGDGTGERKEWERIYDYDVYNDLGNPDSGENHARPVLGGSETYPYPRRGRTGRKPTRKDPNSESRSDYVYLPRDEAFGHLKSSDFLTYGLKAVSQNVVPALESVFFDLNFTPNEFDSFDEVHGLYEGGIKLPTNILSQISPLPVLKEIFRTDGENTLKYPPPKVIQVSRSGWMTDEEFAREMLAGVNPNVICCLQEFPPRSKLDSQIYGDHTSKISKEHLEPNLEGLTVEEAIQNKKLFLLDHHDSIMPYLRRINSTSTKAYATRTILFLNNNQNLKPLAIELSLPHPQGDEHGAVSYVYQPALEGVESSIWLLAKAYVIVNDSCYHQLVSHWLNTHAVVEPFVIATNRHLSCLHPIYKLLYPHYRDTMNINSLARLSLVNDGGIIEKTFLWGRYSMEMSSKVYKNWVFTEQALPADLIKRGMAIEDPSSPCGVKLVVEDYPYAVDGLEIWAIIKTWVQDYVSLYYTSDEKLRQDSELQAWWKELVEVGHGDKKNEPWWPKMQTREDLIEVCSIVIWTASALHAAVNFGQYSYGGLILNRPTLSRRFMPEKGSAEFEELVKSPQKAYLKTITPKFQTLIDLSVIEILSRHASDELYLGERDNPNWTSDKRALEAFKKFGNKLAEIEKKLTQRNNDEKLRNRHGPVEMPYTLLYPSSKEGLTFRGIPNSISI

SEQ ID NO: 8 - protein, 517 aa, Pisum sativum
MAKLLALSLSFCFLLLGGCFALREQPQQNECQLERLDALEPDNRIESEGGLIETWNPNNKQFRCAGVALSRATLQRNALRRPYYSNAPQEIFIQQGNGYFGMVFPGCPETFEEPQESEQGEGRRYRDRHQKVNRFREGDIIAVPTGIVFWMYNDQDTPVIAVSLTDIRSSNNQLDQMPRRFYLAGNHEQEFLQYQHQQGGKQEQENEGNNIFSGFKRDYLEDAFNVNRHIVDRLQGRNEDEEKGAIVKVKGGLSIISPPEKQARHQRGSRQEEDEDEEKQPRHQRGSRQEEEEDEDEERQPRHQRRRGEEEEEDKKERGGSQKGKSRRQGDNGLEETVCTAKLRLNIGPSSSPDIYNPEAGRIKTVTSLDLPVLRWLKLSAEHGSLHKNAMFVPHYNLNANSIIYALKGRARLQVVNCNGNTVFDGELEAGRALTVPQNYAVAAKSLSDRFSYVAFKTNDRAGIARLAGTSSVINNLPLDVVAATFNLQRNEARQLKSNNPFKFLVPARESENRASA

SEQ ID NO: 9 - protein, 275 aa, Pisum sativum
DNAEIEKILLEEHEKETHHRRGLRDKRQQSQEKNVIVKVSKKQIEELSKNAKSSSKKSVSSRSEPFNLKSSDPIYSNQYGKFFEITPKKNPQLQDLDIFVNYVEIKEGSLWLPHYNSRAIVIVTVNEGKGDFELVGQRNENQQGLREEDDEEEEQREEETKNQVQSYKAKLTPGDVFVIPAGHPVAVRASSNLNLLGFGINAENNQRNFLAGEEDNVISQIQKQVKDLTFPGSAQEVDRLLENQKQSYFANAQPQQRETRSQEIKEHLYSILGAF

SEQ ID NO: 10 - protein, 438 aa, Pisum sativum
MAATPIKPLMLLAIAFLASVCVSSRSDQENPFIFKSNRFQTLYENENGHIRLLQKFDKRSKIFENLQNYRLLEYKSKPRTLFLPQYTDADFILVVLSGKATLTVLKSNDRNSFNLERGDTIKLPAGTIAYLANRDDNEDLRVLDLTIPVNKPGQLQSFLLSGTQNQPSLLSGFSKNILEAAFNTNYEEIEKVLLEQQEQEPQHRRSLKDRRQEINEENVIVKVSREQIEELSKNAKSSSKKSVSSESGPFNLRSRNPIYSNKFGKFFEITPEKNQQLQDLDIFVNSVDIKEGSLLLPNYNSRAIVIVTVTEGKGDFELVGQRNENQGKENDKEEEQEEETSKQVQLYRAKLSPGDVFVIPAGHPVAINASSDLNLIGFGINAENNERNFLAGEEDNVISQVERPVKELAFPGSSHEVDRLLKNQKQSYFANAQPLQRE

SEQ ID NO: 11 - protein, 499 aa, Oryza sativa
MASINRPIVFFTVCLFLLCNGSLAQQLLGQSTSQWQSSRRGSPRECRFDRLQAFEPIRSVRSQAGTTEFFDVSNEQFQCTGVSVVRRVIEPRGLLLPHYTNGASLVYIIQGRGITGPTFPGCPESYQQQFQQSGQAQLTESQSQSQKFKDEHQKIHRFRQGDVIALPAGVAHWCYNDGEVPVVAIYVTDLNNGANQLDPRQRDFLLAGNKRNPQAYRREVEERSQNIFSGFSTELLSEALGVSSQVARQLQCQNDQRGEIVRVEHGLSLLQPYASLQEQEQGQVQSRERYQEGQYQQSQYGSGCSNGLDETFCTLRVRQNIDNPNRADTYNPRAGRVTNLNTQNFPILSLVQMSAVKVNLYQNALLSPFWNINAHSVVYITQGRARVQVVNNNGKTVFNGELRRGQLLIIPQHYAVVKKAQREGCAYIAFKTNPNSMVSHIAGKSSIFRALPNDVLANAYRISREEAQRLKHNRGDEFGAFTPIQYKSYQDVYNAAESS

SEQ ID NO: 12 - protein, 499 aa, Oryza sativa
MASINRPIVFFTVCLFLLCDGSLAQQLLGQSTSQWQSSRRGSPRGCRFDRLQAFEPIRSVRSQAGTTEFFDVSNELFQCTGVSVVRRVIEPRGLLLPHYTNGASLVYIIQGRGITGPTFPGCPETYQQQFQQSGQAQLTESQSQSHKFKDEHQKIHRFRQGDVIALPAGVAHWCYNDGEVPVVAIYVTDINNGANQLDPRQRDFLLAGNKRNPQAYRREVEEWSQNIFSGFSTELLSEAFGISNQVARQLQCQNDQRGEIVRVERGLSLLQPYASLQEQEQGQMQSREHYQEGGYQQSQYGSGCPNGLDETFCTMRVRQNIDNPNRADTYNPRAGRVTNLNSQNFPILNLVQMSAVKVNLYQNALLSPFWNINAHSIVYITQGRAQVQVVNNNGKTVFNGELRRGQLLIVPQHYVVVKKAQREGCAYIAFKTNPNSMVSHIAGKSSIFRALPTDVLANAYRISREEAQRLKHNRGDEFGAFTPLQYKSYQDVYNVAESS

SEQ ID NO: 13 - protein, 160 aa, Oryza sativa
MASNKVVFSVLLLAVVSVLAATATMAEYHHQDQVVYTPGPLCQPGMGYPMYPLPRCRALVKRQCVGRGTAAAAEQVRRDCCRQLAAVDDSWCRCEAISHMLGGIYRELGAPDVGHPMSEVFRGCRRGDLERAAASLPAFCNVDIPNGGGGVCYWLARSGY

SEQ ID NO: 14 - protein, 336 aa, Staphylococcus aureus
MKGKFLKVSSLFVATLTTATLVSSPAANALSSKAMDNHPQQTQSSKQQTPKIQKGGNLKPLEQREHANVILPNNDRHQITDTTNGHYAPVTYIQVEAPTGTFIASGVVVGKDTLLTNKHVVDATHGDPHALKAFPSAINQDNYPNGGFTAEQITKYSGEGDLAIVKFSPNEQNKHIGEVVKPATMSNNAETQVNQNITVTGYPGDKPVATMWESKGKITYLKGEAMQYDLSTTGGNSGSPVFNEKNEVIGIHWGGVPNEFNGAVFINENVRNFLKQNIEDIHFANDDQPNNPDNPDNPNNPDNPNNPDEPNNPDNPNNPDNPDNGDNNNSDNPDAA

SEQ ID NO: 15 - protein, 13 aa, synthetic construct
  REGION 1..13: note = Synthetic peptide
QNYLSGFSKNILE

SEQ ID NO: 16 - protein, 20 aa, synthetic construct
  REGION 1..20: note = Synthetic peptide
TIKLPAGTIAYLVNRDDNEE

SEQ ID NO: 17 - protein, 15 aa, synthetic construct
  REGION 1..15: note = Synthetic peptide
LAIPVNRPGQLQSFL

SEQ ID NO: 18 - protein, 11 aa, synthetic construct
  REGION 1..11: note = Synthetic peptide
AIPVNRPGQLQ

SEQ ID NO: 19 - protein, 9 aa, synthetic construct
  REGION 1..9: note = Synthetic peptide
PAGHPVAVK

SEQ ID NO: 20 - protein, 19 aa, synthetic construct
  REGION 1..19: note = Synthetic peptide
VQNYKAKLSSGDVFVIPAG

SEQ ID NO: 21 - protein, 24 aa, synthetic construct
  REGION 1..24: note = Synthetic peptide
NNQRNFLAGDEDNVISQIQRPVKE

SEQ ID NO: 22 - protein, 23 aa, synthetic construct
  REGION 1..23: note = Synthetic peptide
INKQVQNYKAKLSSGDVFVIPAG

SEQ ID NO: 23 - protein, 10 aa, synthetic construct
  REGION 1..10: note = Synthetic peptide
LAIPVNRPGQ

SEQ ID NO: 24 - protein, 20 aa, synthetic construct
  REGION 1..20: note = Synthetic peptide
NFLAGDEDNVISQIQRPVKE

SEQ ID NO: 25 - protein, 15 aa, synthetic construct
  REGION 1..15: note = Synthetic peptide
DLAIPVNRPGQLQSF

SEQ ID NO: 26 - protein, 11 aa, synthetic construct
  REGION 1..11: note = Synthetic peptide
VIPAGHPVAVK

SEQ ID NO: 27 - protein, 21 aa, synthetic construct
  REGION 1..21: note = Synthetic peptide
DTIKLPAGTIAYLVNRDDNEE

SEQ ID NO: 28 - protein, 14 aa, synthetic construct
  REGION 1..14: note = Synthetic peptide
LAIPVNRPGQLQSF

SEQ ID NO: 29 - protein, 21 aa, synthetic construct
  REGION 1..21: note = Synthetic peptide
KQVQNYKAKLSSGDVFVIPAG

SEQ ID NO: 30 - protein, 23 aa, synthetic construct
  REGION 1..23: note = Synthetic peptide
RGDTIKLPAGTIAYLVNRDDNEE

SEQ ID NO: 31 - protein, 19 aa, synthetic construct
  REGION 1..19: note = Synthetic peptide
FLAGDEDNVISQIQRPVKE

SEQ ID NO: 32 - protein, 13 aa, synthetic construct
  REGION 1..13: note = Synthetic peptide
LAIPVNRPGQLQS

SEQ ID NO: 33 - protein, 15 aa, synthetic construct
  REGION 1..15: note = Synthetic peptide
VLDLAIPVNRPGQLQ

SEQ ID NO: 34 - protein, 13 aa, synthetic construct
  REGION 1..13: note = Synthetic peptide
DLAIPVNRPGQLQ

SEQ ID NO: 35 - protein, 13 aa, synthetic construct
  REGION 1..13: note = Synthetic peptide
VFVIPAGHPVAVK

SEQ ID NO: 36 - protein, 20 aa, synthetic construct
  REGION 1..20: note = Synthetic peptide
TIFLPQHTDADYILVVLSGK

SEQ ID NO: 37 - protein, 23 aa, synthetic construct
  REGION 1..23: note = Synthetic peptide
NQRNFLAGDEDNVISQIQRPVKE

SEQ ID NO: 38 - protein, 12 aa, synthetic construct
  REGION 1..12: note = Synthetic peptide
LAIPVNRPGQLQ

SEQ ID NO: 39 - protein, 17 aa, synthetic construct
  REGION 1..17: note = Synthetic peptide
HPVAVKASSNLDLLGFG

SEQ ID NO: 40 - protein, 11 aa, synthetic construct
  REGION 1..11: note = Synthetic peptide
LAIPVNRPGQL

SEQ ID NO: 41 - protein, 12 aa, synthetic construct
  REGION 1..12: note = Synthetic peptide
DLAIPVNRPGQL

SEQ ID NO: 42 - protein, 24 aa, synthetic construct
  REGION 1..24: note = Synthetic peptide
SKPHTIFLPQHTDADYILVVLSGK

SEQ ID NO: 43 - protein, 12 aa, synthetic construct
  REGION 1..12: note = Synthetic peptide
FVIPAGHPVAVK

SEQ ID NO: 44 - protein, 14 aa, synthetic construct
  REGION 1..14: note = Synthetic peptide
DLAIPVNRPGQLQS

SEQ ID NO: 45 - protein, 22 aa, synthetic construct
  REGION 1..22: note = Synthetic peptide
SGDVFVIPAGHPVAVKASSNLD

SEQ ID NO: 46 - protein, 13 aa, synthetic construct
  REGION 1..13: note = Synthetic peptide
AIPVNRPGQLQSF

SEQ ID NO: 47 - protein, 13 aa, synthetic construct
  REGION 1..13: note = Synthetic peptide
ELAFPGSAQEVDR

SEQ ID NO: 48 - protein, 18 aa, synthetic construct
  REGION 1..18: note = Synthetic peptide
LAIPVNRPGQLQSFLLSG

SEQ ID NO: 49 - protein, 24 aa, synthetic construct
  REGION 1..24: note = Synthetic peptide
VFVIPAGHPVAVKASSNLDLLGFG

SEQ ID NO: 50 - protein, 8 aa, synthetic construct
  REGION 1..8: note = Synthetic peptide
AGHPVAVK

SEQ ID NO: 51 - protein, 21 aa, synthetic construct
  REGION 1..21: note = Synthetic peptide
HPVAVKASSNLDLLGFGINAE

SEQ ID NO: 52 - protein, 22 aa, synthetic construct
  REGION 1..22: note = Synthetic peptide
LAIPVNRPGQLQSFLLSGNQNQ

SEQ ID NO: 53 - protein, 10 aa, synthetic construct
  REGION 1..10: note = Synthetic peptide
SGDVFVIPAG

SEQ ID NO: 54 - protein, 20 aa, synthetic construct
  REGION 1..20: note = Synthetic peptide
GSLLLPHYNSRAIVIVTVNE

SEQ ID NO: 55 - protein, 19 aa, synthetic construct
  REGION 1..19: note = Synthetic peptide
NFLAGDEDNVISQIQRPVK

SEQ ID NO: 56 - protein, 14 aa, synthetic construct
  REGION 1..14: note = Synthetic peptide
SGDVFVIPAGHPVA

SEQ ID NO: 57 - protein, 16 aa, synthetic construct
  REGION 1..16: note = Synthetic peptide
GSLLLPHYNSRAIVIV

SEQ ID NO: 58 - protein, 20 aa, synthetic construct
  REGION 1..20: note = Synthetic peptide
RGDTIKLPAGTIAYLVNRDD

SEQ ID NO: 59 - protein, 16 aa, synthetic construct
  REGION 1..16: note = Synthetic peptide
SGDVFVIPAGHPVAVK

SEQ ID NO: 60 - protein, 18 aa, synthetic construct
  REGION 1..18: note = Synthetic peptide
LSSGDVFVIPAGHPVAVK

SEQ ID NO: 61 - protein, 13 aa, synthetic construct
  REGION 1..13: note = Synthetic peptide
LDLAIPVNRPGQL

SEQ ID NO: 62 - protein, 10 aa, synthetic construct
  REGION 1..10: note = Synthetic peptide
AIPVNRPGQL

SEQ ID NO: 63 - protein, 16 aa, synthetic construct
  REGION 1..16: note = Synthetic peptide
LAIPVNRPGQLQSFLL

SEQ ID NO: 64 - protein, 22 aa, synthetic construct
  REGION 1..22: note = Synthetic peptide
PHTIFLPQHTDADYILVVLSGK

SEQ ID NO: 65 - protein, 19 aa, synthetic construct
  REGION 1..19: note = Synthetic peptide
VFVIPAGHPVAVKASSNLD

SEQ ID NO: 66 - protein, 17 aa, synthetic construct
  REGION 1..17: note = Synthetic peptide
LAIPVNRPGQLQSFLLS

SEQ ID NO: 67 - protein, 17 aa, synthetic construct
  REGION 1..17: note = Synthetic peptide
VLDLAIPVNRPGQLQSF

SEQ ID NO: 68 - protein, 12 aa, synthetic construct
  REGION 1..12: note = Synthetic peptide
AIPVNRPGQLQS

SEQ ID NO: 69 - protein, 20 aa, synthetic construct
  REGION 1..20: note = Synthetic peptide
DTIKLPAGTIAYLVNRDDNE

SEQ ID NO: 70 - protein, 17 aa, synthetic construct
  REGION 1..17: note = Synthetic peptide
NYKAKLSSGDVFVIPAG

SEQ ID NO: 71 - protein, 19 aa, synthetic construct
  REGION 1..19: note = Synthetic peptide
GKAILTVLKPDDRNSFNLE

SEQ ID NO: 72 - protein, 17 aa, synthetic construct
  REGION 1..17: note = Synthetic peptide
YKSKPHTIFLPQHTDAD

SEQ ID NO: 73 - protein, 11 aa, synthetic construct
  REGION 1..11: note = Synthetic peptide
ASSNLDLLGFG

SEQ ID NO: 74 - protein, 13 aa, synthetic construct
  REGION 1..13: note = Synthetic peptide
DEEEEQGEEEINK

SEQ ID NO: 75 - protein, 15 aa, synthetic construct
  REGION 1..15: note = Synthetic peptide
YKSKPHTIFLPQHTD

SEQ ID NO: 76 - protein, 10 aa, synthetic construct
  REGION 1..10: note = Synthetic peptide
VLDLAIPVNR

SEQ ID NO: 77 - protein, 25 aa, synthetic construct
  REGION 1..25: note = Synthetic peptide
FFEITPEKNPQLQDLDIFVNSVEIK

SEQ ID NO: 78 - protein, 14 aa, synthetic construct
  REGION 1..14: note = Synthetic peptide
TIFLPQHTDADYIL

SEQ ID NO: 79 - protein, 16 aa, synthetic construct
  REGION 1..16: note = Synthetic peptide
SFLLSGNQNQQNYLSG

SEQ ID NO: 80 - protein, 18 aa, synthetic construct
  REGION 1..18: note = Synthetic peptide
SFLLSGNQNQQNYLSGFS

SEQ ID NO: 81 - protein, 19 aa, synthetic construct
  REGION 1..19: note = Synthetic peptide
NQQEQRKEDDEEEEQGEEE

SEQ ID NO: 82 - protein, 10 aa, synthetic construct
  REGION 1..10: note = Synthetic peptide
EEQGEEEINK

SEQ ID NO: 83 - protein, 9 aa, synthetic construct
  REGION 1..9: note = Synthetic peptide
SRGPIYSNE

SEQ ID NO: 84 - protein, 15 aa, synthetic construct
  REGION 1..15: note = Synthetic peptide
EDDEEEEQGEEEINK

SEQ ID NO: 85 - protein, 14 aa, synthetic construct
  REGION 1..14: note = Synthetic peptide
DDEEEEQGEEEINK

SEQ ID NO: 86 - protein, 15 aa, synthetic construct
  REGION 1..15: note = Synthetic peptide
KEDDEEEEQGEEEIN

SEQ ID NO: 87 - protein, 12 aa, synthetic construct
  REGION 1..12: note = Synthetic peptide
KEDDEEEEQGEE

SEQ ID NO: 88 - protein, 15 aa, synthetic construct
  REGION 1..15: note = Synthetic peptide
QRKEDDEEEEQGEEE

SEQ ID NO: 89 - protein, 16 aa, synthetic construct
  REGION 1..16: note = Synthetic peptide
KEDDEEEEQGEEEINK

SEQ ID NO: 90 - protein, 13 aa, synthetic construct
  REGION 1..13: note = Synthetic peptide
KEDDEEEEQGEEE

SEQ ID NO: 91 - protein, 15 aa, synthetic construct
  REGION 1..15: note = Synthetic peptide
HPVAITASSNLNLLG

SEQ ID NO: 92 - protein, 11 aa, synthetic construct
  REGION 1..11: note = Synthetic peptide
ASSNLNLLGFG

SEQ ID NO: 93 - protein, 13 aa, synthetic construct
  REGION 1..13: note = Synthetic peptide
ITASSNLNLLGFG

SEQ ID NO: 94 - protein, 17 aa, synthetic construct
  REGION 1..17: note = Synthetic peptide
ITASSNLNLLGFGINAE

SEQ ID NO: 95 - protein, 10 aa, synthetic construct
  REGION 1..10: note = Synthetic peptide
SSNLNLLGFG

SEQ ID NO: 96 - protein, 13 aa, synthetic construct
  REGION 1..13: note = Synthetic peptide
VDLVIPVNGPGKF

SEQ ID NO: 97 - protein, 12 aa, synthetic construct
  REGION 1..12: note = Synthetic peptide
LVIPVNGPGKFE

SEQ ID NO: 98 - protein, 13 aa, synthetic construct
  REGION 1..13: note = Synthetic peptide
LVIPVNGPGKFEA

SEQ ID NO: 99 - protein, 17 aa, synthetic construct
  REGION 1..17: note = Synthetic peptide
LRLVDLVIPVNGPGKFE

SEQ ID NO: 100 - protein, 17 aa, synthetic construct
  REGION 1..17: note = Synthetic peptide
YRAKPHTIFLPQHIDAD

SEQ ID NO: 101 - protein, 21 aa, synthetic construct
  REGION 1..21: note = Synthetic peptide
HPVAITASSNLNLLGFGINAE

SEQ ID NO: 102 - protein, 9 aa, synthetic construct
  REGION 1..9: note = Synthetic peptide
SNLNLLGFG

SEQ ID NO: 103 - protein, 24 aa, synthetic construct
  REGION 1..24: note = Synthetic peptide
HPVAITASSNLNLLGFGINAENNE

SEQ ID NO: 104 - protein, 15 aa, synthetic construct
  REGION 1..15: note = Synthetic peptide
LVDLVIPVNGPGKFE

SEQ ID NO: 105 - protein, 11 aa, synthetic construct
  REGION 1..11: note = Synthetic peptide
LVIPVNGPGKF

SEQ ID NO: 106 - protein, 18 aa, synthetic construct
  REGION 1..18: note = Synthetic peptide
TIKLPAGTTSYLVNQDDE

SEQ ID NO: 107 - protein, 18 aa, synthetic construct
  REGION 1..18: note = Synthetic peptide
DLRLVDLVIPVNGPGKFE

SEQ ID NO: 108 - protein, 19 aa, synthetic construct
  REGION 1..19: note = Synthetic peptide
EDLRLVDLVIPVNGPGKFE

SEQ ID NO: 109 - protein, 17 aa, synthetic construct
  REGION 1..17: note = Synthetic peptide
HPVAITASSNLNLLGFG

SEQ ID NO: 110 - protein, 21 aa, synthetic construct
  REGION 1..21: note = Synthetic peptide
LVDLVIPVNGPGKFEAFDLAK

SEQ ID NO: 111 - protein, 13 aa, synthetic construct
  REGION 1..13: note = Synthetic peptide
DNVISQIENPVKE

SEQ ID NO: 112 - protein, 24 aa, synthetic construct
  REGION 1..24: note = Synthetic peptide
VVIIPAGHPVAITASSNLNLLGFG

SEQ ID NO: 113 - protein, 17 aa, synthetic construct
  REGION 1..17: note = Synthetic peptide
LVDLVIPVNGPGKFEAF

SEQ ID NO: 114 - protein, 9 aa, synthetic construct
  REGION 1..9: note = Synthetic peptide
YPQLQDLDL

SEQ ID NO: 115 - protein, 10 aa, synthetic construct
  REGION 1..10: note = Synthetic peptide
VIPVNGPGKF

SEQ ID NO: 116 - protein, 8 aa, synthetic construct
  REGION 1..8: note = Synthetic peptide
SKKSLPSE

SEQ ID NO: 117 - protein, 17 aa, synthetic construct
  REGION 1..17: note = Synthetic peptide
LPQHIDADLILVVLSGK

SEQ ID NO: 118 - protein, 19 aa, synthetic construct
  REGION 1..19: note = Synthetic peptide
RGDTIKLPAGTTSYLVNQD

SEQ ID NO: 119 - protein, 9 aa, synthetic construct
  REGION 1..9: note = Synthetic peptide
IPVNGPGKF

SEQ ID NO: 120 - protein, 9 aa, synthetic construct
  REGION 1..9: note = Synthetic peptide
LPQHIDADL

SEQ ID NO: 121 - protein, 10 aa, synthetic construct
  REGION 1..10: note = Synthetic peptide
LVIPVNGPGK

SEQ ID NO: 122 - protein, 10 aa, synthetic construct
  REGION 1..10: note = Synthetic peptide
IFLPQHIDAD

SEQ ID NO: 123 - protein, 8 aa, synthetic construct
  REGION 1..8: note = Synthetic peptide
LPQHIDAD

SEQ ID NO: 124 - protein, 9 aa, synthetic construct
  REGION 1..9: note = Synthetic peptide
VIPVNGPGK

SEQ ID NO: 125 - protein, 9 aa, synthetic construct
  REGION 1..9: note = Synthetic peptide
IFLPQHIDA

SEQ ID NO: 126 - protein, 19 aa, synthetic construct
  REGION 1..19: note = Synthetic peptide
TIKLPAGTTSYLVNQDDEE

SEQ ID NO: 127 - protein, 21 aa, synthetic construct
  REGION 1..21: note = Synthetic peptide
HGEWRPSYEKEEDEEEGQRER

SEQ ID NO: 128 - protein, 23 aa, synthetic construct
  REGION 1..23: note = Synthetic peptide
EKRHGEWRPSYEKEEDEEEGQRE

SEQ ID NO: 129 - protein, 19 aa, synthetic construct
  REGION 1..19: note = Synthetic peptide
LPAGTTSYLVNQDDEEDLR

SEQ ID NO: 130 - protein, 16 aa, synthetic construct
  REGION 1..16: note = Synthetic peptide
PSYEKEEDEEEGQRER

SEQ ID NO: 131 - protein, 12 aa, synthetic construct
  REGION 1..12: note = Synthetic peptide
EKRHGEWRPSYE

SEQ ID NO: 132 - protein, 20 aa, synthetic construct
  REGION 1..20: note = Synthetic peptide
TIKLPAGTTSYLVNQDDEED

SEQ ID NO: 133 - protein, 17 aa, synthetic construct
  REGION 1..17: note = Synthetic peptide
HGEWRPSYEKQEDEEEK

SEQ ID NO: 134 - protein, 14 aa, synthetic construct
  REGION 1..14: note = Synthetic peptide
EWRPSYEKEEDEEE

SEQ ID NO: 135 - protein, 14 aa, synthetic construct
  REGION 1..14: note = Synthetic peptide
PSYEKEEDEEEGQR

SEQ ID NO: 136 - protein, 11 aa, synthetic construct
  REGION 1..11: note = Synthetic peptide
EKEEDEEEGQR

SEQ ID NO: 137 - protein, 18 aa, synthetic construct
  REGION 1..18: note = Synthetic peptide
EWRPSYEKEEDEEEGQRE

SEQ ID NO: 138 - protein, 10 aa, synthetic construct
  REGION 1..10: note = Synthetic peptide
KEEDEEEGQR

SEQ ID NO: 139 - protein, 19 aa, synthetic construct
  REGION 1..19: note = Synthetic peptide
VQPGRERWEREEDEEQVDE

SEQ ID NO: 140 - protein, 12 aa, synthetic construct
  REGION 1..12: note = Synthetic peptide
DVVIIPAGHPVA

SEQ ID NO: 141 - protein, 14 aa, synthetic construct
  REGION 1..14: note = Synthetic peptide
HGEWRPSYEKQEDE

SEQ ID NO: 142 - protein, 9 aa, synthetic construct
  REGION 1..9: note = Synthetic peptide
EEDEEEGQR

SEQ ID NO: 143 - protein, 19 aa, synthetic construct
  REGION 1..19: note = Synthetic peptide
HGEWRPSYEKEEDEEEGQR

SEQ ID NO: 144 - protein, 14 aa, synthetic construct
  REGION 1..14: note = Synthetic peptide
EEWRGSQRREDPEE

SEQ ID NO: 145 - protein, 23 aa, synthetic construct
  REGION 1..23: note = Synthetic peptide
REEDEEQVDEEWRGSQRREDPEE

SEQ ID NO: 146 - protein, 9 aa, synthetic construct
  REGION 1..9: note = Synthetic peptide
RHGEWRPSY

SEQ ID NO: 147 - protein, 15 aa, synthetic construct
  REGION 1..15: note = Synthetic peptide
HGEWRPSYEKQEDEE

SEQ ID NO: 148 - protein, 11 aa, synthetic construct
  REGION 1..11: note = Synthetic peptide
VVIIPAGHPVA

SEQ ID NO: 149 - protein, 9 aa, synthetic construct
  REGION 1..9: note = Synthetic peptide
HGEWRPSYE

SEQ ID NO: 150 - protein, 12 aa, synthetic construct
  REGION 1..12: note = Synthetic peptide
KEEDEEEGQRER

SEQ ID NO: 151 - protein, 13 aa, synthetic construct
  REGION 1..13: note = Synthetic peptide
VVIIPAGHPVAIT

SEQ ID NO: 152 - protein, 17 aa, synthetic construct
  REGION 1..17: note = Synthetic peptide
EKRHGEWRPSYEKEEDE

SEQ ID NO: 153 - protein, 17 aa, synthetic construct
  REGION 1..17: note = Synthetic peptide
QVDEEWRGSQRREDPEE

SEQ ID NO: 154 - protein, 24 aa, synthetic construct
  REGION 1..24: note = Synthetic peptide
GDTIKLPAGTTSYLVNQDDEEDLR

SEQ ID NO: 155 - protein, 11 aa, synthetic construct
  REGION 1..11: note = Synthetic peptide
GSEPRVPAQRE

SEQ ID NO: 156 - protein, 15 aa, synthetic construct
  REGION 1..15: note = Synthetic peptide
EEKRHGEWRPSYEKE

SEQ ID NO: 157 - protein, 13 aa, synthetic construct
  REGION 1..13: note = Synthetic peptide
EWRPSYEKEEDEE

SEQ ID NO: 158 - protein, 15 aa, synthetic construct
  REGION 1..15: note = Synthetic peptide
NYDEGSEPRVPAQRE

SEQ ID NO: 159 - protein, 12 aa, synthetic construct
  REGION 1..12: note = Synthetic peptide
VIIPAGHPVAIT

SEQ ID NO: 160 - protein, 11 aa, synthetic construct
  REGION 1..11: note = Synthetic peptide
RHGEWRPSYEK

SEQ ID NO: 161 - protein, 9 aa, synthetic construct
  REGION 1..9: note = Synthetic peptide
NYDEGSEPR

SEQ ID NO: 162 - protein, 12 aa, synthetic construct
  REGION 1..12: note = Synthetic peptide
WRPSYEKEEDEE

SEQ ID NO: 163 - protein, 13 aa, synthetic construct
  REGION 1..13: note = Synthetic peptide
WRPSYEKQEDEEE

SEQ ID NO: 164 - protein, 19 aa, synthetic construct
  REGION 1..19: note = Synthetic peptide
EKRHGEWRPSYEKQEDEEE

SEQ ID NO: 165 - protein, 14 aa, synthetic construct
  REGION 1..14: note = Synthetic peptide
VVIIPAGHPVAITA

SEQ ID NO: 166 - protein, 11 aa, synthetic construct
  REGION 1..11: note = Synthetic peptide
KRHGEWRPSYE

SEQ ID NO: 167 - protein, 16 aa, synthetic construct
  REGION 1..16: note = Synthetic peptide
GSDDNVISQIENPVKE

SEQ ID NO: 168 - protein, 10 aa, synthetic construct
  REGION 1..10: note = Synthetic peptide
VVIIPAGHPV

SEQ ID NO: 169 - protein, 8 aa, synthetic construct
  REGION 1..8: note = Synthetic peptide
HGEWRPSY

SEQ ID NO: 170 - protein, 15 aa, synthetic construct
  REGION 1..15: note = Synthetic peptide
RPSYEKEEDEEEGQR

SEQ ID NO: 171 - protein, 10 aa, synthetic construct
  REGION 1..10: note = Synthetic peptide
HGEWRPSYEK

SEQ ID NO: 172 - protein, 14 aa, synthetic construct
  REGION 1..14: note = Synthetic peptide
KRHGEWRPSYEKEE

SEQ ID NO: 173 - protein, 15 aa, synthetic construct
  REGION 1..15: note = Synthetic peptide
VVIIPAGHPVAITAS

SEQ ID NO: 174 - protein, 23 aa, synthetic construct
  REGION 1..23: note = Synthetic peptide
RGDTIKLPAGTTSYLVNQDDEED

SEQ ID NO: 175 - protein, 18 aa, synthetic construct
  REGION 1..18: note = Synthetic peptide
KRHGEWRPSYEKQEDEEE

SEQ ID NO: 176 - protein, 20 aa, synthetic construct
  REGION 1..20: note = Synthetic peptide
DEEQVDEEWRGSQRREDPEE

SEQ ID NO: 177 - protein, 10 aa, synthetic construct
  REGION 1..10: note = Synthetic peptide
RHGEWRPSYE

SEQ ID NO: 178 - protein, 11 aa, synthetic construct
  REGION 1..11: note = Synthetic peptide
HGEWRPSYEKE

SEQ ID NO: 179 - protein, 18 aa, synthetic construct
  REGION 1..18: note = Synthetic peptide
KRHGEWRPSYEKEEDEEE

SEQ ID NO: 180 - protein, 19 aa, synthetic construct
  REGION 1..19: note = Synthetic peptide
EKRHGEWRPSYEKEEDEEE

SEQ ID NO: 181 - protein, 25 aa, synthetic construct
  REGION 1..25: note = Synthetic peptide
TIKLPAGTTSYLVNQDDEEDLRLVD

SEQ ID NO: 182 - protein, 17 aa, synthetic construct
  REGION 1..17: note = Synthetic peptide
WRPSYEKEEDEEEGQRE

SEQ ID NO: 183 - protein, 17 aa, synthetic construct
  REGION 1..17: note = Synthetic peptide
KRHGEWRPSYEKEEDEE

SEQ ID NO: 184 - protein, 12 aa, synthetic construct
  REGION 1..12: note = Synthetic peptide
VVIIPAGHPVAI

SEQ ID NO: 185 - protein, 13 aa, synthetic construct
  REGION 1..13: note = Synthetic peptide
EWRGSQRREDPEE

SEQ ID NO: 186 - protein, 19 aa, synthetic construct
  REGION 1..19: note = Synthetic peptide
HGEWRPSYEKQEDEEEKQK

SEQ ID NO: 187 - protein, 17 aa, synthetic construct
  REGION 1..17: note = Synthetic peptide
SGSDDNVISQIENPVKE

SEQ ID NO: 188 - protein, 17 aa, synthetic construct
  REGION 1..17: note = Synthetic peptide
RPSYEKEEDEEEGQRER

SEQ ID NO: 189 - protein, 13 aa, synthetic construct
  REGION 1..13: note = Synthetic peptide
EKEEDEEEGQRER

SEQ ID NO: 190 - protein, 11 aa, synthetic construct
  REGION 1..11: note = Synthetic peptide
HGEWRPSYEKQ

SEQ ID NO: 191 - protein, 13 aa, synthetic construct
  REGION 1..13: note = Synthetic peptide
WRPSYEKEEDEEE

SEQ ID NO: 192 - protein, 13 aa, synthetic construct
  REGION 1..13: note = Synthetic peptide
LAKNKNQYLRGFS

SEQ ID NO: 193 - protein, 10 aa, synthetic construct
  REGION 1..10: note = Synthetic peptide
NKNQYLRGFS

SEQ ID NO: 194 - protein, 10 aa, synthetic construct
  REGION 1..10: note = Synthetic peptide
LRGFSKNILE

SEQ ID NO: 195 - protein, 15 aa, synthetic construct
  REGION 1..15: note = Synthetic peptide
LAKNKNQYLRGFSKN

SEQ ID NO: 196 - protein, 11 aa, synthetic construct
  REGION 1..11: note = Synthetic peptide
TVLSPNDRNSY

SEQ ID NO: 197 - protein, 12 aa, synthetic construct
  REGION 1..12: note = Synthetic peptide
QYLRGFSKNILE

SEQ ID NO: 198 - protein, 19 aa, synthetic construct
  REGION 1..19: note = Synthetic peptide
GKAILTVLSPNDRNSYNLE

SEQ ID NO: 199 - protein, 9 aa, synthetic construct
  REGION 1..9: note = Synthetic peptide
RGFSKNILE

SEQ ID NO: 200 - protein, 15 aa, synthetic construct
  REGION 1..15: note = Synthetic peptide
NKNQYLRGFSKNILE

SEQ ID NO: 201 - protein, 15 aa, synthetic construct
  REGION 1..15: note = Synthetic peptide
ASSNLNLLGFGINAE

SEQ ID NO: 202 - protein, 10 aa, synthetic construct
  REGION 1..10: note = Synthetic peptide
ASSNLNLLGF

SEQ ID NO: 203 - protein, 14 aa, synthetic construct
  REGION 1..14: note = Synthetic peptide
LAKNKNQYLRGFSK

SEQ ID NO: 204 - protein, 22 aa, synthetic construct
  REGION 1..22: note = Synthetic peptide
RGDTIKLPAGTTSYLVNQDDEE

SEQ ID NO: 205 - protein, 24 aa, synthetic construct
  REGION 1..24: note = Synthetic peptide
ARLSPGDVVIIPAGHPVAITASSN

SEQ ID NO: 206 - protein, 12 aa, synthetic construct
  REGION 1..12: note = Synthetic peptide
VQRYEARLSPGD

SEQ ID NO: 207 - protein, 20 aa, synthetic construct
  REGION 1..20: note = Synthetic peptide
ARLSPGDVVIIPAGHPVAIT

SEQ ID NO: 208 - protein, 21 aa, synthetic construct
  REGION 1..21: note = Synthetic peptide
RGDTIKLPAGTTSYLVNQDDE

SEQ ID NO: 209 - protein, 18 aa, synthetic construct
  REGION 1..18: note = Synthetic peptide
ARLSPGDVVIIPAGHPVA

SEQ ID NO: 210 - protein, 20 aa, synthetic construct
  REGION 1..20: note = Synthetic peptide
GALMLPHYNSRAIVVLLVNE

SEQ ID NO: 211 - protein, 23 aa, synthetic construct
  REGION 1..23: note = Synthetic peptide
ARLSPGDVVIIPAGHPVAITASS

SEQ ID NO: 212 - protein, 37 aa, synthetic construct
  REGION 1..37: note = Synthetic peptide
LSPGDVVIIPAGHPVAITASSNLNLLGFGINAENNER

SEQ ID NO: 213 - protein, 22 aa, synthetic construct
  REGION 1..22: note = Synthetic peptide
ARLSPGDVVIIPAGHPVAITAS

SEQ ID NO: 214 - protein, 23 aa, synthetic construct
  REGION 1..23: note = Synthetic peptide
LSPGDVVIIPAGHPVAITASSNL

SEQ ID NO: 215 - protein, 21 aa, synthetic construct
  REGION 1..21: note = Synthetic peptide
ARLSPGDVVIIPAGHPVAITA

SEQ ID NO: 216 - protein, 28 aa, synthetic construct
  REGION 1..28: note = Synthetic peptide
NWENVLLGLGVAGSAPGIEGDEIAPLAK

SEQ ID NO: 217 - protein, 17 aa, synthetic construct
  REGION 1..17: note = Synthetic peptide
YDQYKDAWDTSVVAEIK

SEQ ID NO: 218 - protein, 16 aa, synthetic construct
  REGION 1..16: note = Synthetic peptide
SSFDFIDGYDTPVEGR

SEQ ID NO: 219 - protein, 13 aa, synthetic construct
  REGION 1..13: note = Synthetic peptide
GPDTGVDYKDNQM

SEQ ID NO: 220 - protein, 11 aa, synthetic construct
  REGION 1..11: note = Synthetic peptide
ILNLNNNPYFK

SEQ ID NO: 221 - protein, 9 aa, synthetic construct
  REGION 1..9: note = Synthetic peptide
VVGTPAYEE

SEQ ID NO: 222 - protein, 11 aa, synthetic construct
  REGION 1..11: note = Synthetic peptide
IDGYDTPVEGR

SEQ ID NO: 223 - protein, 8 aa, synthetic construct
  REGION 1..8: note = Synthetic peptide
VVGTPAYE

SEQ ID NO: 224 - protein, 11 aa, synthetic construct
  REGION 1..11: note = Synthetic peptide
IYGPDTGVDYK

SEQ ID NO: 225 - protein, 12 aa, synthetic construct
  REGION 1..12: note = Synthetic peptide
VAGSAPGIEGDE

SEQ ID NO: 226 - protein, 16 aa, synthetic construct
  REGION 1..16: note = Synthetic peptide
IYGPDTGVDYKDNQMR

SEQ ID NO: 227 - protein, 12 aa, synthetic construct
  REGION 1..12: note = Synthetic peptide
VVGTPAYEEMVR

SEQ ID NO: 228 - protein, 13 aa, synthetic construct
  REGION 1..13: note = Synthetic peptide
DFIDGYDTPVEGR

SEQ ID NO: 229 - protein, 22 aa, synthetic construct
  REGION 1..22: note = Synthetic peptide
LGLGVAGSAPGIEGDEIAPLAK

SEQ ID NO: 230 - protein, 21 aa, synthetic construct
  REGION 1..21: note = Synthetic peptide
FNAPLAHLIMAGADVLAVPSR

SEQ ID NO: 231 - protein, 16 aa, synthetic construct
  REGION 1..16: note = Synthetic peptide
LGLGVAGSAPGIEGDE

SEQ ID NO: 232 - protein, 20 aa, synthetic construct
  REGION 1..20: note = Synthetic peptide
LGLGVAGSAPGIEGDEIAPL

SEQ ID NO: 233 - protein, 18 aa, synthetic construct
  REGION 1..18: note = Synthetic peptide
VLTVSPYYAEELISGIAR

SEQ ID NO: 234 - protein, 13 aa, synthetic construct
  REGION 1..13: note = Synthetic peptide
EALQAEAGLPVDR

SEQ ID NO: 235 - protein, 15 aa, synthetic construct
  REGION 1..15: note = Synthetic peptide
LGLGVAGSAPGIEGD

SEQ ID NO: 236 - protein, 13 aa, synthetic construct
  REGION 1..13: note = Synthetic peptide
IMAGADVLAVPSR

SEQ ID NO: 237 - protein, 15 aa, synthetic construct
  REGION 1..15: note = Synthetic peptide
GLGVAGSAPGIEGDE

SEQ ID NO: 238 - protein, 14 aa, synthetic construct
  REGION 1..14: note = Synthetic peptide
EALQAEAGLPVDRK

SEQ ID NO: 239 - protein, 21 aa, synthetic construct
  REGION 1..21: note = Synthetic peptide
TGGLGDVLGGLPPAMAANGHR

SEQ ID NO: 240 - protein, 11 aa, synthetic construct
  REGION 1..11: note = Synthetic peptide
LEEQKGPDVMA

SEQ ID NO: 241 - protein, 20 aa, synthetic construct
  REGION 1..20: note = Synthetic peptide
LGVAGSAPGIEGDEIAPLAK

SEQ ID NO: 242 - protein, 21 aa, synthetic construct
  REGION 1..21: note = Synthetic peptide
GLGVAGSAPGIEGDEIAPLAK

SEQ ID NO: 243 - protein, 15 aa, synthetic construct
  REGION 1..15: note = Synthetic peptide
TGGLGDVLGGLPPAM

SEQ ID NO: 244 - protein, 19 aa, synthetic construct
  REGION 1..19: note = Synthetic peptide
NVLLGLGVAGSAPGIEGDE

SEQ ID NO: 245 - protein, 12 aa, synthetic construct
  REGION 1..12: note = Synthetic peptide
TVFDGVLRPGQL

SEQ ID NO: 246 - protein, 16 aa, synthetic construct
  REGION 1..16: note = Synthetic peptide
RLQSQNDQRGEIIHVK

SEQ ID NO: 247 - protein, 15 aa, synthetic construct
  REGION 1..15: note = Synthetic peptide
EGYYGEQQQQPGMTR

SEQ ID NO: 248 - protein, 14 aa, synthetic construct
  REGION 1..14: note = Synthetic peptide
GYYGEQQQQPGMTR

SEQ ID NO: 249 - protein, 16 aa, synthetic construct
  REGION 1..16: note = Synthetic peptide
EEGYYGEQQQQPGMTR

SEQ ID NO: 250 - protein, 20 aa, synthetic construct
  REGION 1..20: note = Synthetic peptide
YYGGEGSSSEQGYYGEGSSE

SEQ ID NO: 251 - protein, 19 aa, synthetic construct
  REGION 1..19: note = Synthetic peptide
YGGEGSSSEQGYYGEGSSE

SEQ ID NO: 252 - protein, 18 aa, synthetic construct
  REGION 1..18: note = Synthetic peptide
SSEEGYYGEQQQQPGMTR

SEQ ID NO: 253 - protein, 17 aa, synthetic construct
  REGION 1..17: note = Synthetic peptide
SEEGYYGEQQQQPGMTR

SEQ ID NO: 254 - protein, 11 aa, synthetic construct
  REGION 1..11: note = Synthetic peptide
QGYYGEGSSEE

SEQ ID NO: 255 - protein, 22 aa, synthetic construct
  REGION 1..22: note = Synthetic peptide
YGGEGSSSEQGYYGEGSSEEGY

SEQ ID NO: 256 - protein, 12 aa, synthetic construct
  REGION 1..12: note = Synthetic peptide
YGEQQQQPGMTR

SEQ ID NO: 257 - protein, 13 aa, synthetic construct
  REGION 1..13: note = Synthetic peptide
YYGEQQQQPGMTR

SEQ ID NO: 258 - protein, 20 aa, synthetic construct
  REGION 1..20: note = Synthetic peptide
SYEESMPMPLEQGWSSSSSE

SEQ ID NO: 259 - protein, 23 aa, synthetic construct
  REGION 1..23: note = Synthetic peptide
YYGEGSSEEGYYGEQQQQPGMTR

SEQ ID NO: 260 - protein, 10 aa, synthetic construct
  REGION 1..10: note = Synthetic peptide
QQQQPGMTRV

SEQ ID NO: 261 - protein, 11 aa, synthetic construct
  REGION 1..11: note = Synthetic peptide
GEQQQQPGMTR

SEQ ID NO: 262 - protein, 21 aa, synthetic construct
  REGION 1..21: note = Synthetic peptide
SYEESMPMPLEQGWSSSSSEY

SEQ ID NO: 263 - protein, 23 aa, synthetic construct
  REGION 1..23: note = Synthetic peptide
YYGGEGSSSEQGYYGEGSSEEGY

SEQ ID NO: 264 - protein, 14 aa, synthetic construct
  REGION 1..14: note = Synthetic peptide
YGEQQQQPGMTRVR

SEQ ID NO: 265 - protein, 21 aa, synthetic construct
  REGION 1..21: note = Synthetic peptide
GEGSSEEGYYGEQQQQPGMTR

SEQ ID NO: 266 - protein, 22 aa, synthetic construct
  REGION 1..22: note = Synthetic peptide
YGEGSSEEGYYGEQQQQPGMTR

SEQ ID NO: 267 - protein, 11 aa, synthetic construct
  REGION 1..11: note = Synthetic peptide
QQQQPGMTRVR

SEQ ID NO: 268 - protein, 25 aa, synthetic construct
  REGION 1..25: note = Synthetic peptide
QYAAQLPSMCRVEPQQCSIFAAGQY

SEQ ID NO: 269 - protein, 12 aa, synthetic construct
  REGION 1..12: note = Synthetic peptide
TVFNGVLRPGQL

SEQ ID NO: 270 - protein, 13 aa, synthetic construct
  REGION 1..13: note = Synthetic peptide
TVFNGVLRPGQLL

SEQ ID NO: 271 - protein, 20 aa, synthetic construct
  REGION 1..20: note = Synthetic peptide
SGFNNELLSEALGVNALVAK

SEQ ID NO: 272 - protein, 9 aa, synthetic construct
  REGION 1..9: note = Synthetic peptide
NGVLRPGQL

SEQ ID NO: 273 - protein, 17 aa, synthetic construct
  REGION 1..17: note = Synthetic peptide
ALVAKRLQGQNDQRGEI

SEQ ID NO: 274 - protein, 10 aa, synthetic construct
  REGION 1..10: note = Synthetic peptide
VPRYSNTPGM

SEQ ID NO: 275 - protein, 10 aa, synthetic construct
  REGION 1..10: note = Synthetic peptide
PRYSNTPGMV

SEQ ID NO: 276 - protein, 9 aa, synthetic construct
  REGION 1..9: note = Synthetic peptide
YSNTPGMVY

SEQ ID NO: 277 - protein, 11 aa, synthetic construct
  REGION 1..11: note = Synthetic peptide
LVPRYSNTPGM

SEQ ID NO: 278 - protein, 10 aa, synthetic construct
  REGION 1..10: note = Synthetic peptide
FYNEGDAPVV

SEQ ID NO: 279 - protein, 12 aa, synthetic construct
  REGION 1..12: note = Synthetic peptide
FYNEGDAPVVAL

SEQ ID NO: 280 - protein, 15 aa, synthetic construct
  REGION 1..15: note = Synthetic peptide
FEPLRRVRSEAGVTE

SEQ ID NO: 281 - protein, 13 aa, synthetic construct
  REGION 1..13: note = Synthetic peptide
FYNEGDAPVVALY

SEQ ID NO: 282 - protein, 11 aa, synthetic construct
  REGION 1..11: note = Synthetic peptide
FYNEGDAPVVA

SEQ ID NO: 283 - protein, 16 aa, synthetic construct
  REGION 1..16: note = Synthetic peptide
HGPVEMPYTLLYPSSK

SEQ ID NO: 284 - protein, 9 aa, synthetic construct
  REGION 1..9: note = Synthetic peptide
LDALEPDNR

SEQ ID NO: 285 - protein, 8 aa, synthetic construct
  REGION 1..8: note = Synthetic peptide
DALEPDNR

SEQ ID NO: 286 - protein, 24 aa, synthetic construct
  REGION 1..24: note = Synthetic peptide
HGSLHKNAMFVPHYNLNANSIIYA

SEQ ID NO: 287 - protein, 20 aa, synthetic construct
  REGION 1..20: note = Synthetic peptide
LAGTSSVINNLPLDVVAATF

SEQ ID NO: 288 - protein, 16 aa, synthetic construct
  REGION 1..16: note = Synthetic peptide
FREGDIIAVPTGIVFW

SEQ ID NO: 289 - protein, 24 aa, synthetic construct
  REGION 1..24: note = Synthetic peptide
GTSSVINNLPLDVVAATFNLQRNE

SEQ ID NO: 290 - protein, 18 aa, synthetic construct
  REGION 1..18: note = Synthetic peptide
KGAIVKVKGGLSIISPPE

SEQ ID NO: 291 - protein, 15 aa, synthetic construct
  REGION 1..15: note = Synthetic peptide
RLAGTSSVINNLPLD

SEQ ID NO: 292 - protein, 25 aa, synthetic construct
  REGION 1..25: note = Synthetic peptide
AGTSSVINNLPLDVVAATFNLQRNE

SEQ ID NO: 293 - protein, 12 aa, synthetic construct
  REGION 1..12: note = Synthetic peptide
AGTSSVINNLPL

SEQ ID NO: 294 - protein, 17 aa, synthetic construct
  REGION 1..17: note = Synthetic peptide
LAGTSSVINNLPLDVVA

SEQ ID NO: 295 - protein, 14 aa, synthetic construct
  REGION 1..14: note = Synthetic peptide
AGTSSVINNLPLDV

SEQ ID NO: 296 - protein, 17 aa, synthetic construct
  REGION 1..17: note = Synthetic peptide
AGRIKTVTSLDLPVLRW

SEQ ID NO: 297 - protein, 16 aa, synthetic construct
  REGION 1..16: note = Synthetic peptide
AGRIKTVTSLDLPVLR

SEQ ID NO: 298 - protein, 15 aa, synthetic construct
  REGION 1..15: note = Synthetic peptide
FREGDIIAVPTGIVF

SEQ ID NO: 299 - protein, 13 aa, synthetic construct
  REGION 1..13: note = Synthetic peptide
AGTSSVINNLPLD

SEQ ID NO: 300 - protein, 13 aa, synthetic construct
  REGION 1..13: note = Synthetic peptide
LAGTSSVINNLPL

SEQ ID NO: 301 - protein, 16 aa, synthetic construct
  REGION 1..16: note = Synthetic peptide
LAGTSSVINNLPLDVV

SEQ ID NO: 302 - protein, 13 aa, synthetic construct
  REGION 1..13: note = Synthetic peptide
EGDIIAVPTGIVF

SEQ ID NO: 303 - protein, 15 aa, synthetic construct
  REGION 1..15: note = Synthetic peptide
LAGTSSVINNLPLDV

SEQ ID NO: 304 - protein, 20 aa, synthetic construct
  REGION 1..20: note = Synthetic peptide
AGRALTVPQNYAVAAKSLSD

SEQ ID NO: 305 - protein, 12 aa, synthetic construct
  REGION 1..12: note = Synthetic peptide
AGRALTVPQNYA

SEQ ID NO: 306 - protein, 14 aa, synthetic construct
  REGION 1..14: note = Synthetic peptide
LAGTSSVINNLPLD

SEQ ID NO: 307 - protein, 23 aa, synthetic construct
  REGION 1..23: note = Synthetic peptide
RAGIARLAGTSSVINNLPLDVVA

SEQ ID NO: 308 - protein, 16 aa, synthetic construct
  REGION 1..16: note = Synthetic peptide
RASSNLNLLGFGINAE

SEQ ID NO: 309 - protein, 12 aa, synthetic construct
  REGION 1..12: note = Synthetic peptide
VTVNEGKGDFEL

SEQ ID NO: 310 - protein, 17 aa, synthetic construct
  REGION 1..17: note = Synthetic peptide
VRASSNLNLLGFGINAE

SEQ ID NO: 311 - protein, 13 aa, synthetic construct
  REGION 1..13: note = Synthetic peptide
VRASSNLNLLGFG

SEQ ID NO: 312 - protein, 17 aa, synthetic construct
  REGION 1..17: note = Synthetic peptide
HPVAVRASSNLNLLGFG

SEQ ID NO: 313 - protein, 16 aa, synthetic construct
  REGION 1..16: note = Synthetic peptide
TKNQVQSYKAKLTPGD

SEQ ID NO: 314 - protein, 15 aa, synthetic construct
  REGION 1..15: note = Synthetic peptide
HPVAVRASSNLNLLG

SEQ ID NO: 315 - protein, 15 aa, synthetic construct
  REGION 1..15: note = Synthetic peptide
KAKLTPGDVFVIPAG

SEQ ID NO: 316 - protein, 19 aa, synthetic construct
  REGION 1..19: note = Synthetic peptide
DLTFPGSAQEVDRLLENQK

SEQ ID NO: 317 - protein, 9 aa, synthetic construct
  REGION 1..9: note = Synthetic peptide
PAGHPVAVR

SEQ ID NO: 318 - protein, 18 aa, synthetic construct
  REGION 1..18: note = Synthetic peptide
AKLTPGDVFVIPAGHPVA

SEQ ID NO: 319 - protein, 21 aa, synthetic construct
  REGION 1..21: note = Synthetic peptide
SYKAKLTPGDVFVIPAGHPVA

SEQ ID NO: 320 - protein, 18 aa, synthetic construct
  REGION 1..18: note = Synthetic peptide
LTPGDVFVIPAGHPVAVR

SEQ ID NO: 321 - protein, 19 aa, synthetic construct
  REGION 1..19: note = Synthetic peptide
VQSYKAKLTPGDVFVIPAG

SEQ ID NO: 322 - protein, 20 aa, synthetic construct
  REGION 1..20: note = Synthetic peptide
YKAKLTPGDVFVIPAGHPVA

SEQ ID NO: 323 - protein, 12 aa, synthetic construct
  REGION 1..12: note = Synthetic peptide
FVIPAGHPVAVR

SEQ ID NO: 324 - protein, 16 aa, synthetic construct
  REGION 1..16: note = Synthetic peptide
YKAKLTPGDVFVIPAG

SEQ ID NO: 325 - protein, 13 aa, synthetic construct
  REGION 1..13: note = Synthetic peptide
DLTFPGSAQEVDR

SEQ ID NO: 326 - protein, 20 aa, synthetic construct
  REGION 1..20: note = Synthetic peptide
AKLTPGDVFVIPAGHPVAVR

SEQ ID NO: 327 - protein, 12 aa, synthetic construct
  REGION 1..12: note = Synthetic peptide
LTPGDVFVIPAG

SEQ ID NO: 328 - protein, 17 aa, synthetic construct
  REGION 1..17: note = Synthetic peptide
SYKAKLTPGDVFVIPAG

SEQ ID NO: 329 - protein, 23 aa, synthetic construct
  REGION 1..23: note = Synthetic peptide
SYKAKLTPGDVFVIPAGHPVAVR

SEQ ID NO: 330 - protein, 11 aa, synthetic construct
  REGION 1..11: note = Synthetic peptide
VIPAGHPVAVR

SEQ ID NO: 331 - protein, 20 aa, synthetic construct
  REGION 1..20: note = Synthetic peptide
QVQSYKAKLTPGDVFVIPAG

SEQ ID NO: 332 - protein, 14 aa, synthetic construct
  REGION 1..14: note = Synthetic peptide
AKLTPGDVFVIPAG

SEQ ID NO: 333 - protein, 21 aa, synthetic construct
  REGION 1..21: note = Synthetic peptide
HPVAVRASSNLNLLGFGINAE

SEQ ID NO: 334 - protein, 22 aa, synthetic construct
  REGION 1..22: note = Synthetic peptide
YKAKLTPGDVFVIPAGHPVAVR

SEQ ID NO: 335 - protein, 23 aa, synthetic construct
  REGION 1..23: note = Synthetic peptide
TKNQVQSYKAKLTPGDVFVIPAG

SEQ ID NO: 336 - protein, 12 aa, synthetic construct
  REGION 1..12: note = Synthetic peptide
PFNLKSSDPIYS

SEQ ID NO: 337 - protein, 8 aa, synthetic construct
  REGION 1..8: note = Synthetic peptide
IEKILLEE

SEQ ID NO: 338 - protein, 16 aa, synthetic construct
  REGION 1..16: note = Synthetic peptide
SRSEPFNLKSSDPIYS

SEQ ID NO: 339 - protein, 13 aa, synthetic construct
  REGION 1..13: note = Synthetic peptide
HPVAVRASSNLNL

SEQ ID NO: 340 - protein, 20 aa, synthetic construct
  REGION 1..20: note = Synthetic peptide
TLFLPQYTDADFILVVLSGK

SEQ ID NO: 341 - protein, 10 aa, synthetic construct
  REGION 1..10: note = Synthetic peptide
EVEERSQNIF

SEQ ID NO: 342 - protein, 12 aa, synthetic construct
  REGION 1..12: note = Synthetic peptide
VEERSQNIFSGF

SEQ ID NO: 343 - protein, 12 aa, synthetic construct
  REGION 1..12: note = Synthetic peptide
ASLQEQEQGQVQ

SEQ ID NO: 344 - protein, 11 aa, synthetic construct
  REGION 1..11: note = Synthetic peptide
QEQEQGQVQSR

SEQ ID NO: 345 - protein, 14 aa, synthetic construct
  REGION 1..14: note = Synthetic peptide
ASLQEQEQGQVQSR

SEQ ID NO: 346 - protein, 13 aa, synthetic construct
  REGION 1..13: note = Synthetic peptide
EVEERSQNIFSGF

SEQ ID NO: 347 - protein, 17 aa, synthetic construct
  REGION 1..17: note = Synthetic peptide
VTDLNNGANQLDPRQRD

SEQ ID NO: 348 - protein, 14 aa, synthetic construct
  REGION 1..14: note = Synthetic peptide
VEHGLSLLQPYASL

SEQ ID NO: 349 - protein, 21 aa, synthetic construct
  REGION 1..21: note = Synthetic peptide
IYVTDLNNGANQLDPRQRDFL

SEQ ID NO: 350 - protein, 19 aa, synthetic construct
  REGION 1..19: note = Synthetic peptide
VTDLNNGANQLDPRQRDFL

SEQ ID NO: 351 - protein, 25 aa, synthetic construct
  REGION 1..25: note = Synthetic peptide
VEHGLSLLQPYASLQEQEQGQVQSR

SEQ ID NO: 352 - protein, 14 aa, synthetic construct
  REGION 1..14: note = Synthetic peptide
VTDLNNGANQLDPR

SEQ ID NO: 353 - protein, 19 aa, synthetic construct
  REGION 1..19: note = Synthetic peptide
IYVTDLNNGANQLDPRQRD

SEQ ID NO: 354 - protein, 20 aa, synthetic construct
  REGION 1..20: note = Synthetic peptide
YVTDLNNGANQLDPRQRDFL

SEQ ID NO: 355 - protein, 15 aa, synthetic construct
  REGION 1..15: note = Synthetic peptide
YVTDLNNGANQLDPR

SEQ ID NO: 356 - protein, 17 aa, synthetic construct
  REGION 1..17: note = Synthetic peptide
STELLSEALGVSSQVAR

SEQ ID NO: 357 - protein, 16 aa, synthetic construct
  REGION 1..16: note = Synthetic peptide
HGLSLLQPYASLQEQE

SEQ ID NO: 358 - protein, 20 aa, synthetic construct
  REGION 1..20: note = Synthetic peptide
SGFSTELLSEALGVSSQVAR

SEQ ID NO: 359 - protein, 13 aa, synthetic construct
  REGION 1..13: note = Synthetic peptide
GAFTPLQYKSYQD

SEQ ID NO: 360 - protein, 15 aa, synthetic construct
  REGION 1..15: note = Synthetic peptide
GLLLPHYTNGASLVY

SEQ ID NO: 361 - protein, 16 aa, synthetic construct
  REGION 1..16: note = Synthetic peptide
FLLAGNKRNPQAYRRE

SEQ ID NO: 362 - protein, 12 aa, synthetic construct
  REGION 1..12: note = Synthetic peptide
ALPTDVLANAYR

SEQ ID NO: 363 - protein, 8 aa, synthetic construct
  REGION 1..8: note = Synthetic peptide
DFLLAGNK

SEQ ID NO: 364 - protein, 8 aa, synthetic construct
  REGION 1..8: note = Synthetic peptide
DVLANAYR

SEQ ID NO: 365 - protein, 9 aa, synthetic construct
  REGION 1..9: note = Synthetic peptide
GAFTPLQYK

SEQ ID NO: 366 - protein, 14 aa, synthetic construct
  REGION 1..14: note = Synthetic peptide
QGDVIALPAGVAHW

SEQ ID NO: 367 - protein, 12 aa, synthetic construct
  REGION 1..12: note = Synthetic peptide
FGAFTPLQYKSY

SEQ ID NO: 368 - protein, 14 aa, synthetic construct
  REGION 1..14: note = Synthetic peptide
FLLAGNKRNPQAYR

SEQ ID NO: 369 - protein, 13 aa, synthetic construct
  REGION 1..13: note = Synthetic peptide
FGAFTPLQYKSYQ

SEQ ID NO: 370 - protein, 15 aa, synthetic construct
  REGION 1..15: note = Synthetic peptide
GLSLLQPYASLQEQE

SEQ ID NO: 371 - protein, 8 aa, synthetic construct
  REGION 1..8: note = Synthetic peptide
AFTPLQYK

SEQ ID NO: 372 - protein, 14 aa, synthetic construct
  REGION 1..14: note = Synthetic peptide
FGAFTPLQYKSYQD

SEQ ID NO: 373 - protein, 13 aa, synthetic construct
  REGION 1..13: note = Synthetic peptide
GDEFGAFTPLQYK

SEQ ID NO: 374 - protein, 15 aa, synthetic construct
  REGION 1..15: note = Synthetic peptide
FGAFTPLQYKSYQDV

SEQ ID NO: 375 - protein, 10 aa, synthetic construct
  REGION 1..10: note = Synthetic peptide
FGAFTPLQYK

SEQ ID NO: 376 - protein, 11 aa, synthetic construct
  REGION 1..11: note = Synthetic peptide
FGAFTPLQYKS

SEQ ID NO: 377 - protein, 14 aa, synthetic construct
  REGION 1..14: note = Synthetic peptide
VYIIQGRGITGPTF

SEQ ID NO: 378 - protein, 13 aa, synthetic construct
  REGION 1..13: note = Synthetic peptide
YIIQGRGITGPTF

SEQ ID NO: 379 - protein, 14 aa, synthetic construct
  REGION 1..14: note = Synthetic peptide
KTNPNSMVSHIAGK

SEQ ID NO: 380 - protein, 13 aa, synthetic construct
  REGION 1..13: note = Synthetic peptide
TNPNSMVSHIAGK

SEQ ID NO: 381 - protein, 12 aa, synthetic construct
  REGION 1..12: note = Synthetic peptide
PNSMVSHIAGKS

SEQ ID NO: 382 - protein, 20 aa, synthetic construct
  REGION 1..20: note = Synthetic peptide
NIDNPNRADTYNPRAGRVTN

SEQ ID NO: 383 - protein, 11 aa, synthetic construct
  REGION 1..11: note = Synthetic peptide
QRDFLLAGNKR

SEQ ID NO: 384 - protein, 12 aa, synthetic construct
  REGION 1..12: note = Synthetic peptide
LLQPYASLQEQE

SEQ ID NO: 385 - protein, 10 aa, synthetic construct
  REGION 1..10: note = Synthetic peptide
QRDFLLAGNK

SEQ ID NO: 386 - protein, 11 aa, synthetic construct
  REGION 1..11: note = Synthetic peptide
QEQEQGQMQSR

SEQ ID NO: 387 - protein, 13 aa, synthetic construct
  REGION 1..13: note = Synthetic peptide
SLLQPYASLQEQE

SEQ ID NO: 388 - protein, 11 aa, synthetic construct
  REGION 1..11: note = Synthetic peptide
ASLQEQEQGQM

SEQ ID NO: 389 - protein, 14 aa, synthetic construct
  REGION 1..14: note = Synthetic peptide
ASLQEQEQGQMQSR

SEQ ID NO: 390 - protein, 9 aa, synthetic construct
  REGION 1..9: note = Synthetic peptide
DFLLAGNKR

SEQ ID NO: 391 - protein, 18 aa, synthetic construct
  REGION 1..18: note = Synthetic peptide
QAFEPIRSVRSQAGTTEF

SEQ ID NO: 392 - protein, 18 aa, synthetic construct
  REGION 1..18: note = Synthetic peptide
KTNPNSMVSHIAGKSSIF

SEQ ID NO: 393 - protein, 21 aa, synthetic construct
  REGION 1..21: note = Synthetic peptide
VRRVIEPRGLLLPHYTNGASL

SEQ ID NO: 394 - protein, 17 aa, synthetic construct
  REGION 1..17: note = Synthetic peptide
FGAFTPLQYKSYQDVYN

SEQ ID NO: 395 - protein, 10 aa, synthetic construct
  REGION 1..10: note = Synthetic peptide
IALPAGVAHW

SEQ ID NO: 396 - protein, 25 aa, synthetic construct
  REGION 1..25: note = Synthetic peptide
RVRQNIDNPNRADTYNPRAGRVTNL

SEQ ID NO: 397 - protein, 21 aa, synthetic construct
  REGION 1..21: note = Synthetic peptide
NIDNPNRADTYNPRAGRVTNL

SEQ ID NO: 398 - protein, 16 aa, synthetic construct
  REGION 1..16: note = Synthetic peptide
GAFTPLQYKSYQDVYN

SEQ ID NO: 399 - protein, 16 aa, synthetic construct
  REGION 1..16: note = Synthetic peptide
PNSMVSHIAGKSSIFR

SEQ ID NO: 400 - protein, 19 aa, synthetic construct
  REGION 1..19: note = Synthetic peptide
RLQAFEPIRSVRSQAGTTE

SEQ ID NO: 401 - protein, 18 aa, synthetic construct
  REGION 1..18: note = Synthetic peptide
TNPNSMVSHIAGKSSIFR

SEQ ID NO: 402 - protein, 11 aa, synthetic construct
  REGION 1..11: note = Synthetic peptide
ELGAPDVGHPM

SEQ ID NO: 403 - protein, 10 aa, synthetic construct
  REGION 1..10: note = Synthetic peptide
LGAPDVGHPM

SEQ ID NO: 404 - protein, 15 aa, synthetic construct
  REGION 1..15: note = Synthetic peptide
ELGAPDVGHPMSEVF

SEQ ID NO: 405 - protein, 12 aa, synthetic construct
  REGION 1..12: note = Synthetic peptide
ELGAPDVGHPMS

SEQ ID NO: 406 - protein, 16 aa, synthetic construct
  REGION 1..16: note = Synthetic peptide
ELGAPDVGHPMSEVFR

SEQ ID NO: 407 - protein, 14 aa, synthetic construct
  REGION 1..14: note = Synthetic peptide
ELGAPDVGHPMSEV

SEQ ID NO: 408 - protein, 13 aa, synthetic construct
  REGION 1..13: note = Synthetic peptide
ELGAPDVGHPMSE

SEQ ID NO: 409 - protein, 12 aa, synthetic construct
  REGION 1..12: note = Synthetic peptide
LGAPDVGHPMSE

SEQ ID NO: 410 - protein, 15 aa, synthetic construct
  REGION 1..15: note = Synthetic peptide
YRELGAPDVGHPMSE

SEQ ID NO: 411 - protein, 13 aa, synthetic construct
  REGION 1..13: note = Synthetic peptide
LGAPDVGHPMSEV

SEQ ID NO: 412 - protein, 14 aa, synthetic construct
  REGION 1..14: note = Synthetic peptide
RELGAPDVGHPMSE

SEQ ID NO: 413 - protein, 16 aa, synthetic construct
  REGION 1..16: note = Synthetic peptide
APTGTFIASGVVVGKD

SEQ ID NO: 414 - protein, 17 aa, synthetic construct
  REGION 1..17: note = Synthetic peptide
LAIVKFSPNEQNKHIGE

SEQ ID NO: 415 - protein, 21 aa, synthetic construct
  REGION 1..21: note = Synthetic peptide
RHQITDTTNGHYAPVTYIQVE

SEQ ID NO: 416 - protein, 19 aa, synthetic construct
  REGION 1..19: note = Synthetic peptide
GDLAIVKFSPNEQNKHIGE

SEQ ID NO: 417 - protein, 18 aa, synthetic construct
  REGION 1..18: note = Synthetic peptide
NPDNPDNPNNPDNPNNPD

SEQ ID NO: 418 - protein, 9 aa, synthetic construct
  REGION 1..9: note = Synthetic peptide
QSFILSGNE

SEQ ID NO: 419 - protein, 9 aa, synthetic construct
  REGION 1..9: note = Synthetic peptide
ESFLLSGNQ

SEQ ID NO: 420 - protein, 9 aa, synthetic construct
  REGION 1..9: note = Synthetic peptide
QSYLLSGNQ

SEQ ID NO: 421 - protein, 9 aa, synthetic construct
  REGION 1..9: note = Synthetic peptide
QSFLLSGDQ

SEQ ID NO: 422 - protein, 9 aa, synthetic construct
  REGION 1..9: note = Synthetic peptide
QSYLLSGNE

SEQ ID NO: 423 - protein, 9 aa, synthetic construct
  REGION 1..9: note = Synthetic peptide
ESFLLSGNE

SEQ ID NO: 424 - protein, 9 aa, synthetic construct
  REGION 1..9: note = Synthetic peptide
ESYLLSGNQ

SEQ ID NO: 425 - protein, 9 aa, synthetic construct
  REGION 1..9: note = Synthetic peptide
QSFLLSGDE

SEQ ID NO: 426 - protein, 9 aa, synthetic construct
  REGION 1..9: note = Synthetic peptide
QSYLLSGDQ

SEQ ID NO: 427 - protein, 9 aa, synthetic construct
  REGION 1..9: note = Synthetic peptide
QSFRLSGNQ

SEQ ID NO: 428 - protein, 9 aa, synthetic construct
  REGION 1..9: note = Synthetic peptide
QSFLLSYNQ

SEQ ID NO: 429 - protein, 9 aa, synthetic construct
  REGION 1..9: note = Synthetic peptide
QFFLLSGNQ

SEQ ID NO: 430 - protein, 9 aa, synthetic construct
  REGION 1..9: note = Synthetic peptide
QSFLLSGAQ

SEQ ID NO: 431 - protein, 9 aa, synthetic construct
  REGION 1..9: note = Synthetic peptide
QSFLLSGNP

SEQ ID NO: 432 - protein, 9 aa, synthetic construct
  REGION 1..9: note = Synthetic peptide
QSFRRSGNQ

SEQ ID NO: 433 - protein, 9 aa, synthetic construct
  REGION 1..9: note = Synthetic peptide
QSFLLSYIQ

SEQ ID NO: 434 - protein, 9 aa, synthetic construct
  REGION 1..9: note = Synthetic peptide
QFFLLSGNL

SEQ ID NO: 435 - protein, 10 aa, synthetic construct
  REGION 1..10: note = Synthetic peptide
QSFLLSGAQA

SEQ ID NO: 436 - protein, 9 aa, synthetic construct
  REGION 1..9: note = Synthetic peptide
QSFLLSGNP

SEQ ID NO: 437 - protein, 10 aa, synthetic construct
  REGION 1..10: note = Synthetic peptide
QSFLLSGNQQ

SEQ ID NO: 438 - protein, 10 aa, synthetic construct
  REGION 1..10: note = Synthetic peptide
QSFLLLSGNQ

SEQ ID NO: 439 - protein, 11 aa, synthetic construct
  REGION 1..11: note = Synthetic peptide
AQSFGLLSGNQ

SEQ ID NO: 440 - protein, 11 aa, synthetic construct
  REGION 1..11: note = Synthetic peptide
RQSFLLISGNQ

SEQ ID NO: 441 - protein, 10 aa, synthetic construct
  REGION 1..10: note = Synthetic peptide
QSFLLSGNQK

SEQ ID NO: 442 - protein, 8 aa, synthetic construct
  REGION 1..8: note = Synthetic peptide
QFLLSGNQ

SEQ ID NO: 443 - protein, 8 aa, synthetic construct
  REGION 1..8: note = Synthetic peptide
SFLLSGNQ

SEQ ID NO: 444 - protein, 8 aa, synthetic construct
  REGION 1..8: note = Synthetic peptide
QSFLLSGN

SEQ ID NO: 445 - protein, 9 aa, synthetic construct
  REGION 1..9: note = Synthetic peptide
QSFLLSGNQ

SEQ ID NO: 446 - protein, 8 aa, synthetic construct
  REGION 1..8: note = Synthetic peptide
QSFLSGNQ

SEQ ID NO: 447 - protein, 8 aa, synthetic construct
  REGION 1..8: note = Synthetic peptide
QSLLSGNQ

SEQ ID NO: 448 - protein, 8 aa, synthetic construct
  REGION 1..8: note = Synthetic peptide
SFLLSGNQ

SEQ ID NO: 449 - protein, 8 aa, synthetic construct
  REGION 1..8: note = Synthetic peptide
QSFLLSGN

SEQ ID NO: 450 - protein, 7 aa, synthetic construct
  REGION 1..7: note = Synthetic peptide
SFLLSGN

SEQ ID NO: 451 - protein, 7 aa, synthetic construct
  REGION 1..7: note = Synthetic peptide
QSFSGNQ

SEQ ID NO: 452 - protein, 11 aa, synthetic construct
  REGION 1..11: note = Synthetic peptide
HPRPPKPDAPR

SEQ ID NO: 453 - protein, 10 aa, synthetic construct
  REGION 1..10: note = Synthetic peptide
LQQAPPPPQR

SEQ ID NO: 454 - protein, 11 aa, synthetic construct
  REGION 1..11: note = Synthetic peptide
VGWGEQPWSPY

SEQ ID NO: 455 - protein, 5 aa, synthetic construct
  REGION 1..5: note = Synthetic peptide
FHMPP

SEQ ID NO: 456 - protein, 4 aa, synthetic construct
  REGION 1..4: note = Synthetic peptide
FRRP

SEQ ID NO: 457 - protein, 4 aa, synthetic construct
  REGION 1..4: note = Synthetic peptide
FWMA

SEQ ID NO: 458 - protein, 5 aa, synthetic construct
  REGION 1..5: note = Synthetic peptide
HMPPS

SEQ ID NO: 459 - protein, 11 aa, synthetic construct
  REGION 1..11: note = Synthetic peptide
PVEMPTLLYPS

SEQ ID NO: 460 - protein, 5 aa, synthetic construct
  REGION 1..5: note = Synthetic peptide
HMPSS

SEQ ID NO: 461 - protein, 4 aa, synthetic construct
  REGION 1..4: note = Synthetic peptide
HRFR

SEQ ID NO: 462 - protein, 4 aa, synthetic construct
  REGION 1..4: note = Synthetic peptide
HRRS

SEQ ID NO: 463 - protein, 4 aa, synthetic construct
  REGION 1..4: note = Synthetic peptide
HSPR

SEQ ID NO: 464 - protein, 4 aa, synthetic construct
  REGION 1..4: note = Synthetic peptide
HWFA

SEQ ID NO: 465 - protein, 4 aa, synthetic construct
  REGION 1..4: note = Synthetic peptide
MFRR

SEQ ID NO: 466 - protein, 5 aa, synthetic construct
  REGION 1..5: note = Synthetic peptide
MFRRP

SEQ ID NO: 467 - protein, 4 aa, synthetic construct
  REGION 1..4: note = Synthetic peptide
MPPS

SEQ ID NO: 468 - protein, 4 aa, synthetic construct
  REGION 1..4: note = Synthetic peptide
MPRR

SEQ ID NO: 469 - protein, 4 aa, synthetic construct
  REGION 1..4: note = Synthetic peptide
NMPS

SEQ ID NO: 470 - protein, 4 aa, synthetic construct
  REGION 1..4: note = Synthetic peptide
PHMP

SEQ ID NO: 471 - protein, 5 aa, synthetic construct
  REGION 1..5: note = Synthetic peptide
PHMPS

SEQ ID NO: 472 - protein, 4 aa, synthetic construct
  REGION 1..4: note = Synthetic peptide
PRRF

SEQ ID NO: 473 - protein, 4 aa, synthetic construct
  REGION 1..4: note = Synthetic peptide
WMKA

SEQ ID NO: 474 - protein, 11 aa, synthetic construct
  REGION 1..11: note = Synthetic peptide
DSINALEPDHR

SEQ ID NO: 475 - protein, 9 aa, synthetic construct
  REGION 1..9: note = Synthetic peptide
ELTFPGSVQ

SEQ ID NO: 476 - protein, 10 aa, synthetic construct
  REGION 1..10: note = Synthetic peptide
ELTFPGSVQE

SEQ ID NO: 477 - protein, 11 aa, synthetic construct
  REGION 1..11: note = Synthetic peptide
IFEDAITIPGR

SEQ ID NO: 478 - protein, 13 aa, synthetic construct
  REGION 1..13: note = Synthetic peptide
LDALEPDNRIESE

SEQ ID NO: 479 - protein, 10 aa, synthetic construct
  REGION 1..10: note = Synthetic peptide
KTLDYWPSLR

SEQ ID NO: 480 - protein, 9 aa, synthetic construct
  REGION 1..9: note = Synthetic peptide
RHGEWGPSY

SEQ ID NO: 481 - protein, 11 aa, synthetic construct
  REGION 1..11: note = Synthetic peptide
ILVDGSHDIER

SEQ ID NO: 482 - protein, 11 aa, synthetic construct
  REGION 1..11: note = Synthetic peptide
ILVDGSHDIER

SEQ ID NO: 483 - protein, 11 aa, synthetic construct
  REGION 1..11: note = Synthetic peptide
NLAQAPAQALL

SEQ ID NO: 484 - protein, 7 aa, synthetic construct
  REGION 1..7: note = Synthetic peptide
FLPQHTD

SEQ ID NO: 485 - protein, 6 aa, synthetic construct
  REGION 1..6: note = Synthetic peptide
LEPDNR

SEQ ID NO: 486 - protein, 6 aa, synthetic construct
  REGION 1..6: note = Synthetic peptide
LQSQND

SEQ ID NO: 487 - protein, 11 aa, synthetic construct
  REGION 1..11: note = Synthetic peptide
LQSQNDQRGEI

SEQ ID NO: 488 - protein, 14 aa, synthetic construct
  REGION 1..14: note = Synthetic peptide
QSQNDQRGEIIHVK

SEQ ID NO: 489 - protein, 8 aa, synthetic construct
  REGION 1..8: note = Synthetic peptide
RGEIIHVK

SEQ ID NO: 490 - protein, 8 aa, synthetic construct
  REGION 1..8: note = Synthetic peptide
RLQSQNDQ

SEQ ID NO: 491 - protein, 10 aa, synthetic construct
  REGION 1..10: note = Synthetic peptide
RLQSQNDQRG

SEQ ID NO: 492 - protein, 14 aa, synthetic construct
  REGION 1..14: note = Synthetic peptide
RLQSQNDQRGEIIH

SEQ ID NO: 493 - protein, 9 aa, synthetic construct
  REGION 1..9: note = Synthetic peptide
VFDGVLRPG

SEQ ID NO: 494 - protein, 4 aa, synthetic construct
  REGION 1..4: note = Synthetic peptide
HNPR

SEQ ID NO: 495 - protein, 4 aa, synthetic construct
  REGION 1..4: note = Synthetic peptide
HPMS

SEQ ID NO: 496 - protein, 4 aa, synthetic construct
  REGION 1..4: note = Synthetic peptide
HPSF

SEQ ID NO: 497 - protein, 4 aa, synthetic construct
  REGION 1..4: note = Synthetic peptide
MPMP

SEQ ID NO: 498 - protein, 4 aa, synthetic construct
  REGION 1..4: note = Synthetic peptide
PMPL

SEQ ID NO: 499 - protein, 4 aa, synthetic construct
  REGION 1..4: note = Synthetic peptide
PNSM

SEQ ID NO: 500 - protein, 4 aa, synthetic construct
  REGION 1..4: note = Synthetic peptide
WDPA

SEQ ID NO: 501 - protein, 6 aa, synthetic construct
  REGION 1..6: note = Synthetic peptide
LRGFSK

SEQ ID NO: 502 - protein, 6 aa, synthetic construct
  REGION 1..6: note = Synthetic peptide
RSQNIF

SEQ ID NO: 503 - protein, 6 aa, synthetic construct
  REGION 1..6: note = Synthetic peptide
YLRGFS

SEQ ID NO: 504 - protein, 9 aa, synthetic construct
  REGION 1..9: note = Synthetic peptide
GALMLPHYN

SEQ ID NO: 505 - protein, 11 aa, synthetic construct
  REGION 1..11: note = Synthetic peptide
GALMLPHYNSR

SEQ ID NO: 506 - protein, 463 aa, Vicia faba
MAATTLKDSFPLLTLLGIAFLASVCLSSRSDQDNPFVFESNRFQTLFENENGHIRLLQKFDQHSKLLENLQNYRLLEYKSKPHTIFLPQQTDADFILVVLSGKAILTVLLPNDRNSFSLERGDTIKLPAGTIGYLVNRDDEEDLRVLDLVIPVNRPGEPQSFLLSGNQNQPSILSGFSKNILEASFNTDYKEIEKVLLEEHGKEKYHRRGLKDRRQRGQEENVIVKISRKQIEELNKNAKSSSKKSTSSESEPFNLRSREPIYSNKFGKFFEITPKRNPQLQDLNIFVNYVEINEGSLLLPHYNSRAIVIVTVNEGKGDFELVGQRNENQQGLREEYDEEKEQGEEEIRKQVQNYKAKLSPGDVLVIPAGYPVAIKASSNLNLVGFGINAENNQRYFLAGEEDNVISQIHKPVKELAFPGSAQEVDTLLENQKQSHFANAQPRERERGSQEIKDHLYSILGSF

SEQ ID NO: 507 - protein, 446 aa, Cicer arietinum
MAIKARFPLLVLLGIVFLASVCAKSDKENPFFFKSNNCQTLFENENGHVRLLQRFDKRSQLFENLQNYRLMEYNSKPHTLFLPQHNDADFILVVLRGRAILTVLNPNDRNTFKLERGDTIKLPAGTIAYLANRDDNEDLRVLDLAIPVNRPGQFQSFSLSGNENQQSYFQGFSKKILEASFNSDYEEIERVLLEEQEQKPEQRRGHKGRQQSQETDVIVKISREQIEELSKNAKSNCKKSVSSESEPFNLRSRSPIYSNRFGNFFEITPEKNPQLKDLDIFVNSVEIKEGSLLLPHFNSRATVILVVNEGKGEVELVGLRNENEQENKKEDEEEEEDRNVQVQRFQSKLSSGDVVVIPASHPFSINASSDLFLLGFGINAQNNQRNFLAGEEDNVISQIQRPVKEVAFPGSAEEVDRLLKNQRQSHFANAQPQQKRKGSQRIRSPF

SEQ ID NO: 508 - protein, 418 aa, Lens culinaris
SRSDQENPFIFKSNRFQTIYENENGHIRLLQRFDKRSKIFENLQNYRLLEYKSKPHTIFLPQFTDADFILVVLSGKAILTVLNSNDRNSFNLERGDTIKLPAGTIAYLANRDDNEDLRVLDLAIPVNRPGQLQSFLLSGTQNQPSFLSGFSKNILEAAFNTEYEEIEKVLLEEQEQKSQHRRSLRDKRQEITNEDVIVKVSREQIEELSKNAKSSSKKSVSSESEPFNLRSRNPIYSNKFGKFFEITPEKNPQLQDLDIFVNSVEIKEGSLLLPNYNSRAIVIVTVNEGKGDFELVGQRNENQQEQREENDEEEGQEEETTKQVQRYRARLSPGDVLVIPAGHPVAINASSDLNLIGFGINAKNNQRNFLAGEEDNVISQIQRPVKELAFPGSSREVDRLLTNQKQSHFANAQPLQIE

SEQ ID NO: 509 - protein, 526 aa, Pisum sativum
  source 1..526: note = The subspecies is elatius.
MATTVESRFPLLLFPGIIFLASVCVTYANYDEGSETRVPGQRERGRQEGEKEEKRHGEWRPSYEKEEDEEEKQKYRYQREKEDEEEKQKYRYQREKKEEKEVQPGRERWEREEDEEQVDEEWRGSQRRQDPEERARLRHREERTKRDRRHKREGEEEERSSESQEQRNPFLFKSNKFLTLFENENGHIRRLQRFDKRSDLFENLQNYRLVEYRAKPHTIFLPQHIDADLILVVLNGKAILTVLSPNDRNSYNLERGDTIKIPAGTTSYLVNQDDEEDLRVVDFVIPVNRPGKFEAFGLSENKNQYLRGFSKNILEASLNTKYETIEKVLLEEQEKKPQQLRDRKRRQQGGERDAIIKVSREQIEELRKLAKSSSKKSLPSEFEPFNLRSHKPEYSNKFGKLFEITPEKKYPQLQDLDILVSCVEINKGALMLPHYNSRAIVVLLVNEGKGNLELLGLKNEQQEREDRKERNNEVQRYEARLSPGDVVIIPAGHPVAISASSNLNLLGFGTNAENNQRNFLSGSDDN

SEQ ID NO: 510 - protein, 541 aa, Lathyrus aphaca
MATTIKSRFPLLLLLGIIFLASVCVTWANYDEGSEPRVPGQRERGRQEGEKEEKRHGEWRPSYEEEYDEGLEPKVPGKRERGRQEGEKEEKRHEEWRPSYEKEEDEEEKQKYNYQREKKEHKEVQPGRERWERKQDEKQVEEDEEPGEEQWRGSKRHEDPEERARLRHREEKTKSYVEDNEETSSKEGRNPFLFKSNKFLTLFENENGHIRRLQRFDERSDIFENLQNYRLVEYRAKPHTMFLPQHIDADLILVVLNGKAILTVLSPNDRNSYNLERGDTVKLPAGTTSYLVNQDDEEDLRVVDLAIPVNRPGKFEAFGLSANKNQYLRGFSKNILEASLNTKYETIEKVLLEERRDQKGRQQGQETNAIVKVSREQIEELRKLAKSSSKKSLLSESEPLNLRSQNPKYSNKFGKFFEITPQKKYPQLQDLDVSISCVEINKGALLLPHYNSRSIGILLVNEGKGNLELVGFKNEQQRQRENEETNKKLQRYEARLSSGDVVVIPEGHPVAISASSNLNLLGFGINAANNQRNFLTGSDDN

SEQ ID NO: 511 - protein, 589 aa, Vicia villosa
MATTIKSRFPVLLLLGIIFLTSVCVTYANYDEGREPSVPGQRERGRQEGEKEEKRHGEWRPSEEDEEEKYKYEEGRVPGQRERGRQEGEKEEKRHGKWRPSEEEDEEEKYRYEEGSEPRGPGQRETGRQEGEKEKQRPEREPSYEKEEDEEEKQKYQYHREKKEQREVRPGRERFERHEDEEQWRGIQRHEDPEERARERYRAEIAKRQVEEEREERDIPHEREQRNPFLFKSNKFQTLFQNENGYIRRLQRFDKRSDLFENLQNYRLVEYRAKPHTIFLPQHIDADLIIVVLSGRAILTVLSPDDRNSYNLERGDTIKLPAGTTSYLVNQDDEEDLRVVDLAIPVNRPGKVESFLLSGNKNQYLRGFSKNILEASFNTNYETIERVLLEEQDKESQQSIGQKRRSQRQETNALVKVSREQLEDLKRLAKSSSQEGLSSQFEPINLRSQNPKYSNKFGKVFEITPEKKYPQLQDLDLFVSSVDIKEGALMLPHYNSRAIVVLLVNEGRGNLELVGLKNEQQEQREKEDEQQERNNQVQRYEARLSPGDVVIIPAGHPVAVRASSDLNLLAFGINAENNQRNFLAGSDDN

SEQ ID NO: 512 - protein, 861 aa, Medicago truncatula
MFSGVTGILNRGHKIKGTVVLMRKNVLDINSLTSVGGVIGQGFDILGSTLDNLTAFLGRSVSLQLISATKPDANGKGKLGKATFLEGIITSLPTLGAGQSAFKIHFEWDDDMGIPGAFYIKNFMQTEFFLVSLTLEDIPNHGSIYFVCNSWIYNAKHHKLDRIFFANKAYLPSETPAPLVHYREEELNNLRGDGTGERKEWERIYDYDVYNDLGNPEKGDNHARPVLGGSDTYPYPRRGRTGRKPNPKDPKSESRSDFVYLPRDEAFGHLKSSDFLTYGLKAVSQNVVPALESVIFDLNFTPNEFDSFDEVHGLYEGGIKLPTDVLSKISPLPVLKEIFRTDGEQFLKYPPPKVLQVSRSAWMTDEEFAREMLAGVNPNVICCLQEFPPRSKLDSQVYGDHTSKITKEHLEPNLEGLTVEEAIQNKKLFLLDHHDSIMPYLRRINSTPTKAYATRTILFLSSDKTLKPLAIELSLPHPDGDEHGAVSHVYQPALEGVESTIWLLAKAYVVVNDSCYHQLVSHWLNTHAVVEPFVIATNRHLSYLHPIYKLLYPHYRDTMNINSLARQSLVNDGGIIEKTFLWGRYSMEMSSKVYKNWTLPGQALPADLIKRGMAIEEPSSPCGVKLVVEDYPYAHDGLEIWAAIKTWVQDYVSLYYTTDDILRQDSELQAWWKELVEVGHGDKKNEPWWPKMQAREELVEVCTTVIWIASALHAAVNFGQYSYGGLILNRPTLSRRFMPEKGSAEYNELVKSPQKAYLKTITPKFQTLIDLSVIEILSRHASDEVYLGERDNPNWTSDTRALEAFKKFGNKLAEIEKNLAQRNNDEKLRHRLGPVQMPYTLLHPSSEEGLTFRGIPNSISI

SEQ ID NO: 513 - protein, 857 aa, Glycine soja
MLGGLLHRGHKIKGTVVLMRKNVLDVNSVTSVGGIIGQGLDLVGSTLDTLTAFLGRSVSLQLISATKADANGKGKLGKATFLEGIITSLPTLGAGQSAFKINFEWDDGSGIPGAFYIKNFMQTEFFLVSLTLEDIPNHGSIHFVCNSWIYNAKLFKSDRIFFANQTYLPSETPAPLVKYREEELHNLRGDGTGERKEWERIYDYDVYNDLGDPDKGENHARPVLGGNDTFPYPRRGRTGRKPTRKDPNSESRSNDVYLPRDEAFGHLKSSDFLTYGLKSVSQNVLPLLQSAFDLNFTPREFDSFDEVHGLYSGGIKLPTDIISKISPLPVLKEIFRTDGEQALKFPPPKVIQVSKSAWMTDEEFAREMLAGVNPNLIRCLKDFPPRSKLDSQVYGDHTSQITKEHLEPNLEGLTVDEAIQNKRLFLLDHHDPIMPYLRRINATSTKAYATRTILFLKNDGTLRPLAIELSLPHPQGDQSGAFSQVFLPADEGVESSIWLLAKAYVVVNDSCYHQLVSHWLNTHAVVEPFIIATNRHLSVVHPIYKLLHPHYRDTMNINGLARLSLVNDGGVIEQTFLWGRYSVEMSAVVYKDWVFTDQALPADLIKRGMAIEDPSCPHGIRLVIEDYPYAVDGLEIWDAIKTWVHEYVFLYYKSDDTLREDPELQACWKELVEVGHGDKKNEPWWPKMQTREELVESCAIIIWTASALHAAVNFGQYPYGGLILNRPTLSRRFMPEKGSAEYEELRKNPQKAYLKTITPKFQTLIDLSVIEILSRHASDEVYLGERDNPNWTSDTRALEAFKRFGNKLAQIENKLSERNNDEKLRNRCGPVQMPYTLLLPSSKEGLTFRGIPNSISI

SEQ ID NO: 514 - protein, 860 aa, Phaseolus vulgaris
MFSGVSGLINRGHKLKGTVVLMRKNVLDVNSVTSVGGIVGQGLDILGSTVDNLTAFLGRSVSLQLISATKPDANGKGKLGKATFLEGIITSLPTLGAGQSAFKIHFEWDDEMGIPGAFYIKNFMQTEFYLVSLTLEDIPNHGSLHFLCNSWIYNAKHFKNDRIFFVNQIYLPSETPAPLVKYREEELVNMRGDGTGERKEWDRIYDYDVYNDLGDPDKGENNARPILGGSDTLPYPRRGRTGRRPTRKDPKSESRSSDIYLPRDEAFGHLKSSDFLTYGLKSVSQNFLPALQSAFDLNFTPNEFDSFEEVHGLYSGGIKLPTDVLSKISPLPVLKEIFRTDGEQTLKFPPPKVVQDSKSAWMTDEEFAREMICGVNPNLIRLLQDFPPQSKLDSQVYGDHTSQITKENLEPNLEGLTVDEAIQSKRLFLLDHHDSIMPYLRRINATSSKAYATRTILFLKKDRTLKPLAIELSLPHPGGDKSGVVSQVFLPADEGVESSVWLLAKSYVIVNDSSYHQLVSHWLNTHAVVEPFVIATNRHLSVVHPIYKLLHPHYRDTMNINALARGDLVNHGGIIEKTFVWGRYSMEMSAVIYKDWVFTDQALPADLIKRGIATEDPECPHGLRLFIEDYPYAVDGLEIWDAIKTWVHEYVFLYYKSDDTLKEDPELQAWWKELVEVGHGDKKNEPWWPKMQTREELVEACSIVIWTASALHAAVNFGQYPYGGLILNRPTLSRRFMPEEGSAEYEELKKSPQKALLKTITPKFQTLVDLSVIEILSRHASDEVYLGERDNPNWTSDTRALEAFKRFGKKLSEIEKKLSQRNNDEKLRNRYGPVMMPYTLLFPSSDEGLTFRGIPNSISI

SEQ ID NO: 515 - protein, 498 aa, Vicia sativa
MAKLLALSLSFCFLLFSSCFALREQSQQNECQLERINALEPDNRIESEGGLIETWNPNNRQFRCARVALSRATLQRNALRRPYYSNAPQEIYIQQGNGYFGMVFPGCPETHEEPQQSEQGEGRRYRDSHQKVNRFREGDIIAVPTGIAFWMYNDQDTPVIAISLTDTGSSNNQLDQMPRRFYLAGNQEQEFLRYQHQQGGKQEQDNDGNNIFSGFKRDFLEDAFNVNRHIVDRLQGRNEDEEKGAIVKVKGGLSIIAPPERQARHERGSRQEEDEDEKEERQPSHHKSRRDEDEDDKEKRHSQKGQSRRQGDNGLEETVCTAKLRANIGSSPSPDIYNPQAGRIKTVTSLDLPVLRWLKLSAEHGSLHKNAMFVPHYNLNANSVIYALKGRARLQVVNCNGNTVFDGELEAGRALTVPQNYAVAAKSLSERFTYVAFKTDDRASIARLAGTSSVIDDLPLDVVAATFNMQRNEARQLKSNNPFKFLVPPRQSEMRASA

SEQ ID NO: 516 - protein, 482 aa, Vicia narbonensis
MAKLLALSLSLCFLLFSNSFALREQSQQNECQLERLDALEPDNRIESEGGLIETWNPNNRQFRCAGVALSRVTLQRNALRRPYYSNAPQEIYIQQGNGYFGVVFPGCPETFEEPQESEQRERRRYRDSHQKVNRFREGDIIAVPTGNVLWMYNDQDTPVIAISLTDTGSSNNQLDQIPRRFYLAGNQEQEFLRYQREQGGKQEQENDGNNIFSGFKRDFLEDALNVNRHIVDRLQGRNEDEEKGAIVKVKGGLSIITPPERQRGSRQEEDEDEKEERQPSRRRDESQKGESRRHGDNGLEETVCTAKLRVNIGSSPSPDIYNPQAGRINTVTSLDLPVLRWLKLSAEHGSLRKNALIVPHYNRNANSVIYALKGRARLQVVNCNGNTVFDGELEAGRALTVPQNYAVAAKSLSERFTYVAFKTNDRDGIARLAGTSSVINDLPLDVVAATFNLQRNEARQLKSNNPFKLLVPPRESEKRASA

SEQ ID NO: 517 - protein, 499 aa, Cicer arietinum
  REGION 189..196: note = MISC_FEATURE - Xaa can be any naturally occurring amino acid
MAKLLALSLSFCFLLFGSCFALRDQPEQNECQLEHLNALEPDNRIKSEGGLIETWNPSNKQFRCAGVALSRATLQPNSLRRPFYTNAPQEIFIQQGNGYFGMVFPGCVETFEEPRESEQGEGSKFRDSHQKVNRFREGDIIAVPTGVVFWMFNDQDTPVIAVSLIDTSSFQNQLDQMPRRFYLAGNHEXXXXXXXXQQEGSEEEENEGGNIFSGFKRDFLEDALNVNRRIVNKLQGRNEDEEKGAIVKVKGGLSIITPPEKEPRQKRGSRQEEDEDEDEKRQPHRHSRQDEDEDEKRQPRRHSRGGSKSQRDNGFEETICTARLHQNIGSSSSPDIYNPQAGRIKTVTSFDLPALRFLKLSAEFGSLHKNAMFVPHYNLNANSILYALKGRARLQIVNCKGNSVFDGELEAGRALIVPQNFAIAAKSLSDRFSYVAFKTNDRAAIGRLLGASSLINGMPEEVVAAAFNMERNEARQLKFNSPFSFLVPPRSDSDNKAAA

SEQ ID NO: 518 - protein, 576 aa, Lathyrus hirsutus
MAIIIKSRFPLLLLLGIIFLASVCATWANYDEGSEPRVPGQRERGRQEGEKAEKSHEKWRPSYEEEYDEGSEPRVPGKRERGRQEGEKEEKRHGEWRPSHEEEYDEGSEPRVPTHGERGRQEGEKEEKRHEEWRPSYEKEEDEEEKEKYKYQREKKEQKEVQPGREKWERKQDEKHVEEDEDQEEEQWRGSKRREDPEERARLRYREERTKSNVEEETEERRNPFLFKSNKFLTLFENENGHIRRLQRFDERSDIFENLQNYRLVEYKAKPHTMFLPQHIDADLIIVVLNGKAILTVLSPNDRNSYNLERGDTIKLPAGTTSYLVNQDDEEDLRVVDLAIPVNRPGKFEAFGLSANKNQYLRGFSKNILEAFLNTKYETIEKVLLEEQERRDRKGRQQGQETNAIVKVSREQIEELRKLAKSSSKKSLLSESEPINLRSQNPKYSNKFGKLFEITPEKKYPQLQDLDVSISCVEINEGAPLLPHYNSRAIVLLLVNEGKGNLELVGFKNEQQRQRENEERNKKVQRYEARLSPGDVVVIPAGHPVAISASLNLNLVGFGVNAENNQRNFLTGSDDN

SEQ ID NO: 519 - protein, 564 aa, Lathyrus cicera
MATIIKSRFPLLLLLGIIFLASVCVTLANYDEGSEPRVPAQRERGRQEGEKEEKRHGEWRPSHEKEYDEGSEPRVPGRRERGRQEGEKEEKRHGEWRPSYEKEYDEGSEPRVPGRRERGRQEGEKEEKRHGEWRPSYEKEYDEEEKQKYQYEREKEEQKEVQPGRERWERKEDEEKEEDQWRGSQRHEDPEERARLRYRKERTKKYVEEDTEETSSESQGRRNPFLFKSNKFLTLFENENGYIRRLQRFDERSDIFENLQNYRLVEYRAKPHTIFLPQHIDADLILVILNGKAILTVLSPNDRNSYNLERGDTIKLPAGTTSYLVNEDDEEDLRVVDLVIPVNRPGKFEAFDLNQYLGGFSKSVLEASLNTKYETIEKVLLEEQQKQGQETNAIVKVSREQIEELRKLAKSSSKKSLLSELEPVNLRSHSPKYSNKFGKFFEITPEKKYPQLQDLDVSISCVEINEGALLLPHYNSRAIVVVLVNEGKGNLELLGVQNEDEQQERKERNKEVQRYEARLSPGDVVIIPSGHPVAVSASSNLNLLGFGINAENNQRNFLSGSDDN

SEQ ID NO: 520 - protein, 527 aa, Lathyrus sativus
MATIIKSRFPLLLLLGIIFLASVCVTYANYDEGSEPRVPAQRERGRQEGEKEEKRHGEWRPSSEKEYDEGSEPRVPGRRERGRQEGEKEEKRHGEWRPSYEKEYDEEEKQKYQYEREKKEQKEVEPGRERWERKEDEEKEEDQWRGSQRHEDPEERARLRYRKERTKKYVEEDTEETSSESQGRRNPFLFKSNKFLTLFENENGYIRRLQRFDERSDLFENLQNYRLVEYRAKPHTIFLPQHIDADLILVILNGKAILTVLSPNDRNSYNLERGDTIKLPAGTTSYLVNEDDEEDLRVVDLVIPVNRPGKFEAFDLNQYLGGFSKSVLKASLNTKYETIEKVLLEEQQKQGQETNAIVKVSREQIEELRKLAKSSSKKSLLSELEPVNLRSHSPKYSNKFGKFFEITPEKKYPQLQDLDVSISCVEINEGALLLPHYNSRAIVVLLVNEGKGNLELLGVQDEDEQQERKKRNKEVQRYEARLSPSDVVIIPAGHPVAVSASSNLNLLGFGINAENNERNFLSGSDDN

SEQ ID NO: 521 - protein, 463 aa, Medicago truncatula
MAIKAPFQLLMLLGIFFLASVCVSSRDDRHDQENPFFFNANHFQTLFENENGHIRLLQRFDKRSKIFENLQNYRLLEYHSKPHTLFLPQHNDADFILAVLSGKAILTVLNPDNRNSFNLERGDTIKLPAGSIAYLANRDDNEDLRVLDLAIPVNRPGKFQSFSLSGSQNQQSFFSGFSKNILEAAFNANYEEIERVLIEEHEQEPQHRRGLRKDRRQQSQDSNVIVKVSREQIEELSRHAKSSSRRSGSSESAPFNLRSREPIYSNEFGNFFEITPEKNPQLKDLDILVNYAEIREGSLLLPHFNSRATVIVVVDEGKGEFELVGQRNENQQEQREEDEQQEEERSQQVQRYRARLSPGDVYVIPAGHPTVVSASSDLSLLGFGINAENNERNFLAGEEDNVISQIERPVKEVAFPGSAQDVESLLKNQRQSYFANAQPQQREREEGRSQRQRELISSILGVF

SEQ ID NO: 522 - protein, 499 aa, Vicia pannonica
MATTFKSRFSLLLLLGIIFLAFVCVTCANYDEGSEPRVPGQRERGRQEGEKEEQSRERHPQREPSREKEEDEEEKQKYDEGTEPRVPGQRERGRQEGEKEEQRRERHPGQREPSQEEDEEREESDRRQEGSSKSEEQRNPFLFKSNKFLTLFQNGNGHIRLLQRFDKRSDLFENLQNYRLLEYRAKPHTIFLPQHIDADLILVVLSGRAILTVLSPDDRNSYNLERGDTIKLPAGTTSYPLNQDDEEDLRVVDLAISVNRPGKVESFNLSGNKNQYLRGFSENILEASFNTKYETIEKVLLEEQDKESQQPRGQRLQRQETNALVKVSREQVEELKRLARTSSKKGVSSEFEPFNLRSHGPKYSNKFGKFFEITPEKKYPQLQDLDISVSSVEINEGALFLPHYNSRAIVVVLVDEGKGNLELVGFKNEQQEQREKEDEQEERNKQVQRYEAKLSPGDVVIIPAGHPVAVSASSNLNLLGFGINAENNQRNFLTGSDDN

SEQ ID NO: 523 - protein, 515 aa, Vicia ludoviciana
MATTIKLRFPLLLLLGVILLASVCVTCANYDEGSEPRVPGRPEGEKEEKHRGKLRPSYEKEEDEGEKQRYHYEKKEQKEAQPRREKKEQKEEEKQVEEESRESQRYEDPGERARERYRAEIIKRQVEKEREERDRRHQREGEEEEGSSKSRNPFLFKSNNFLTLFENENGHIRLLQRFDKRSDLFENLQNYRLVEYRAKPHTIFLPQHIDADLILVVLSGKAILTVLSPNNRNSYNLKRGDTIKLPAGTTSYLLNSDDEEDLRMVDLAISVNRPGKVESFNLSGNKNQYLRGFSKNILEASFNTKYETIEKVLLEEQDKESQQSIGQKRISQRQETNALVKVSREQIEEPKRLARSSSRKGVSSEFEPINLRSQRPKYSNKFGKFYEISPEKKYPQLQDLDVSVSSVEINEGALLLPHYNSRAIVTVLVNEGKGNLELIGFQNEQQGQREKEDEQQHERNKQVQRYDARLSSGDVVIIPAGHPVAVSASSNLDLLGFGINAENSQRNFLTGSDDN

SEQ ID NO: 524 - protein, 454 aa, Oryza sativa
CRFDRLQAFEPIRSVRSQAGTTEFFDVSNEQFQCTGVSAVRRVIEPRGLLLPHYTNGASLVYIIQGRGITGPTFPGCPESYQQQFQQSGQAQLTESQSQSHKFKDEHQKIHRFRQGDVIALPAGVAHWCYNDGEVPVVAIYVTDLNNGANQLDPRQRDFLLAGNKRNPQAYRREVEERSQNIFSGFSTELLSEALGVSSQVARQLQCQNDQRGEIVRVEHGLSLLQPYASLQEQEQGQVQSRERYQEGQYQQSQYGSGCSNGLDETFCTMKVRQNIDNPNRADTYNPRAGRVTNLNTQNFPILNLVQMSAVKVNLYQNALLSPFWNINAHSVVYITQGRARVQVVNNNGKTVFNGELRRGQLLIIPQHYAVVKKAQREGCAYIAFKTNPNSMVSHIAGKSSIFRALPNDVLANAYRISREEAQRLKHNRGDEFGAFTPIQYKSYQDVYNAAESS

SEQ ID NO: 525 - protein, 500 aa, Zizania latifolia
MNMATINGPTIFFTVCLFLLCHGSLAQLLGQSTSQWQSSHRGSSRQCRFDRLQAFEPVRSVRSQAGTTEFFDASNELFQCAGVSIVRRIIEPRGLLLPQYTNGATIMYIIQGRGITGQTFPGCPESYQQQFQQSMQAQLTGSQSQSQKFKDEHQKINRFRQGDVIALPAGVAHWCYNDGEVPVVAIYVIDINNAANQLDPRQRDFLLAGNMRSPQAYRREVENQSQNIFSGFSAELLSEALGISTGVARQLQCQNDQRGEIVRVEHGLSLLQPYASLQEQEQKQEQPRERYQVTQHQQSQYGGGCSNGLDETFCAMRIWQNIDNPNLADTYNPRAGRVTNLNSQKFPILNLIQMSAVKVNLYQNALLSPFWNINSHSVVYVTQGCARVQVVNNNGKTVFNGELRRGQLLIIPQHYVVVKKAQREGCAYIAFKTNPNSMVSHIVGKSSIFRALPTDVLANAYRISREDAQRLKHNRGDELGAFTPLQYKSYQDVSSVAASS

SEQ ID NO: 526 - protein, 527 aa, Avena sativa
MATTSFPSMLFYFCIFLLFHGSMAQLFGQSSTPWQSSRQGGLRGCRFDRLQAFEPLRQVRSQAGITEYFDEQNEQFRCTGVSVIRRVIEPQGLVLPQYHNAPALVYILQGRGFTGLTFPGCPATFQQQFQPFDQSQFAQGQRQSQTIKDEHQRVQRFKQGDVVALPAGIVHWCYNDGDAPIVAIYVFDVNNNANQLEPRQKEFLLAGNNKREQQSGNNIFSGLSVQLLSEALGISQQAAQRIQSQNDQRGEIIRVSQGLQFLKPIVSQQVPGEQQVYQPIQTQEGQATQYQVGQSTQYQVGKSTPYQGGQSSQYQAGQSWDQSFNGLEENFCSLEARKNIENPQHADTYNPRAGRITRLNSKNFPILNIVQMSATRVNLYQNAILSPFWNINAHSVIYMIQGHARVQVVNNNGQTVFNDILRRGQLLIVPQHFVVLKKAEREGCQYISFKTNPNSMVSHIAGKSSILRALPIDVLANAYRISRQEARNLKNNRGEEFGAFTPKLTQKGFQSYQDIEEGSSSPVRASE

SEQ ID NO: 527 - protein, 495 aa, Oryza sativa
MTISVFSRFSIYFCVLLLCNGSMAQLFDPATNQWQTHRQGSFRECRFERLQAFEPLQNVRSEAGVTEYFDETNELFQCTGTFVIRRVIQPQGLLIPRYANTPGMVYIIQGRGSMGLTFPGCPATYQQQSQQFLFQGESQSQKFIDEHQKIHQFRQGDIVVLPTGVAHWFYNDGDTPVVALYVYDINNSANQLEPRHREFLLAGKNNRVQQVYGRSIQQHSGQNIFNGFSVEPLSEALNINTVTTKRLQSQNDQRGEIIHVKNGLQLLKPTLTQRQEQEQAQYQEVQYSEKPQTSSRWNGLEENLCTIKTRLNIENPSRADSYDPRAGRITSLDSQKFPILNIIQMSATRVNLYQNAILTPFWNVNAHSLMYVIRGRARVQVVSNFGKTVFDGVLRPEQLLIIPQNYVVLKKAQHEGCQYIAINTNANAFVSHLAGVDSVFHALPVDVIANAYCISREEARRLKNNRGDEYGPFPPRLQQQIYPEFSNESKGETSE

SEQ ID NO: 528 - protein, 480 aa, Oryza sativa
LLCHGSMAQIFSLGINPWQNPRQGGSRECRFDRLQAFEPLRKVRHEAGVTEYFDEKNEQFQCTGTLVIRRIIEPQGLLLPRYSNTPGLVYIIQGTGVLGLTFPGCPATYQKQFRHFGLEGGSQRQGKKLRDENQKIHQFRQGDVVALPSGIPHWFYNEGDTPVVALFVFDVNNNANQLEPRQKEFLLAGNNIEQQVSNPSINKHSGQNIFNGFNTKLLSEALGVNIEVTRRLQSQNDRRGDIIRVKNGLRLIKPTITQQQEQTQDQYQQIQYHREQRSTSKYNGLDENFCAIRARLNIENPNHADTYNPRAGRITNLNSQKFSILNLVQMSATRVNLYQNAILSPFWNINAHSLVYTIQGRARVQVVSNHGKAVFNGVLRPGQLLIIPQNYVVMKKAELEGFQFIAFKTNPNAMVNHIAGKNSVLRAMPVDVIANAYRISRQEARSLKNNRGEEIGAFTPRYQQQKIHQEYSNPNESETQ

SEQ ID NO: 529 - protein, 518 aa, Oryza sativa
MATTRFPSLLFYSCIFLLCNGSMAQLFGQSFTPWQSSRQGGLRGCRFDRLQAFEPLRQVRSQAGITEYFDEQNEQFRCAGVSVIRRVIEPQGLLLPQYHNAPGLVYILQGRGFTGLTFPGCPATFQQQFQPFDQARFAQGQSKSQNLKDEHQRVHHIKQGDVVALPAGIVHWCYNDGDAPIVAVYVFDVNNNANQLEPRQKEFLLAGNNKREQQFGQNIFSGFSVQLLSEALGISQQAAQKIQSQNDQRGEIIRVSQGLQFLKPFVSQQGPVEHQAYQPIQSQQEQSTQYQVGQSPQYQEGQSTQYQSGQSWDQSFNGLEENFCSLEARQNIENPKRADTYNPRAGRITHLNSKNFPTLNLVQMSATRVNLYQNAILSPYWNINAHSVMHMIQGRARVQVVNNHGQTVFNDILRRGQLLIIPQHYVVLKKAEREGCQYISFKTTPNSMVSYIAGKTSILRALPVDVLANAYRISRQESQNLKNNRGEEFGAFTPKFAQTGSQSYQDEGESSSTEKASE

SEQ ID NO: 530 - protein, 498 aa, Oryza sativa
MASTNRPIVFFTVCLFLLCDGSLAQQLLGQSTSQWQSSRRGSPRGCRFDRLQAFEPIRSVRSQAGTTEFFDVSNELFQCTGVSVVRRVIEPRGLLLPHYTNGASLVYIIQGRGITGPTFPGCPETYQQQFQQSGQAGLTESQSQSHKFKDEHQKIHRFRQGDVIALPAGVAHWCYNDCEVPVVAIYVTDINNGANQLDPRQRDFLLAGNKRNPQAYRREVEEWSQNIFSGFSTELLSEAFGISNQVARQLQCQNDQKGEIVRVERGLSLLQPYASLQEQEQGQMQSREHYQEGGYQQSQYGSGCPNGLDETFCVNKVRQNIDNPNRADTYNPRAGRVTNLSQNFPILNLVQMSAVKVNLYQNTDTWISMGQEENALLSPFWNINAHSIVYITQGRAQVQVLRRGQLLIVPQHYVVVKKAQREGCAYIAFKTNPNSMVSHIAGKSSIFRALPTDVLANAYRISREEAQRLKHNRGDEFGAFTPLQYKSYQDVYNVAESS

SEQ ID NO: 531 - protein, 491 aa, Oryza brachyantha
MKSSIVFSTICLVLLCHGSLAQLLSQSTSQWQSSRRGSPRQCRFDQLQAFEPIRTVRSQAGVTEFYDVSNELFQCTGVSVVRRVIEPRGLLLPHYSNGATLVYIIQGRGITGPTFPGCPETYQQQFQQSGEAQPFEGQSHKFRDEHQKIHRFRQGDVVALPAGVAHWCYNDGEVPIVAIYVTDIYNSANQLDPRHRDFFLAGNNKVAQQLYRSEARENSKNIFGGFSVELLSEALGISRGVARQLQCQNDQRGEIVRVEHGLALLQPYASVQEQQQEQVQSRDYEQTQYQQKQPQGSCSNGLDETFCTMRLRQNIDNPNLADTYNPKAGRITYLNGQKFPILNLVQMSAVKVNLYQNAVLSPFWNINAHSVVYITQGRARVQVVNNNGKTVFDGELRQGQLLIIPQHHVVLKKAQREGCSYIALKTNPNSIVSHIAGKNSIFRALPGDVVTNAYRISREEAKRIKHNRGDESGVFAPSHAYRSYQDMSVAA

SEQ ID NO: 532 - protein, 492 aa, Brachypodium distachyon
MAHTSFSSFLSYFCLFLLFHGSMAQVLGQVSTWQSSRQGGSRDCSFDRLQAIEPVTQVRSQAGLTEYFDEQNEQFRCAGVFVIRRVIEPRGLLLPRYHNTPGLVYILQGNGFVGLTFPGCPETFREQFQQFRQTQSTLGQSQCQSQKLGDVHQRVHQFTQGDVVALPTGVAHWIYNGGDAPVVIVYVFDVNNNANQLEPRQKEFLLGGNYNGVLQYGQNIFSGFNAQLLSQAFGINEQTSQRIQNQNDGRGDIIRVDNGLQFLKPVVTQQQPEQPFMPIQHQTGQSSRNGLEENFCSLEPRQNIEDPNRADTYNPRAGSITRLNGQNFPILNLVQMSATRVNLQKNAILSPFWNINAHSVVYVIQGHALVQVVNNQGHNVFNGLLHRGQLLIIPQNYVVLKKAESEGYQYIAFKTNANSMVSHIAGKNSILRALPVDVIANAYRISRQEAQNLKNNRGEETGVLTPNFSQSTCQSYQTEDVQSLRPMSHWSE

SEQ ID NO: 533 - protein, 166 aa, Oryza sativa
  SITE 120: note = MISC_FEATURE - Xaa can be any naturally occurring amino acid
MASNKVVFSALLLIIVSVLAATATMADHHKDQVVYSLGERCQPGMGYPMYSLPRCRAVVKRQCVGHGAPGGAVDEQLRQDCCRQLAAVDDSWCRCSALNHMVGGIYRELGATDVGHPMAXVFPGCRRGDLERAAASLPAFCNVDIPNGTGGVCYWLGYPRTPRTGH

SEQ ID NO: 534 - protein, 165 aa, Oryza sativa
MASNKVVFSALLLIIVSVLRRDGTMADHHKDQVVYSLGERCQPGMGYPMYSLPRCRAVVKRQCVGHGAPGAVDEQLRQDCCRQLAAVDDSWCRCSALNHMVGGIYRELGATDVGHPMAEVFPGCRRGDLERAAASLPAFCNVDIPNGTGGVCYWLGYPRTPRTGH

SEQ ID NO: 535 - protein, 157 aa, Oryza sativa
MASNKVVISALLVVVVSVLAATTTMADHHQEQVVYTPGQLCQPGIGYPTYPLPRCRAFVKRQCVAPGTVDEQVRRGCCRQLAAIDSSWCRCDALNHMLRIIYRESGAADAGHPMAEVFRGCRRGDIERAAASLPAFCNVDIPNGVGGVCYWLPGTGY

SEQ ID NO: 536 - protein, 605 aa, Oryza rufipogon
MSALTTSQLATSATGFGIADRSAPSSLLRHGFQGLKPRSPAGGDATSLSVTTSARATPKQQRSVQRGSRRFPSVVVYATGAGMNVVFVGAEMAPWSKTGGLGDVLGGLPPAMAANGHRVMVISPRYDQYKDAWDTSVVAEIKVADRYERVRFFHCYKRGVDRVFVDHPSFLEKVWGKTGEKIYGPDTGVDYKDNQMRFSLLCQAPRILNLNNNPYFKGTYGEDVVFVCNDWHTGPLASYLKNNYQPNGIYRNAKVAFCIHNISYQGRFAFEDYPELNLSERFRSSFDFIDGYDTPVEGRKINWMKAGILEADRVLTVSPYYAEELISGIARGCELDNIMRLTGITGIVNGMDVSEWDPSKDKYITAKYDATTAIEAKALNKEALQAEAGLPVDRKIPLIAFIGRLEEQKGPDVMAAAIPELMQEDVQIVLLGTGKKKFEKLLKSMEEKYPGKVRAVVKFNAPLAHLIMAGADVLAVPSRFEPCGLIQLQGMRYGTPCACASTGGLVDTVIEGKTGFHMGRLSVDCKVVEPSDVKKVAATLKRAIKVVGTPAYEEMVRNCMNQDLSWKGPAKNWENVLLGLGVAGSAPGIEGDEIAPLAKENVAAP

SEQ ID NO: 537 - protein, 609 aa, Oryza nivarra
MSALTTSQLATSATGFGIADRSAPSSLLRHGFQGLKPRSPAGGDASSLSVTTSARATPKQQRSVQRGSRRFPSVVVYATGAGMNVVFVGAEMAPWSKTGGLGDVLGGLPPAMAANGHRVMVISPRHDQYKDAWDTSVVAEIKVADRYERVRFFHCYKRGVDRVFIDHPSFLEKVWGKTGEKIYGPDTGVDYKDNQMRFSLLCQAALEAPRILNLNNNPYFKGTYGEDVVFVCNDWHTGPLPSYLKNNYQPNGIYRNAKVAFCIHNISYQGRFAFEDYPELNLSERFRSSFDFIDGYDTPVEGRKINWMKAGILESDRVLTVSPYYAEELISGIARGCELDNIMRLTGITGIVNGMDVSEWDPSKDKYIAAKYDATTAIEAKALNKEALQAEAGLPVDRKIPLIAFIGRLEEQKGPDVMAAAIPELMQENVQIVLLGTGKKKFEKLLKSMEEKYPGKVRAVVKFNAPLAHLIMAGADVLAVPSRFEPCGLIQLQGMRYGTPCACASTGGLVDTVIEGKTGFHMGRLSVDCKVVEPSDVQKVATTLKRAIKIVGTPAYNEMVRNCMNQDLSWKGPAKNWENVLLGLGVAGSAPGVEGEEIAPLAKENVAAP

SEQ ID NO: 538 - protein, 603 aa, Hordeum vulgare
MAALATSQLATSGTVLGVTDRFRRPGFQGLRPRNPADAALGMRTIGASAAPKQSRKAHRGSRRCLSVVVRATGSGMNLVFVGAEMAPWSKTGGLGDVLGGLPPAMAANGHRVMVVSPRYDQYKDAWDTSVISEIKVADEYERVRFFHCYKRGVDRVFIDHPWFLEKVRGKTKEKIYGPDAGTDYEDNQQRFSLLCQAALEAPRILNLNNNPYFSGPYGEDVVFVCNDWHTGLLACYLKSNYQSNGIYRTAKVAFCIHNISYQGRFSFDDFAQLNLPDRFKSSFDFIDGYDKPVEGRKINWMKAGILQADKVLTVSPYYAEELISDEARGCELDNIMRLTGITGIVNGMDVSEWDPTKDKFLAVNYDITTALEAKALNKEALQAEVGLPVDRKVPLVAFIGRLEEQKGPDVMIAAIPEILKEEDVQIILLGTGKKKFEKLLKSMEEKFPGKVRAVVRFNAPLAHQMMAGADLLAVTSRFEPCGLIQLQGMRYGTPCVCASTGGLVDTIVEGKTGFHMGRLSVDCNVVEPADVKKVATTLKRAVKVVGTPAYQEMVKNCMIQDLSWKGPAKNWEDVLLELGVEGSEPGIVGEEIAPLAMENVAAP

SEQ ID NO: 539 - protein, 495 aa, Oryza brachyantha
MATTVFSRFSTYFCVLLLCHGSMAQLFNPSTNPWHNPRQGSSRECRFDRLQPFEPLRKVRSEAGVTEYFDEKNELFQCTGTFVIRRVIQPQGLLVPRYTNAPGLVYIIQGRGSIGLTFPGCPATYQQQFQQFLPQEQSQSQKFRDEHQKIHQFRQGDIVALPAGVAHWFYNDGDAPVVAVYVYDVKNSANQLEPRQREFLLGGNNMRAQQVYGSSAEQHSRQNIFSGFGVEILSEALGISTVTTKRLQSQNDQRGEIIHVKNGLQFLKPTLTQQQEQAQAQYQEVQYSEQQQTSSRWNGLDENFCTIKARMNIENTSRADTYNPRAGRTTSLNSQKFPILNLVQMSATRVNLYQNAILSTFWNVNAHSLVYTIQGRARVQVVSNFGKTVFDGELRPGQLLIIPQHYVVLKKAQREGFRYIAIKTNANAFVSQLVGKNSVFRSLPVDVIANVYRISREQARSLKNNRGEEHGAFAPRSQQQSYPGFSNQSESETSE

SEQ ID NO: 540 - protein, 494 aa, Oryza brachyantha
MATTTFSRFSIYFCVLLLCHGSMAQLFSPTLNPWHSSRRGGSRDCRFDRLQAFEPLRRVRSEAGVTEYFDERNEQFQCTGTFVIRRVIEPQGLLVPRYTNTPGVVYIMQGRGSMGLTFPGCPATYQQQFQQFLPEGQSQSQKFRDEHQKIHQFRQGDIVALPAGVAHWFYNEGDTPVVALYVFDINNSANQLEPRQKDFLLAGNNNREQQVYGRSIEKHSGQNIFSGFNHELLSEALGISTLAAKRLQGQNDHRGEIIRVRNGLQLLKPTFTQQQEQAQSQYQVQYSEKQQESTRCNGLDENFCTINARLNIENPSRADTYNPRAGRITHLNNQKFPILNLVQMSATRVNLYQNAILSPYWNVNAHSLVYMVQGHARVQVVSNLGKTVFNSVLRPGQLLIIPQHYVVLKKAEREGCQYIAFKTNANSIVSQLAGKNSILRAMPVDVVANAYRISREQARDLKNNRGEELGAFTPKFEQQSYPGLSNESESEASE

SEQ ID NO: 541 - protein, 500 aa, Zizania latifolia; identical to SEQ ID NO: 525

SEQ ID NO: 542 - protein, 197 aa, Zea mays
MAKIAAAAAAAAALCFAALVAVAVCQGEVERQRLRDLQCWQEVQESPLDACRQVLDRQLTGGGVGGPFRWGTGLRMRCCQQLQDVSRECRCAAIRSMVRGYEEAMPPLEKGWWPWGRQQQPPPQGGGGGQGGYYYPCSRAGEGYQTQMYPPCRPGTTGPRIGRVRLTKAREYAAGLPMMCRLSEPQECSIFSGGDQY

SEQ ID NO: 543 - protein, 192 aa, Sorghum bicolor
MAKIAAVAATAALCLAALVAVAVGQGVVERQRLKDLQCWQEVQENPLGACRQVLDRQLTGGMRYGIGPFRWGTGLRMRCCQQLQDVSRECRCAAIRSMVRGYEETMPPLEKGWWGQQPQPGYDYPCSQAGEGYGYGESGQQQMYPPCRPGTGQKIARVKLTKARQYAAGMPMMCRLSEPQECSVFSGGDQYY

SEQ ID NO: 544 - protein, 177 aa, Setaria italica
MAKFVVAAATAALCLAALVAMAAGQSGFERQRLRDLRCQREVEENPLWACRQVLDRQLTGGMRYGVGPFRWGTGLRMRCCQQLQDVSRECRCSAVRRMVRGYEEAMPPLEEGPYGYGGEQGEGYYGGGEGGEGYLPFPPRRIGRVRLTKARQYAAGLPMMCRLEPQECSVFSGDQYK

SEQ ID NO: 545 - protein, 336 aa, Staphylococcus aureus
MKGKFLKVSSLFVATLTTATLVSSPAANALSSKAMDNHPQQSQSSKQQTPKIQKGGNLKPLEQREHANVILPNNDRHQITDTTNGHYAPVTYIQVEAPTGTFIASGVVVGKDTLLTNKHVVDATHGDPHALKAFPSAINQDNYPNGGFTAEQITKYSGEGDLAIVKFSPNEQNKHIGEVVKPATMSNNAETQVNQNITVTGYPGDKPVATMWESKGKITYLKGEAMQYDLSTTGGNSGSPVFNEKNEVIGIHWGGVPNEFNGAVFINENVRNFLKQNIEDIHFANDDQPNNPDNPDNPNNPDNPNNPDEPNNPDNPNNPDNPDNGDNNNSDNPDAA

SEQ ID NO: 546 - protein, 11 aa, synthetic construct
  REGION 1..11: note = Synthetic peptide
SRAIVIVTVNE

SEQ ID NO: 547 - protein, 8 aa, synthetic construct
  REGION 1..8: note = Synthetic peptide
AKLTPGDV

SEQ ID NO: 548 - protein, 10 aa, synthetic construct
  REGION 1..10: note = Synthetic peptide
IVIVTVNEGK

SEQ ID NO: 549 - protein, 16 aa, synthetic construct
  REGION 1..16: note = Synthetic peptide
LDALEPDNRIESEGGL

SEQ ID NO: 550 - protein, 10 aa, synthetic construct
  REGION 1..10: note = Synthetic peptide
RPYYSNAPQE

SEQ ID NO: 551 - protein, 25 aa, synthetic construct
  REGION 1..25: note = Synthetic peptide
LDALEPDNRIESEGGLIETWNPNNK

SEQ ID NO: 552 - protein, 11 aa, synthetic construct
  REGION 1..11: note = Synthetic peptide
AIVIVTVNEGK

SEQ ID NO: 553 - protein, 16 aa, synthetic construct
  REGION 1..16: note = Synthetic peptide
LQVVNCNGNTVFDGEL

SEQ ID NO: 554 - protein, 15 aa, synthetic construct
  REGION 1..15: note = Synthetic peptide
QVVNCNGNTVFDGEL

SEQ ID NO: 555 - protein, 10 aa, synthetic construct
  REGION 1..10: note = Synthetic peptide
IIAVPTGIVF

SEQ ID NO: 556 - protein, 16 aa, synthetic construct
  REGION 1..16: note = Synthetic peptide
GRRYRDRHQKVNRFRE

SEQ ID NO: 557 - protein, 11 aa, synthetic construct
  REGION 1..11: note = Synthetic peptide
RPYYSNAPQEI

SEQ ID NO: 558 - protein, 12 aa, synthetic construct
  REGION 1..12: note = Synthetic peptide
RLDALEPDNRIE

SEQ ID NO: 559 - protein, 14 aa, synthetic construct
  REGION 1..14: note = Synthetic peptide
RLDALEPDNRIESE

SEQ ID NO: 560 - protein, 20 aa, synthetic construct
  REGION 1..20: note = Synthetic peptide
LDALEPDNRIESEGGLIETW

SEQ ID NO: 561 - protein, 11 aa, synthetic construct
  REGION 1..11: note = Synthetic peptide
LDALEPDNRIE

SEQ ID NO: 562 - protein, 18 aa, synthetic construct
  REGION 1..18: note = Synthetic peptide
LDALEPDNRIESEGGLIE

SEQ ID NO: 563 - protein, 16 aa, synthetic construct
  REGION 1..16: note = Synthetic peptide
LDALEPDNRIESEGGL

SEQ ID NO: 564 - protein, 10 aa, synthetic construct
  REGION 1..10: note = Synthetic peptide
RPYYSNAPQE

SEQ ID NO: 565 - protein, 25 aa, synthetic construct
  REGION 1..25: note = Synthetic peptide
LDALEPDNRIESEGGLIETWNPNNK

SEQ ID NO: 566 - protein, 27 aa, synthetic construct
  REGION 1..27: note = Synthetic peptide
VEHGLSLLQPYASLQEQEQGQVQSRER

SEQ ID NO: 567 - protein, 9 aa, synthetic construct
  REGION 1..9: note = Synthetic peptide
RSQNIFSGF

SEQ ID NO: 568 - protein, 14 aa, synthetic construct
  REGION 1..14: note = Synthetic peptide
GITGPTFPGCPESY

SEQ ID NO: 569 - protein, 4 aa, synthetic construct
  REGION 1..4: note = Synthetic peptide
CNGS

SEQ ID NO: 570 - protein, 5 aa, synthetic construct
  REGION 1..5: note = Synthetic peptide
SPREC

SEQ ID NO: 571 - protein, 4 aa, synthetic construct
  REGION 1..4: note = Synthetic peptide
PREC

SEQ ID NO: 572 - protein, 5 aa, synthetic construct
  REGION 1..5: note = Synthetic peptide
PRECR

SEQ ID NO: 573 - protein, 4 aa, synthetic construct
  REGION 1..4: note = Synthetic peptide
CPES

SEQ ID NO: 574 - protein, 4 aa, synthetic construct
  REGION 1..4: note = Synthetic peptide
SGCS

SEQ ID NO: 575 - protein, 4 aa, synthetic construct
  REGION 1..4: note = Synthetic peptide
CSNG

SEQ ID NO: 576 - protein, 12 aa, synthetic construct
  REGION 1..12: note = Synthetic peptide
RSQNIFSGFSTE

SEQ ID NO: 577 - protein, 14 aa, synthetic construct
  REGION 1..14: note = Synthetic peptide
VEEWSQNIFSGFST

SEQ ID NO: 578 - protein, 13 aa, synthetic construct
  REGION 1..13: note = Synthetic peptide
WSQNIFSGFSTEL

SEQ ID NO: 579 - protein, 12 aa, synthetic construct
  REGION 1..12: note = Synthetic peptide
WSQNIFSGFSTE

SEQ ID NO: 580 - protein, 10 aa, synthetic construct
  REGION 1..10: note = Synthetic peptide
STSQWQSSRR

SEQ ID NO: 581 - protein, 4 aa, synthetic construct
  REGION 1..4: note = Synthetic peptide
NRPI

SEQ ID NO: 582 - protein, 4 aa, synthetic construct
  REGION 1..4: note = Synthetic peptide
CDGS

SEQ ID NO: 583 - protein, 4 aa, synthetic construct
  REGION 1..4: note = Synthetic peptide
PRGC

SEQ ID NO: 584 - protein, 5 aa, synthetic construct
  REGION 1..5: note = Synthetic peptide
PRGCR

SEQ ID NO: 585 - protein, 4 aa, synthetic construct
  REGION 1..4: note = Synthetic peptide
RGCR

SEQ ID NO: 586 - protein, 4 aa, synthetic construct
  REGION 1..4: note = Synthetic peptide
GCRF

SEQ ID NO: 587 - protein, 4 aa, synthetic construct
  REGION 1..4: note = Synthetic peptide
PTFP

SEQ ID NO: 588 - protein, 5 aa, synthetic construct
  REGION 1..5: note = Synthetic peptide
PGCPE

SEQ ID NO: 589 - protein, 4 aa, synthetic construct
  REGION 1..4: note = Synthetic peptide
GCPE

SEQ ID NO: 590 - protein, 4 aa, synthetic construct
  REGION 1..4: note = Synthetic peptide
CPET

SEQ ID NO: 591 - protein, 4 aa, synthetic construct
  REGION 1..4: note = Synthetic peptide
AHWC

SEQ ID NO: 592 - protein, 4 aa, synthetic construct
  REGION 1..4: note = Synthetic peptide
HWCY

SEQ ID NO: 593 - protein, 4 aa, synthetic construct
  REGION 1..4: note = Synthetic peptide
SGCP

SEQ ID NO: 594 - protein, 5 aa, synthetic construct
  REGION 1..5: note = Synthetic peptide
SGCPN

SEQ ID NO: 595 - protein, 4 aa, synthetic construct
  REGION 1..4: note = Synthetic peptide
GCPN

SEQ ID NO: 596 - protein, 4 aa, synthetic construct
  REGION 1..4: note = Synthetic peptide
CPNG

SEQ ID NO: 597 - protein, 5 aa, synthetic construct
  REGION 1..5: note = Synthetic peptide
TFCTM

SEQ ID NO: 598 - protein, 4 aa, synthetic construct
  REGION 1..4: note = Synthetic peptide
FCTM

SEQ ID NO: 599 - protein, 5 aa, synthetic construct
  REGION 1..5: note = Synthetic peptide
FCTMR

SEQ ID NO: 600 - protein, 4 aa, synthetic construct
  REGION 1..4: note = Synthetic peptide
CTMR

SEQ ID NO: 601 - protein, 4 aa, synthetic construct
  REGION 1..4: note = Synthetic peptide
EGCA

SEQ ID NO: 602 - protein, 13 aa, synthetic construct
  REGION 1..13: note = Synthetic peptide
SQNIFSGFSTELL

SEQ ID NO: 603 - protein, 11 aa, synthetic construct
  REGION 1..11: note = Synthetic peptide
SQNIFSGFSTE

SEQ ID NO: 604 - protein, 10 aa, synthetic construct
  REGION 1..10: note = Synthetic peptide
QNDQRGEIVR

SEQ ID NO: 605 - protein, 12 aa, synthetic construct
  REGION 1..12: note = Synthetic peptide
SQNIFSGFSTEL

SEQ ID NO: 606 - protein, 12 aa, synthetic construct
  REGION 1..12: note = Synthetic peptide
QLQCQNDQRGEI

SEQ ID NO: 607 - protein, 12 aa, synthetic construct
  REGION 1..12: note = Synthetic peptide
LGQSTSQWQSSR

SEQ ID NO: 608 - protein, 15 aa, synthetic construct
  REGION 1..15: note = Synthetic peptide
QQLLGQSTSQWQSSR

SEQ ID NO: 609 - protein, 13 aa, synthetic construct
  REGION 1..13: note = Synthetic peptide
LLGQSTSQWQSSR

SEQ ID NO: 610 - protein, 9 aa, synthetic construct
  REGION 1..9: note = Synthetic peptide
NDQRGEIVR

SEQ ID NO: 611 - protein, 11 aa, synthetic construct
  REGION 1..11: note = Synthetic peptide
GQSTSQWQSSR

SEQ ID NO: 612 - protein, 9 aa, synthetic construct
  REGION 1..9: note = Synthetic peptide
STSQWQSSR

SEQ ID NO: 613 - protein, 13 aa, synthetic construct
  REGION 1..13: note = Synthetic peptide
GITGPTFPGCPET

SEQ ID NO: 614 - protein, 14 aa, synthetic construct
  REGION 1..14: note = Synthetic peptide
GITGPTFPGCPETY

SEQ ID NO: 615 - protein, 12 aa, synthetic construct
  REGION 1..12: note = Synthetic peptide
SQNIFSGFSTEL

SEQ ID NO: 616 - protein, 12 aa, synthetic construct
  REGION 1..12: note = Synthetic peptide
QLQCQNDQRGEI

SEQ ID NO: 617 - protein, 12 aa, synthetic construct
  REGION 1..12: note = Synthetic peptide
LGQSTSQWQSSR

SEQ ID NO: 618 - protein, 15 aa, synthetic construct
  REGION 1..15: note = Synthetic peptide
QQLLGQSTSQWQSSR

SEQ ID NO: 619 - protein, 13 aa, synthetic construct
  REGION 1..13: note = Synthetic peptide
LLGQSTSQWQSSR

SEQ ID NO: 620 - protein, 9 aa, synthetic construct
  REGION 1..9: note = Synthetic peptide
IFFANQTYL

SEQ ID NO: 621 - protein, 14 aa, synthetic construct
  REGION 1..14: note = Synthetic peptide
EHLEPNLEGLTVEE

SEQ ID NO: 622 - protein, 36 aa, synthetic construct
  REGION 1..36: note = Synthetic peptide
IFFANQTYLPSETPAPLVHYREEELNNLRGDGTGER

SEQ ID NO: 623 - protein, 18 aa, synthetic construct
  REGION 1..18: note = Synthetic peptide
IHFEWDDDMGIPGAFYIK

SEQ ID NO: 624 - protein, 29 aa, synthetic construct
  REGION 1..29: note = Synthetic peptide
IFFANQTYLPSETPAPLVHYREEELNNLR

SEQ ID NO: 625 - protein, 11 aa, synthetic construct
  REGION 1..11: note = Synthetic peptide
TEQALPADLIK

SEQ ID NO: 626 - protein, 20 aa, synthetic construct
  REGION 1..20: note = Synthetic peptide
EHLEPNLEGLTVEEAIQNKK

SEQ ID NO: 627 - protein, 23 aa, synthetic construct
  REGION 1..23: note = Synthetic peptide
ISKEHLEPNLEGLTVEEAIQNKK

SEQ ID NO: 628 - protein, 16 aa, synthetic construct
  REGION 1..16: note = Synthetic peptide
LSLPHPQGDEHGAVSY

SEQ ID NO: 629 - protein, 22 aa, synthetic construct
  REGION 1..22: note = Synthetic peptide
ISKEHLEPNLEGLTVEEAIQNK

SEQ ID NO: 630 - protein, 19 aa, synthetic construct
  REGION 1..19: note = Synthetic peptide
EHLEPNLEGLTVEEAIQNK

SEQ ID NO: 631 - protein, 18 aa, synthetic construct
  REGION 1..18: note = Synthetic peptide
LSTTGGNSGSPVFNEKNE

SEQ ID NO: 632 - protein, 17 aa, synthetic construct
  REGION 1..17: note = Synthetic peptide
QSFLLSGNQNQQNYLSG

SEQ ID NO: 633 - protein, 16 aa, synthetic construct
  REGION 1..16: note = Synthetic peptide
VLDLAIPVNRPGQLQS

SEQ ID NO: 634 - protein, 18 aa, synthetic construct
  REGION 1..18: note = Synthetic peptide
VLDLAIPVNRPGQLQSFL

SEQ ID NO: 635 - protein, 15 aa, synthetic construct
  REGION 1..15: note = Synthetic peptide
FLLSGNQNQQNYLSG

SEQ ID NO: 636 - protein, 18 aa, synthetic construct
  REGION 1..18: note = Synthetic peptide
FLLSGNQNQQNYLSGFSK

SEQ ID NO: 637 - protein, 18 aa, synthetic construct
  REGION 1..18: note = Synthetic peptide
DPQNPFIFKSNKFQTLFE

SEQ ID NO: 638 - protein, 19 aa, synthetic construct
  REGION 1..19: note = Synthetic peptide
ELAFPGSAQEVDRILENQK

SEQ ID NO: 639 - protein, 11 aa, synthetic construct
  REGION 1..11: note = Synthetic peptide
AIVIVTVNEGK

SEQ ID NO: 640 - protein, 18 aa, synthetic construct
  REGION 1..18: note = Synthetic peptide
INAVAAKRLQSQNDQRGE

SEQ ID NO: 641 - protein, 14 aa, synthetic construct
  REGION 1..14: note = Synthetic peptide
NRAQQQQVYGSSIE

SEQ ID NO: 642 - protein, 10 aa, synthetic construct
  REGION 1..10: note = Synthetic peptide
PSTNPWHSPR

SEQ ID NO: 643 - protein, 4 aa, synthetic construct
  REGION 1..4: note = Synthetic peptide
CHGS

SEQ ID NO: 644 - protein, 5 aa, synthetic construct
  REGION 1..5: note = Synthetic peptide
CHGSM

SEQ ID NO: 645 - protein, 4 aa, synthetic construct
  REGION 1..4: note = Synthetic peptide
PWHS

SEQ ID NO: 646 - protein, 4 aa, synthetic construct
  REGION 1..4: note = Synthetic peptide
FREC

SEQ ID NO: 647 - protein, 4 aa, synthetic construct
  REGION 1..4: note = Synthetic peptide
RECR

SEQ ID NO: 648 - protein, 4 aa, synthetic construct
  REGION 1..4: note = Synthetic peptide
ECRF

SEQ ID NO: 649 - protein, 4 aa, synthetic construct
  REGION 1..4: note = Synthetic peptide
CRFD

SEQ ID NO: 650 - protein, 5 aa, synthetic construct
  REGION 1..5: note = Synthetic peptide
CRFDR

SEQ ID NO: 651 - protein, 4 aa, synthetic construct
  REGION 1..4: note = Synthetic peptide
CTGT

SEQ ID NO: 652 - protein, 4 aa, synthetic construct
  REGION 1..4: note = Synthetic peptide
FPGC

SEQ ID NO: 653 - protein, 5 aa, synthetic construct
  REGION 1..5: note = Synthetic peptide
FPGCP

SEQ ID NO: 654 - protein, 4 aa, synthetic construct
  REGION 1..4: note = Synthetic peptide
PGCP

SEQ ID NO: 655 - protein, 5 aa, synthetic construct
  REGION 1..5: note = Synthetic peptide
PGCPA

SEQ ID NO: 656 - protein, 6 aa, synthetic construct
  REGION 1..6: note = Synthetic peptide
PGCPAT

SEQ ID NO: 657 - protein, 4 aa, synthetic construct
  REGION 1..4: note = Synthetic peptide
GCPA

SEQ ID NO: 658 - protein, 4 aa, synthetic construct
  REGION 1..4: note = Synthetic peptide
CPAT

SEQ ID NO: 659 - protein, 4 aa, synthetic construct
  REGION 1..4: note = Synthetic peptide
ENFC

SEQ ID NO: 660 - protein, 4 aa, synthetic construct
  REGION 1..4: note = Synthetic peptide
NFCT

SEQ ID NO: 661 - protein, 5 aa, synthetic construct
  REGION 1..5: note = Synthetic peptide
NFCTI

SEQ ID NO: 662 - protein, 4 aa, synthetic construct
  REGION 1..4: note = Synthetic peptide
FCTI

SEQ ID NO: 663 - protein, 14 aa, synthetic construct
  REGION 1..14: note = Synthetic peptide
AQQQQVYGSSIEQH

SEQ ID NO: 664 - protein, 23 aa, synthetic construct
  REGION 1..23: note = Synthetic peptide
AQQQQVYGSSIEQHSGQNIFSGF

SEQ ID NO: 665 - protein, 14 aa, synthetic construct
  REGION 1..14: note = Synthetic peptide
AAKRLQSQNDQRGE

SEQ ID NO: 666 - protein, 12 aa, synthetic construct
  REGION 1..12: note = Synthetic peptide
QARSLKNNRGEE

SEQ ID NO: 667 - protein, 15 aa, synthetic construct
  REGION 1..15: note = Synthetic peptide
FNPSTNPWHSPRQGS

SEQ ID NO: 668 - protein, 12 aa, synthetic construct
  REGION 1..12: note = Synthetic peptide
QARSLKNNRGEE

SEQ ID NO: 669 - protein, 26 aa, synthetic construct
  REGION 1..26: note = Synthetic peptide
AAASLPAFCNVDIPNGGGGVCYWLAR

SEQ ID NO: 670 - protein, 18 aa, synthetic construct
  REGION 1..18: note = Synthetic peptide
VAGSAPGIEGDEIAPLAK

SEQ ID NO: 671 - protein, 22 aa, synthetic construct
  REGION 1..22: note = Synthetic peptide
LGVAGSAPGIEGDEIAPLAKEN

SEQ ID NO: 672 - protein, 17 aa, synthetic construct
  REGION 1..17: note = Synthetic peptide
GSAPGIEGDEIAPLAKE

SEQ ID NO: 673 - protein, 15 aa, synthetic construct
  REGION 1..15: note = Synthetic peptide
VAGSAPGIEGDEIAP

SEQ ID NO: 674 - protein, 19 aa, synthetic construct
  REGION 1..19: note = Synthetic peptide
GVAGSAPGIEGDEIAPLAK

SEQ ID NO: 675 - protein, 21 aa, synthetic construct
  REGION 1..21: note = Synthetic peptide
GVAGSAPGIEGDEIAPLAKEN

SEQ ID NO: 676 - protein, 20 aa, synthetic construct
  REGION 1..20: note = Synthetic peptide
VAGSAPGIEGDEIAPLAKEN

SEQ ID NO: 677 - protein, 15 aa, synthetic construct
  REGION 1..15: note = Synthetic peptide
SAPGIEGDEIAPLAK

SEQ ID NO: 678 - protein, 16 aa, synthetic construct
  REGION 1..16: note = Synthetic peptide
GSAPGIEGDEIAPLAK

SEQ ID NO: 679 - protein, 17 aa, synthetic construct
  REGION 1..17: note = Synthetic peptide
SAPGIEGDEIAPLAKEN

SEQ ID NO: 680 - protein, 15 aa, synthetic construct
  REGION 1..15: note = Synthetic peptide
FNPSTNPWHSPRQGS

SEQ ID NO: 681 - protein, 12 aa, synthetic construct
  REGION 1..12: note = Synthetic peptide
VDLVIPVNGPGK

SEQ ID NO: 682 - protein, 13 aa, synthetic construct
  REGION 1..13: note = Synthetic peptide
LVDLVIPVNGPGK

SEQ ID NO: 683 - protein, 10 aa, synthetic construct
  REGION 1..10: note = Synthetic peptide
IKLPAGTTSY

SEQ ID NO: 684 - protein, 11 aa, synthetic construct
  REGION 1..11: note = Synthetic peptide
IKLPAGTTSYL

SEQ ID NO: 685 - protein, 12 aa, synthetic construct
  REGION 1..12: note = Synthetic peptide
RRNPFLFKSNKF

SEQ ID NO: 686 - protein, 19 aa, synthetic construct
  REGION 1..19: note = Synthetic peptide
IENPVKELTFPGSVQEINR

SEQ ID NO: 687 - protein, 14 aa, synthetic construct
  REGION 1..14: note = Synthetic peptide
RRNPFLFKSNKFLT

SEQ ID NO: 688 - protein, 14 aa, synthetic construct
  REGION 1..14: note = Synthetic peptide
AKPHTIFLPQHIDA

SEQ ID NO: 689 - protein, 15 aa, synthetic construct
  REGION 1..15: note = Synthetic peptide
AKPHTIFLPQHIDAD

SEQ ID NO: 690 - protein, 9 aa, synthetic construct
  REGION 1..9: note = Synthetic peptide
KQKYRYQRE

SEQ ID NO: 691 - protein, 9 aa, synthetic construct
  REGION 1..9: note = Synthetic peptide
KQKYQYQRE

SEQ ID NO: 692 - protein, 4 aa, synthetic construct
  REGION 1..4: note = Synthetic peptide
MLPH

SEQ ID NO: 693 - protein, 19 aa, synthetic construct
  REGION 1..19: note = Synthetic peptide
RRNPFLFKSNKFLTLFENE

SEQ ID NO: 694 - protein, 14 aa, synthetic construct
  REGION 1..14: note = Synthetic peptide
PFLFKSNKFLTLFE

SEQ ID NO: 695 - protein, 20 aa, synthetic construct
  REGION 1..20: note = Synthetic peptide
SQERRNPFLFKSNKFLTLFE

SEQ ID NO: 696 - protein, 17 aa, synthetic construct
  REGION 1..17: note = Synthetic peptide
RRNPFLFKSNKFLTLFE

SEQ ID NO: 697 - protein, 22 aa, synthetic construct
  REGION 1..22: note = Synthetic peptide
SQERRNPFLFKSNKFLTLFENE

SEQ ID NO: 698 - protein, 9 aa, synthetic construct
  REGION 1..9: note = Synthetic peptide
LTFPGSVQE

SEQ ID NO: 699 - protein, 13 aa, synthetic construct
  REGION 1..13: note = Synthetic peptide
ELTFPGSVQEINR

SEQ ID NO: 700 - protein, 18 aa, synthetic construct
  REGION 1..18: note = Synthetic peptide
KNPQLQDLDIFVNYVEIK

SEQ ID NO: 701 - protein, 24 aa, synthetic construct
  REGION 1..24: note = Synthetic peptide
PGQLQSFLLSGNQNQQNYLSGFSK

SEQ ID NO: 702 - protein, 14 aa, synthetic construct
  REGION 1..14: note = Synthetic peptide
RGPQQYAEWQINEK

SEQ ID NO: 703 - protein, 14 aa, synthetic construct
  REGION 1..14: note = Synthetic peptide
VLDLAIPVNRPGQL

SEQ ID NO: 704 - protein, 24 aa, synthetic construct
  REGION 1..24: note = Synthetic peptide
GYVGLTFPGCPATHQQQFQLFEQR

SEQ ID NO: 705 - protein, 820 aa, Oryza sativa
MLCLTSSSSSAPAPLLPSLADRPSPGIAGGGGNVRLSVVSSPRRSWPGKVKTNFSVPATARKNKTMVTVVEEVDHLPIYDLDPKLEEFKDHFNYRIKRYLDQKCLIEKHEGGLEEFSKGYLKFGINTVDGATIYREWAPAAQEAQLIGEFNNWNGAKHKMEKDKFGIWSIKISHVNGKPAIPHNSKVKFRFRHGGGAWVDRIPAWIRYATFDASKFGAPYDGVHWDPPACERYVFKHPRPPKPDAPRIYEAHVGMSGEEPEVSTYREFADNVLPRIRANNYNTVQLMAIMEHSYYASFGYHVTNFFAVSSRSGTPEDLKYLVDKAHSLGLRVLMDVVHSHASNNVTDGLNGYDVGQNTHESYFHTGDRGYHKLWDSRLFNYANWEVLRFLLSNLRYWMDEFMFDGFRFDGVTSMLYHHHGINKGFTGNYKEYFSLDTDVDAIVYMMLANHLMHKLLPEATIVAEDVSGMPVLCRPVDEGGVGFDFRLAMAIPDRWIDYLKNKEDRKWSMSEIVQTLTNRRYTEKCIAYAESHDQSIVGDKTIAFLLMDKEMYTGMSDLQPASPTINRGIALQKMIHFITMALGGDGYLNFMGNEFGHPEWIDFPREGNNWSYDKCRRQWSLVDTDHLRYKYMNAFDQAMNALEEEFSFLSSSKQIVSDMNEKDKVIVFERGDLVFVFNFHPNKTYKGYKVGCDLPGKYRVALDSDALVFGGHGRVGHDVDHFTSPEGMPGVPETNFNNRPNSFKVLSPPRTCVAYYRVDEDREELRRGGAVASGKIVTEYIDVEATSGETISGGWKGSEKDDCGKKGMKFVFRSSDEDCK

SEQ ID NO: 706 - protein, 296 aa, Oryza sativa
MDRYQRVERPRPESAIEENEIRITAQGLIRNYVSYATSLLQDRRIKEIVLKAMGQAISKSVAVAEIIKKRVPGLYQDTNISSVSITDVWEPIEEGLVPLEMTRHVSMISITLSPRDLDKNSPGYQTPVYVEQPRQQPRLQQAPPPPQRQVRQPPPDYEDSYVRGRGRGRGRGRGRGWGRGGYGGYGGYGNNQGGYNQGGGYYDNQGGYGGYDNQGGYGGYDNQGGYGGGGYGYNQGRYGNYQENGGYNRGRGGMRGRGNWNYRGGYERGRGGGFPGGRGYGGRGRGRMGGRGGRGN

SEQ ID NO: 707 - protein, 184 aa, Oryza sativa
MALEHLGRRNVAGSLLLLNLLMYVFLLGFAGWALNSSIKNAGADVGVGWGEQPWSPYYRQSAWFASRFHLATFAALAGALGVAAKASAAYHGGRSGASWRPQGLAAAASLGTAAWAATALAFGVACREIHDAAAAGPAGAARGWRMRALEGLTVTLAFTQLLYVLLLHAAVAGERCGLACAADA

SEQ ID NO: 708 - protein, 483 aa, Pisum sativum
MAIKTKLSLTIFLFFLLALLCSNLAVGRKEKDPELTTCKDQCDMQRQYDEEDKRICMERCDDYIKKKQERQKHKEHEEEEEQEQEEDENPYVFEDNDFETKIDTKDGRVLILNKFNEKSKLLKNIENYGLAVLEIKANAFLSPHHYDSEAILFNIKGRGIIGLVAEDRTERFNLEEGDIMRVPAGTPMYLVNRDENEKLYIAAFHMPPSSGSAPVNLEPFFESAGRKPESVLNTFSSKVLQAALKSSKGELETVLDEQKKGRIFKIEKEDVRGLAPKKSLWPFGGPFKSPFNIFSNNPAFSNKFGSLFEVGPSQEKSGLEGLNLMLTLANITKGSMSTIHYNTNANKIALVIDGEGELEMACPHMPSSSSNSRQKKSSISYHNINAKLRPGVMFVVPAGHPFVNIASKKKNLIVVCFEVNAQRNKKLALAGKKNIVSALDKAAKEVAFDIAAEKVDEVFERKEEFFFPYDNEERKEEHGRAVV

SEQ ID NO: 709 - protein, 197 aa, Pisum sativum
MFRRATSTFLSRASATRRFSTDVATPATNSSFVEAWRKVSPNIDPPKTPLEFLKTRPPVPSTIPTKLTVNFVLPYSSQLAAKEVDSVIIPATTGEMGVLPGHVATIAELKPGVLTVQEGTDTTKYFVSSGFRFIHANSVADIIAVEAVPVNQLDRDLVQKGLQEFTQKLNSATTDLEKREAQIGIDVDSALNSALTG

SEQ ID NO: 710 - protein, 156 aa, Oryza sativa
MKIIFFFALLAIAACSASAQFDAVTQVYRQYQLQPHLMLQQQMLSPCGEFVRQQCSTVATPFFQSPVFQLRNCQVMQQQCCQQLRMIAQQSHCQAISSVQAIVQQLRLQQFASVYFDQSQAQAQAMLALNMPSICGIYPSYNTAPCSIPTVGGIWY

SEQ ID NO: 711 - protein, 704 aa, Pisum sativum
MQDSSTMKFSPLDLMTAIIKGKFNPSNDSSQAPASIIFENREFVMILTTSIAVLIGCVVVLIWRRSNSNKSKQIEVPKLVIKKLPELDVDDGKKKVTVFFGTQTGTAEGFAKAIAEEAKARYEKAKFRVVDMDDYAADDDEYLEKLKRETMALFFLATYGDGEPTDNAARFYKWFTEEYEGEEDSFKNLSYGVFGLGNRQYEHFNKVAKVVDDKLLEQGGKRLVPVGLGDDDQCIEDDFTAWKEELWPALDQLLRDEDDTPVATPYTAAVSEYRVVIHDPLDATVDEKKRHNVNGHAVVDAQHPVRANVAVRRELHTPASDRSCTHLEFDISGTGVVYETGDHVGVYCENLSDTVEEAERILGLSPDTYLSIHTDDEEGKPLGGSSLPPPFPPCTLRTALTKYADLLSSPKKSALVALAAHASDPSEADRLRHLASPAGKDEYAEWVISSQRSLLEVMAEFSSAKPPIGVFFAAVAPRLQPRYYSISSSPRMAPSRIHVTCALVHDKMPTGRIHKGVCSTWMKNSVPLEKNQDCSWAPIFVRQSNFRLPADNKVPVIMIGPGTGLAPFRGFLQERLALKEDGAELGPSVLFFGCRNRQVDYIYEDELNHFVNGGALSELIVAFSRDGPTKEYVQHKMMEKASDIWNMISQGAYVYVCGDAKGMARDVHRTLHTILQEQGSLDSSKTESMVKNLQMTGRYLRDVW

SEQ ID NO: 712 - protein, 358 aa, Oryza sativa
MSAYCGKYKDELIKNAAYIGTPGKGILAADESTGTIGKRFASINVENVEENRRSLRELLFTTPGALQHLSGVILFEETLYQKTKDGKPFVDVLKEGGVLPGIKVDKGTVEVAGTNKETTTQGHDDLGKRCAKYYEAGARFAKWRAVLKIGPNEPSQLSIDLNAQGLARYAIICQENGLVPIVEPEILVDGSHDIERCAYVTEKVLAACYKALNEHHVLLEGSLLKPNMVTPGSESKKVSPQLIAEYTVRALQRTVPAAVPAIVFLSGGQSEEEATVNLNAMNKLSTKKPWALSFSFGRALQQSTLKAWGGKTENVVKAQKAFITRCKANSEATLGTYQGDAVLGEGASESLHVKDYKY

SEQ ID NO: 713 - protein, 275 aa, Pisum sativum
MASLQTQMISFYAIFLSILLTTILFFKVNSTETTSFLITKFSPDQQNLIFQGDGYTTKEKLTLTKAVKNTVGRALYSSPIHIWDRETGNVANFVTSFTFVINAPNSYNVADGFTFFIAPVDTKPQTGGGYLGVFNSAEYDKTTQTVAVEFDTFYNAAWDPSNRDRHIGIDVNSIKSVNTKSWKLQNGEEANVVIAFNAATNVLTVSLTYPNSLEEENVTSYTLSDVVSLKDVVPEWVRIGFSATTGAEYAAHEVLSWSFHSELSGTSSSKQAADA

SEQ ID NO: 714 - protein, 484 aa, Oryza sativa
MATTTSLLSSCLCALLLAPLFSQGVDAWESRQGASRQCRFDRLQAFEPLRKVRSEAGDTEYFDERNEQFRCAGVFVIRRVIEPQGLVVPRYSNTPALAYIIQGKGYVGLTFPGCPATHQQQFQLFEQRQSDQAHKFRDEHQKIHEFRQGDVVALPASVAHWFYNGGDTPAVVVYVYDIKSFANQLEPRQKEFLLAGNNQRGQQIFEHSIFQHSGQNIFSGFNTEVLSEALGINTEASKRLQSQNDQRGDIIRVKHGLQLLKPTLTQRQEEHRQYQQVQYREGQYNGLDENFCTIKARVNIENPSRADYYNPRAGRITLLNNQKFPILNLIGMGAARVNLYQNALLSPFWNINAHSVVYIIQGSVRVQVANNQGRSVFNGVLHQGQLLIIPQNHAVIKKAEHNGCQYVAIKTISDPTVSWVAGKNSILRALPVDVIANAYRISRDEARRLKNNRADEIGPFTPRFPQKSQRGYQFLTEGLSLIGM

SEQ ID NO: 715 - protein, 123 aa, Pisum sativum
QKAPRKQLATKAARKSAPATGGVKKPHRFRPGTVALREIRKYQKSTELLIRKLPFQRLVREIAQDFKTDLRFQSSAVSALQEAAEAYLVGLFEDTNLCAIHAKRVTIMPKDIQLARRIRGERA

SEQ ID NO: 716 - protein, 232 aa, Pisum sativum
MAQSVSLSTIASPILSQKPGSSVKSTPPCMASFPLRRQLPRLGLRNVRAQAGGDGDNKDNSVEVHRVNKDDQGTAVERKPRRSSIDISPFGLLDPWSPMRSMRQMLDTMDRIFEDAITIPGRNIGGGEIRVPWEIKDEEHEIRMRFDMPGVSKEDVKVSVEDDVLVIKSDHREENGGEDCWSRKSYSCYDTRLKLPDNCEKEKVKAELKDGVLYITIPKTKIERTVIDVQIQ

SEQ ID NO: 717 - protein, 10 aa, synthetic construct
  REGION 1..10: note = Synthetic peptide
LDVTPLSLGL

SEQ ID NO: 718 - protein, 12 aa, synthetic construct
  REGION 1..12: note = Synthetic peptide
EEGIQLVAEAIR

SEQ ID NO: 719 - protein, 9 aa, synthetic construct
  REGION 1..9: note = Synthetic peptide
YSLKPLVPR

SEQ ID NO: 720 - protein, 4 aa, synthetic construct
  REGION 1..4: note = Synthetic peptide
WHTA

SEQ ID NO: 721 - protein, 4 aa, synthetic construct
  REGION 1..4: note = Synthetic peptide
WHNA

SEQ ID NO: 722 - protein, 4 aa, synthetic construct
  REGION 1..4: note = Synthetic peptide
NNPF

SEQ ID NO: 723 - protein, 4 aa, synthetic construct
  REGION 1..4: note = Synthetic peptide
MRFR

SEQ ID NO: 724 - protein, 5 aa, synthetic construct
  REGION 1..5: note = Synthetic peptide
MPPSS

SEQ ID NO: 725 - protein, 4 aa, synthetic construct
  REGION 1..4: note = Synthetic peptide
HMPS

SEQ ID NO: 726 - protein, 5 aa, synthetic construct
  REGION 1..5: note = Synthetic peptide
HMPPS

SEQ ID NO: 727 - protein, 4 aa, synthetic construct
  REGION 1..4: note = Synthetic peptide
GHPM

SEQ ID NO: 728 - protein, 4 aa, synthetic construct
  REGION 1..4: note = Synthetic peptide
FWNA

SEQ ID NO: 729 - protein, 4 aa, synthetic construct
  REGION 1..4: note = Synthetic peptide
FHMP

SEQ ID NO: 730 - protein, 11 aa, synthetic construct
  REGION 1..11: note = Synthetic peptide
WTIVQGLPIDE

SEQ ID NO: 731 - protein, 9 aa, synthetic construct
  REGION 1..9: note = Synthetic peptide
GYPMYPLPR

SEQ ID NO: 732 - protein, 9 aa, synthetic construct
  REGION 1..9: note = Synthetic peptide
HGGEGGRPY

SEQ ID NO: 733 - protein, 6 aa, synthetic construct
  REGION 1..6: note = Synthetic peptide
LRGFSK

SEQ ID NO: 734 - protein, 9 aa, synthetic construct
  REGION 1..9: note = Synthetic peptide
GALMLPHYN

SEQ ID NO: 735 - protein, 11 aa, synthetic construct
  REGION 1..11: note = Synthetic peptide
GALMLPHYNSR

SEQ ID NO: 736 - protein, 9 aa, synthetic construct
  REGION 1..9: note = Synthetic peptide
VFDGVLRPG

SEQ ID NO: 737 - protein, 6 aa, synthetic construct
  REGION 1..6: note = Synthetic peptide
LQSQND

SEQ ID NO: 738 - protein, 11 aa, synthetic construct
  REGION 1..11: note = Synthetic peptide
LQSQNDQRGEI

SEQ ID NO: 739 - protein, 14 aa, synthetic construct
  REGION 1..14: note = Synthetic peptide
QSQNDQRGEIIHVK

SEQ ID NO: 740 - protein, 8 aa, synthetic construct
  REGION 1..8: note = Synthetic peptide
RGEIIHVK

SEQ ID NO: 741 - protein, 8 aa, synthetic construct
  REGION 1..8: note = Synthetic peptide
RLQSQNDQ

SEQ ID NO: 742 - protein, 10 aa, synthetic construct
  REGION 1..10: note = Synthetic peptide
RLQSQNDQRG

SEQ ID NO: 743 - protein, 14 aa, synthetic construct
  REGION 1..14: note = Synthetic peptide
RLQSQNDQRGEIIH

SEQ ID NO: 744 - protein, 4 aa, synthetic construct
  REGION 1..4: note = Synthetic peptide
MPMP

SEQ ID NO: 745 - protein, 4 aa, synthetic construct
  REGION 1..4: note = Synthetic peptide
PMPL

SEQ ID NO: 746 - protein, 6 aa, synthetic construct
  REGION 1..6: note = Synthetic peptide
LEPDNR

SEQ ID NO: 747 - protein, 13 aa, synthetic construct
  REGION 1..13: note = Synthetic peptide
GIARLAGTSSVIN

SEQ ID NO: 748 - protein, 6 aa, synthetic construct
  REGION 1..6: note = Synthetic peptide
RSQNIF

SEQ ID NO: 749 - protein, 4 aa, synthetic construct
  REGION 1..4: note = Synthetic peptide
PNSM

SEQ ID NO: 750 - protein, 4 aa, synthetic construct
  REGION 1..4: note = Synthetic peptide
GHPM

SEQ ID NO: 751 - protein, 4 aa, synthetic construct
  REGION 1..4: note = Synthetic peptide
HPMS

SEQ ID NO: 752 - protein, 7 aa, synthetic construct
  REGION 1..7: note = Synthetic peptide
FLPQHTD

SEQ ID NO: 753 - protein, 7 aa, synthetic construct
  REGION 1..7: note = Synthetic peptide
EWQINEK

SEQ ID NO: 754 - protein, 13 aa, synthetic construct
  REGION 1..13: note = Synthetic peptide
GPQQYAEWQINEK

SEQ ID NO: 755 - protein, 8 aa, synthetic construct
  REGION 1..8: note = Synthetic peptide
PQQYAEWQ

SEQ ID NO: 756 - protein, 7 aa, synthetic construct
  REGION 1..7: note = Synthetic peptide
RGPQQYA

SEQ ID NO: 757 - protein, 4 aa, synthetic construct
  REGION 1..4: note = Synthetic peptide
HNPR

SEQ ID NO: 758 - protein, 4 aa, synthetic construct
  REGION 1..4: note = Synthetic peptide
WHNR

SEQ ID NO: 759 - protein, 4 aa, synthetic construct
  REGION 1..4: note = Synthetic peptide
WDPA

SEQ ID NO: 760 - protein, 4 aa, synthetic construct
  REGION 1..4: note = Synthetic peptide
HPSF

SEQ ID NO: 761 - protein, 22 aa, synthetic construct
  REGION 1..22: note = Synthetic peptide
PGQLQSFLLSGNQNQQNYLSGF

SEQ ID NO: 762 - protein, 22 aa, synthetic construct
  REGION 1..22: note = Synthetic peptide
QLQSFLLSGNQNQQNYLSGFSK

SEQ ID NO: 763 - protein, 12 aa, synthetic construct
  REGION 1..12: note = Synthetic peptide
QSFLLSGNQNQQ

SEQ ID NO: 764 - protein, 12 aa, synthetic construct
  REGION 1..12: note = Synthetic peptide
PGQLQSFLLSGN

SEQ ID NO: 765 - protein, 9 aa, synthetic construct
  REGION 1..9: note = Synthetic peptide
QSFLLSGNQ

SEQ ID NO: 766 - protein, 12 aa, synthetic construct
  REGION 1..12: note = Synthetic peptide
QNQQNYLSGFSK

SEQ ID NO: 767 - protein, 6 aa, synthetic construct
  REGION 1..6: note = Synthetic peptide
YLRGFS

SEQ ID NO: 768 - protein, 11 aa, synthetic construct
  REGION 1..11: note = Synthetic peptide
PVEMPTLLYPS

SEQ ID NO: 769 - protein, 13 aa, synthetic construct
  REGION 1..13: note = Synthetic peptide
RGPQQYAEWQINE

SEQ ID NO: 770 - protein, 24 aa, synthetic construct
  REGION 1..24: note = Synthetic peptide
GYVGLTFPGCPATHQQQFQLFEQR

SEQ ID NO: 771 - protein, 18 aa, synthetic construct
  REGION 1..18: note = Synthetic peptide
KNPQLQDLDIFVNYVEIK

SEQ ID NO: 772 - protein, 24 aa, synthetic construct
  REGION 1..24: note = Synthetic peptide
PGQLQSFLLSGNQNQQNYLSGFSK

SEQ ID NO: 773 - protein, 14 aa, synthetic construct
  REGION 1..14: note = Synthetic peptide
RGPQQYAEWQINEK

SEQ ID NO: 774 - protein, 14 aa, synthetic construct
  REGION 1..14: note = Synthetic peptide
VLDLAIPVNRPGQL

SEQ ID NO: 775 - protein, 14 aa, synthetic construct
  REGION 1..14: note = Synthetic peptide
RGPQQYAEWQINEK

SEQ ID NO: 776 - protein, 12 aa, synthetic construct
  REGION 1..12: note = Description of Artificial Sequence: Synthetic peptide
RPYYSNAPQEIF

SEQ ID NO: 777 - protein, 12 aa, synthetic construct
  REGION 1..12: note = Description of Artificial Sequence: Synthetic peptide
VLLEQQEQEPQH

SEQ ID NO: 778 - protein, 15 aa, synthetic construct
  REGION 1..15: note = Description of Artificial Sequence: Synthetic peptide
QQYGIAASPFLQSAA